SEQUENCE LISTING (135 sequences)

SEQ ID NO: 1 - DNA, 2277 bp, Homo sapiens
gactgcgcggcggcaacagcagacatgtcgggggtccggggcctgtcgcggctgctgagcgctcggcgcctggcgctggccaaggcgtggccaacagtgttgcaaacaggaacccgaggttttcacttcactgttgatgggaacaagagggcatctgctaaagtttcagattccatttctgctcagtatccagtagtggatcatgaatttgatgcagtggtggtaggcgctggaggggcaggcttgcgagctgcatttggcctttctgaggcagggtttaatacagcatgtgttaccaagctgtttcctaccaggtcacacactgttgcagcgcagggaggaatcaatgctgctctggggaacatggaggaggacaactggaggtggcatttctacgacaccgtgaagggctccgactggctgggggaccaggatgccatccactacatgacggagcaggcccccgccgccgtggtcgagctagaaaattatggcatgccgtttagcagaactgaagatgggaagatttatcagcgtgcatttggtggacagagcctcaagtttggaaagggcgggcaggcccatcggtgctgctgtgtggctgatcggactggccactcgctattgcacaccttatatggacggtctctgcgatatgataccagctattttgtggagtattttgccttggatctcctgatggagaacggggagtgccgtggtgtcatcgcactgtgcatagaggacgggtccatccatcgcataagagcaaagaacactgttgttgccacaggaggctacgggcgcacctacttcagctgcacgtctgcccacaccagcactggcgacggcacggccatgatcaccagggcaggccttccttgccaggacctagagtttgttcagttccaccccacaggcatatatggtgctggttgtctcattacggaaggatgtcgtggagagggaggcattctcattaacagtcaaggcgaaaggtttatggagcgatacgcccctgtcgcgaaggacctggcgtctagagatgtggtgtctcggtcgatgactctggagatccgagaaggaagaggctgtggccctgagaaagatcacgtctacctgcagctgcaccacctacctccagagcagctggccacgcgcctgcctggcatttcagagacagccatgatcttcgctggcgtggacgtcacgaaggagccgatccctgtcctccccaccgtgcattataacatgggcggcattcccaccaactacaaggggcaggtcctgaggcacgtgaatggccaggatcagattgtgcccggcctgtacgcctgtggggaggccgcctgtgcctcggtacatggtgccaaccgcctcggggcaaactcgcgcggattgatgagtacgattactccaagcccatccaggggcaacagaagaagccctttgaggagcactggaggaagcacaccctgtcctttgtggacgttggcactgggaaggtcactctggaatatagacccgtaatcgacaaaactttgaacgaggctgactgtgccaccatcccgccagccattcgctcctactgatgagacaagatgtggtgatgacagaatcagcttttgtaattatgtataatagctcatgcatgtgtccatgtcataactgtcttcatacgcttctgcactctggggaagaaggagtacattgaagggagattggcacctagtggctgggagcttgccaggaacccagtggccagggagcgtggcacttacctttgtcccttgcttcattcttgtgagatgataaaactgggcacagctcttaaataaaatataaatgagtcttggacctggttgtctttggtcgggcatgtgccctgagcatcgaagagtcatgcaggcctggagataaagtccctccaattaaaccaaacgctggggaagaatctgtcatgaatcttgacaaattgagatttgctgatggaagcataagaacatcggaactgcgactcagcatgcagaagtcaatgcaaaatcatgctgccgtgttccgtgtgggaagcgtgttgcaagaaggttgtgggaaaatcagcaagctctatggagacctaaagcacctgaagacgttcgaccggggaatggtctggaacacagacctggtggagaccctggagctgcagaacctgatgctgtgtgcgctgcagaccatctacggagcagaggcgcggaaggagtcacggggcgcgcatgccagggaagactacaaggt

SEQ ID NO: 2 - DNA, 20 bp, Artificial Sequence
  Synthetic oligonucleotide
tgggaacaagagggcatctg

SEQ ID NO: 3 - DNA, 22 bp, Artificial Sequence
  Synthetic oligonucleotide
ccaccactgcatcaaattcatg

SEQ ID NO: 4 - DNA, 86 bp, Artificial Sequence
  Synthetic oligonucleotide
tgggaacaagagggcatctgctaaagtttcagattccatttctgctcagtatccagtagtggatcatgaatttgatgcagtggtgg

SEQ ID NO: 5 - DNA, 1536 bp, Homo sapiens
gtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtaaaaaaaaaaaaaaaaaaaaaaaaaaaaaaaaaaa

SEQ ID NO: 6 - DNA, 19 bp, Artificial Sequence
  Synthetic oligonucleotide
tgagagtgattcgcgtggg

SEQ ID NO: 7 - DNA, 21 bp, Artificial Sequence
  Synthetic oligonucleotide
ccagggtacgaggctttcaat

SEQ ID NO: 8 - DNA, 91 bp, Artificial Sequence
  Synthetic oligonucleotide
tgagagtgattcgcgtgggtacccgcaagagccagcttgctcgcatacagacggacagtgtggtggcaacattgaaagcctcgtaccctgg

SEQ ID NO: 9 - DNA, 1331 bp, Homo sapiens
tcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaattt

SEQ ID NO: 10 - DNA, 21 bp, Artificial Sequence
  Synthetic oligonucleotide
tgacactggcaaaacaatgca

SEQ ID NO: 11 - DNA, 21 bp, Artificial Sequence
  Synthetic oligonucleotide
ggtccttttcaccagcaagct

SEQ ID NO: 12 - DNA, 94 bp, Artificial Sequence
  Synthetic oligonucleotide
tgacactggcaaaacaatgcagactttgctttccttggtcaggcagtataatccaaagatggtcaaggtcgcaagcttgctggtgaaaaggacc

SEQ ID NO: 13 - DNA, 1261 bp, Homo sapiens
cttttgcgtcgccagccgagccacatcgctcagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctcctgcaccaccaactgcttagcacccctggccaaggtcatccatgacaactttggtatcgtggaaggactcatgaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgacaacgaatttggctacagcaacagggtggtggacctcatggcccacatggcctccaaggagtaagacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccaaaaaaaaaaaaaaa

SEQ ID NO: 14 - DNA, 20 bp, Artificial Sequence
  Synthetic oligonucleotide
tgcaccaccaactgcttagc

SEQ ID NO: 15 - DNA, 19 bp, Artificial Sequence
  Synthetic oligonucleotide
ccatcacgccacagtttcc

SEQ ID NO: 16 - DNA, 116 bp, Artificial Sequence
  Synthetic oligonucleotide
tgcaccaccaactgcttagcacccctggccaaggtcatccatgacaactttggtatcgtggaaggactcatgaccacagtccatgccatcactgccacccagaagactgtggatgg

SEQ ID NO: 17 - DNA, 2395 bp, Homo sapiens
agaggcaggggctggcctgggatgcgcgcgcacctgccctcgccccgccccgcccgcacgaggggtggtggccgaggccccgccccgcacgcctcgcctgaggcgggtccgctcagcccaggcgcccgcccccgcccccgccgattaaatgggccggcggggctcagcccccggaaacggtcgtacacttcggggctgcgagcgcggagggcgacgacgacgaagcgcagacagcgtcatggcagagcaggtggccctgagccggacccaggtgtgcgggatcctgcgggaagagcttttccagggcgatgccttccatcagtcggatacacacatattcatcatcatgggtgcatcgggtgacctggccaagaagaagatctaccccaccatctggtggctgttccgggatggccttctgcccgaaaacaccttcatcgtgggctatgcccgttcccgcctcacagtggctgacatccgcaaacagagtgagcccttcttcaaggccaccccagaggagaagctcaagctggaggacttctttgcccgcaactcctatgtggctggccagtacgatgatgcagcctcctaccagcgcctcaacagccacatgaatgccctccacctggggtcacaggccaaccgcctcttctacctggccttgcccccgaccgtctacgaggccgtcaccaagaacattcacgagtcctgcatgagccagataggctggaaccgcatcatcgtggagaagcccttcgggagggacctgcagagctctgaccggctgtccaaccacatctcctccctgttccgtgaggaccagatctaccgcatcgaccactacctgggcaaggagatggtgcagaacctcatggtgctgagatttgccaacaggatcttcggccccatctggaaccgggacaacatcgcctgcgttatcctcaccttcaaggagccctttggcactgagggtcgcgggggctatttcgatgaatttgggatcatccgggacgtgatgcagaaccacctactgcagatgctgtgtctggtggccatggagaagcccgcctccaccaactcagatgacgtccgtgatgagaaggtcaaggtgttgaaatgcatctcagaggtgcaggccaacaatgtggtcctgggccagtacgtggggaaccccgatggagagggcgaggccaccaaagggtacctggacgaccccacggtgccccgcgggtccaccaccgccacttttgcagccgtcgtcctctatgtggagaatgagaggtgggatggggtgcccttcatcctgcgctgcggcaaggccctgaacgagcgcaaggccgaggtgaggctgcagttccatgatgtggccggcgacatcttccaccagcagtgcaagcgcaacgagctggtgatccgcgtgcagcccaacgaggccgtgtacaccaagatgatgaccaagaagccgggcatgttcttcaaccccgaggagtcggagctggacctgacctacggcaacagatacaagaacgtgaagctccctgacgcctacgagcgcctcatcctggacgtcttctgcgggagccagatgcacttcgtgcgcagcgacgagctccgtgaggcctggcgtattttcaccccactgctgcaccagattgagctggagaagcccaagcccatcccctatatttatggcagccgaggccccacggaggcagacgagctgatgaagagagtgggtttccagtatgagggcacctacaagtgggtgaacccccacaagctctgagccctgggcacccacctccacccccgccacggccaccctccttcccgccgcccgaccccgagtcgggaggactccgggaccattgacctcagctgcacattcctggccccgggctctggccaccctggcccgcccctcgctgctgctactacccgagcccagctacattcctcagctgccaagcactcgagaccatcctggcccctccagaccctgcctgagcccaggagctgagtcacctcctccactcactccagcccaacagaaggaaggaggagggcgcccattcgtctgtcccagagcttattggccactgggtctcactcctgagtggggccagggtgggagggagggacaagggggaggaaaggggcgagcacccacgtgagagaatctgcctgtggccttgcccgccagcctcagtgccacttgacattccttgtcaccagcaacatctcgagccccctggatgtcccctgtcccaccaactctgcactccatggccaccccgtgccacccgtaggcagcctctctgctataagaaaagcagacgcagcagctgggacccctcccaacctcaatgccctgccattaaatccgcaaacagcc

SEQ ID NO: 18 - DNA, 20 bp, Artificial Sequence
  Synthetic oligonucleotide
gaggccgtcaccaagaacat

SEQ ID NO: 19 - DNA, 19 bp, Artificial Sequence
  Synthetic oligonucleotide
ggacagccggtcagagctc

SEQ ID NO: 20 - DNA, 111 bp, Artificial Sequence
  Synthetic oligonucleotide
gaggccgtcaccaagaacattcacgagtcctgcatgagccagataggctggaaccgcatcatcgtggagaagcccttcgggagggacctgcagagctctgaccggctgtcc

SEQ ID NO: 21 - DNA, 541 bp, Homo sapiens
cttttcgatccgccatctgcggtggagccgccaccaaaatgcagattttcgtgaaaacccttacggggaagaccatcaccctcgaggttgaaccctcggatacgatagaaaatgtaaaggccaagatccaggataaggaaggaattcctcctgatcagcagagactgatctttgctggcaagcagctggaagatggacgtactttgtctgactacaatattcaaaaggagtctactcttcatcttgtgttgagacttcgtggtggtgctaagaaaaggaagaagaagtcttacaccactcccaagaagaataagcacaagagaaagaaggttaagctggctgtcctgaaatattataaggtggatgagaatggcaaaattagtcgccttcgtcgagagtgcccttctgatgaatgtggtgctggggtgtttatggcaagtcactttgacagacattattgtggcaaatgttgtctgacttactgtttcaacaaaccagaagacaagtaactgtatgagttaataaaagacatgaactaaca

SEQ ID NO: 22 - DNA, 20 bp, Artificial Sequence
  Synthetic oligonucleotide
ctggcaagcagctggaagat

SEQ ID NO: 23 - DNA, 23 bp, Artificial Sequence
  Synthetic oligonucleotide
tttcttagcaccaccacgaagtc

SEQ ID NO: 24 - DNA, 101 bp, Artificial Sequence
  Synthetic oligonucleotide
ctggcaagcagctggaagatggacgtactttgtctgactacaatattcaaaaggagtctactcttcatcttgtgttgagacttcgtggtggtgctaagaaa

SEQ ID NO: 25 - DNA, 2201 bp, Homo sapiens
cgggatttgggtcgcggttcttgtttgtggatcgctgtgatcgtcacttgacaatgcagatcttcgtgaagactctgactggtaagaccatcaccctcgaggttgagcccagtgacaccatcgagaatgtcaaggcaaagatccaagataaggaaggcatccctcctgaccagcagaggctgatctttgctggaaaacagctggaagatgggcgcaccctgtctgactacaacatccagaaagagtccaccctgcacctggtgctccgtctcagaggtgggatgcaaatcttcgtgaagacactcactggcaagaccatcacccttgaggtggagcccagtgacaccatcgagaacgtcaaagcaaagatccaggacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcattcctcctgatcagcagaggttgatctttgccggaaaacagctggaagatggtcgtaccctgtctgactacaacatccagaaagagtccaccttgcacctggtactccgtctcagaggtgggatgcaaatcttcgtgaagacactcactggcaagaccatcacccttgaggtcgagcccagtgacactatcgagaacgtcaaagcaaagatccaagacaaggaaggcattcctcctgaccagcagaggttgatctttgccggaaagcagctggaagatgggcgcaccctgtctgactacaacatccagaaagagtctaccctgcacctggtgctccgtctcagaggtgggatgcagatcttcgtgaagaccctgactggtaagaccatcaccctcgaagtggagccgagtgacaccattgagaatgtcaaggcaaagatccaagacaaggaaggcatccctcctgaccagcagaggttgatctttgccggaaaacagctggaagatggtcgtaccctgtctgactacaacatccagaaagagtccaccttgcacctggtgctccgtctcagaggtgggatgcagatcttcgtgaagaccctgactggtaagaccatcactctcgaggtggagccgagtgacaccattgagaatgtcaaggcaaagatccaagacaaggaaggcatccctcctgatcagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtcttagaggtgggatgcagatcttcgtgaagaccctgactggtaagaccatcactctcgaagtggagccgagtgacaccattgagaatgtcaaggcaaagatccaagacaaggaaggcatccctcctgaccagcagaggttgatctttgctgggaaacagctggaagatggacgcaccctgtctgactacaacatccagaaagagtccaccctgcacctggtgctccgtctcagaggtgggatgcagatcttcgtgaagaccctgactggtaagaccatcaccctcgaggtggagcccagtgacaccatcgagaatgtcaaggcaaagatccaagataaggaaggcatccctcctgatcagcagaggttgatctttgctgggaaacagctggaagatggacgcaccctgtctgactacaacatccagaaagagtccactctgcacttggtcctgcgcttgagggggggtgtctaagtttccccttttaaggtttcaacaaatttcattgcactttcctttcaataaagttgttgcattcccaaaaaaaaaaaaaaaaaaaaaaaa

SEQ ID NO: 26 - DNA, 19 bp, Artificial Sequence
  Synthetic oligonucleotide
atttgggtcgcggttcttg

SEQ ID NO: 27 - DNA, 21 bp, Artificial Sequence
  Synthetic oligonucleotide
tgccttgacattctcgatggt

SEQ ID NO: 28 - DNA, 133 bp, Artificial Sequence
  Synthetic oligonucleotide
atttgggtcgcggttcttgtttgtggatcgctgtgatcgtcacttgacaatgcagatcttcgtgaagactctgactggtaagaccatcaccctcgaggttgagcccagtgacaccatcgagaatgtcaaggca

SEQ ID NO: 29 - DNA, 748 bp, Homo sapiens
gcagataatgggaggagccgggcccgagcgagctctttcctttcgctgctgcggccgcagccatgagtatgctcaggcttcagaagaggctcgcctctagtgtcctccgctgtggcaagaagaaggtctggttagaccccaatgagaccaatgaaatcgccaatgccaactcccgtcagcagatccggaagctcatcaaagatgggctgatcatccgcaagcctgtgacggtccattcccgggctcgatgccggaaaaacaccttggcccgccggaagggcaggcacatgggcataggtaagcggaagggtacagccaatgcccgaatgccagagaaggtcacatggatgaggagaatgaggattttgcgccggctgctcagaagataccgtgaatctaagaagatcgatcgccacatgtatcacagcctgtacctgaaggtgaaggggaatgtgttcaaaaacaagcggattctcatggaacacatccacaagctgaaggcagacaaggcccgcaagaagctcctggctgaccaggctgaggcccgcaggtctaagaccaaggaagcacgcaagcgccgtgaagagcgcctccaggccaagaaggaggagatcatcaagactttatccaaggaggaagagaccaagaaataaaacctcccactttgtctgtacatactggcctctgtgattacatagatcagccattaaaataaaacaagccttaatctgcaaaaaaaaaaaaaaaa

SEQ ID NO: 30 - DNA, 23 bp, Artificial Sequence
  Synthetic oligonucleotide
tggcaagaagaaggtctggttag

SEQ ID NO: 31 - DNA, 22 bp, Artificial Sequence
  Synthetic oligonucleotide
tgatcagcccatctttgatgag

SEQ ID NO: 32 - DNA, 101 bp, Artificial Sequence
  Synthetic oligonucleotide
tggcaagaagaaggtctggttagaccccaatgagaccaatgaaatcgccaatgccaactcccgtcagcagatccggaagctcatcaaagatgggctgatca

SEQ ID NO: 33 - DNA, 1867 bp, Homo sapiens
ggttcgctgtggcgggcgcctgggccgccggctgtttaacttcgcttccgctggcccatagtgatctttgcagtgacccagcagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttaaaaaaaaaaaaaaaaaaaaaa

SEQ ID NO: 34 - DNA, 20 bp, Artificial Sequence
  Synthetic oligonucleotide
cggtttgctgcggtaatcat

SEQ ID NO: 35 - DNA, 21 bp, Artificial Sequence
  Synthetic oligonucleotide
tttcttgctgccagtctggac

SEQ ID NO: 36 - DNA, 122 bp, Artificial Sequence
  Synthetic oligonucleotide
cggtttgctgcggtaatcatgaggataagagagccacgaaccacggcactgattttcagttctgggaaaatggtgtgcacaggagccaagagtgaagaacagtccagactggcagcaagaaa

SEQ ID NO: 37 - DNA, 50 bp, Homo sapiens
tgccttgtgtcaagaagaaggcagactgggccttgcgctggattggggac

SEQ ID NO: 38 - DNA, 3682 bp, Homo sapiens
gctcctcacgcaatcctaaacggttcccgggcgaaccggggcccgcgcgcgccaaggccgccgagaccctcaggggctgcggccctggtcccgcgggacctgtgggggcctgggcggcggcgcccccgacccagccagcggacgggccggggggggaaccgggaggtcccggggggcgtccacgggggtgtccccgggggtctccggaaggcgccggcggaggctcccgcgctgcgcttgaaaatcgcgcgcggccccgcggccagcctgggtaggggcaaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgccgccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggtggacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgtttgtactcacaaggcgataatagcttgatttatttggtttctacaccaaatacattctcctgaccactaatgggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcatacttcagtcacccagtgtcttattctggcattgtctaaatctgagcattgtctagggggatcttaaactttagtaggaaaccatgagctgttaatacagtttccattcaaatattaatttcagaatgaaacataatttttttttttttttttgagatggagtctcgctctgttgcccaggctggagtgcagtggcgcgattttggctcactgtaacctccatctcctgggttcaagcaattctcctgtctcagcctccctagtagctgggactgcaggtatgtgctaccacacctggctaatttttgtatttttagtagagatggagtttcaccatattggtcaggctggtcttgaactcctgacctcaggtgatccacccacctcggcctcccaaagtgctgggattgcaggcgtgataaacaaatattcttaatagggctactttgaattaatctgcctttatgtttgggagaagaaagctgagacattgcatgaaagatgatgagagataaatgttgatcttttggccccatttgttaattgtattcagtatttgaacgtcgtcctgtttattgttagttttcttcatcatttattgtatagacaatttttaaatctctgtaatatgatacattttcctatcttttaagttattgttacctaaagttaatccagattatatggtccttatatgtgtacaacattaaaatgaaaggctttgtcttgcattgtgaggtacaggcggaagttggaatcaggttttaggattctgtctctcattagctgaataatgtgaggattaacttctgccagctcagaccatttcctaatcagttgaaagggaaacaagtatttcagtctcaaaattgaataatgcacaagtcttaagtgattaaaataaaactgttcttatgtcagtttcttgattgg

SEQ ID NO: 39 - DNA, 1732 bp, Homo sapiens
gctcctcacgcaatcctaaacggttcccgggcgaaccggggcccgcgcgcgccaaggccgccgagaccctcaggggctgcggccctggtcccgcgggacctgtgggggcctgggcggcggcgcccccgacccagccagcggacgggccggggggggaaccgggaggtcccggggggcgtccacgggggtgtccccgggggtctccggaaggcgccggcggaggctcccgcgctgcgcttgaaaatcgcgcgcggccccgcggccagcctgggtaggggcaaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgccgccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggtggacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggtgctgggagaccgtgaagtgcaaagcagatggagtccaggccctcggggagacagcacgccagtgagggagatggagaccaatcacccacccagtgtgcggggatgagaagctcgggaaggtctgaccagccttacgtcttgaaagggaatcccctgcttctgcgtgtcaggtgttactcggggtgcgccagtggaagcgggaggcagttgcagctttcagagatggggttttgcatgttggc

SEQ ID NO: 40 - DNA, 1410 bp, Homo sapiens
gctcctcacgcaatcctaaacggttcccgggcgaaccggggcccgcgcgcgccaaggccgccgagaccctcaggggctgcggccctggtcccgcgggacctgtgggggcctgggcggcggcgcccccgacccagccagcggacgggccggggggggaaccgggaggtcccggggggcgtccacgggggtgtccccgggggtctccggaaggcgccggcggaggctcccgcgctgcgcttgaaaatcgcgcgcggccccgcggccagcctgggtaggggcaaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgccgccactatgctctccctccgtgtcccgctcgcgcccatcacggacccgcagcagctgcagctctcgccgctgaaggggctcagcttggtcgacaaggagaacacgccgccggccctgagcgggacccgcgtcctggccagcaagaccgcgaggaggatcttccaggagcccacggagccgaaaactaaagcagctgcccccggcgtggaggatgagccgctgctgagagaaaacccccgccgctttgtcatcttccccatcgagtaccatgatatctggcagatgtataagaaggcagaggcttccttttggaccgccgaggaggtggacctctccaaggacattcagcactgggaatccctgaaacccgaggagagatattttatatcccatgttctggctttctttgcagcaagcgatggcatagtaaatgaaaacttggtggagcgatttagccaagaagttcagattacagaagcccgctgtttctatggcttccaaattgccatggaaaacatacattctgaaatgtatagtcttcttattgacacttacataaaagatcccaaagaaagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgaggtgagtctaagtcaaataatagggtgacctaaaccccaaacacaactcgggcatgctcttgtgttcactgacggggacctgagatgctagatggcatatatccacatttaatgtgtgagttcaaccatacacatacttgac

SEQ ID NO: 41 - DNA, 1380 bp, Homo sapiens
caaatttaaattctatgattcttcaaattgaacaatgatctggacttgaaataattttaaaggcaacaaatgtccctgctgtactggactatgttttactgtctgtagaccctgaagctcaatatgaactacagaatacccaaacttgtattaatgtaaatcaagtgttgaggtttttaaaagaacactggagggaaaaactgaccagtaaaaataaaacatttcggtgtgagttcttcctttaggaagaggattggcaaatacttgaatttggcctttgtcccagagctcttatctagcagttggtaatcggaggtcttttactgtaatgcttcaattgctgataccgtatgtgcctactagggaatttctcttcaatgccattgaaacgatgccttgtgtcaagaagaaggcagactgggccttgcgctggattggggacaaagaggctacctatggtgaacgtgttgtagcctttgctgcagtggaaggcattttcttttccggttcttttgcgtcgatattctggctcaagaaacgaggactgatgcctggcctcacattttctaatgaacttattagcagagatgagggtttacactgtgattttgcttgcctgatgttcaaacacctggtacacaaaccatcggaggagagagtaagagaaataattatcaatgctgttcggatagaacaggagttcctcactgaggccttgcctgtgaagctcattgggatgaattgcactctaatgaagcaatacattgagtttgtggcagacagacttatgctggaactgggttttagcaaggttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgattttttttttccatctcataagaaaaatcagctgaagtgttaccaactagccacaccatgaattgtccgtaatgttcattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgtttgtactcacaagg

SEQ ID NO: 42 - DNA, 570 bp, Homo sapiens
gctcctcacgcaatcctaaacggttcccgggcgaaccggggcccgcgcgcgccaaggccgccgagaccctcaggggctgcggccctggtcccgcgggacctgtgggggcctgggcggcggcgcccccgacccagccagcggacgggccggggggggaaccgggaggtcccggggggcgtccacgggggtgtccccgggggtctccggaaggcgccggcggaggctcccgcgctgcgcttgaaaatcgcgcgcggccccgcggccagcctgggtaggggcaaggcgcagccaatgggaagggtcggaggcatggcacagccaatgggaagggccggggcaccaaagccaatgggaagggccgggagcgcgcggcgcgggagatttaaaggctgctggagtgaggggtcgcccgtgcaccctgtcccagccgtcctgtcctggctgctcgctctgcttcgctgcgccgccactatgctctccctccgtgtcccgctcgcgcccatcacggacccgcagcagctgcagctctcgccgctgaaggggctcagcttggtcgacaaggagaacacg

SEQ ID NO: 43 - DNA, 360 bp, Homo sapiens
caaatttaaattctatgattcttcaaattgaacaatgatctggacttgaaataattttaaaggcaacaaatgtccctgctgtactggactatgttttactgtctgtagaccctgaagctcaatatgaactacagaatacccaaacttgtattaatgtaaatcaagtgttgaggtttttaaaagaacactggagggaaaaactgaccagtaaaaataaaacatttcggtgtgagttcttcctttaggaagaggattggcaaatacttgaatttggcctttgtcccagagctcttatctagcagttggtaatcggaggtcttttactgtaatgcttcaattgctgataccgtatgtgcctac

SEQ ID NO: 44 - DNA, 134 bp, Homo sapiens
gtgaacgtgttgtagcctttgctgcagtggaaggcattttcttttccggttcttttgcgtcgatattctggctcaagaaacgaggactgatgcctggcctcacattttctaatgaacttattagcagagatgag

SEQ ID NO: 45 - DNA, 141 bp, Homo sapiens
gtgagtctaagtcaaataatagggtgacctaaaccccaaacacaactcgggcatgctcttgtgttcactgacggggacctgagatgctagatggcatatatccacatttaatgtgtgagttcaaccatacacatacttgac

SEQ ID NO: 46 - DNA, 260 bp, Homo sapiens
gttttcagagtagagaacccatttgactttatggagaatatttcactggaaggaaagactaacttctttgagaagagagtaggcgagtatcagaggatgggagtgatgtcaagtccaacagagaattcttttaccttggatgctgacttctaaatgaactgaagatgtgcccttacttggctgattttttttttccatctcataagaaaaatcagctgaagtgttaccaactagccacaccatgaattgtccgtaatgtt

SEQ ID NO: 47 - DNA, 362 bp, Homo sapiens
cattaacagcatctttaaaactgtgtagctacctcacaaccagtcctgtctgtttatagtgctggtagtatcaccttttgccagaaggcctggctggctgtgacttaccatagcagtgacaatggcagtcttggctttaaagtgaggggtgaccctttagtgagcttagcacagcgggattaaacagtcctttaaccagcacagccagttaaaagatgcagcctcactgcttcaacgcagattttaatgtttacttaaatataaacctggcactttacaaacaaataaacattgtttgtactcacaaggcgataatagcttgatttatttggtttctacaccaaatacattctcctgaccac

SEQ ID NO: 48 - DNA, 1572 bp, Homo sapiens
taatgggagccaattcacaattcactaagtgactaaagtaagttaaacttgtgtagactaagcatgtaatttttaagttttattttaatgaattaaaatatttgttaaccaactttaaagtcagtcctgtgtatacctagatattagtcagttggtgccagatagaagacaggttgtgtttttatcctgtggcttgtgtagtgtcctgggattctctgccccctctgagtagagtgttgtgggataaaggaatctctcagggcaaggagcttcttaagttaaatcactagaaatttaggggtgatctgggccttcatatgtgtgagaagccgtttcattttatttctcactgtattttcctcaacgtctggttgatgagaaaaaattcttgaagagttttcatatgtgggagctaaggtagtattgtaaaatttcaagtcatccttaaacaaaatgatccacctaagatcttgcccctgttaagtggtgaaatcaactagaggtggttcctacaagttgttcattctagttttgtttggtgtaagtaggttgtgtgagttaattcatttatatttactatgtctgttaaatcagaaattttttattatctatgttcttctagattttacctgtagttcatacttcagtcacccagtgtcttattctggcattgtctaaatctgagcattgtctagggggatcttaaactttagtaggaaaccatgagctgttaatacagtttccattcaaatattaatttcagaatgaaacataatttttttttttttttttgagatggagtctcgctctgttgcccaggctggagtgcagtggcgcgattttggctcactgtaacctccatctcctgggttcaagcaattctcctgtctcagcctccctagtagctgggactgcaggtatgtgctaccacacctggctaatttttgtatttttagtagagatggagtttcaccatattggtcaggctggtcttgaactcctgacctcaggtgatccacccacctcggcctcccaaagtgctgggattgcaggcgtgataaacaaatattcttaatagggctactttgaattaatctgcctttatgtttgggagaagaaagctgagacattgcatgaaagatgatgagagataaatgttgatcttttggccccatttgttaattgtattcagtatttgaacgtcgtcctgtttattgttagttttcttcatcatttattgtatagacaatttttaaatctctgtaatatgatacattttcctatcttttaagttattgttacctaaagttaatccagattatatggtccttatatgtgtacaacattaaaatgaaaggctttgtcttgcattgtgaggtacaggcggaagttggaatcaggttttaggattctgtctctcattagctgaataatgtgaggattaacttctgccagctcagaccatttcctaatcagttgaaagggaaacaagtatttcagtctcaaaattgaataatgcacaagtcttaagtgattaaaataaaactgttcttatgtcagtttcttgattgg

SEQ ID NO: 49 - DNA, 121 bp, Homo sapiens
gggatgagaagctcgggaaggtctgaccagccttacgtcttgaaagggaatcccctgcttctgcgtgtcaggtgttactcggggtgcgccagtggaagcgggaggcagttgcagctttcag

SEQ ID NO: 50 - DNA, 28 bp, Homo sapiens
ccgccggccctgagcgggacccgcgtcc

SEQ ID NO: 51 - DNA, 36 bp, Homo sapiens
tggccagcaagaccgcgaggaggatcttccaggagc

SEQ ID NO: 52 - DNA, 11 bp, Homo sapiens
ccacggagccg

SEQ ID NO: 53 - DNA, 19 bp, Homo sapiens
aaaactaaagcagctgccc

SEQ ID NO: 54 - DNA, 20 bp, Homo sapiens
ccggcgtggaggatgagccg

SEQ ID NO: 55 - DNA, 10 bp, Homo sapiens
ctgctgagag

SEQ ID NO: 56 - DNA, 5 bp, Homo sapiens
aaaac

SEQ ID NO: 57 - DNA, 22 bp, Homo sapiens
ccccgccgctttgtcatcttcc

SEQ ID NO: 58 - DNA, 68 bp, Homo sapiens
ccatcgagtaccatgatatctggcagatgtataagaaggcagaggcttccttttggaccgccgaggag

SEQ ID NO: 59 - DNA, 22 bp, Homo sapiens
gtggacctctccaaggacattc

SEQ ID NO: 60 - DNA, 21 bp, Homo sapiens
agcactgggaatccctgaaac

SEQ ID NO: 61 - DNA, 74 bp, Homo sapiens
ccgaggagagatattttatatcccatgttctggctttctttgcagcaagcgatggcatagtaaatgaaaacttg

SEQ ID NO: 62 - DNA, 53 bp, Homo sapiens
gtggagcgatttagccaagaagttcagattacagaagcccgctgtttctatgg

SEQ ID NO: 63 - DNA, 26 bp, Homo sapiens
cttccaaattgccatggaaaacatac

SEQ ID NO: 64 - DNA, 55 bp, Homo sapiens
attctgaaatgtatagtcttcttattgacacttacataaaagatcccaaagaaag

SEQ ID NO: 65 - DNA, 3 bp, Homo sapiens
tag

SEQ ID NO: 66 - DNA, 95 bp, Homo sapiens
ggaatttctcttcaatgccattgaaacgatgccttgtgtcaagaagaaggcagactgggccttgcgctggattggggacaaagaggctacctatg

SEQ ID NO: 67 - DNA, 105 bp, Homo sapiens
ggtttacactgtgattttgcttgcctgatgttcaaacacctggtacacaaaccatcggaggagagagtaagagaaataattatcaatgctgttcggatagaacag

SEQ ID NO: 68 - DNA, 114 bp, Homo sapiens
gagttcctcactgaggccttgcctgtgaagctcattgggatgaattgcactctaatgaagcaatacattgagtttgtggcagacagacttatgctggaactgggttttagcaag

SEQ ID NO: 69 - DNA, 101 bp, Homo sapiens
gtgctgggagaccgtgaagtgcaaagcagatggagtccaggccctcggggagacagcacgccagtgagggagatggagaccaatcacccacccagtgtgcg

SEQ ID NO: 70 - DNA, 22 bp, Homo sapiens
agatggggttttgcatgttggc

SEQ ID NO: 71 - protein, 389 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 72 - protein, 33 aa, Homo sapiens
MLSLRVPLAPITDPQQLQLSPLKGLSLVDKENT

SEQ ID NO: 73 - protein, 172 aa, Homo sapiens
EATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 74 - protein, 449 aa, Homo sapiens [sequence omitted]

SEQ ID NO: 75 - protein, 211 aa, Homo sapiens
MLQLLIPYVPTREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 76 - protein, 374 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVLGDREVQSRWSPGPRGDSTPVREMETNHPPSVRG

SEQ ID NO: 77 - protein, 274 aa, Homo sapiens
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEVSLSQIIG

SEQ ID NO: 78 - DNA, 21 bp, Artificial Sequence
  Synthetic oligonucleotide
tcagattacagaagcccgctg

SEQ ID NO: 79 - DNA, 33 bp, Artificial Sequence
  Synthetic oligonucleotide
tgtaagtgtcaataagaagactatacatttcag

SEQ ID NO: 80 - DNA, 92 bp, Artificial Sequence
  Synthetic oligonucleotide
tcagattacagaagcccgctgtttctatggcttccaaattgccatggaaaacatacattctgaaatgtatagtcttcttattgacacttaca

SEQ ID NO: 81 - DNA, 23 bp, Artificial Sequence
  Synthetic oligonucleotide
aacatttcggtgtgagttcttcc

SEQ ID NO: 82 - DNA, 24 bp, Artificial Sequence
  Synthetic oligonucleotide
gacctccgattaccaactgctaga

SEQ ID NO: 83 - DNA, 101 bp, Artificial Sequence
  Synthetic oligonucleotide
aacatttcggtgtgagttcttcctttaggaagaggattggcaaatacttgaatttggcctttgtcccagagctcttatctagcagttggtaatcggaggtc

SEQ ID NO: 84 - DNA, 50 bp, Homo sapiens
tcatgcccaagttcaaacaacgaagacgaaagctaaaagccaaagccgaa

SEQ ID NO: 85 - DNA, 6444 bp, Homo sapiens
gacgtaacacaccttctttcccctccggctttccttccccttctctcccgccttctccttattcataccagaagcgcctcagctctgattggctggagctctgtgctatctcagccaatcacaagccgggctgtgctcctacaccatccgaagagcgaatcgtgcagagaccgtgtctacgattggcctctccctgacaaggatttaattttgaatttttctttatggcgtgggagaggccacagcccggactccatcgactcccccggctcttagactaaaatcatgcccaagttcaaacaacgaagacgaaagctaaaagccaaagccgaaagattattcaaaaaaaaagaagcctctcactttcagtccaagctaattacacctcctcctccaccaccctcaccagaaagagtcggtatttcttcaatagatatatctcaaagcagaagctggctaacatcatcctggaacttcaattttcctaacatcagagatgcaataaaactttggacaaatagagtatggtctatatacagctggtgccagaactgcataacccagagtttagaagtattgaaagacaccatctttccatctcgtatctgccaccgagaactttacagtgtaaaacaacagttttgcattttggaaagtaaattatgcaagctccaggaagcactgaagaccatctcagaaagttcttcctgtccaagctgtggtcaaacatgtcacatgagtggtaaacttacaaatgtgcctgcctgcgttctgatcacccctggagactccaaagctgtgcttcctcccacactgccacagccagccagccattttcctcctcctcctccacctccacctctgccacctcctccaccaccactagcacctgtgttgctcagaaaacccagtctcgctaaagcacttcaggctggaccattaaaaaaagatggacccatgcagataacagttaaagatctgctgactgtaaaattaaagaagacacagagtttagatgaaaagaggaagcttataccatcgccgaaagcacggaatccactagttaccgtctctgacttgcagcatgttaccctgaaacctaactccaaagtgttatcgactcgagttacaaacgtcttaatcactcctggaaaaagtcagatggatctgcggaaactgcttagaaaagtcgatgtagagaggagcccaggtggaacccctcttaccaataaggaaaatatggaaacaggaacaggactgactccagtgatgacgcaggccttaaggagaaagtttcagctggctcaccctagaagcccaactccaactctgccactttctacaagcagctttgatgaacaaaactgatgccaactctgcctcactccatgagatgatccataacaaatacagataaaaacacccagctgggtgcagtggctcacacctgtaatcccagcactttgggaggctgaggcaggtggatcacctgaggtcaggagtttgagaccagcctggccaacatggtgaaactccttccccactaaaaatacagaaattagctgggcatagtggcgggtgcctgtaatcccagctatgcgggaggctgaggcaggagaattgcttgaacccaagaggcggaggttgcagtgagccaggatcgcgtcattgcactccagcctgagcaacaagagcgaaacaaaaacaaacaaacaaacaaaaaaaacccacccaaatccttttttttaatgtagtagggtttatatagatatactaatataattgcatttggagaattagagtatgtatggagcccacacatactgtgatataaagtgtatatacagatatttggatattttctagtttgcatgatgattaagagaaccagatgggaaaatacaatctccaaagtgatgtttatcctggaattacccaatttagattagagaggttgttcaaatttaactagataactctagtttgtactgtataggtgcagttatgacagtaaaaaaatagcctcttggctcatacctgtaatcccaccactttgggaggccaaggtgggaggattgcttgagcccaggaattcaagactagcctgggcaacataatacagggagaccccgtttctattaaaaatacaaaaatcagccagctgtggtgacacatgcctatagtcccagctacttaggaagctgagaagggaggatcacttgaggctgtagtgcactataattatgcctgtgaatagccactctactccagcctgggcaacatagcaagaccccatctctaaaaattaaaaaaaaaatttaaattagaatatcatttctagcatcttaggtaggtacttatatctggcttacagaagtctaaggtattccttatttttatatctgctgtccacatttatacagctacataaaaattttatgacaacttcaacatgaaccttatattttcgacaatgccttgccaaggaatctctgaagtccatagcaggtcactgtgagacctagttccctgttgtcactgacctatgtaatcaaagacagtaatacagcctgggaaacatagcgagaccctgtctctatcaaaaatttaaaattacccaggcatggtgacgcacacctgtagtctaagtgtccaagttacttgtgaagctgaggtgggaggatcacttgagcccaagagtttgaagctatggtgagctatgattgttccactatactccagcattggcaacagagcaagaactcatctctaaaaagtaaaaagcaactccccagaaagactgtatttctacagataaatattgcattgagatgccaaatagagtgttgttgtaaagtcatcagactagaaagcagacctggggacagtgtttaccacctaagaggcagtcctgtttttgagacccacatctatatatagagatttttgtttgtttgtttgtttgttttgttttgttttgagatgaagtctcactctgtcacccaggctggagtgcggtggcgcaatcttggctcactgcaacctccacctcccaggttcaagcaattctcctgcctcggcctcctgagtagctgggattacaggtgcgcactgccacgcctgactaatttttgtatttctagtagagatggggtttcactatgttggccaggctggtcttgaactcctcacctcaggtgatccacccatctcagcctcccaaagtgctgagattacaggcgtgagccaccacgcccggccagagatccacatctatatttataacacatttatggatgaaaattaaacaggtgtccgggtgcggttactcatgcctgtaatccgagcacttgtggaggccgaggcaggcggattacttgaggtcaggacttcgaaaccagcctggccaacatggtgaaatcccatctctactaaaaatacaaaattagccaggtgtggtgtcacgcacctgtagtcccagttacttgaacctgggaggcagaggttgcagtgagccgagattgcaacactgccactccagcctgggtgacagagcaagagaccctgtctcaaaaaaaaaaaaaaaaaattaaatgggtagtgacgttaagagatatatatcagcttctagtaaaagtttttttttttaaacctgctagctacatttacattatgtaaaaataaagggaataatcactgagaataaagcagttgagtatttataacaataatattttatggggcgcttataatgtttataatattgtaaaccactgtgtactctattcatttaatgctaaatgacttgaccattcttgtgggataagagatcattaaaaaaatgctagggccgggcaccatggctcacgcctgtaatcccaggactttaggaggccaaggcaggtggatgacttgagctcaggagtttgagaccagcctgggcaacatgatgaaaactccgtctctaccaaaaatcaaaaaaattagccaggtgtgatgttgtgtgcctgtagtcccagctacttgtgaggctgaggtgggaggatggtttgggccccggaggtagaggttgcagtgagctgagattgtgccactgtattccatcctgggcaacagagccagaccctgcctcaaaaaaaaaaaaaaaaaaaaaaaaggcctggcacagtggctcacgcctgtaatcccagcactttgggaggctgaggcgggcaaaacacttgaggtcagacgtttgagaccagcctggccaacatggtgaaaccccgtctctaccaaaaataaaaataaaaattatccaggcgtggtggcatgtgcctatagccccagctactcaggaagctaaagcacgagaattatgtgcatcctggaggtgaaggttgcagtgagccaagatcacaccactccactccagtctggacaacagagtgagaccctgtgtcaaaaaaaaaaaaaagaaagaaagaaagaaaaaatgctttatggcccagtgcagtggctcacacctataatcctagcactttgggaggctaaggcaggaggattgcttgaggccaagagttcaaaacaagcctgggcaacattgcaagaccccaactctacaaaaaacgaaaaattagccgggagcggtgatgtgtgcctgtagtcccagctactcaggagactgaggtgggagaatcacttgagcctgggaggttgaggctgcagtgagctgtgtttataccactgcactccagcctgctgggtaacagagcaagactccatctcaaaaagaaaagaaaaaatgctttgctacataatgaggccaggcaaaaaaaaaaaagtcctgtggaaatcatatagacaaacatttgcaaagctgctactgccattgtaccagtgttaaactgtgttctaccttgcatcttttactgatttttatgacagattttatattgtaaccattcgagaactctgtaagtgctatggcttccttaaactacgatttatcatatgctcccggtgtttactttgagactgaatggcaaccagagaatgtaaacaaccaaggtgcatctggttatgttttaaaataaagattaataaaagttttggtttttctctttctaaccttctttttaaactagaaatataatacctatagaaaagtaaatgttggccgggtgcggtggctcatgcctataatcccagcactttgggaggccgaggcaggcagatcacctgaggtcaagagttcgagaccagtctggccaacatggtgaaaccctgtctctactaaaaatacaaaaattagccgggcatagtggcagacacctgtaatcccagctactgggaaggctgagccagaatggcttgaacctgggaggcagaggttgcagtgagccgagatcgccccattgcactccagcttgagcaacaagagcgaaactgtctcaaaaaaaaaaaaaaagtatatgttaagtatagaaaaatatataaattaaaaaattataggcttgggggccaggcacagtggctcacacctgtaatcccagcactttgggaggccgaggtgggtgtatcacttgaggtcaggagtttgagaccagcctggccaacatggtgaaaccccgtctctaccaaaaaaaatatataaaaattatctgggtgtggtggcatgtgcctatagtctcagcttctcagaagactgaggcgtgagaatcacttgaacgatggaagtggaggttgcagtgagctgagatcacactactgcactacagtctgggcaacagagtgagaccttgtctcaaaaaaaaaagaaagaaaaagaaaaatgctttatggcccagtgcagtggctcacacctataatcctagcactttgggaggctaaggcaggaggttcgcttgaggccaagagttcaaaacaagcctgggcaacactgcaagaccccaactctacaaaaaacgaaaaattagccgggagcggtgatgtgtgcctgtagtcccagctactcaggagactgaggtggaagaatcacttgagcccgggaggttgaggctgcagtgagctgtgtttataccactgcactccagcctgctgggtaacagagcaagactccatctcaaaaagaaaagaaaaaatgctttgctacataatgaggccaggcaaaaaaaaaaagtcctgtggaaatcatatagacaaacatttgcaaagctgctactgccattgtaccagtgttaaaatgtgttctaccttgcatcttttactgatttttatgacagattttatattgtaaccatttgagaactctgtaagtgctatggcttccttaaactacgatttatcatatgctcccagtgtttactttgagactgaatggcaaccagagaatgtaaacaaccaaggtgcatctggttatgttttaaaataaagattaataaaagttttggtttttctctttct

SEQ ID NO: 86 - DNA, 1220 bp, Homo sapiens
gacgtaacacaccttctttcccctccggctttccttccccttctctcccgccttctccttattcataccagaagcgcctcagctctgattggctggagctctgtgctatctcagccaatcacaagccgggctgtgctcctacaccatccgaagagcgaatcgtgcagagaccgtgtctacgattggcctctccctgacaaggatttaattttgaatttttctttatggcgtgggagaggccacagcccggactccatcgactcccccggctcttagactaaaatcatgcccaagttcaaacaacgaagacgaaagctaaaagccaaagccgaaagattattcaaaaaaaaagaagcctctcactttcagtccaagctaattacacctcctcctccaccaccctcaccagaaagagtcggtatttcttcaatagatatatctcaaagcagaagctggctaacatcatcctggaacttcaattttcctaacatcagagatgcaataaaactttggacaaatagagtatggtctatatacagctggtgccagaactgcataacccagagtttagaagtattgaaagacaccatctttccatctcgtatctgccaccgagaactttacagtgtaaaacaacagttttgcattttggaaagtaaattatgcaagctccaggaagcactgaagaccatctcagaaagttcttcctgtccaagctgtggtcaaacatgtcacatgagtggtaaacttacaaatgtgcctgcctgcgttctgatcacccctggagactccaaagctgtgcttcctcccacactgccacagccagccagccattttcctcctcctcctccacctccacctctgccacctcctccaccaccactagcacctgtgttgctcagaaaacccagtctcgctaaagcacttcaggctggaccattaaaaaaagatggacccatgcagataacagttaaagatctgctgactgtaaaattaaagaagacacagagtttagatgaaaagaggaagcttataccatcgccgaaagcacggaatccactagttaccgtctctgacttgcagcatgttaccctgaaacctaactccaaagtgttatcgactcgagttacaaacgtcttaatgtaaggaactgcacagtactatgctctactactacttaaaagaatgatattgttgtacaaaggcctaaagaaaaggaa

SEQ ID NO: 87 - DNA, 280 bp, Homo sapiens
gacgtaacacaccttctttcccctccggctttccttccccttctctcccgccttctccttattcataccagaagcgcctcagctctgattggctggagctctgtgctatctcagccaatcacaagccgggctgtgctcctacaccatccgaagagcgaatcgtgcagagaccgtgtctacgattggcctctccctgacaaggatttaattttgaatttttctttatggcgtgggagaggccacagcccggactccatcgactcccccggctcttagacta

SEQ ID NO: 88 - DNA, 151 bp, Homo sapiens
agtcggtatttcttcaatagatatatctcaaagcagaagctggctaacatcatcctggaacttcaattttcctaacatcagagatgcaataaaactttggacaaatagagtatggtctatatacagctggtgccagaactgcataacccag

SEQ ID NO: 89 - DNA, 123 bp, Homo sapiens
agtttagaagtattgaaagacaccatctttccatctcgtatctgccaccgagaactttacagtgtaaaacaacagttttgcattttggaaagtaaattatgcaagctccaggaagcactgaag

SEQ ID NO: 90 - DNA, 243 bp, Homo sapiens
accatctcagaaagttcttcctgtccaagctgtggtcaaacatgtcacatgagtggtaaacttacaaatgtgcctgcctgcgttctgatcacccctggagactccaaagctgtgcttcctcccacactgccacagccagccagccattttcctcctcctcctccacctccacctctgccacctcctccaccaccactagcacctgtgttgctcagaaaacccagtctcgctaaagcacttcag

SEQ ID NO: 91 - DNA, 758 bp, Homo sapiens
ctggccaacatggtgaaactccttccccactaaaaatacagaaattagctgggcatagtggcgggtgcctgtaatcccagctatgcgggaggctgaggcaggagaattgcttgaacccaagaggcggaggttgcagtgagccaggatcgcgtcattgcactccagcctgagcaacaagagcgaaacaaaaacaaacaaacaaacaaaaaaaacccacccaaatccttttttttaatgtagtagggtttatatagatatactaatataattgcatttggagaattagagtatgtatggagcccacacatactgtgatataaagtgtatatacagatatttggatattttctagtttgcatgatgattaagagaaccagatgggaaaatacaatctccaaagtgatgtttatcctggaattacccaatttagattagagaggttgttcaaatttaactagataactctagtttgtactgtataggtgcagttatgacagtaaaaaaatagcctcttggctcatacctgtaatcccaccactttgggaggccaaggtgggaggattgcttgagcccaggaattcaagactagcctgggcaacataatacagggagaccccgtttctattaaaaatacaaaaatcagccagctgtggtgacacatgcctatagtcccagctacttaggaagctgagaagggaggatcacttgaggctgtagtgcactataattatgcctgtgaatagccactctactccagc

SEQ ID NO: 92 - DNA, 370 bp, Homo sapiens
ctgggcaacatagcaagaccccatctctaaaaattaaaaaaaaaatttaaattagaatatcatttctagcatcttaggtaggtacttatatctggcttacagaagtctaaggtattccttatttttatatctgctgtccacatttatacagctacataaaaattttatgacaacttcaacatgaaccttatattttcgacaatgccttgccaaggaatctctgaagtccatagcaggtcactgtgagacctagttccctgttgtcactgacctatgtaatcaaagacagtaatacagcctgggaaacatagcgagaccctgtctctatcaaaaatttaaaattacccaggcatggtgacgcacacctgta

SEQ ID NO: 93 - DNA, 236 bp, Homo sapiens
gtctaagtgtccaagttacttgtgaagctgaggtgggaggatcacttgagcccaagagtttgaagctatggtgagctatgattgttccactatactccagcattggcaacagagcaagaactcatctctaaaaagtaaaaagcaactccccagaaagactgtatttctacagataaatattgcattgagatgccaaatagagtgttgttgtaaagtcatcagactagaaagcagac

SEQ ID NO: 94 - DNA, 185 bp, Homo sapiens
ctggggacagtgtttaccacctaagaggcagtcctgtttttgagacccacatctatatatagagatttttgtttgtttgtttgtttgttttgttttgttttgagatgaagtctcactctgtcacccaggctggagtgcggtggcgcaatcttggctcactgcaacctccacctcccaggttcaag

SEQ ID NO: 95 - DNA, 137 bp, Homo sapiens
atccacatctatatttataacacatttatggatgaaaattaaacaggtgtccgggtgcggttactcatgcctgtaatccgagcacttgtggaggccgaggcaggcggattacttgaggtcaggacttcgaaaccagc

SEQ ID NO: 96 - DNA, 122 bp, Homo sapiens
gtgaaatcccatctctactaaaaatacaaaattagccaggtgtggtgtcacgcacctgtagtcccagttacttgaacctgggaggcagaggttgcagtgagccgagattgcaacactgccac

SEQ ID NO: 97 - DNA, 352 bp, Homo sapiens
cctgggtgacagagcaagagaccctgtctcaaaaaaaaaaaaaaaaaattaaatgggtagtgacgttaagagatatatatcagcttctagtaaaagtttttttttttaaacctgctagctacatttacattatgtaaaaataaagggaataatcactgagaataaagcagttgagtatttataacaataatattttatggggcgcttataatgtttataatattgtaaaccactgtgtactctattcatttaatgctaaatgacttgaccattcttgtgggataagagatcattaaaaaaatgctagggccgggcaccatggctcacgcctgtaatcccaggactttaggag

SEQ ID NO: 98 - DNA, 283 bp, Homo sapiens
gccaaggcaggtggatgacttgagctcaggagtttgagaccagcctgggcaacatgatgaaaactccgtctctaccaaaaatcaaaaaaattagccaggtgtgatgttgtgtgcctgtagtcccagctacttgtgaggctgaggtgggaggatggtttgggccccggaggtagaggttgcagtgagctgagattgtgccactgtattccatcctgggcaacagagccagaccctgcctcaaaaaaaaaaaaaaaaaaaaaaaaggcctggcacagtggctcac

SEQ ID NO: 99 - DNA, 2075 bp, Homo sapiens
gcctgtaatcccagcactttgggaggctgaggcgggcaaaacacttgaggtcagacgtttgagaccagcctggccaacatggtgaaaccccgtctctaccaaaaataaaaataaaaattatccaggcgtggtggcatgtgcctatagccccagctactcaggaagctaaagcacgagaattatgtgcatcctggaggtgaaggttgcagtgagccaagatcacaccactccactccagtctggacaacagagtgagaccctgtgtcaaaaaaaaaaaaaagaaagaaagaaagaaaaaatgctttatggcccagtgcagtggctcacacctataatcctagcactttgggaggctaaggcaggaggattgcttgaggccaagagttcaaaacaagcctgggcaacattgcaagaccccaactctacaaaaaacgaaaaattagccgggagcggtgatgtgtgcctgtagtcccagctactcaggagactgaggtgggagaatcacttgagcctgggaggttgaggctgcagtgagctgtgtttataccactgcactccagcctgctgggtaacagagcaagactccatctcaaaaagaaaagaaaaaatgctttgctacataatgaggccaggcaaaaaaaaaaaagtcctgtggaaatcatatagacaaacatttgcaaagctgctactgccattgtaccagtgttaaactgtgttctaccttgcatcttttactgatttttatgacagattttatattgtaaccattcgagaactctgtaagtgctatggcttccttaaactacgatttatcatatgctcccggtgtttactttgagactgaatggcaaccagagaatgtaaacaaccaaggtgcatctggttatgttttaaaataaagattaataaaagttttggtttttctctttctaaccttctttttaaactagaaatataatacctatagaaaagtaaatgttggccgggtgcggtggctcatgcctataatcccagcactttgggaggccgaggcaggcagatcacctgaggtcaagagttcgagaccagtctggccaacatggtgaaaccctgtctctactaaaaatacaaaaattagccgggcatagtggcagacacctgtaatcccagctactgggaaggctgagccagaatggcttgaacctgggaggcagaggttgcagtgagccgagatcgccccattgcactccagcttgagcaacaagagcgaaactgtctcaaaaaaaaaaaaaaagtatatgttaagtatagaaaaatatataaattaaaaaattataggcttgggggccaggcacagtggctcacacctgtaatcccagcactttgggaggccgaggtgggtgtatcacttgaggtcaggagtttgagaccagcctggccaacatggtgaaaccccgtctctaccaaaaaaaatatataaaaattatctgggtgtggtggcatgtgcctatagtctcagcttctcagaagactgaggcgtgagaatcacttgaacgatggaagtggaggttgcagtgagctgagatcacactactgcactacagtctgggcaacagagtgagaccttgtctcaaaaaaaaaagaaagaaaaagaaaaatgctttatggcccagtgcagtggctcacacctataatcctagcactttgggaggctaaggcaggaggttcgcttgaggccaagagttcaaaacaagcctgggcaacactgcaagaccccaactctacaaaaaacgaaaaattagccgggagcggtgatgtgtgcctgtagtcccagctactcaggagactgaggtggaagaatcacttgagcccgggaggttgaggctgcagtgagctgtgtttataccactgcactccagcctgctgggtaacagagcaagactccatctcaaaaagaaaagaaaaaatgctttgctacataatgaggccaggcaaaaaaaaaaagtcctgtggaaatcatatagacaaacatttgcaaagctgctactgccattgtaccagtgttaaaatgtgttctaccttgcatcttttactga

SEQ ID NO: 100 - DNA, 181 bp, Homo sapiens
attttatattgtaaccatttgagaactctgtaagtgctatggcttccttaaactacgatttatcatatgctcccagtgtttactttgagactgaatggcaaccagagaatgtaaacaaccaaggtgcatctggttatgttttaaaataaagattaataaaagttttggtttttctctttct

SEQ ID NO: 101 - DNA, 95 bp, Homo sapiens
aaatcatgcccaagttcaaacaacgaagacgaaagctaaaagccaaagccgaaagattattcaaaaaaaaagaagcctctcactttcagtccaag

SEQ ID NO: 102 - DNA, 38 bp, Homo sapiens
ctaattacacctcctcctccaccaccctcaccagaaag

SEQ ID NO: 103 - DNA, 99 bp, Homo sapiens
gctggaccattaaaaaaagatggacccatgcagataacagttaaagatctgctgactgtaaaattaaagaagacacagagtttagatgaaaagaggaag

SEQ ID NO: 104 - DNA, 113 bp, Homo sapiens
cttataccatcgccgaaagcacggaatccactagttaccgtctctgacttgcagcatgttaccctgaaacctaactccaaagtgttatcgactcgagttacaaacgtcttaat

SEQ ID NO: 105 - DNA, 78 bp, Homo sapiens
gtaaggaactgcacagtactatgctctactactacttaaaagaatgatattgttgtacaaaggcctaaagaaaaggaa

SEQ ID NO: 106 - DNA, 60 bp, Homo sapiens
cactcctggaaaaagtcagatggatctgcggaaactgcttagaaaagtcgatgtagagag

SEQ ID NO: 107 - DNA, 97 bp, Homo sapiens
gagcccaggtggaacccctcttaccaataaggaaaatatggaaacaggaacaggactgactccagtgatgacgcaggccttaaggagaaagtttcag

SEQ ID NO: 108 - DNA, 19 bp, Homo sapiens
ctggctcaccctagaagcc

SEQ ID NO: 109 - DNA, 17 bp, Homo sapiens
caactccaactctgcca

SEQ ID NO: 110 - DNA, 58 bp, Homo sapiens
ctttctacaagcagctttgatgaacaaaactgatgccaactctgcctcactccatgag

SEQ ID NO: 111 - DNA, 54 bp, Homo sapiens
atgatccataacaaatacagataaaaacacccagctgggtgcagtggctcacac

SEQ ID NO: 112 - DNA, 24 bp, Homo sapiens
ctgtaatcccagcactttgggagg

SEQ ID NO: 113 - DNA, 9 bp, Homo sapiens
ctgaggcag

SEQ ID NO: 114 - DNA, 34 bp, Homo sapiens
gtggatcacctgaggtcaggagtttgagaccagc

SEQ ID NO: 115 - DNA, 109 bp, Homo sapiens
caattctcctgcctcggcctcctgagtagctgggattacaggtgcgcactgccacgcctgactaatttttgtatttctagtagagatggggtttcactatgttggccag

SEQ ID NO: 116 - DNA, 25 bp, Homo sapiens
gctggtcttgaactcctcacctcag

SEQ ID NO: 117 - DNA, 68 bp, Homo sapiens
gtgatccacccatctcagcctcccaaagtgctgagattacaggcgtgagccaccacgcccggccagag

SEQ ID NO: 118 - DNA, 12 bp, Homo sapiens
ctggccaacatg

SEQ ID NO: 119 - DNA, 5 bp, Homo sapiens
tccag

SEQ ID NO: 120 - DNA, 12 bp, Homo sapiens
tttttatgacag

SEQ ID NO: 121 - protein, 360 aa, Homo sapiens
MPKFKQRRRKLKAKAERLFKKKEASHFQSKLITPPPPPPSPERVGISSIDISQSRSWLTSSWNFNFPNIRDAIKLWTNRVWSIYSWCQNCITQSLEVLKDTIFPSRICHRELYSVKQQFCILESKLCKLQEALKTISESSSCPSCGQTCHMSGKLTNVPACVLITPGDSKAVLPPTLPQPASHFPPPPPPPPLPPPPPPLAPVLLRKPSLAKALQAGPLKKDGPMQITVKDLLTVKLKKTQSLDEKRKLIPSPKARNPLVTVSDLQHVTLKPNSKVLSTRVTNVLITPGKSQMDLRKLLRKVDVERSPGGTPLTNKENMETGTGLTPVMTQALRRKFQLAHPRSPTPTLPLSTSSFDEQN

SEQ ID NO: 122 - protein, 210 aa, Homo sapiens
MSGKLTNVPACVLITPGDSKAVLPPTLPQPASHFPPPPPPPPLPPPPPPLAPVLLRKPSLAKALQAGPLKKDGPMQITVKDLLTVKLKKTQSLDEKRKLIPSPKARNPLVTVSDLQHVTLKPNSKVLSTRVTNVLITPGKSQMDLRKLLRKVDVERSPGGTPLTNKENMETGTGLTPVMTQALRRKFQLAHPRSPTPTLPLSTSSFDEQN

SEQ ID NO: 123 - protein, 360 aa, Homo sapiens
MPKFKQRRRKLKAKAERLFKKKEASHFQSKLITPPPPPPSPERVGISSIDISQSRSWLTSSWNFNFPNIRDAIKLWTNRVWSIYSWCQNCITQSLEVLKDTIFPSRICHRELYSVKQQFCILESKLCKLQEALKTISESSSCPSCGQTCHMSGKLTNVPACVLITPGDSKAVLPPTLPQPASHFPPPPPPPPLPPPPPPLAPVLLRKPSLAKALQAGPLKKDGPMQITVKDLLTVKLKKTQSLDEKRKLIPSPKARNPLVTVSDLQHVTLKPNSKVLSTRVTNVLITPGKSQMDLRKLLRKVDVERRPGGTPLTNKENMETGTGLTPVMTQALRRKFQLAHPRSPTPTLPLSTSSFDEQN

SEQ ID NO: 124 - protein, 286 aa, Homo sapiens [sequence omitted]

SEQ ID NO: 125 - DNA, 23 bp, Artificial Sequence
  Synthetic oligonucleotide
cgtctctgacttgcagcatgtta

SEQ ID NO: 126 - DNA, 36 bp, Artificial Sequence
  Synthetic oligonucleotide
caacaatatcattcttttaagtagtagtagagcata

SEQ ID NO: 127 - DNA, 130 bp, Artificial Sequence
  Synthetic oligonucleotide
cgtctctgacttgcagcatgttaccctgaaacctaactccaaagtgttatcgactcgagttacaaacgtcttaatgtaaggaactgcacagtactatgctctactactacttaaaagaatgatattgttg

SEQ ID NO: 128 - protein, 60 aa, Artificial Sequence
  Synthetic peptide
MGRVGGMAQPMGRAGAPKPMGRAGSARRGRFKGCWSEGSPVHPVPAVLSWLLALLRCAAT

SEQ ID NO: 129 - protein, 11 aa, Artificial Sequence
  Synthetic peptide
MLQLLIPYVPT

SEQ ID NO: 130 - protein, 39 aa, Artificial Sequence
  Synthetic peptide
MLQLLIPYVPTREFLFNAIETMPCVKKKADWALRWIGDK

SEQ ID NO: 131 - protein, 34 aa, Artificial Sequence
  Synthetic peptide
LGDREVQSRWSPGPRGDSTPVREMETNHPPSVRG

SEQ ID NO: 132 - protein, 8 aa, Artificial Sequence
  Synthetic peptide
VSLSQIIG

SEQ ID NO: 133 - protein, 150 aa, Artificial Sequence
  Synthetic peptide
MPKFKQRRRKLKAKAERLFKKKEASHFQSKLITPPPPPPSPERVGISSIDISQSRSWLTSSWNFNFPNIRDAIKLWTNRVWSIYSWCQNCITQSLEVLKDTIFPSRICHRELYSVKQQFCILESKLCKLQEALKTISESSSCPSCGQTCH

SEQ ID NO: 134 - protein, 329 aa, Homo sapiens
KAAAPGVEDEPLLRENPRRFVIFPIEYHDIWQMYKKAEASFWTAEEVDLSKDIQHWESLKPEERYFISHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 135 - protein, 360 aa, Homo sapiens; identical to SEQ ID NO: 121

(19) United States
(12) Patent Application Publication
(10) Pub. No.: US 2009/0075257 A1
(43) Pub. Date: Mar. 19, 2009

(54) NOVEL NUCLEIC ACID SEQUENCES AND METHODS OF USE THEREOF FOR DIAGNOSIS

(75) Inventors: Sarah Pollock, Tel-Aviv (IL); Osnat Sella-Tavor, Kfar Kish (IL); Amit Novik, Tel Mond (IL); Elena Tsypkin, Tel-Aviv (IL); Dan Sztybel, Tel-Aviv (IL); Shira Walach, Hod Hasharon (IL)

Correspondence Address: FENNEMORE CRAIG, 3003 NORTH CENTRAL AVENUE, SUITE 2600, PHOENIX, AZ, 85012, US

(73) Assignee: Compugen Ltd., Tel-Aviv (IL)

(21) Appl. No.: 11/831,404

(22) Filed: Jul. 31, 2007

Related U.S. Application Data
(63) Continuation-in-part of application No. 11/043,860, filed on Jan. 27, 2005, now abandoned.
(60) Provisional application No. 60/539,129, filed on Jan. 27, 2004.

(30) Foreign Application Priority Data
Jul. 31, 2006 (IL) 177186

(51) Int. Cl. C12Q 1/68; C07H 21/00; G01N 33/53

(52) U.S. Cl. 435/6; 536/23.1; 436/501; 536/24.33

(57) ABSTRACT

Novel splice variant nucleic acid and amino acid sequences are provided. The novel splice variants and their nucleic acid sequences may be used for diagnosis of variant-detectable diseases particularly cancerous diseases.

### RELATED APPLICATION DATA

This application is a continuation-in-part of and claims priority to U.S. application Ser. No. 11/043,860 filed on Jan. 27, 2005, which claims the benefit of priority from U.S. Provisional Application No. 60/539,129 filed on Jan. 27, 2004, and this application also claims priority to Israeli Application No. 177186 filed Jul. 31, 2006. The content of each of the aforementioned applications is expressly incorporated herein in their entirety by reference hereto.

### FIELD OF THE INVENTION

The present invention is related to novel nucleotide acid sequences, and assays and methods of use thereof particularly for diagnosis.

### BACKGROUND OF THE INVENTION

Diagnostic markers are important for early diagnosis of many diseases, as well as predicting response to treatment, monitoring treatment and determining prognosis of such diseases.

Nucleic Acid Testing (NAT) is a subset of molecular diagnostic markers, based on testing for the presence of a nucleic acid sequence in a sample, associated with a certain condition (most often a clinical pathology). The sample could be a body fluid, a tissue sample, a body secretion or any other sample obtained from a patient which could contain the targeted nucleic acids.

Traditionally, NAT diagnosis has been used for the diagnosis of infectious diseases. Particularly, it has been used for the diagnosis of HIV, Hepatitis C Virus (HCV), Hepatitis B Virus (HBV), Chlamydia trachomatis, Neisseria gonorrhoeae and Mycobacteria tuberculosis. In recent years NAT diagnosis has expanded to noninfectious diseases, for example, for the diagnosis of prostate cancer based on DD3 (PCA3). DD3 (PCA3) is a very prostate cancer-specific gene. It has shown a great diagnostic value for prostate cancer by measuring quantitavely the DD3 (PCA3) transcript in urine sediments obtained after prostatic massage. DD3 (PCA3) is a non-coding transcript, therefore diagnosis in the protein level is not possible. More NAT markers for more cancers in addition to prostate cancer are currently pursued.

NAT diagnostic markers have at least four advantages on protein based diagnostic modalities:

1. They are likely to be more sensitive and specific (as has been shown for diagnostic kits for HIV and HCV). This finding could be related to at least two parameters:
a. The test analyte could be amplified (e.g. with PCR)
b. The detection method is sequence specific rather than epitope specific

2. They allow diagnosis even if a differentially expressed transcript is non-coding (as in the case of DD3 (PCA3))

3. The research tools for the discovery of novel NAT markers are much more advanced and robust than for protein markers (e.g. advanced DNA chip technology compared with protein chip technology)

4. NAT analytes are sometimes found in body secretions and/or body fluids and therefore could replace the need for a tissue biopsy when a serum marker is not available.

However, NAT markers suffer from a few disadvantages including:

1. The analyte itself is quite an unstable molecule (certainly when compared with a protein).

2. The analyte itself is by nature not physiologically secreted, therefore it is not always easily found in samples.

NAT markers development for noninfectious diseases was not pursued for a long time, which was mostly a result of expensive and not fully developed detection methods on one hand and intellectual property barriers on the other. With the advance in technology and expiration of key patents in the field, the industry is investing more and more resources in that direction and it seems that NAT based tests are going to be much more prevalent for noninfectious diseases in the future.

### SUMMARY OF THE INVENTION

The present invention overcomes deficiencies of the background art by providing novel variants that are suitable for use with NAT and/or nucleic acid hybridization methods and assays, which may optionally be used as diagnostic markers. Collectively, methods and assays that are suitable for detecting a nucleic acid sequence (oligonucleotides) are referred to herein as “oligonucleotide detection technologies”, including but not limited to NAT and hybridization technologies. The markers of the present invention may optionally be used with any such oligonucleotide detection technology.

The markers are useful for detecting variant-detectable diseases (marker-detectable diseases), wherein these diseases and/or pathological states and/or conditions are described in greater detail below with regard to the different clusters (genes) below.

Preferably these variants are useful as diagnostic markers for variant-detectable diseases.

According to one embodiment of the present invention markers are specifically released to the bloodstream under disease conditions according to one of the above variant-detectable diseases or conditions.

The present invention therefore also relates to diagnostic assays for disease detection optionally and preferably in a sample taken from a subject (patient), which is more preferably some type of blood sample or body secretion sample. The assays are optionally NAT (nucleic acid amplification technology)-based assays, such as PCR for example (or variations thereof such as real-time PCR for example). The assays may also optionally encompass nucleic acid hybridization assays. The assays may optionally be qualitative or quantitative.

Variant T29749_T1 (SEQ ID NO:38) encoding the protein T29749_P0 (SEQ ID NO:74), variant T29749_T14 (SEQ ID NO:40) encoding the protein T29749_P34 (SEQ ID NO:76), variant T29749_P13 (SEQ ID NO:75), and variant T29749_P40 (SEQ ID NO:77), were previously disclosed by the inventors of the present invention in published PCT application no WO2005/071058 and in US patent application publication no. US-2006-0068405, hereby incorporated by reference as if fully set forth herein (from the later priority for this application is claimed). T29749_T1 (SEQ ID NO:38), T29749_T14 (SEQ ID NO:40), T29749_P0 (SEQ ID NO:74), T29749_P13 (SEQ ID NO:75), T29749_P34 (SEQ ID NO:76) and T29749_P40 (SEQ ID NO:77), corresponding to previously described HSRR2SS_T0, HSR2SS_T13, HSRR2SS_P1, H67307_{—}9, HSRR2SS_P2_{—}4 and HSRR2SS_P8_{—}4, respectively, were demonstrated (through analysis of EST expression levels) by the inventors of the present invention in published PCT application no WO2005/071058 and in US patent application publication no US-2006-0068405 to be differentially expressed in skin cancer, liver cancer, lung cancer and epithelial cancers.

Surprisingly, new transcripts, T29749_T7 (SEQ ID NO:39) encoding

T29749_P13 (SEQ ID NO:75) and T29749_T20 (SEQ ID NO:41) encoding T29749_P40 (SEQ ID NO:77), were identified. These new transcript variants are shown now to have novel and surprising diagnostic uses in breast cancer, lung cancer and ovarian cancer. In particular, RT-PCR results showed overexpression in lung, breast and ovarian cancers for T29749_T20. Furthermore it will be demonstrated that other transcripts from T29749 (including T29749_T1, T29749_T14 and T29749_T7) are also overexpressed in breast and ovarian cancers.

Variant F13779_T16 (SEQ ID NO:86) encoding the protein F13779_P5 (SEQ ID NO:124), and variant F13779_P0 (SEQ ID NO:121), were previously disclosed by the inventors of the present invention in published PCT application no WO2005/071058 and in US patent application publication no. US-2006-0068405, hereby incorporated by reference as if fully set forth herein (from the later priority for this application is also claimed) F13779_T16 (SEQ ID NO:86), F13779_P5 (SEQ ID NO:124) and F13779_P0 (SEQ ID NO:121), which are identical to previously described F13779_T15, F13779_P5 (SEQ ID NO:124) and F13779_P1 respectively, were demonstrated (through analysis of EST expression levels) by the inventors of the present invention in published PCT application no WO2005/071058 and in US patent application US-2006-0068405 to be differentially expressed in epithelial cancers.

Surprisingly F13779_T16 is shown now to have novel diagnostic uses in lung cancer, breast cancer and ovarian cancer.

Surprisingly, new transcript, F13779_T0 (SEQ ID NO:85) encoding F13779_P0 (SEQ ID NO:121) was identified. This new transcript is now shown to have novel and surprising diagnostic uses in lung cancer, breast cancer and ovarian cancer. In particular, RT-PCR results showed overexpression in lung, breast and ovarian cancers for F13779_T16. Furthermore it will be demonstrated that other transcripts from F13779 (including F13779_T0) are also overexpressed in lung, breast and ovarian cancers.

According to certain embodiments, the present invention provides a diagnostic marker comprising a novel splice variant of a known protein or a polynucleotide encoding same, wherein the protein is selected from the group consisting of Ribonucleoside-diphosphate reductase M2 chain (SwissProt accession identifier RIR2_HUMAN; known also according to the synonyms EC 1.17.4.1; Ribonucleotide reductase small chain) and FLJ11029 protein (SwissProt accession identifier Q96HE9_HUMAN (SEQ ID NO:135)). According to certain embodiments, the diagnostic marker is found in a body fluid or secretion.

According to one embodiment, the novel splice variant is an isolated polynucleotide comprising a nucleic acid having a nucleic acid sequence as set forth in any one of SEQ. ID NOs: 38-41, 85, 86, or a sequence homologous thereto. According to one embodiment, the isolated polynucleotide is at least about 85% identical to any one of SEQ. ID NOs: 38-41, 85, 86. According to one embodiment, the isolated polynucleotide is at least about 95% identical to any one of SEQ. ID NOs: 38-41, 85, 86.

According to another embodiment, the novel splice variant is an isolated polynucleotide comprising a nucleic acid having a nucleic acid sequence as set forth in any one of SEQ. ID NOs: 42-70, 87-120, or a sequence homologous thereto. According to one embodiment, the isolated polynucleotide is at least about 85% identical to any one of SEQ. ID NOs: 42-70, 87-120. According to one embodiment, the isolated polynucleotide is at least about 95% identical to any one of SEQ. ID NOs: 42-70, 87-120.

According to another embodiment the present invention also encompasses an isolated oligonucleotide comprising an amplicon selected from the group consisting of SEQ ID NOs:80, 83, 127.

According to another embodiment the present invention also encompasses a primer pair, comprising a pair of isolated oligonucleotides capable of amplifying said amplicon of SEQ ID NOs:80, 83, 127.

According to another embodiment the present invention also encompasses a primer pair comprising a pair of isolated oligonucleotides having a sequence selected from the group consisting of SEQ ID NOs:78 and 79; 81 and 82; 125 and 126.

According to another embodiment the present invention also encompasses isolated polypeptide comprising a polypeptide having a sequence selected from the group consisting of SEQ ID NOs:74-77, 121, and 124.

According to certain embodiments, the present invention also encompasses isolated polynucleotides having a sequence complementary to any one of the nucleic acid sequences listed herein. According to other embodiments, this invention provides an oligonucleotide of at least about 12 nucleotides, specifically hybridizable with the polynucleotides of this invention. The present invention further provides vectors, cells, liposomes and compositions comprising the isolated polynucleotides of this invention.

According to some embodiments, the sample taken from a subject (patient) to perform the diagnostic assay according to the present invention is selected from the group consisting of a body fluid or secretion including but not limited to blood, serum, urine, stool, plasma, prostatic fluid, seminal fluid, semen, the external secretions of the skin, respiratory, intestinal, and genitourinary tracts, tears, cerebrospinal fluid, sputum, saliva, milk, peritoneal fluid, pleural fluid, cyst fluid, secretions of the breast ductal system (and/or lavage thereof), broncho alveolar lavage, lavage of the reproductive system and lavage of any other part of the body or system in the body; samples of any organ including isolated cell(s) or tissue(s), wherein the cell or tissue can be obtained from an organ selected from, but not limited to lung, colon, ovarian and/or breast tissue; or a tissue sample, or any combination thereof. In some embodiments, the term encompasses samples of in vivo cell culture constituents. Prior to be subjected to the diagnostic assay, the sample can optionally be diluted with a suitable eluant.

The term “homology”, as used herein, refers to a degree of sequence similarity in terms of shared amino acid or nucleotide sequences. There may be partial homology or complete homology. For amino acid sequence homology amino acid similarity matrices may be used as are known in different bioinformatics programs (e.g. BLAST, Smith Waterman). Different results may be obtained when performing a particular search with a different matrix. Homologous peptide or polypeptides are characterized by one or more amino acid substitutions, insertions or deletions, such as, but not limited to, conservative substitutions, provided that these changes do not affect the biological activity of the peptide or polypeptide as described herein.

Degrees of homology (or identity) for nucleotide sequences are based upon identity matches with penalties made for gaps or insertions required to optimize the alignment, as is well known in the art (e.g. Altschul S. F. et al., 1990, J Mol Biol 215(3):403-10; Altschul S. F. et al., 1997, Nucleic Acids Res. 25:3389-3402). The degree of sequence homology is presented in terms of percentage, e.g. “70% homology” or “70% identity”. As used herein, the term “at least” with regard to a certain degree of homology encompasses any degree of homology from the specified percentage up to 100%.

The terms “correspond” or “corresponding to” or “correspondence with” are used herein to indicate identity between two corresponding amino acid or nucleic acid sequences.

According to certain embodiments, the present invention now discloses a cluster designated herein T29749, comprising novel nucleic acid sequences and protein sequences that are variants of the known Ribonucleoside-diphosphate reductase M2 chain. The polynucleotides and polypeptides described by the present invention are useful as diagnostic markers.

Surprisingly, the present invention now shows that novel T29749 variants of invention are overexpressed in lung cancer, ovarian cancer and breast cancer, and thus can be used for the diagnosis, prognosis, treatment selection, and treatment monitoring and/or assessment of lung cancer, ovarian cancer and breast cancer, as is described in a greater detail below.

According to certain embodiments, the present invention now discloses a cluster designated herein F13779 comprising novel nucleic acid sequences and protein sequences that are variants of the known FLJ11029. The novel variant polynucleotides and polypeptides described by the present invention are useful as diagnostic markers.

Surprisingly, the present invention now shows that F13779 variants of invention are overexpressed in lung cancer, ovarian cancer and breast cancer, and thus can be used for the diagnosis, prognosis, treatment selection, and treatment monitoring and/or assessment of lung cancer, ovarian cancer and breast cancer, as is described in a greater detail below.

According to the present invention, the splice variant nucleic acid sequences described herein are non-limiting examples of markers for diagnosing the below described disease condition(s). Each splice variant nucleic acid sequence marker of the present invention can be used alone or in combination, for various uses, including but not limited to, prognosis, prediction, screening, early diagnosis, determination of progression, therapy selection and treatment monitoring of one of the above-described diseases.

According to optional but preferred embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

According to other preferred embodiments of the present invention, a splice variant nucleic acid sequence or a fragment thereof, may be featured as a biomarker for detecting a variant-detectable disease, such that a biomarker may optionally comprise any of the above.

According to additional aspect the present invention provides a diagnostic kit for detecting a disease, comprising markers and reagents for detecting qualitative and/or quantitative changes in the expression of a polypeptide or a polynucleotide of this invention.

According to one embodiment, the kit comprises markers and reagents for detecting the changes by employing a NAT-based technology. In one embodiment, the NAT-based assay is selected from the group consisting of a PCR, Real-Time PCR, LCR, Self-Sustained Synthetic Reaction, Q-Beta Replicase, Cycling Probe Reaction, Branched DNA, RFLP analysis, DGGE/TGGE, Single-Strand Conformation Polymorphism, Dideoxy Fingerprinting, Microarrays, Fluorescence, In Situ Hybridization or Comparative Genomic Hybridization.

According to certain currently preferred embodiments, the kit comprises at least one nucleotide probe or primer. In one embodiment, the kit comprises at least one primer pair capable of selectively hybridizing to a nucleic acid sequence according to the teaching of the present invention. In another embodiment, the kit comprises at least one oligonucleotide capable of selectively hybridizing to a nucleic acid sequence according to the teaching of the present invention.

The present invention further provides diagnostic methods for screening for a disease, disorder or conditions, comprising the detection of a polypeptide or polynucleotide of this invention, whereby expression, or relative changes in expression of the polypeptide or polynucleotide herald the onset, severity, or prognosis of an individual with regard to a particular disease, disorder or condition. The detection may comprise detection of the expression of a specific variant of this invention, via any means known in the art, and as described herein.

As used herein, the term “screening for a disease” encompasses diagnosing the presence of a disease, its prognosis and/or severity, as well as selecting a treatment and monitoring the treatment of the disease. According to certain currently preferred embodiments, the disease is a marker-detectable disease, wherein the marker is a polynucleotide according to the present invention.

Thus, according to certain aspects, the present invention provides methods for screening for a marker detectable disease, comprising detecting in a subject or in a sample obtained from the subject at least one transcript and/or protein or polypeptide being a member of a cluster selected from the group consisting of cluster T29749, cluster F13779, or any combination thereof. According to certain currently preferred embodiments, the method comprises detecting the expression of a splice variant transcript or a product thereof.

According to one aspect, the present invention provide a method for screening for a marker detectable disease in a subject, comprising (a) obtaining a sample from the subject and (b) detecting in the sample at least one polynucleotide and/or polypeptide being a member of a cluster selected from the group consisting of cluster T29749, cluster F13779, or any combination thereof. According to one embodiment, the presence of the polynucleotide and/or polypeptide in the sample is indicative of the presence of the disease and/or its severity and/or its progress. According to another embodiment, a change in the level of the polynucleotide and/or polypeptide in the sample compared to its level in a sample obtained from a healthy subject is indicative of the presence of the disease and/or its severity. According to another embodiment, a change in the level of the polynucleotide and/or polypeptide in the sample compared to its level in a sample previously obtained from said subject is indicative of the presence of the disease, its severity and/or the progress of the disease.

Thus, according to another embodiment, the present invention provides a method for screening for a breast cancer, lung cancer, ovarian cancer and/or ovarian, breast or lung cancer invasion and metastasis in a subject, comprising detecting in the subject differential expression of at least one polynucleotide or polypeptide of the invention being a member of cluster T29749.

According to yet another embodiment, the present invention provides a method for screening for a breast cancer, lung cancer, ovarian cancer and/or ovarian, breast or lung cancer invasion and metastasis in a subject, comprising detecting in the subject a polynucleotide comprising a nucleic acid sequence at least 95% identical to the nucleic acid sequence set forth in any one of SEQ. ID NOs: 38-41. According to one embodiment, the method comprises detecting a polynucleotide comprising a nucleic acid sequence as set forth in any one of SEQ. ID NOs: 38-41.

According to this embodiment, the method for screening for a T29749 marker detectable disease is optionally performed in vitro with a sample obtained from the subject.

According to yet another aspect, the present invention provides a method for screening for a lung cancer, breast cancer, ovarian cancer and/or lung, ovarian or breast cancer invasion and metastasis in a subject, comprising detecting in the subject a polypeptide comprising an amino acid sequence as set forth in any one of SEQ. ID NOs:74-77.

Each polynucleotide or polypeptide of the T29749 variants described herein as a marker for breast cancer, lung cancer or ovarian cancer, can be used alone or in combination with one or more other variant markers described herein, and/or in combination with known markers for breast cancer, lung cancer or ovarian cancer.

The present invention further discloses that surprisingly, detecting in a subject differential expression of at least one polynucleotide or polypeptide of F13779 variants is indicative of lung cancer, ovarian cancer and breast cancer. Detecting the presence of the polynucleotide or polypeptide in the subject or detecting a relative change in their expression and/or level compared to a healthy subject or compared to their expression and/or level in said subject at an earlier stage is indicative of the presence, onset, severity or prognosis, and/or staging, and/or progression, of lung cancer, ovarian cancer and breast cancer, in said subject. These polynucleotides or polypeptides of cluster F13779 are also useful for treatment selection and treatment monitoring of lung cancer, ovarian cancer, breast cancer, and/or ovarian, breast or lung cancer invasion and metastasis.

Thus, according to certain embodiments, the present invention provides a method for screening for lung cancer, ovarian cancer, breast cancer, and/or ovarian, breast or lung cancer invasion and metastasis in a subject, comprising detecting in the subject at least one polynucleotide or polypeptide being a member of cluster F13779.

According to this embodiment, the method for screening for a lung cancer, ovarian cancer, breast cancer, and/or ovarian, breast or lung cancer invasion and metastasis is performed in vitro with a sample obtained from the subject.

According to yet another embodiment, the present invention provides a method for screening for lung cancer, ovarian cancer, breast cancer, and/or ovarian, breast or lung cancer invasion and metastasis in a subject, comprising detecting in the subject a polynucleotide comprising a nucleic acid sequence at least 95% identical to the nucleic acid sequence set forth in any one of SEQ. ID NOs:85-86. According to one embodiment, the method comprises detecting a polynucleotide comprising a nucleic acid sequence as set forth in any one of SEQ. ID NOs: 85-86.

Thus, according to another embodiment, the present invention provides a method for screening for lung cancer, ovarian cancer, breast cancer, and/or ovarian, breast or lung cancer invasion and metastasis in a subject, comprising detecting in the subject at least one polypeptide comprising an amino acid sequence as set forth in any one of SEQ. ID NOs: 121, 124.

With regard to lung cancer, the disease is selected from the group consisting of invasive or metastatic lung cancer; squamous cell lung carcinoma, lung adenocarcinoma, carcinoid, small cell lung cancer or non-small cell lung cancer. The method for screening lung cancer includes, but is not limited to, detection of overexpression in lung metastasis (vs. primary tumor); detection of overexpression in lung cancer, for example non small cell lung cancer, for example adenocarcinoma, squamous cell cancer or carcinoid, or large cell carcinoma; identification of a metastasis of unknown origin which originated from a primary lung cancer; assessment of a malignant tissue residing in the lung that is from a non-lung origin, including but not limited to: osteogenic and soft tissue sarcomas; colorectal, uterine, cervix and corpus tumors; head and neck, breast, testis and salivary gland cancers; melanoma; and bladder and kidney tumors; distinguishing between different types of lung cancer, therefore potentially affecting treatment choice (e.g. small cell vs. non small cell tumors); analysis of unexplained dyspnea and/or chronic cough and/or hemoptysis; differential diagnosis of the origin of a pleural effusion; diagnosis of conditions which have similar symptoms, signs and complications as lung cancer and where the differential diagnosis between them and lung cancer is of clinical importance including but not limited to: non-malignant causes of lung symptoms and signs, including but not limited to: lung lesions and infiltrates, wheeze, stridor, tracheal obstruction, esophageal compression, dysphagia, recurrent laryngeal nerve paralysis, hoarseness, phrenic nerve paralysis with elevation of the hemidiaphragm and Horner syndrome; or detecting a cause of any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

The polypeptides and/or polynucleotides of cluster F13779 and/or cluster T29749 used as markers for lung cancer can be used alone or in combination with one or more alternative polynucleotides or polypeptides described herein, and/or in combination with known markers for lung cancer, including but not limited to CEA, CA15-3, Beta-2-microglobulin, CA19-9, TPA, and/or in combination with the known protein(s) for the variant marker as described herein.

With regard to ovarian cancer, the polypeptides and/or polynucleotide of clusters F13779 and T29749 of the present invention can be used in the diagnosis, treatment or prognostic assessment of invasive or metastatic ovarian cancer; correlating stage and malignant potential; identification of a metastasis of unknown origin which originated from a primary ovarian cancer; differential diagnosis between benign and malignant ovarian cysts; diagnosing a cause of infertility, for example differential diagnosis of various causes thereof; detecting of one or more non-ovarian cancer conditions that may elevate serum levels of ovary related markers, including but not limited to: cancers of the endometrium, cervix, fallopian tubes, pancreas, breast, lung and colon; nonmalignant conditions such as pregnancy, endometriosis, pelvic inflammatory disease and uterine fibroids; diagnosing conditions which have similar symptoms, signs and complications as ovarian cancer and where the differential diagnosis between them and ovarian cancer is of clinical importance including but not limited to: non-malignant causes of pelvic mass, including, but not limited to: benign (functional) ovarian cyst, uterine fibroids, endometriosis, benign ovarian neoplasms and inflammatory bowel lesions; determining a cause of any condition suggestive of a malignant tumor including but not limited to anorexia, cachexia, weight loss, fever, hypercalcemia, skeletal or abdominal pain, paraneoplastic syndrome, or ascites.

The polypeptides and/or polynucleotides of cluster F13779 and T29749 used in the diagnosis, treatment or prognostic assessment of ovarian cancer can be used alone or in combination with one or more polypeptides and/or polynucleotides of this invention, and/or in combination with known markers for ovarian cancer, including but not limited to CEA, CA125 (Mucin 16), CA72-4TAG, CA-50, CA 54-61, CA-195 and CA 19-9 in combination with CA-125, and/or in combination with the known protein(s) associated with the indicated polypeptide or polynucleotide, as described herein.

With regard to breast cancer, the polypeptides and/or polynucleotides of cluster F13779 and T29749 are useful in determining a probable outcome in breast cancer; identification of a metastasis of unknown origin which originated from a primary breast cancer tumor; assessing lymphadenopathy, and in particular axillary lymphadenopathy; distinguishing between different types of breast cancer, therefore potentially affect treatment choice (e.g. as HER-2); differentially diagnosing between a benign and malignant breast mass; as a tool in the assessment of conditions affecting breast skin (e.g. Paget's disease) and their differentiation from breast cancer; differential diagnosis of breast pain or discomfort resulting from either breast cancer or other possible conditions (e.g. mastitis, Mondors syndrome); non-breast cancer conditions which have similar symptoms, signs and complications as breast cancer and where the differential diagnosis between them and breast cancer is of clinical importance including but not limited to: abnormal mammogram and/or nipple retraction and/or nipple discharge due to causes other than breast cancer, including but not limited to benign breast masses, melanoma, trauma and technical and/or anatomical variations; determining a cause of any condition suggestive of a malignant tumor including but not limited to anorexia, cachexia, weight loss, fever, hypercaleemia, paraneoplastic syndrome; or determining a cause of lymphadenopathy, weight loss and other signs and symptoms associated with breast cancer but originate from diseases different from breast cancer including but not limited to other malignancies, infections and autoimmune diseases.

Each variant marker of the present invention described herein as potential marker for breast cancer can be used alone or in combination with one or more other variant breast cancer described herein, and/or in combination with known markers for breast cancer, including but not limited to Calcitonin, CA15-3 (Mucin1), CA27-29, TPA, a combination of CA 15-3 and CEA, CA 27.29 (monoclonal antibody directed against MuC), Estrogen 2 (beta), HER-2 (c-erbB2), and/or in combination with the known protein(s) for the variant marker as described herein.

It is to be understood that any polynucleotide of this invention may be useful as a marker for a disease, disorder or condition, and such use is to be considered a part of this invention.

According to certain embodiments, detecting the expression of a polynucleotide or polypeptide according to the teaching of the present invention is performed by employing a NAT-based technology (optionally by employing at least one nucleotide probe or primer), or by employing an immunoassay (optionally by employing an antibody according to any of the embodiments described herein), respectively.

In some embodiments, this invention provides a method for screening for a disease in a subject, comprising detecting in the subject or in a sample obtained from said subject at least one polypeptide or polynucleotide selected from the group consisting of:
a. a polypeptide having an amino acid sequence as set forth in any one of SEQ. ID NOs: 74-77, 121, 124, or a homologue or a fragment thereof;
b. a polypeptide comprising a bridge, edge portion, tail, or head portion, of any one of SEQ. ID NOs: 128-133, or a homologue or a fragment thereof;
c. a polynucleotide having a nucleic acid sequence as set forth in any one of SEQ. ID NOs: 38-41, 85, 86, or a homologue or a fragment thereof;
d. a polynucleotide comprising a node having a nucleic acid sequence as set forth in any one of SEQ. ID NOs:42-70, 87-120;
e. an oligonucleotide having a nucleic acid sequence as set forth in SEQ. ID NOs: 37, 80, 83, 84, 127.

According to one embodiment, detecting the presence of the polypeptide or polynucleotide is indicative of the presence of the disease and/or its severity and/or its progress. According to another embodiment, a change in the expression and/or the level of the polynucleotide or polypeptide compared to its expression and/or level in a healthy subject or a sample obtained therefrom is indicative of the presence of the disease and/or its severity and/or its progress. According to a further embodiment, a change in the expression and/or level of the polynucleotide or polypeptide compared to its level and/or expression in said subject or in a sample obtained therefrom at earlier stage is indicative of the progress of the disease. According to still further embodiment, detecting the presence and/or relative change in the expression and/or level of the polynucleotide or polypeptide is useful for selecting a treatment and/or monitoring a treatment of the disease.

According to one embodiment, detecting a polynucleotide of the invention comprises employing a primer pair, comprising a pair of isolated oligonucleotides capable of specifically hybridizing to at least a portion of a polynucleotide having a nucleic acid sequence as set forth in SEQ. ID NOs:38-41, 85-86 or polynucleotides homologous thereto.

According to another embodiment, detecting a polynucleotide of the invention comprises employing a primer pair, comprising a pair of isolated oligonucleotides as set forth in SEQ. ID NOs:78-79, 81-82.

According to further embodiment, detecting a polypeptide of the invention comprises employing an antibody capable of specifically binding to at least one epitope of a polypeptide comprising an amino acid sequence as set forth in any one of SEQ. ID NOs: 74-77, 121, 124, or of a polypeptide comprising a bridge, edge portion, tail, or head portion of any one of SEQ. ID NOs: 128-133.

In some embodiments, a method of this invention may make use of a polynucleotide, polypeptide, vector, antibody, biomarker, or combination thereof, as described herein, including any combination thereof.

In some embodiments, the methods of this invention are conducted on a whole body. According to other embodiments, the methods of the present invention are conducted with a sample isolated from a subject having, predisposed to, or suspected of having the disease, disorder or condition. According to certain embodiments, the sample is a cell or tissue or a body fluid sample. In some embodiments, the methods are directed to the monitoring of disease progression and/or treatment efficacy and/or relapse of the indicated disease, disorder or condition.

In another embodiment, this invention provides methods for the selection of a particular therapy, or optimization of a given therapy for a disease, disorder or condition, the method comprising quantitatively and/or qualitatively determining or assessing expression of the polypeptides and/or polynucleotides, whereby differences in expression from an index sample, or a sample taken from a subject prior to the initiation of the therapy, or during the course of therapy, is indicative of the efficacy, or optimal activity of the therapy.

According to still further aspect, the present invention provides a method for detecting a splice variant nucleic acid sequence in a biological sample, comprising: hybridizing the isolated splice variant nucleic acid molecules or oligonucleotide fragments thereof of at least about 12 nucleotides to a nucleic acid material of the biological sample and detecting a hybridization complex; wherein the presence of the hybridization complex correlates with the presence of said splice variant nucleic acid sequence in the biological sample.

According to one aspect the present invention provides an isolated polynucleotide comprising a nucleic acid sequence set forth in a member selected from the group consisting of SEQ ID NOs:38-41, 85, 86, or a sequence at least about 95% identical thereto.

According to another aspect the present invention provides an isolated polynucleotide comprising a nucleic acid sequence as set forth in any one of SEQ ID NOs:42-70, 87-120.

According to another aspect the present invention provides an isolated polypeptide comprising an amino acid sequence set forth in a member selected from the group consisting of SEQ ID NOs:74-77, 121 and 124, or a sequence at least about 95% homologous thereto.

According to another aspect the present invention provides an isolated oligonucleotide comprising an amplicon selected from the group consisting of SEQ ID NOs:80, 83, 127.

According to another aspect the present invention provides a primer pair, comprising a pair of isolated oligonucleotides capable of amplifying said amplicon selected from the group consisting of SEQ ID NOs:80, 83, 127.

According to one embodiment the present invention provides the primer pair as above, comprising a pair of isolated oligonucleotides having a sequence selected from the group consisting of SEQ ID NOs:78 and 79; 81 and 82; 125 and 126.

According to another aspect the present invention provides a kit for detecting a disease, comprising a marker as set forth in any one of SEQ ID NOs: 38-41, 42-70, 80, 83, 85-120, 127, and a detecting agent for detecting said marker.

According to one embodiment the present invention provides the kit as above, wherein said detecting agent comprises at least one nucleotide probe or primer.

According to another embodiment the present invention provides the kit as above, wherein said detecting agent comprises at least one oligonucleotide capable of selectively hybridizing to a nucleic acid sequence as set forth in any one of SEQ ID NOs: 38-41, 42-70, 80, 83, 85-120, 127.

According to another embodiment the present invention provides the kit as above, wherein said oligonucleotide is selected from the group consisting of SEQ ID NOs: 37, 84.

According to another embodiment the present invention provides the kit as above, wherein said detecting agent comprises at least one primer pair capable of selectively amplifying a nucleic acid sequence as set forth in any one of SEQ ID NOs: 38-41, 42-70, 80, 83, 85-120, 127.

According to another embodiment the present invention provides the kit as above, wherein said primer pair is selected from the group consisting of SEQ ID NOs: 78, 79, 81, 82, 125, 126.

According to another embodiment the present invention provides the kit as above, wherein said kit further comprises at least one reagent for performing a NAT (nucleic acid amplification technology)-based assay.

According to another embodiment the present invention provides the kit as above, wherein said NAT-based assay is selected from the group consisting of a PCR, Real-Time PCR, LCR, Self-Sustained Synthetic Reaction, Q-Beta Replicase, Cycling probe reaction, Branched DNA, RFLP analysis, DGGE/TGGE, Single-Strand Conformation Polymorphism, Dideoxy fingerprinting, microarrays, Fluorescence In Situ Hybridization or Comparative Genomic Hybridization.

According to another aspect the present invention provides a kit for detecting a disease, comprising a marker as set forth in any one of SEQ ID NOs; 74-77, 121 and 124 and an antibody for detecting said marker.

According to one embodiment the present invention provides the kit as above, wherein said kit further comprises at least one reagent for performing an immunoassay.

According to another embodiment the present invention provides the kit as above, wherein said immunoassay is selected from the group consisting of an ELISA, a RIA, a slot blot, immunohistochemical assay, FACS, a radio-imaging assay or a Western blot.

According to another aspect the present invention provides a method for screening for a cancerous disease, disorder or condition in a subject, comprising detecting in the subject or in a sample obtained from said subject a polynucleotide having a sequence as set forth in any one of SEQ. ID NOs: 38-41, 42-70, 80, 83, 85-120, 127.

According to one embodiment the present invention provides the method as above, wherein screening for a disease comprises detecting the presence or severity of the disease, disorder or condition, or prognosis of the subject, or treatment selection for said subject, or treatment monitoring.

According to another embodiment the present invention provides the method as above, wherein the cancer is invasive or metastatic.

According to another embodiment the present invention provides the method as above, wherein said cancerous disease, disorder or condition comprises lung cancer and said polynucleotide has a sequence selected from the group consisting of SEQ ID NOs: 38-41, 42-70, 80, 83, 85-120, 127.

According to another embodiment the present invention provides the method as above, wherein said cancerous disease, disorder or condition comprises breast cancer and said polynucleotide has a sequence selected from the group consisting of SEQ ID NOs: 38-41, 42-70, 80, 83, 85-120, 127.

According to another embodiment the present invention provides the method as above, wherein said cancerous disease, disorder or condition comprises ovarian cancer and said polynucleotide has a sequence selected from the group consisting of SEQ ID NOs: 38-41, 42-70, 80, 83, 85-120, 127.

According to another embodiment the present invention provides the method as above, wherein screening for a disease comprises detecting the presence or severity of the disease, disorder or condition, or prognosis of the subject, or treatment selection for said subject, or treatment monitoring.

According to another aspect the present invention provides a method for screening for a disease, disorder or condition in a subject, comprising detecting a polypeptide having a sequence as set forth in SEQ ID NOs: 74-77, 121 and 124.

According to one embodiment the present invention provides the method as above, wherein screening for a disease comprises detecting the presence or severity of the disease, disorder or condition, or prognosis of the subject, or treatment selection for said subject, or treatment monitoring of said subject.

According to another embodiment the present invention provides the method as above, wherein the disease is lung cancer, breast cancer or ovarian cancer.

According to another embodiment the present invention provides the method as above, wherein said cancer is invasive or metastatic.

According to another embodiment the present invention provides the method as above, wherein said detecting is conducted by immunoassay.

According to another embodiment the present invention provides the method as above, wherein the immunoassay utilizes an antibody which specifically interacts with said polypeptide.

According to another embodiment the present invention provides the method as above, wherein said cancerous disease, disorder or condition comprises ovarian cancer and said polypeptide has a sequence selected from the group consisting of SEQ ID NOs:74-77, 121 and 124.

According to another embodiment the present invention provides the method as above, wherein said cancerous disease disorder or condition comprises lung cancer and said polypeptide has a sequence selected from the group consisting of SEQ ID NOs:74-77, 121 and 124.

According to another embodiment the present invention provides the method as above, wherein said cancerous disease, disorder or condition comprises breast cancer and said polypeptide has a sequence selected from the group consisting of SEQ ID NOs:74-77, 121 and 124.

According to another embodiment the present invention provides the method as above, wherein screening for a disease comprises detecting the presence or severity of the disease, disorder or condition, or prognosis of the subject, or treatment selection for said subject, or treatment monitoring of said subject.

According to another embodiment the present invention provides the method as above, wherein said detecting is conducted by immunoassay.

According to another embodiment the present invention provides the method as above, wherein the immunoassay utilizes an antibody which specifically interacts with said polypeptide.

The nucleic acid sequences and/or amino acid sequences shown herein as embodiments of the present invention relate, in some embodiments, to their isolated form, as isolated polynucleotides (including for all transcripts), oligonucleotides (including for all segments, amplicons and primers), peptides (including for all tails, bridges, insertions or heads, optionally including other antibody epitopes as described herein) and/or polypeptides (including for all proteins). It should be noted that the terms “oligonucleotide” and “polynucleotide” and “nucleic acid molecule”, or “peptide” and “polypeptide” and “protein”, may optionally be used interchangeably.

All technical and scientific terms used herein should be understood to have the meaning commonly understood by a person skilled in the art to which this invention belongs, as well as any other specified description. The following references provide one of skill in the art with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows a schematic description of the cancer biomarker selection engine.

FIG. 2 shows a schematic illustration, depicting grouping of transcripts of a given cluster based on presence or absence of unique sequence regions.

FIG. 3 shows a schematic summary of quantitative real-time PCR analysis.

FIG. 4 is schematic presentation of the oligonucleotide based microarray fabrication.

FIG. 5 is schematic summary of the oligonucleotide based microarray experimental flow.

FIG. 6 shows cancer and cell-line vs. normal tissue expression for cluster T29749, demonstrating overexpression (at least at a minimum level) in the following pathological conditions: brain malignant tumors, lung malignant tumors, pancreas carcinoma, hepatocellular carcinoma, a mixture of malignant tumors from different tissues, skin malignancies, myosarcoma, colorectal cancer, gastric carcinoma and epithelial malignant tumors.

FIG. 7 shows cancer and cell-line vs. normal tissue expression for cluster F13779 demonstrating overexpression (at least at a minimum level) in the following pathological conditions: brain malignant tumors, a mixture of malignant tumors from different tissues, skin malignancies and epithelial malignant tumors.

FIG. 8 is a histogram showing over expression of Homo sapiens ribonucleotide reductase M2 polypeptide (RRM2) T29749 transcripts which are detectable by amplicon as depicted in sequence name T29749_seg22-24WT (SEQ ID NO: 80) in normal and cancerous Lung tissues.

FIG. 9 is a histogram showing Expression of Homo sapiens ribonucleotide reductase M2 polypeptide (RRM2) T29749 transcripts which are detectable by amplicon as depicted in sequence name T29749_seg22-24WT (SEQ ID NO: 80) in different normal tissues.

FIG. 10 is a histogram showing Expression of Homo sapiens ribonucleotide reductase M2 polypeptide (RRM2) T29749 transcripts which are detectable by amplicon as depicted in sequence name T29749_seg29 (SEQ ID NO: 83) in normal and cancerous Breast tissues.

FIG. 11 is a histogram showing Expression of Homo sapiens ribonucleotide reductase M2 polypeptide (RRM2) T29749 transcripts which are detectable by amplicon as depicted in sequence name T29749_seg29 (SEQ ID NO: 83) in normal and cancerous Lung tissues.

FIG. 12 is a histogram showing over expression of Homo sapiens ribonucleotide reductase M2 polypeptide (RRM2) T29749 transcripts which are detectable by amplicon as depicted in sequence name T29749_seg29 (SEQ ID NO: 83) in cancerous ovary samples relative to the normal samples.

FIG. 13 demonstrates Expression of Homo sapiens ribonucleotide reductase M2 polypeptide (RRM2) T29749 transcripts which are detectable by amplicon as depicted in sequence name T29749_seg29 (SEQ ID NO: 83) in different normal tissues. FIG. 13A represents relative expression of each sample relative to median of the lung samples; FIG. 13B represents relative expression of each sample relative to median of the breast samples; and

FIG. 13C represents relative expression of each sample relative to median of the ovary samples.

FIG. 14 is a histogram showing over expression of the Homo sapiens hypothetical protein FLJ11029 (FLJ11029) F13779 transcripts which are detectable by amplicon as depicted in sequence name F13779_seg17-18 (SEQ ID NO: 127) in normal and cancerous breast tissues.

FIG. 15 is a histogram showing over expression of the Homo sapiens hypothetical protein FLJ11029 (FLJ11029) F13779 transcripts which are detectable by amplicon as depicted in sequence name F13779 seg17-18 (SEQ ID NO: 127) in normal and cancerous Lung tissues.

FIG. 16 is a histogram showing over expression of the Homo sapiens hypothetical protein FLJ11029 (FLJ11029) F13779 transcripts which are detectable by amplicon as depicted in sequence name F13779_seg17-18 (SEQ ID NO: 127) in normal and cancerous ovary tissues.

FIG. 17 presents a histogram showing over expression of the Homo sapiens hypothetical protein FLJ11029 (FLJ11029) F13779 transcripts which are detectable by amplicon as depicted in sequence name F13779_seg17-18 (SEQ ID NO: 127) in different normal tissues.

FIG. 17A presents the expression of each sample relative to median of the lung samples;

FIG. 17B presents the expression of each sample relative to median of the breast samples;

FIG. 17C presents the expression of each sample relative to median of the ovary samples.

### DESCRIPTION OF PREFERRED EMBODIMENTS

The present invention provides variants, which may optionally be used as diagnostic markers.

Preferably these variants are useful as diagnostic markers for certain diseases, and as such the term “marker-detectable” or “variant-detectable” with regard to a disease is to be understood as encompassing use of the described polynucleotides for diagnosis.

In some embodiments, certain diseases are associated with differential expression, qualitatively or quantitatively, of the polynucleotides of this invention. Assessment of such expression, in turn, can therefore serve as a marker for a particular disease state, susceptibility to a disease, pathogenesis, etc., including any desired disease-specific event, whose analysis is useful, as will be appreciated by one skilled in the art. In one embodiment, such use as a marker is also referred to herein as the polynucleotides being “variant disease markers”.

The markers of the present invention, alone or in combination, can be used for prognosis, prediction, screening, early diagnosis, staging, therapy selection and treatment monitoring of a marker-detectable disease. For example, optionally and preferably, these markers may be used for staging the disease in patients (for example if the disease features cancer) and/or monitoring the progression of the disease. Furthermore, the markers of the present invention, alone or in combination, can be used for detection of the source of metastasis found in anatomical places other than the originating tissue, again in the example of cancer. Also, one or more of the markers may optionally be used in combination with one or more other disease markers (other than those described herein).

Biomolecular sequences (nucleic acid sequences) uncovered using the methodology of the present invention and described herein can be efficiently utilized as tissue or pathological markers and/or as drugs or drug targets for treating or preventing a disease.

These markers are specifically released to the bloodstream under conditions of a particular disease, and/or are otherwise expressed at a much higher level and/or specifically expressed in tissue or cells afflicted with or demonstrating the disease. The measurement of these markers, alone or in combination, in patient samples provides information that the diagnostician can correlate with a probable diagnosis of a particular disease and/or a condition that is indicative of a higher risk for a particular disease.

The present invention provides, in some embodiments, diagnostic assays for a marker-detectable disease and/or an indicative condition, and methods of use of such markers for detection of marker-detectable disease and/or an indicative condition, for example in a sample taken from a subject (patient), which in some embodiments, is a blood sample.

Information is given in the text with regard to SNPs (single nucleotide polymorphisms). A description of the abbreviations is as follows. “T->C”, for example, means that the SNP results in a change at the position given in the table from T to C. Similarly, “M->Q”, for example, means that the SNP has caused a change in the corresponding amino acid sequence, from methionine (M) to glutamine (Q). If, in place of a letter at the right hand side for the nucleotide sequence SNP, there is a space, it indicates that a frameshift has occurred. A frameshift may also be indicated with a hyphen (-). A stop codon is indicated with an asterisk at the right hand side (*). As part of the description of an SNP, a comment may be found in parentheses after the above description of the SNP itself. This comment may include an FTId, which is an identifier to a SwissProt entry that was created with the indicated SNP. An FTId is a unique and stable feature identifier, which allows to construct links directly from position-specific annotation in the feature table to specialized protein-related databases. The FTId is always the last component of a feature in the description field, as follows: FTId=XXX_number, in which XXX is the 3-letter code for the specific feature key, separated by an underscore from a 6-digit number.

Information is given with regard to overexpression of a cluster in cancer based on ESTs. A key to the p values with regard to the analysis of such overexpression is as follows:
library-based statistics: P-value without including the level of expression in cell-lines (P1)
library based statistics: P-value including the level of expression in cell-lines (P2)
EST clone statistics: P-value without including the level of expression in cell-lines (SP1)
EST clone statistics: predicted overexpression ratio without including the level of expression in cell-lines (R3)
EST clone statistics: P-value including the level of expression in cell-lines (SP2)
EST clone statistics: predicted overexpression ratio including the level of expression in cell-lines (R4)

Library-based statistics refer to statistics over an entire library, while EST clone statistics refer to expression only for ESTs from a particular tissue or cancer.

Information is given with regard to overexpression of a cluster in cancer based on microarrays. As a microarray reference, in the specific segment paragraphs, the unabbreviated tissue name was used as the reference to the type of chip for which expression was measured. The microarray fabrication procedure is described in detail in Materials and Experimental Procedures section herein.

There are two types of microarray results: those from microarrays prepared according to a design by the present inventors, for which the microarray fabrication procedure is described in detail in Materials and Experimental Procedures section herein; and those results from microarrays using Affymetrix technology. As a microarray reference, in the specific segment paragraphs, the unabbreviated tissue name was used as the reference to the type of chip for which expression was measured. For microarrays prepared according to a design by the present inventors, the probe name begins with the name of the cluster (gene), followed by an identifying number. These probes are listed below with their respective sequences.

Oligonucleotide microarray results taken from Affymetrix data were from chips available from Affymetrix Inc, Santa Clara, Calif., USA (see for example data regarding the Human Genome U133 (HG-U133) Set at www.affymetrix.com/products/arrays/specific/hgul33.affx; GeneChip Human Genome U133A 2.0 Array at www.affymetrix.com/products/arrays/specific/hgu133av2.affx; and Human Genome U133 Plus 2.0 Array at www.affymetrix.com/products/arrays/specific/hgu133plus.affx). The probe names follow the Affymetrix naming convention. The data is available from NCBI Gene Expression Omnibus (see www.ncbi.nlm.nih.gov/projects/geo/ and Edgar et al, Nucleic Acids Research, 2002, Vol. 30, No. 1 207-210). The dataset (including results) is available from www.ncbi.nlm.nih.gov/geo/query/acc.cgi?acc=GSE1133 for the Series GSE1133 database (published on March 2004); a reference to these results is as follows: Su et al (Proc Natl Acad Sci USA. 2004 Apr. 20; 101(16):6062-7. Epub 2004 Apr. 09).

The following list of abbreviations for tissues was used in the TAA histograms. The term “TAA” stands for “Tumor Associated Antigen”, and the TAA histograms, given in the text, represent the cancerous tissue expression pattern as predicted by the biomarkers selection engine, as described in detail in examples 1-5 below:

“BONE” for “bone”;

“COL” for “colon”;

“EPI” for “epithelial”;

“GEN” for “general”;

“LIVER” for “liver”;

“LUN” for “lung”;

“LYMPH” for “lymph nodes”;

“MARROW” for “bone marrow”;

“OVA” for “ovary”;

“PANCREAS” for “pancreas”;

“PRO” for “prostate”;

“STOMACH” for “stomach”;

“TCELL” for “T cells”;

“THYROID” for “Thyroid”;

“MAM” for “breast”;

“BRAIN” for “brain”;

“UTERUS” for “uterus”;

“SKIN” for “skin”;

“KIDNEY” for “kidney”;

“MUSCLE” for “muscle”;

“ADREN” for “adrenal”;

“HEAD” for “head and neck”;

“BLADDER” for “bladder”;

It should be noted that the terms “segment”, “seg” and “node” are used interchangeably in reference to nucleic acid sequences of the present invention; they refer to portions of nucleic acid sequences that were shown to have one or more properties as described below. They are also the building blocks that were used to construct complete nucleic acid sequences as described in greater detail below. Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). All of these are hereby incorporated by reference as if fully set forth herein. As used herein, the following terms have the meanings ascribed to them unless specified otherwise.

As used herein the phrase “disease” includes any type of pathology and/or damage, including both chronic and acute damage, as well as a progress from acute to chronic damage.

The term “marker” in the context of the present invention refers to a nucleic acid fragment, a peptide, or a polypeptide, which is differentially present in a sample taken from patients (subjects) having one of the herein-described diseases or conditions, as compared to a comparable sample taken from subjects who do not have one the above-described diseases or conditions.

The phrase “differentially present” refers to differences in the quantity of a marker present in a sample taken from patients having one of the herein-described diseases or conditions as compared to a comparable sample taken from patients who do not have one of the herein-described diseases or conditions. For example, a nucleic acid fragment may optionally be differentially present between the two samples if the amount of the nucleic acid fragment in one sample is significantly different from the amount of the nucleic acid fragment in the other sample, for example as measured by hybridization and/or NAT-based assays. It should be noted that if the marker is detectable in one sample and not detectable in the other, then such a marker can be considered to be differentially present. Optionally, a relatively low amount of up-regulation may serve as the marker, as described herein. One of ordinary skill in the art could easily determine such relative levels of the markers; further guidance is provided in the description of each individual marker below.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above.

Diagnosis of a disease according to the present invention can be effected by determining a level of a polynucleotide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

As used herein, the term “level” refers to expression levels of RNA and/or to DNA copy number of a marker of the present invention.

Typically the level of the marker in a biological sample obtained from the subject is different (i.e., increased or decreased) from the level of the same variant in a similar sample obtained from a healthy individual (examples of biological samples are described herein).

Numerous well known tissue or fluid collection methods can be utilized to collect the biological sample from the subject in order to determine the level of DNA and/or RNA of the variant of interest in the subject.

Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

Determining the level of the same variant in normal tissues of the same origin is preferably effected along-side to detect an elevated expression and/or amplification and/or a decreased expression, of the variant as opposed to the normal tissues.

A “test amount” of a marker refers to an amount of a marker in a subject's sample that is consistent with a diagnosis of a particular disease or condition. A test amount can be either in absolute amount (e.g., microgram/ml) or a relative amount (e.g., relative intensity of signals).

A “control amount” of a marker can be any amount or a range of amounts to be compared against a test amount of a marker. For example, a control amount of a marker can be the amount of a marker in a patient with a particular disease or condition or a person without such a disease or condition. A control amount can be either in absolute amount (e.g., microgram/ml) or a relative amount (e.g., relative intensity of signals).

“Detect” refers to identifying the presence, absence or amount of the object to be detected.

A “detecting agent” includes any agent which can detect the presence of a polypeptide or polynucleotide according to the present invention, or a portion thereof, and includes without limitation antibodies (particularly for polypeptides or portions thereof), primers (for amplifying at least a portion of a polynucleotide), probes (for detecting a polypeptide and/or a polynucleotide, or a portion thereof) and hybridizable oligonucleotides which are capable of hybridizing to at least a portion of a polynucleotide. Non-limiting examples of these detecting agents are described herein. According to preferred embodiments of the present invention, such a detecting agent may optionally be used to detect a marker as described herein, for example optionally as part of a kit for detecting a disease.

A “label” includes any moiety or item detectable by spectroscopic, photo chemical, biochemical, immunochemical, or chemical means. For example, useful labels include 32P, 35S, fluorescent dyes, electron-dense reagents, enzymes (e.g., as commonly used in an ELISA), biotin-streptavadin, dioxigenin, haptens and proteins for which nucleic acid molecules with a sequence complementary to a target are available. The label often generates a measurable signal, such as a radioactive, chromogenic, or fluorescent signal, that can be used to quantify the amount of bound label in a sample. The label can be incorporated in or attached to a primer or probe either covalently, or through ionic, van der Waals or hydrogen bonds, e.g., incorporation of radioactive nucleotides, or biotinylated nucleotides that are recognized by streptavadin. The label may be directly or indirectly detectable. Indirect detection can involve the binding of a second label to the first label, directly or indirectly. For example, the label can be the ligand of a binding partner, such as biotin, which is a binding partner for streptavadin, or a nucleotide sequence, which is the binding partner for a complementary sequence, to which it can specifically hybridize. The binding partner may itself be directly detectable. The binding partner also may be indirectly detectable, for example, a nucleic acid having a complementary nucleotide sequence can be a part of a branched DNA molecule that is in turn detectable through hybridization with other labeled nucleic acid molecules (see, e.g., P. D. Fahrlander and A. Klausner, Bio/Technology 6:1165 (1988)). Quantitation of the signal is achieved by, e.g., scintillation counting, densitometry, or flow cytometry.

Exemplary detectable labels, optionally and preferably for use with immunoassays, include but are not limited to magnetic beads, fluorescent dyes, radiolabels, enzymes (e.g., horse radish peroxide, alkaline phosphatase and others commonly used in an ELISA), and calorimetric labels such as colloidal gold or colored glass or plastic beads. Alternatively, the marker in the sample can be detected using an indirect assay, and/or in a competition or inhibition assay wherein, for example, a monoclonal antibody which binds to a distinct epitope of the marker are incubated simultaneously with the mixture.

In another embodiment, this invention provides an isolated nucleic acid molecule encoding for a splice variant according to the present invention, having a nucleotide sequence as set forth in any one of the sequences listed herein, or a sequence complementary thereto. In another embodiment, this invention provides an isolated nucleic acid molecule, having a nucleotide sequence as set forth in any one of the sequences listed herein, or a sequence complementary thereto. In another embodiment, this invention provides an oligonucleotide of at least about 12 nucleotides, specifically hybridizable with the nucleic acid molecules of this invention. In another embodiment, this invention provides vectors, cells, liposomes and compositions comprising the isolated nucleic acids of this invention.

In another embodiment, this invention provides a method for detecting a splice variant nucleic acid sequences in a biological sample, comprising: hybridizing the isolated nucleic acid molecules or oligonucleotide fragments of at least about a minimum length to a nucleic acid material of a biological sample and detecting a hybridization complex; wherein the presence of a hybridization complex correlates with the presence of a splice variant nucleic acid sequence in the biological sample.

In some embodiments of the present invention, the splice variants described herein are non-limiting examples of markers for diagnosing marker-detectable disease and/or an indicative condition. Each splice variant marker of the present invention can be used alone or in combination, for various uses, including but not limited to, prognosis, prediction, screening, early diagnosis, determination of progression, therapy selection and treatment monitoring of marker-detectable disease and/or an indicative condition, including a transition from an indicative condition to marker-detectable disease.

According to some embodiments of the present invention, any marker according to the present invention may optionally be used alone or combination. Such a combination may optionally comprise a plurality of markers described herein, optionally including any subcombination of markers, and/or a combination featuring at least one other marker, for example a known marker. Furthermore, such a combination may optionally and preferably be used as described above with regard to determining a ratio between a quantitative or semi-quantitative measurement of any marker described herein to any other marker described herein, and/or any other known marker, and/or any other marker. With regard to such a ratio between any marker described herein (or a combination thereof) and a known marker, more preferably the known marker comprises the “known protein” as described in greater detail below with regard to each cluster or gene.

In some embodiments of the present invention, there are provided of methods, uses, devices and assays for the diagnosis of a disease or condition. Optionally a plurality of biomarkers (or markers) may be used with the present invention. The plurality of markers may optionally include a plurality of markers described herein, and/or one or more known markers. The plurality of markers is preferably then correlated with the disease or condition. For example, such correlating may optionally comprise determining the concentration of each of the plurality of markers, and individually comparing each marker concentration to a threshold level. Optionally, if the marker concentration is above or below the threshold level (depending upon the marker and/or the diagnostic test being performed), the marker concentration correlates with the disease or condition. Optionally and preferably, a plurality of marker concentrations correlates with the disease or condition.

Alternatively, such correlating may optionally comprise determining the concentration of each of the plurality of markers, calculating a single index value based on the concentration of each of the plurality of markers, and comparing the index value to a threshold level.

Also alternatively, such correlating may optionally comprise determining a temporal change in at least one of the markers, and wherein the temporal change is used in the correlating step.

Also alternatively, such correlating may optionally comprise determining whether at least “X” number of the plurality of markers has a concentration outside of a predetermined range and/or above or below a threshold (as described above). The value of “X” may optionally be one marker, a plurality of markers or all of the markers; alternatively or additionally, rather than including any marker in the count for “X”, one or more specific markers of the plurality of markers may optionally be required to correlate with the disease or condition (according to a range and/or threshold).

Also alternatively, such correlating may optionally comprise determining whether a ratio of marker concentrations for two markers is outside a range and/or above or below a threshold. Optionally, if the ratio is above or below the threshold level and/or outside a range, the ratio correlates with the disease or condition.

Optionally, a combination of two or more these correlations may be used with a single panel and/or for correlating between a plurality of panels.

Optionally, the method distinguishes a disease or condition with a sensitivity of at least 70% at a specificity of at least 85% when compared to normal subjects. As used herein, sensitivity relates to the number of positive (diseased) samples detected out of the total number of positive samples present; specificity relates to the number of true negative (non-diseased) samples detected out of the total number of negative samples present. Preferably, the method distinguishes a disease or condition with a sensitivity of at least 80% at a specificity of at least 90% when compared to normal subjects. More preferably, the method distinguishes a disease or condition with a sensitivity of at least 90% at a specificity of at least 90% when compared to normal subjects. Also more preferably, the method distinguishes a disease or condition with a sensitivity of at least 70% at a specificity of at least 85% when compared to subjects exhibiting symptoms that mimic disease or condition symptoms.

A marker panel may be analyzed in a number of fashions well known to those of skill in the art. For example, each member of a panel may be compared to a “normal” value, or a value indicating a particular outcome. A particular diagnosis/prognosis may depend upon the comparison of each marker to this value; alternatively, if only a subset of markers are outside of a normal range, this subset may be indicative of a particular diagnosis/prognosis. The skilled artisan will also understand that diagnostic markers, differential diagnostic markers, prognostic markers, time of onset markers, disease or condition differentiating markers, etc., may be combined in a single assay or device. Markers may also be commonly used for multiple purposes by, for example, applying a different threshold or a different weighting factor to the marker for the different purpose(s).

In one embodiment, the panels comprise markers for the following purposes: diagnosis of a disease; diagnosis of disease and indication if the disease is in an acute phase and/or if an acute attack of the disease has occurred; diagnosis of disease and indication if the disease is in a non-acute phase and/or if a non-acute attack of the disease has occurred; indication whether a combination of acute and non-acute phases or attacks has occurred; diagnosis of a disease and prognosis of a subsequent adverse outcome; diagnosis of a disease and prognosis of a subsequent acute or non-acute phase or attack; disease progression (for example for cancer, such progression may include for example occurrence or recurrence of metastasis).

The above diagnoses may also optionally include differential diagnosis of the disease to distinguish it from other diseases, including those diseases that may feature one or more similar or identical symptoms.

In certain embodiments, one or more diagnostic or prognostic indicators are correlated to a condition or disease by merely the presence or absence of the indicator(s). In other embodiments, threshold level(s) of a diagnostic or prognostic indicator(s) can be established, and the level of the indicator(s) in a patient sample can simply be compared to the threshold level(s). The sensitivity and specificity of a diagnostic and/or prognostic test depends on more than just the analytical “quality” of the test—they also depend on the definition of what constitutes an abnormal result. In practice, Receiver Operating Characteristic curves, or “ROC” curves, are typically calculated by plotting the value of a variable versus its relative frequency in “normal” and “disease” populations, and/or by comparison of results from a subject before, during and/or after treatment. For any particular marker, a distribution of marker levels for subjects with and without a disease will likely overlap. Under such conditions, a test does not absolutely distinguish normal from disease with 100% accuracy, and the area of overlap indicates where the test cannot distinguish normal from disease. A threshold is selected, above which (or below which, depending on how a marker changes with the disease) the test is considered to be abnormal and below which the test is considered to be normal. The area under the ROC curve is a measure of the probability that the perceived measurement will allow correct identification of a condition.

The horizontal axis of the ROC curve represents (1-specificity), which increases with the rate of false positives. The vertical axis of the curve represents sensitivity, which increases with the rate of true positives. Thus, for a particular cutoff selected, the value of (1-specificity) may be determined, and a corresponding sensitivity may be obtained. The area under the ROC curve is a measure of the probability that the measured marker level will allow correct identification of a disease or condition. Thus, the area under the ROC curve can be used to determine the effectiveness of the test.

ROC curves can be used even when test results don't necessarily give an accurate number. As long as one can rank results, one can create an ROC curve. For example, results of a test on “disease” samples might be ranked according to degree (say 1=low, 2=normal, and 3=high). This ranking can be correlated to results in the “normal” population, and a ROC curve created. These methods are well known in the art (see for example Hanley et al., Radiology 143: 29-36 (1982), incorporated by reference as if fully set forth herein).

One or more markers may lack diagnostic or prognostic value when considered alone, but when used as part of a panel, such markers may be of great value in determining a particular diagnosis/prognosis. In preferred embodiments, particular thresholds for one or more markers in a panel are not relied upon to determine if a profile of marker levels obtained from a subject are indicative of a particular diagnosis/prognosis. Rather, the present invention may utilize an evaluation of the entire marker profile by plotting ROC curves for the sensitivity of a particular panel of markers versus 1-(specificity) for the panel at various cutoffs. In these methods, a profile of marker measurements from a subject is considered together to provide a global probability (expressed either as a numeric score or as a percentage risk) that an individual has had a disease, is at risk for developing such a disease, optionally the type of disease which the individual has had or is at risk for, and so forth etc. In such embodiments, an increase in a certain subset of markers may be sufficient to indicate a particular diagnosis/prognosis in one patient, while an increase in a different subset of markers may be sufficient to indicate the same or a different diagnosis/prognosis in another patient. Weighting factors may also be applied to one or more markers in a panel, for example, when a marker is of particularly high utility in identifying a particular diagnosis/prognosis, it may be weighted so that at a given level it alone is sufficient to signal a positive result. Likewise, a weighting factor may provide that no given level of a particular marker is sufficient to signal a positive result, but only signals a result when another marker also contributes to the analysis.

In some embodiments, markers and/or marker panels are selected to exhibit at least 70% sensitivity, more preferably at least 80% sensitivity, even more preferably at least 85% sensitivity, still more preferably at least 90% sensitivity, and most preferably at least 95% sensitivity, combined with at least 70% specificity, more preferably at least 80% specificity, even more preferably at least 85% specificity, still more preferably at least 90% specificity, and most preferably at least 95% specificity. In particularly preferred embodiments, both the sensitivity and specificity are at least 75%, more preferably at least 80%, even more preferably at least 85%, still more preferably at least 90%, and most preferably at least 95%. Sensitivity and/or specificity may optionally be determined as described above, with regard to the construction of ROC graphs and so forth, for example.

According to preferred embodiments of the present invention, individual markers and/or combinations (panels) of markers may optionally be used for diagnosis of time of onset of a disease or condition. Such diagnosis may optionally be useful for a wide variety of conditions, preferably including those conditions with an abrupt onset.

The phrase “determining the prognosis” as used herein refers to methods by which the skilled artisan can predict the course or outcome of a condition in a patient. The term “prognosis” does not refer to the ability to predict the course or outcome of a condition with 100% accuracy, or even that a given course or outcome is more likely to occur than not. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. For example, in individuals not exhibiting the condition, the chance of a given outcome may be about 3%. In preferred embodiments, a prognosis is about a 5% chance of a given outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, and about a 95% chance. The term “about” in this context refers to +/−1%.

The skilled artisan will understand that associating a prognostic indicator with a predisposition to an adverse outcome is a statistical analysis. For example, a marker level of greater than 80 pg/mL may signal that a patient is more likely to suffer from an adverse outcome than patients with a level less than or equal to 80 pg/mL, as determined by a level of statistical significance. Additionally, a change in marker concentration from baseline levels may be reflective of patient prognosis, and the degree of change in marker level may be related to the severity of adverse events. Statistical significance is often determined by comparing two or more populations, and determining a confidence interval and/or a p value. See, e.g., Dowdy and Wearden, Statistics for Research, John Wiley & Sons, New York, 1983. Preferred confidence intervals of the invention are 90%, 95%, 97.5%, 98%, 99%, 99.5%, 99.9% and 99.99%, while preferred p values are 0.1, 0.05, 0.025, 0.02, 0.01, 0.005, 0.001, and 0.0001. Exemplary statistical tests for associating a prognostic indicator with a predisposition to an adverse outcome are described hereinafter.

In other embodiments, a threshold degree of change in the level of a prognostic or diagnostic indicator can be established, and the degree of change in the level of the indicator in a patient sample can simply be compared to the threshold degree of change in the level. A preferred threshold change in the level for markers of the invention is about 5%, about 10%, about 15%, about 20%, about 25%, about 30%, about 50%, about 75%, about 100%, and about 150%. The term “about” in this context refers to +/−10%. In yet other embodiments, a “nomogram” can be established, by which a level of a prognostic or diagnostic indicator can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

Exemplary, non-limiting methods and systems for identification of suitable biomarkers for marker panels are now described. Methods and systems for the identification of one or more markers for the diagnosis, and in particular for the differential diagnosis, of disease have been described previously. Suitable methods for identifying markers useful for the diagnosis of disease states are described in detail in U.S. patent application no. 2004-0126767, entitled METHOD AND SYSTEM FOR DISEASE DETECTION USING MARKER COMBINATIONS, filed Dec. 27, 2002, hereby incorporated by reference in its entirety as if fully set forth herein. One skilled in the art will also recognize that univariate analysis of markers can be performed and the data from the univariate analyses of multiple markers can be combined to form panels of markers to differentiate different disease conditions.

In developing a panel of markers useful in diagnosis, data for a number of potential markers may be obtained from a group of subjects by testing for the presence or level of certain markers. The group of subjects is divided into two sets, and preferably the first set and the second set each have an approximately equal number of subjects. The first set includes subjects who have been confirmed as having a disease or, more generally, being in a first condition state. For example, this first set of patients may be those that have recently had a disease and/or a particular type of the disease. The confirmation of this condition state may be made through more rigorous and/or expensive testing, preferably according to a previously defined diagnostic standard. Hereinafter, subjects in this first set will be referred to as “diseased”.

The second set of subjects are simply those who do not fall within the first set. Subjects in this second set may be “non-diseased;” that is, normal subjects. Alternatively, subjects in this second set may be selected to exhibit one symptom or a constellation of symptoms that mimic those symptoms exhibited by the “diseased” subjects.

The data obtained from subjects in these sets includes levels of a plurality of markers. Preferably, data for the same set of markers is available for each patient. This set of markers may include all candidate markers which may be suspected as being relevant to the detection of a particular disease or condition. Actual known relevance is not required. Embodiments of the methods and systems described herein may be used to determine which of the candidate markers are most relevant to the diagnosis of the disease or condition. The levels of each marker in the two sets of subjects may be distributed across a broad range, e.g., as a Gaussian distribution. However, no distribution fit is required.

As noted above, a marker often is incapable of definitively identifying a patient as either diseased or non-diseased. For example, if a patient is measured as having a marker level that falls within the overlapping region, the results of the test will be useless in diagnosing the patient. An artificial cutoff may be used to distinguish between a positive and a negative test result for the detection of the disease or condition. Regardless of where the cutoff is selected, the effectiveness of the single marker as a diagnosis tool is unaffected. Changing the cutoff merely trades off between the number of false positives and the number of false negatives resulting from the use of the single marker. The effectiveness of a test having such an overlap is often expressed using a ROC (Receiver Operating Characteristic) curve as described above.

As discussed above, the measurement of the level of a single marker may have limited usefulness. The measurement of additional markers provides additional information, but the difficulty lies in properly combining the levels of two potentially unrelated measurements. In the methods and systems according to embodiments of the present invention, data relating to levels of various markers for the sets of diseased and non-diseased patients may be used to develop a panel of markers to provide a useful panel response. The data may be provided in a database such as Microsoft Access, Oracle, other SQL databases or simply in a data file. The database or data file may contain, for example, a patient identifier such as a name or number, the levels of the various markers present, and whether the patient is diseased or non-diseased.

Next, an artificial cutoff region may be initially selected for each marker. The location of the cutoff region may initially be selected at any point, but the selection may affect the optimization process described below. In this regard, selection near a suspected optimal location may facilitate faster convergence of the optimizer. In a preferred method, the cutoff region is initially centered about the center of the overlap region of the two sets of patients. In one embodiment, the cutoff region may simply be a cutoff point. In other embodiments, the cutoff region may have a length of greater than zero. In this regard, the cutoff region may be defined by a center value and a magnitude of length. In practice, the initial selection of the limits of the cutoff region may be determined according to a pre-selected percentile of each set of subjects. For example, a point above which a pre-selected percentile of diseased patients are measured may be used as the right (upper) end of the cutoff range.

Each marker value for each patient may then be mapped to an indicator. The indicator is assigned one value below the cutoff region and another value above the cutoff region. For example, if a marker generally has a lower value for non-diseased patients and a higher value for diseased patients, a zero indicator will be assigned to a low value for a particular marker, indicating a potentially low likelihood of a positive diagnosis. In other embodiments, the indicator may be calculated based on a polynomial. The coefficients of the polynomial may be determined based on the distributions of the marker values among the diseased and non-diseased subjects.

The relative importance of the various markers may be indicated by a weighting factor. The weighting factor may initially be assigned as a coefficient for each marker. As with the cutoff region, the initial selection of the weighting factor may be selected at any acceptable value, but the selection may affect the optimization process. In this regard, selection near a suspected optimal location may facilitate faster convergence of the optimizer. In a preferred method, acceptable weighting coefficients may range between zero and one, and an initial weighting coefficient for each marker may be assigned as 0.5. In a preferred embodiment, the initial weighting coefficient for each marker may be associated with the effectiveness of that marker by itself For example, a ROC curve may be generated for the single marker, and the area under the ROC curve may be used as the initial weighting coefficient for that marker.

Next, a panel response may be calculated for each subject in each of the two sets. The panel response is a function of the indicators to which each marker level is mapped and the weighting coefficients for each marker. One advantage of using an indicator value rather than the marker value is that an extraordinarily high or low marker levels do not change the probability of a diagnosis of diseased or non-diseased for that particular marker. Typically, a marker value above a certain level generally indicates a certain condition state. Marker values above that level indicate the condition state with the same certainty. Thus, an extraordinarily high marker value may not indicate an extraordinarily high probability of that condition state. The use of an indicator which is constant on one side of the cutoff region eliminates this concern.

The panel response may also be a general function of several parameters including the marker levels and other factors including, for example, race and gender of the patient. Other factors contributing to the panel response may include the slope of the value of a particular marker over time. For example, a patient may be measured when first arriving at the hospital for a particular marker. The same marker may be measured again an hour later, and the level of change may be reflected in the panel response. Further, additional markers may be derived from other markers and may contribute to the value of the panel response. For example, the ratio of values of two markers may be a factor in calculating the panel response.

Having obtained panel responses for each subject in each set of subjects, the distribution of the panel responses for each set may now be analyzed. An objective function may be defined to facilitate the selection of an effective panel. The objective function should generally be indicative of the effectiveness of the panel, as may be expressed by, for example, overlap of the panel responses of the diseased set of subjects and the panel responses of the non-diseased set of subjects. In this manner, the objective function may be optimized to maximize the effectiveness of the panel by, for example, minimizing the overlap.

In some embodiment, the ROC curve representing the panel responses of the two sets of subjects may be used to define the objective function. For example, the objective function may reflect the area under the ROC curve. By maximizing the area under the curve, one may maximize the effectiveness of the panel of markers. In other embodiments, other features of the ROC curve may be used to define the objective function. For example, the point at which the slope of the ROC curve is equal to one may be a useful feature. In other embodiments, the point at which the product of sensitivity and specificity is a maximum, sometimes referred to as the “knee,” may be used. In an embodiment, the sensitivity at the knee may be maximized. In further embodiments, the sensitivity at a predetermined specificity level may be used to define the objective function. Other embodiments may use the specificity at a predetermined sensitivity level may be used. In still other embodiments, combinations of two or more of these ROC-curve features may be used.

It is possible that one of the markers in the panel is specific to the disease or condition being diagnosed. When such markers are present at above or below a certain threshold, the panel response may be set to return a “positive” test result. When the threshold is not satisfied, however, the levels of the marker may nevertheless be used as possible contributors to the objective function.

An optimization algorithm may be used to maximize or minimize the objective function. Optimization algorithms are well-known to those skilled in the art and include several commonly available minimizing or maximizing functions including the Simplex method and other constrained optimization techniques. It is understood by those skilled in the art that some minimization functions are better than others at searching for global minimums, rather than local minimums. In the optimization process, the location and size of the cutoff region for each marker may be allowed to vary to provide at least two degrees of freedom per marker. Such variable parameters are referred to herein as independent variables. In a preferred embodiment, the weighting coefficient for each marker is also allowed to vary across iterations of the optimization algorithm. In various embodiments, any permutation of these parameters may be used as independent variables.

In addition to the above-described parameters, the sense of each marker may also be used as an independent variable. For example, in many cases, it may not be known whether a higher level for a certain marker is generally indicative of a diseased state or a non-diseased state. In such a case, it may be useful to allow the optimization process to search on both sides. In practice, this may be implemented in several ways. For example, in one embodiment, the sense may be a truly separate independent variable which may be flipped between positive and negative by the optimization process. Alternatively, the sense may be implemented by allowing the weighting coefficient to be negative.

The optimization algorithm may be provided with certain constraints as well. For example, the resulting ROC curve may be constrained to provide an area-under-curve of greater than a particular value. ROC curves having an area under the curve of 0.5 indicate complete randomness, while an area under the curve of 1.0 reflects perfect separation of the two sets. Thus, a minimum acceptable value, such as 0.75, may be used as a constraint, particularly if the objective function does not incorporate the area under the curve. Other constraints may include limitations on the weighting coefficients of particular markers. Additional constraints may limit the sum of all the weighting coefficients to a particular value, such as 1.0.

The iterations of the optimization algorithm generally vary the independent parameters to satisfy the constraints while minimizing or maximizing the objective function. The number of iterations may be limited in the optimization process. Further, the optimization process may be terminated when the difference in the objective function between two consecutive iterations is below a predetermined threshold, thereby indicating that the optimization algorithm has reached a region of a local minimum or a maximum.

Thus, the optimization process may provide a panel of markers including weighting coefficients for each marker and cutoff regions for the mapping of marker values to indicators. In order to develop lower-cost panels which require the measurement of fewer marker levels, certain markers may be eliminated from the panel. In this regard, the effective contribution of each marker in the panel may be determined to identify the relative importance of the markers. In one embodiment, the weighting coefficients resulting from the optimization process may be used to determine the relative importance of each marker. The markers with the lowest coefficients may be eliminated.

Individual panel response values may also be used as markers in the methods described herein. For example, a panel may be constructed from a plurality of markers, and each marker of the panel may be described by a function and a weighting factor to be applied to that marker (as determined by the methods described above). Each individual marker level is determined for a sample to be tested, and that level is applied to the predetermined function and weighting factor for that particular marker to arrive at a sample value for that marker. The sample values for each marker are added together to arrive at the panel response for that particular sample to be tested. For a “diseased” and “non-diseased” group of patients, the resulting panel responses may be treated as if they were just levels of another disease marker.

Measures of test accuracy may be obtained as described in Fischer et al., Intensive Care Med. 29: 1043-51, 2003 (hereby incorporated by reference as if fully set forth herein), and used to determine the effectiveness of a given marker or panel of markers. These measures include sensitivity and specificity, predictive values, likelihood ratios, diagnostic odds ratios, and ROC curve areas. As discussed above, suitable tests may exhibit one or more of the following results on these various measures: at least 75% sensitivity, combined with at least 75% specificity; ROC curve area of at least 0.7, more preferably at least 0.8, even more preferably at least 0.9, and most preferably at least 0.95; and/or a positive likelihood ratio (calculated as sensitivityl(1-specificity)) of at least 5, more preferably at least 10, and most preferably at least 20, and a negative likelihood ratio (calculated as (1-sensitivity)/specificity) of less than or equal to 0.3, more preferably less than or equal to 0.2, and most preferably less than or equal to 0.1.

According to other preferred embodiments of the present invention, a splice variant nucleic acid sequence or a fragment thereof may be featured as a biomarker for detecting marker-detectable disease and/or an indicative condition, such that a biomarker may optionally comprise any of the above.

The present invention also optionally and preferably encompasses any nucleic acid sequence or fragment thereof corresponding to a splice variant of the present invention as described above, optionally for any application.

Non-limiting examples of methods or assays are described below.

The present invention also relates to kits based upon such diagnostic methods or assays.

### Nucleic Acid Sequences and Oligonucleotides

Various embodiments of the present invention encompass nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or artificially induced, either randomly or in a targeted fashion.

The present invention encompasses nucleic acid sequences described herein; fragments thereof sequences hybridizable therewith, sequences homologous thereto [e.g., at least 50%, at least 55%, at least 60%, at least 65%, at least 70%, at least 75%, at least 80%, at least 85%, at least 95% or more say 100% identical to the nucleic acid sequences set forth below], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention) which include sequence regions unique to the polynucleotides of the present invention.

In cases where the polynucleotide sequences of the present invention encode previously unidentified polypeptides, the present invention also encompasses novel polypeptides or portions thereof, which are encoded by the isolated polynucleotide and respective nucleic acid fragments thereof described hereinabove.

A “nucleic acid fragment” or an “oligonucleotide” or a “polynucleotide” are used herein interchangeably to refer to a polymer of nucleic acids. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is composed of genomic and cDNA sequences. A composite sequence can include some exonal sequences required to encode a polypeptide, as well as some intronic sequences interposing therebetween. The intronic sequences can be of any source, including of other genes, and typically will include conserved splicing signal sequences. Such intronic sequences may further include cis acting expression regulatory elements.

Preferred embodiments of the present invention encompass oligonucleotide probes.

An example of an oligonucleotide probe which can be utilized by the present invention is a single stranded polynucleotide which includes a sequence complementary to the unique sequence region of any variant according to the present invention, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Alternatively, an oligonucleotide probe of the present invention can be designed to hybridize with a nucleic acid sequence encompassed by any of the above nucleic acid sequences, particularly the portions specified above, including but not limited to a nucleotide sequence coding for an amino sequence of a bridge, tail, head and/or insertion according to the present invention, and/or the equivalent portions of any nucleotide sequence given herein (including but not limited to a nucleotide sequence of a node, segment or amplicon described herein).

Oligonucleotides designed according to the teachings of the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

Oligonucleotides used according to this aspect of the present invention are those having a length selected from a range of about 10 to about 200 bases preferably about 15 to about 150 bases, more preferably about 20 to about 100 bases, most preferably about 20 to about 50 bases. Preferably, the oligonucleotide of the present invention features at least 17, at least 18, at least 19, at least 20, at least 22, at least 25, at least 30 or at least 40, bases specifically hybridizable with the biomarkers of the present invention.

The oligonucleotides of the present invention may comprise heterocylic nucleosides consisting of purines and the pyrimidines bases, bonded in a 3′ to 5′ phosphodiester linkage.

Preferably used oligonucleotides are those modified at one or more of the backbone, internucleoside linkages or bases, as is broadly described hereinunder.

Specific examples of preferred oligonucleotides useful according to this aspect of the present invention include oligonucleotides containing modified backbones or non-natural internucleoside linkages. Oligonucleotides having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No. 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases particularly useful for increasing the binding affinity of the oligomeric compounds of the invention include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

Another modification of the oligonucleotides of the invention involves chemically linking to the oligonucleotide one or more moieties or conjugates, which enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety, as disclosed in U.S. Pat. No. 6,303,374.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that oligonucleotides of the present invention may include further modifications for more efficient use as diagnostic agents and/or to increase bioavailability, therapeutic efficacy and reduce cytotoxicity.

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element”¹ refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific, lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., includingRetro-X vectors pLNCX and pLXSN, which permit cloning into multiple cloning sites and the trasgene is transcribed from CMV promoter. Vectors derived from Mo-MuLV are also included such as pbabe, where the transgene will be transcribed from the 5′ LTR promoter.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al, Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

### Variant Recombinant Expression Vectors and Host Cells

Another aspect of the invention pertains to vectors, preferably expression vectors, containing a nucleic acid encoding a variant protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors arc capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., variant proteins, mutant forms of variant proteins, fusion proteins, etc.).

The recombinant expression vectors of the invention can be designed for production of variant proteins in prokaryotic or eukaryotic cells. For example, variant proteins can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or carboxyl terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) and pTrcHis (Invitrogen Life Technologies) that fuse glutathione S-transferase (GST), maltose E binding protein, protein A or 6×His, respectively, to the target recombinant protein.

Examples of suitable inducible non-fusion E. coli expression vectors include pTrc (Amrann et al., (1988) Gene 69:301-315).

One strategy to maximize recombinant protein expression in E. coli is to express the protein in host bacteria with an impaired capacity to proteolytically cleave the recombinant protein. See, e.g., Gottesman, Gene Expression Technology: Methods in Enzymology 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques. Another optional strategy to solve codon bias is by using BL21-codon plus bacterial strains (Invitrogen) or Rosetta bacterial strain (Novagen), as these strains contain extra copies of rare E. coli tRNA genes.

In another embodiment, the expression vector encoding for the variant protein is a yeast expression vector. Examples of vectors for expression in yeast Saccharomyces cerivisae include pYepSec1 (Baldari, et al., 1987. EMBO J. 6: 229-234), pea (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, variant protein can be produced in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195), pIRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory Manual. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Carnpes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to mRNA encoding for variant protein. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, variant protein can be produced in bacterial cells such as E. coli, insect cells, yeast or mammalian cells (such as Chinese hamster ovary cells (CHO) or COS or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, puromycin, blasticidin and methotrexate. Nucleic acids encoding a selectable marker can be introduced into a host cell on the same vector as that encoding variant protein or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) variant protein. Accordingly, the invention further provides methods for producing variant protein using the host cells of the invention. In one embodiment, the method comprises culturing the host cell of the present invention (into which a recombinant expression vector encoding variant protein has been introduced) in a suitable medium such that variant protein is produced. In another embodiment, the method further comprises isolating variant protein from the medium or the host cell.

For efficient production of the protein, it is preferable to place the nucleotide sequences encoding the variant protein under the control of expression control sequences optimized for expression in a desired host. For example, the sequences may include optimized transcriptional and/or translational regulatory sequences (such as altered Kozak sequences).

### Hybridization Assays

Detection of a nucleic acid of interest in a biological sample may optionally be effected by hybridization-based assays using an oligonucleotide probe (non-limiting examples of probes according to the present invention were previously described).

Traditional hybridization assays include PCR, RT-PCR, Real-time PCR, RNase protection, in-situ hybridization, primer extension, Southern blots (DNA detection), dot or slot blots (DNA, RNA), and Northern blots (RNA detection) (NAT type assays are described in greater detail below). More recently, PNAs have been described (Nielsen et al. 1999, Current Opin. Biotechnol. 10:71-75). Other detection methods include kits containing probes on a dipstick setup and the like.

Hybridization based assays which allow the detection of a variant of interest (i.e., DNA or RNA) in a biological sample rely on the use of oligonucleotides which can be 10, 15, 20, or 30 to 100 nucleotides long preferably from 10 to 50, more preferably from 40 to 50 nucleotides long.

Thus, the isolated polynucleotides (oligonucleotides) of the present invention are preferably hybridizable with any of the herein described nucleic acid sequences under moderate to stringent hybridization conditions.

Moderate to stringent hybridization conditions are characterized by a hybridization solution such as containing 10% dextrane sulfate, 1 M NaCl, 1% SDS and 5×10⁶ cpm ³²P labeled probe, at 65° C., with a final wash solution of 0.2×SSC and 0.1% SDS and final wash at 65° C. and whereas moderate hybridization is effected using a hybridization solution containing 10% dextrane sulfate, 1 M NaCl, 1% SDS and 5×10⁶ cpm ³²P labeled probe, at 65° C., with a final wash solution of 1×SSC and 0.1% SDS and final wash at 50° C.

More generally, hybridization of short nucleic acids (below 200 bp in length, e.g. 17-40 bp in length) can be effected using the following exemplary hybridization protocols which can be modified according to the desired stringency; (i) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 1-1.5° C. below the Tₘ, final wash solution of 3 M TMAC1, 0.01 M sodium phosphate (pH 6.8), 1 mM EDTA (pH 7.6), 0.5% SDS at 1-1.5° C. below the Tₘ; (ii) hybridization solution of 6× SSC and 0.11% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature of 2-2.5° C. below the Tₘ, final wash solution of 3 M TMACI, 0.01 M sodium phosphate (pH 6.8), 1 mM EDTA (pH 7.6), 0.5% SDS at 1-1.5° C. below the Tₘ, final wash solution of 6×SSC, and final wash at 22° C.; (iii) hybridization solution of 6×SSC and 1% SDS or 3 M TMACI, 0.01 M sodium phosphate (pH 6.8), 1 mM EDTA (pH 7.6), 0.5% SDS, 100 μg/ml denatured salmon sperm DNA and 0.1% nonfat dried milk, hybridization temperature.

The detection of hybrid duplexes can be carried out by a number of methods. Typically, hybridization duplexes are separated from unhybridized nucleic acids and the labels bound to the duplexes are then detected. Such labels refer to radioactive, fluorescent, biological or enzymatic tags or labels of standard use in the art. A label can be conjugated to either the oligonucleotide probes or the nucleic acids derived from the biological sample.

Probes can be labeled according to numerous well known methods. Non-limiting examples of radioactive labels include 3H, 14C, 32P, and 35S, Non-limiting examples of detectable markers include ligands, fluorophores, chemiluminescent agents, enzymes, and antibodies. Other detectable markers for use with probes, which can enable an increase in sensitivity of the method of the invention, include biotin and radio-nucleotides. It will become evident to the person of ordinary skill that the choice of a particular label dictates the manner in which it is bound to the probe.

For example, oligonucleotides of the present invention can be labeled subsequent to synthesis, by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent. Alternatively, when fluorescently-labeled oligonucleotide probes are used, fluorescein, lissamine, phycoerythrin, rhodamine (Perkin Elmer Cetus), Cy2, Cy3, Cy3.5, Cy5, Cy5.5, Cy7, Fluor X (Amersham) and others [e.g., Kricka et al. (1992), Academic Press San Diego, Calif.] can be attached to the oligonucleotides.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays. For instance, samples may be hybridized to an irrelevant probe and treated with RNAse A prior to hybridization, to assess false hybridization.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods.

As commonly known, radioactive nucleotides can be incorporated into probes of the invention by several methods. Non-limiting examples of radioactive labels include ³H, ¹⁴C, ³²P, and ³⁵S.

Those skilled in the art will appreciate that wash steps may be employed to wash away excess target DNA or probe as well as unbound conjugate. Further, standard heterogeneous assay formats are suitable for detecting the hybrids using the labels present on the oligonucleotide primers and probes.

It will be appreciated that a variety of controls may be usefully employed to improve accuracy of hybridization assays.

Probes of the invention can be utilized with naturally occurring sugar-phosphate backbones as well as modified backbones including phosphorothioates, dithionates, alkyl phosphonates and a-nucleotides and the like. Probes of the invention can be constructed of either ribonucleic acid (RNA) or deoxyribonucleic acid (DNA), and preferably of DNA.

### NAT Assays

Detection of a nucleic acid of interest in a biological sample may also optionally be effected by NAT-based assays, which involve nucleic acid amplification technology, such as PCR for example (or variations thereof such as real-time PCR for example).

As used herein, a “primer” defines an oligonucleotide which is capable of annealing to (hybridizing with) a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14 Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the q3 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra).

The terminology “amplification pair” (or “primer pair”) refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

In one particular embodiment, amplification of a nucleic acid sample from a patient is amplified under conditions which favor the amplification of the most abundant differentially expressed nucleic acid. In one preferred embodiment, RT-PCR is carried out on an mRNA sample from a patient under conditions which favor the amplification of the most abundant mRNA. In another preferred embodiment, the amplification of the differentially expressed nucleic acids is carried out simultaneously. It will be realized by a person skilled in the art that such methods could be adapted for the detection of differentially expressed proteins instead of differentially expressed nucleic acid sequences.

The nucleic acid (i.e. DNA or RNA) for practicing the present invention may be obtained according to well known methods.

Oligonucleotide primers of the present invention may be of any suitable length, depending on the particular assay format and the particular needs and targeted genomes employed. Optionally, the oligonucleotide primers are at least 12 nucleotides in length, preferably between 15 and 24 molecules, and they may be adapted to be especially suited to a chosen nucleic acid amplification system. As commonly known in the art, the oligonucleotide primers can be designed by taking into consideration the melting point of hybridization thereof with its targeted sequence (Sambrook et al., 1989, Molecular Cloning-A Laboratory Manual, 2nd Edition, CSH Laboratories; Ausubel et al., 1989, in Current Protocols in Molecular Biology, John Wiley & Sons Inc., N.Y.).

It will be appreciated that antisense oligonucleotides may be employed to quantify expression of a splice isoform of interest. Such detection is effected at the pre-mRNA level. Essentially the ability to quantitate transcription from a splice site of interest can be effected based on splice site accessibility. Oligonucleotides may compete with splicing factors for the splice site sequences. Thus, low activity of the antisense oligonucleotide is indicative of splicing activity.

The polymerase chain reaction and other nucleic acid amplification reactions are well known in the art (various non-limiting examples of these reactions are described in greater detail below). The pair of oligonucleotides according to this aspect of the present invention are preferably selected to have compatible melting temperatures (Tm), e.g., melting temperatures which differ by less than that 7° C., preferably less than 5° C., more preferably less than 4° C., most preferably less than 3° C., ideally between 3° C. and 0° C.

Polymerase Chain Reaction (PCR): The polymerase chain reaction (PCR), as described in U.S. Pat. Nos. 4,683,195 and 4,683,202 to Mullis and Mullis et al., is a method of increasing the concentration of a segment of target sequence in a mixture of genomic DNA without cloning or purification. This technology provides one approach to the problems of low target sequence concentration. PCR can be used to directly increase the concentration of the target to an easily detectable level. This process for amplifying the target sequence involves the introduction of a molar excess of two oligonucleotide primers which are complementary to their respective strands of the double-stranded target sequence to the DNA mixture containing the desired target sequence. The mixture is denatured and then allowed to hybridize. Following hybridization, the primers are extended with polymerase so as to form complementary strands. The steps of denaturation, hybridization (annealing), and polymerase extension (elongation) can be repeated as often as needed, in order to obtain relatively high concentrations of a segment of the desired target sequence.

The length of the segment of the desired target sequence is determined by the relative positions of the primers with respect to each other, and, therefore, this length is a controllable parameter. Because the desired segments of the target sequence become the dominant sequences (in terms of concentration) in the mixture, they are said to be “PCR-amplified.”

Ligase Chain Reaction (LCR or LAR): The ligase chain reaction [LCR; sometimes referred to as “Ligase Amplification Reaction” (LAR)] has developed into a well-recognized alternative method of amplifying nucleic acids. In LCR, four oligonucleotides, two adjacent oligonucleotides which uniquely hybridize to one strand of target DNA, and a complementary set of adjacent oligonucleotides, which hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, and ligation amplify a short segment of DNA. LCR has also been used in combination with PCR to achieve enhanced detection of single-base changes: see for example Segev, PCT Publication No. WO9001069 A1 (1990). However, because the four oligonucleotides used in this assay can pair to form two short ligatable fragments, there is the potential for the generation of target-independent background signal. The use of LCR for mutant screening is limited to the examination of specific nucleic acid positions.

Self-Sustained Synthetic Reaction (3SR/NASBA): The self-sustained sequence replication reaction (3SR) is a transcription-based in vitro amplification system that can exponentially amplify RNA sequences at a uniform temperature. The amplified RNA can then be utilized for mutation detection. In this method, an oligonucleotide primer is used to add a phage RNA polymerase promoter to the 5′ end of the sequence of interest. In a cocktail of enzymes and substrates that includes a second primer, reverse transcriptase, RNase H, RNA polymerase and ribo- and deoxyribonucleoside triphosphates, the target sequence undergoes repeated rounds of transcription, cDNA synthesis and second-strand synthesis to amplify the area of interest. The use of 3SR to detect mutations is kinetically limited to screening small segments of DNA (e.g., 200-300 base pairs).

Q-Beta (Qβ) Replicase: In this method, a probe which recognizes the sequence of interest is attached to the replicatable RNA template for Qβ replicase. A previously identified major problem with false positives resulting from the replication of unhybridized probes has been addressed through use of a sequence-specific ligation step. However, available thermostable DNA ligases are not effective on this RNA substrate, so the ligation must be performed by T4 DNA ligase at low temperatures (37 degrees C.). This prevents the use of high temperature as a means of achieving specificity as in the LCR, the ligation event can be used to detect a mutation at the junction site, but not elsewhere.

A successful diagnostic method must be very specific. A straight-forward method of controlling the specificity of nucleic acid hybridization is by controlling the temperature of the reaction. While the 3SR/NASBA, and Qβ systems are all able to generate a large quantity of signal, one or more of the enzymes involved in each cannot be used at high temperature (i.e., >55 degrees C.). Therefore the reaction temperatures cannot be raised to prevent non-specific hybridization of the probes. If probes are shortened in order to make them melt more easily at low temperatures, the likelihood of having more than one perfect match in a complex genome increases. For these reasons, PCR and LCR currently dominate the research field in detection technologies.

The basis of the amplification procedure in the PCR and LCR is the fact that the products of one cycle become usable templates in all subsequent cycles, consequently doubling the population with each cycle. The final yield of any such doubling system can be expressed as: (1+X)ⁿ=y, where “X” is the mean efficiency (percent copied in each cycle), “n” is the number of cycles, and “y” is the overall efficiency, or yield of the reaction. If every copy of a target DNA is utilized as a template in every cycle of a polymerase chain reaction, then the mean efficiency is 100%. If 20 cycles of PCR are performed, then the yield will be 220, or 1,048,576 copies of the starting material. If the reaction conditions reduce the mean efficiency to 85%, then the yield in those 20 cycles will be only 1.8520, or 220,513 copies of the starting material. In other words, a PCR running at 85% efficiency will yield only 21% as much final product, compared to a reaction running at 100% efficiency. A reaction that is reduced to 50% mean efficiency will yield less than 1% of the possible product.

In practice, routine polymerase chain reactions rarely achieve the theoretical maximum yield, and PCRs are usually ran for more than 20 cycles to compensate for the lower yield. At 50% mean efficiency, it would take 34 cycles to achieve the million-fold amplification theoretically possible in 20, and at lower efficiencies, the number of cycles required becomes prohibitive. In addition, any background products that amplify with a better mean efficiency than the intended target will become the dominant products.

Also, many variables can influence the mean efficiency of PCR, including target DNA length and secondary structure, primer length and design, primer and dNTP concentrations, and buffer composition, to name but a few. Contamination of the reaction with exogenous DNA (e.g., DNA spilled onto lab surfaces) or cross-contamination is also a major consideration. Reaction conditions must be carefully optimized for each different primer pair and target sequence, and the process can take days, even for an experienced investigator. The laboriousness of this process, including numerous technical considerations and other factors, presents a significant drawback to using PCR in the clinical setting. Indeed, PCR has yet to penetrate the clinical market in a significant way The same concerns arise with LCR, as LCR must also be optimized to use different oligonucleotide sequences for each target sequence. In addition, both methods require expensive equipment, capable of precise temperature cycling.

Many applications of nucleic acid detection technologies, such as in studies of allelic variation, involve not only detection of a specific sequence in a complex background, but also the discrimination between sequences with few, or single, nucleotide differences. One method of the detection of allele-specific variants by PCR is based upon the fact that it is difficult for Taq polymerase to synthesize a DNA strand when there is a mismatch between the template strand and the 3′ end of the primer. An allele-specific variant may be detected by the use of a priner that is perfectly matched with only one of the possible alleles; the mismatch to the other allele acts to prevent the extension of the primer, thereby preventing the amplification of that sequence. This method has a substantial limitation in that the base composition of the mismatch influences the ability to prevent extension across the mismatch, and certain mismatches do not prevent extension or have only a minimal effect.

A similar 3′-mismatch strategy is used with greater effect to prevent ligation in the LCR. Any mismatch effectively blocks the action of the rmostable ligase, but LCR still has the drawback of target-independent background ligation products initiating the amplification. Moreover, the combination of PCR with subsequent LCR to identify the nucleotides at individual positions is also a clearly cumbersome proposition for the clinical laboratory.

The direct detection method according to various preferred embodiments of the present invention may be, for example a cycling probe reaction (CPR) or a branched DNA analysis.

When a sufficient amount of a nucleic acid to be detected is available, there are advantages to detecting that sequence directly, instead of making more copies of that target, (e.g., as in PCR and LCR). Most notably, a method that does not amplify the signal exponentially is more amenable to quantitative analysis. Even if the signal is enhanced by attaching multiple dyes to a single oligonucleotide, the correlation between the final signal intensity and amount of target is direct. Such a system has an additional advantage that the products of the reaction will not themselves promote further reaction, so contamination of lab surfaces by the products is not as much of a concern. Recently devised techniques have sought to eliminate the use of radioactivity and/or improve the sensitivity in automatable formats. Two examples are the “Cycling Probe Reaction” (CPR), and “Branched DNA” (bDNA).

Cycling probe reaction (CPR): The cycling probe reaction (CPR), uses a long chimeric oligonucleotide in which a central portion is made of RNA while the two termini are made of DNA. Hybridization of the probe to a target DNA and exposure to a thermostable RNase H causes the RNA portion to be digested. This destabilizes the remaining DNA portions of the duplex, releasing the remainder of the probe from the target DNA and allowing another probe molecule to repeat the process. The signal, in the form of cleaved probe molecules, accumulates at a linear rate. While the repeating process increases the signal, the RNA portion of the oligonucleotide is vulnerable to RNases that may carried through sample preparation.

Branched DNA: Branched DNA (bDNA), involves oligonucleotides with branched structures that allow each individual oligonucleotide to carry 35 to 40 labels (e.g., alkaline phosphatase enzymes). While this enhances the signal from a hybridization event, signal from non-specific binding is similarly increased.

The detection of at least one sequence change according to various preferred embodiments of the present invention may be accomplished by, for example restriction fragment length polymorphism (RFLP analysis), allele specific oligonucleotide (ASO) analysis, Denaturing/Temperature Gradient Gel Electrophoresis (DGGE/TGGE), Single-Strand Conformation Polymorphism (SSCP) analysis or Dideoxy fingerprinting (ddF).

The demand for tests which allow the detection of specific nucleic acid sequences and sequence changes is growing rapidly in clinical diagnostics. As nucleic acid sequence data for genes from humans and pathogenic organisms accumulates, the demand for fast, cost-effective, and easy-to-use tests for as yet mutations within specific sequences is rapidly increasing.

A handful of methods have been devised to scan nucleic acid segments for mutations. One option is to determine the entire gene sequence of each test sample (e.g., a bacterial isolate). For sequences under approximately 600 nucleotides, this may be accomplished using amplified material (e.g., PCR reaction products). This avoids the time and expense associated with cloning the segment of interest. However, specialized equipment and highly trained personnel are required, and the method is too labor-intense and expensive to be practical and effective in the clinical setting.

In view of the difficulties associated with sequencing, a given segment of nucleic acid may be characterized on several other levels. At the lowest resolution, the size of the molecule can be determined by electrophoresis by comparison to a known standard run on the same gel. A more detailed picture of the molecule may be achieved by cleavage with combinations of restriction enzymes prior to electrophoresis, to allow construction of an ordered map. The presence of specific sequences within the fragment can be detected by hybridization of a labeled probe, or the precise nucleotide sequence can be determined by partial chemical degradation or by primer extension in the presence of chain-terminating nucleotide analogs.

Restriction fragment length polymorphism (RFLP): For detection of single-base differences between like sequences, the requirements of the analysis are often at the highest level of resolution. For cases in which the position of the nucleotide in question is known in advance, several methods have been developed for examining single base changes without direct sequencing. For example, if a mutation of interest happens to fall within a restriction recognition sequence, a change in the pattern of digestion can be used as a diagnostic tool (e.g., restriction fragment length polymorphism [RFLP] analysis).

Single point mutations have been also detected by the creation or destruction of RFLPs. Mutations are detected and localized by the presence and size of the RNA fragments generated by cleavage at the mismatches. Single nucleotide mismatches in DNA heteroduplexes are also recognized and cleaved by some chemicals, providing an alternative strategy to detect single base substitutions, generically named the “Mismatch Chemical Cleavage” (MCC). However, this method requires the use of osmium tetroxide and piperidine, two highly noxious chemicals which are not suited for use in a clinical laboratory.

RFLP analysis suffers from low sensitivity and requires a large amount of sample. When RFLP analysis is used for the detection of point mutations, it is, by its nature, limited to the detection of only those single base changes which fall within a restriction sequence of a known restriction endonuclease. Moreover, the majority of the available enzymes have 4 to 6 base-pair recognition sequences, and cleave too frequently for many large-scale DNA manipulations. Thus, it is applicable only in a small fraction of cases, as most mutations do not fall within such sites.

A handful of rare-cutting restriction enzymes with 8 base-pair specificities have been isolated and these are widely used in genetic mapping, but these enzymes are few in number, are limited to the recognition of G+C-rich sequences, and cleave at sites that tend to be highly clustered. Recently, endonucleases encoded by group I introns have been discovered that might have greater than 12 base-pair specificity, but again, these are few in number.

Allele specific oligonucleotide (ASO): If the change is not in a recognition sequence, then allele-specific oligonucleotides (ASOs), can be designed to hybridize in proximity to the mutated nucleotide, such that a primer extension or ligation event can bused as the indicator of a match or a mis-match. Hybridization with radioactively labeled allelic specific oligonucleotides (ASO) also has been applied to the detection of specific point mutations. The method is based on the differences in the melting temperature of short DNA fragments differing by a single nucleotide. Stringent hybridization and washing conditions can differentiate between mutant and wild-type alleles. The ASO approach applied to PCR products also has been extensively utilized by various researchers to detect and characterize point mutations in ras genes and gsp/gip oncogenes. Because of the presence of various nucleotide changes in multiple positions, the ASO method requires the use of many oligonucleotides to cover all possible oncogenic mutations.

With either of the techniques described above (i.e., RFLP and ASO), the precise location of the suspected mutation must be known in advance of the test. That is to say, they are inapplicable when one needs to detect the presence of a mutation within a gene or sequence of interest.

Denaturing/Temperature Gradient Gel Electrophoresis (DGGE/TGGE): Two other methods rely on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of mutations in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE. Attaching a GC clamp to one primer is critical to ensure that the amplified sequence has a low dissociation temperature. Modifications of the technique have been developed, using temperature gradients, and the method can be also applied to RNA:RNA duplexes.

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE). CDGE requires that gels be performed under different denaturant conditions in order to reach high efficiency for the detection of mutations.

A technique analogous to DGGE, termed temperature gradient gel electrophoresis (TGGE), uses a thermal gradient rather than a chemical denaturant gradient. TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Single-Strand Conformation Polymorphism (SSCP): Another common method, called “Single-Strand Conformation Polymorphism” (SSCP) was developed by Hayashi, Sekya and colleagues and is based on the observation that single strands of nucleic acid can take on characteristic conformations in non-denaturing conditions, and these conformations influence electrophoretic mobility. The complementary strands assume sufficiently different structures that one strand may be resolved from the other. Changes in sequences within the fragment will also change the conformation, consequently altering the mobility and allowing this to be used as an assay for sequence variations.

The SSCP process involves denaturing a DNA segment (e.g., a PCR product) that is labeled on both strands, followed by slow electrophoretic separation on a non-denaturing polyacrylamide gel, so that intra-molecular interactions can form and not be disturbed during the run. This technique is extremely sensitive to variations in gel composition and temperature. A serious limitation of this method is the relative difficulty encountered in comparing data generated in different laboratories, under apparently similar conditions.

Dideoxy fingerprinting (ddF): The dideoxy fingerprinting (ddF) is another technique developed to scan genes for the presence of mutations. The ddF technique combines components of Sanger dideoxy sequencing with SSCP. A dideoxy sequencing reaction is performed using one dideoxy terminator and then the reaction products are electrophoresed on nondenaturing polyacrylamide gels to detect alterations in mobility of the termination segments as in SSCP analysis. While ddF is an improvement over SSCP in terms of increased sensitivity, ddF requires the use of expensive dideoxynucleotides and this technique is still limited to the analysis of fragments of the size suitable for SSCP (i.e., fragments of 200-300 bases for optimal detection of mutations).

In addition to the above limitations, all of these methods are limited as to the size of the nucleic acid fragment that can be analyzed. For the direct sequencing approach, sequences of greater than 600 base pairs require cloning, with the consequent delays and expense of either deletion sub-cloning or primer walking, in order to cover the entire fragment. SSCP and DGGE have even more severe size limitations. Because of reduced sensitivity to sequence changes, these methods are not considered suitable for larger fragments. Although SSCP is reportedly able to detect 90% of single-base substitutions within a 200 base-pair fragment, the detection drops to less than 50% for 400 base pair fragments. Similarly, the sensitivity of DGGE decreases as the length of the fragment reaches 500 base-pairs. The ddF technique, as a combination of direct sequencing and SSCP, is also limited by the relatively small size of the DNA that can be screened.

According to a presently preferred embodiment of the present invention the step of searching for any of the nucleic acid sequences described here, in tumor cells or in cells derived from a cancer patient is effected by any suitable technique, including, but not limited to, nucleic acid sequencing, polymerase chain reaction, ligase chain reaction, self-sustained synthetic reaction, Qβ-Replicase, cycling probe reaction, branched DNA, restriction fragment length polymorphism analysis, mismatch chemical cleavage, heteroduplex analysis, allele-specific oligonucleotides, denaturing gradient gel electrophoresis, constant denaturant gel electrophoresis, temperature gradient gel electrophoresis and dideoxy fingerprinting.

Detection may also optionally be performed with a chip or other such device. The nucleic acid sample which includes the candidate region to be analyzed is preferably isolated, amplified and labeled with a reporter group. This reporter group can be a fluorescent group such as phycoerythrin. The labeled nucleic acid is then incubated with the probes immobilized on the chip using a fluidics station. describe the fabrication of fluidics devices and particularly microcapillary devices, in silicon and glass substrates.

Once the reaction is completed, the chip is inserted into a scanner and patterns of hybridization are detected. The hybridization data is collected, as a signal emitted from the reporter groups already incorporated into the nucleic acid, which is now bound to the probes attached to the chip. Since the sequence and position of each probe immobilized on the chip is known, the identity of the nucleic acid hybridized to a given probe can be determined.

It will be appreciated that when utilized along with automated equipment, the above described detection methods can be used to screen multiple samples for a disease and/or pathological condition both rapidly and easily.

### Radio-Imaging Methods

These methods include but are not limited to, positron emission tomography (PET) single photon emission computed tomography (SPECT). Both of these techniques are non-invasive, and can be used to detect and/or measure a wide variety of tissue events and/or functions, such as detecting cancerous cells for example. Unlike PET, SPECT can optionally be used with two labels simultaneously. SPECT has some other advantages as well, for example with regard to cost and the types of labels that can be used. For example, U.S. Pat. No. 6,696,686 describes the use of SPECT for detection of breast cancer, and is hereby incorporated by reference as if fully set forth herein.

### Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest and Herceptin (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

### Surrogate Markers:

A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, nucleic acid tests and imaging modalities).

Surrogate endpoints were used first mainly in the cardiovascular area. For example, antihypertensive drugs have been approved based on their effectiveness in lowering blood pressure. Similarly, in the past, cholesterol-lowering agents have been approved based on their ability to decrease serum cholesterol, not on the direct evidence that they decrease mortality from atherosclerotic heart disease. The measurement of cholesterol levels is now an accepted surrogate marker of atherosclerosis. In addition, currently two commonly used surrogate markers in HIV studies are CD4+ T cell counts and quantitative plasma HIV RNA (viral load). In some embodiments of this invention, the polypeptide/polynucleotide expression pattern may serve as a surrogate marker for a particular disease, as will be appreciated by one skilled in the art.

The following sections relate to Candidate Marker Examples.

### Candidate Marker Examples Section

This Section relates to Examples of sequences according to the present invention, including illustrative methods of selection thereof with regard to cancer; other markers were selected as described below for the individual markers.

Description of the methodology undertaken to uncover the biomolecular sequences of the present invention

Human ESTs and cDNAs were obtained from GenBank versions 136 (Jun. 15, 2003 ftp.ncbi.nih.gov/genbank/release.notes/gb136.release.notes); NCBI genome assembly of April 2003; RefSeq sequences from June 2003; Genbank version 139 (December 2003); Human Genome from NCBI (Build 34) (from October 2003); and RefSeq sequences from December 2003. With regard to GenBank sequences, the human EST sequences from the EST (GBEST) section and the human mRNA sequences from the primate (GBPRI) section were used; also the human nucleotide RefSeq mRNA sequences were used (see for example www.ncbi.nlm.nih.gov/Genbank/GenbankOverview.html and for a reference to the EST section, see www.ncbi.nlm.nih.gov/dbEST/; a general reference to dbEST, the EST database in GenBank, may be found in Boguski et al, Nat. Genet. 1993 August; 4(4):332-3; all of which are hereby incorporated by reference as if fully set forth herein).

Novel splice variants were predicted using the LEADS clustering and assembly system as described in Sorek, R., Ast, G. & Graur, D. Alu-containing exons are alternatively spliced. Genome Res 12, 1060-7 (2002); U.S. Pat. No. 6,625,545; and U.S. patent application Ser. No. 10/426,002, published as US20040101876 on May 27, 2004; all of which are hereby incorporated by reference as if fully set forth herein. Briefly, the software cleans the expressed sequences from repeats, vectors and immunoglobulins. It then aligns the expressed sequences to the genome taking alternatively splicing into account and clusters overlapping expressed sequences into “clusters” that represent genes or partial genes.

These were annotated using the GeneCarta (Compugen, Tel-Aviv, Israel) platform. The GeneCarta platform includes a rich pool of annotations, sequence information (particularly of spliced sequences), chromosomal information, alignments, and additional information such as SNPs, gene ontology terms, expression profiles, functional analyses, detailed domain structures, known and predicted proteins and detailed homology reports.

A brief explanation is provided with regard to the method of selecting the candidates. However, it should be noted that this explanation is provided for descriptive purposes only, and is not intended to be limiting in any way. The potential markers were identified by a computational process that was designed to find genes and/or their splice variants that are specifically expressed in cardiac tissue, as opposed to other types of tissues and also particularly as opposed to muscle tissue, by using databases of expressed sequences. Various parameters related to the information in the EST libraries, determined according to classification by library annotation, were used to assist in locating genes and/or splice variants thereof that are specifically and/or differentially expressed in heart tissues. The detailed description of the selection method and of these parameters is presented in Example 1 below.

### Selecting Candidates with Regard to Cancer

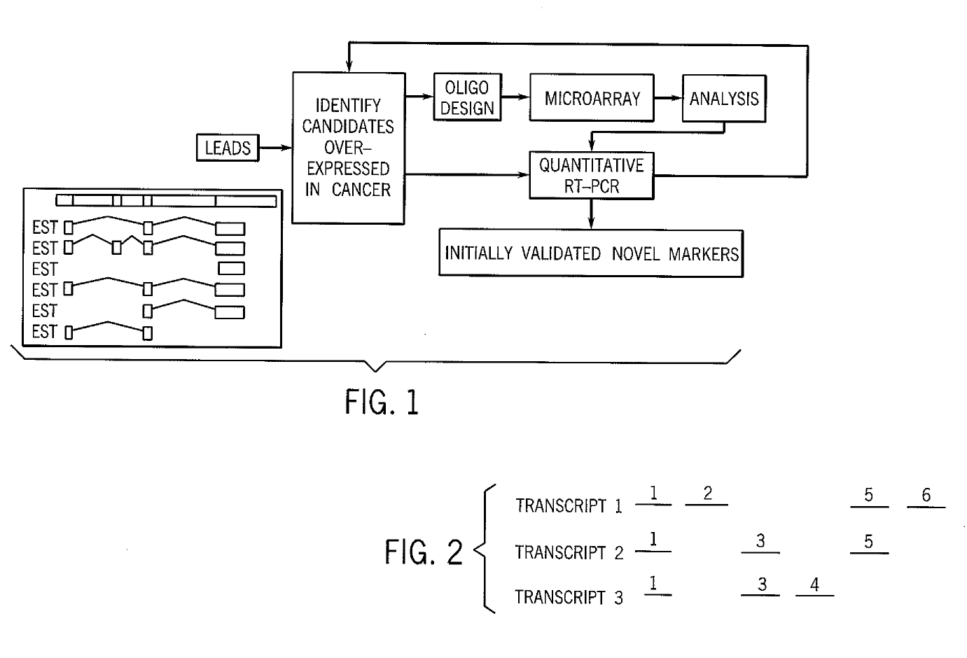

A brief explanation is provided with regard to a non-limiting method of selecting the candidates for cancer diagnostics. However, it should noted that this explanation is provided for descriptive purposes only, and is not intended to be limiting in any way. The potential markers were identified by a computational process that was designed to find genes and/or their splice variants that are over-expressed in tumor tissues, by using databases of expressed sequences. Various parameters related to the information in the EST libraries, determined according to a manual classification process, were used to assist in locating genes and/or splice variants thereof that are over-expressed in cancerous tissues. The detailed description of the selection method is presented in Example 1 below. The cancer biomarkers selection engine and the following wet validation stages are schematically summarized in FIG. 1.

### PART II—Cancer Markers

### EXAMPLE 1

### Identification of Differentially Expressed Gene Products

### Algorithm

In order to distinguish between differentially expressed gene products and constitutively expressed genes (i.e., house keeping genes) an algorithm based on an analysis of frequencies was configured. A specific algorithm for identification of transcripts over expressed in cancer is described hereinbelow.

Dry Analysis

Library Annotation—EST Libraries are Manually Classified According to:
(i) Tissue origin
(ii) Biological source—Examples of frequently used biological sources for construction of EST libraries include cancer cell-lines; normal tissues; cancer tissues; fetal tissues; and others such as normal cell lines and pools of normal cell-lines, cancer cell-lines and combinations thereof. A specific description of abbreviations used below with regard to these tissues/cell lines etc is given above.
(iii) Protocol of library construction—various methods are known in the art for library construction including normalized library construction; non-normalized library construction; subtracted libraries; ORESTES and others. It will be appreciated that at times the protocol of library construction is not indicated.

The following rules are followed:

EST libraries originating from identical biological samples are considered as a single library.

EST libraries which include above-average levels of DNA contamination are eliminated.

Dry computation—development of engines which are capable of identifying genes and splice variants that arc temporally and spatially expressed.

Clusters (genes) having at least five sequences including at least two sequences from the tissue of interest are analyzed.

### EXAMPLE 2

### Identification of Genes Over Expressed in Cancer

Two different scoring algorithms were developed.

Libraries score—candidate sequences which are supported by a number of cancer libraries, are more likely to serve as specific and effective diagnostic markers.

The basic algorithm—for each cluster the number of cancer and normal libraries contributing sequences to the cluster was counted. Fisher exact test was used to check if cancer libraries are significantly over-represented in the cluster as compared to the total number of cancer and normal libraries

Library counting: Small libraries (e.g., less than 1000 sequences) were excluded from consideration unless they participate in the cluster. For this reason, the total number of libraries is actually adjusted for each cluster.

Clones no. score—Generally, when the number of ESTs is much higher in the cancer libraries relative to the normal libraries it might indicate actual over-expression.

The algorithm—

Clone counting: For counting EST clones each library protocol class was given a weight based on an assessment of how much the protocol reflects actual expression levels:

(i) non-normalized 1

(ii) normalized: 0.2

(iii) all other classes: 0.1

Clones number score—The total weighted number of EST clones from cancer libraries was compared to the EST clones from normal libraries. To avoid cases where one library contributes to the majority of the score, the contribution of the library that gives most clones for a given cluster was limited to 2 clones.

The score was computed as

$$\frac{c + 1}{C} / \frac{n + 1}{N}$$

Where:

c—weighted number of “cancer” clones in the cluster.

C— weighted number of clones in all “cancer” libraries.

n—weighted number of “normal” clones in the cluster.

N— weighted number of clones in all “normal” libraries.

Clones number score significance—Fisher exact test was used to check if EST clones from cancer libraries are significantly over-represented in the cluster as compared to the total number of EST clones from cancer and normal libraries.

Two search approaches were used to find either general cancer-specific candidates or tumor specific candidates.
Libraries/sequences originating from tumor tissues are counted as well as libraries originating from cancer cell-lines (“normal” cell-lines were ignored).
Only libraries/sequences originating from tumor tissues are counted

### EXAMPLE 3

### Identification of Tissue Specific Genes

For detection of tissue specific clusters, tissue libraries/sequences were compared to the total number of libraries/sequences in cluster. Similar statistical tools to those described in above were employed to identify tissue specific genes. Tissue abbreviations are the same as for cancerous tissues, but are indicated with the header “normal tissue”.

The algorithm—for each tested tissue T and for each tested cluster the following were examined:

1. Each cluster includes at least 2 libraries from the tissue T. At least 3 clones (weighed—as described above) from tissue T in the cluster; and

2. Clones from the tissue T are at least 40% from all the clones participating in the tested cluster

Fisher exact test P-values were computed both for library and weighted clone counts to check that the counts are statistically significant.

### EXAMPLE 4

### Identification of Splice Variants Over Expressed in Cancer of Clusters which are not Over Expressed in Cancer

Cancer-specific splice variants containing a unique region were identified.

Identification of unique sequence regions in splice variants

A. Region is defined as a group of adjacent exons that always appear or do not appear together in each splice variant.

A “segment” (sometimes referred also as “seg” or “node”) is defined as the shortest contiguous transcribed region without known splicing inside.

Only reliable ESTs were considered for region and segment analysis. An EST was defined as unreliable if:

(i) Unspliced;

(ii) Not covered by RNA;

(iii) Not covered by spliced ESTs; and

(iv) Alignment to the genome ends in proximity of long poly-A stretch or starts in proximity of long poly-T stretch.

Only reliable regions were selected for further scoring. Unique sequence regions were considered reliable if:

(i) Aligned to the genome; and

(ii) Regions supported by more than 2 ESTs.

The algorithm

Each unique sequence region divides the set of transcripts into 2 groups:

(i) Transcripts containing this region (group TA).

(ii) Transcripts not containing this region (group TB).

The set of EST clones of every cluster is divided into 3 groups:

(i) Supporting (originating from) transcripts of group TA (S1).

(ii) Supporting transcripts of group TB (S2).

(iii) Supporting transcripts from both groups (S3).

Library and clones number scores described above were given to S1 group.

Fisher Exact Test P-values were used to check if:

S1 is significantly enriched by cancer EST clones compared to S2; and

S1 is significantly enriched by cancer EST clones compared to cluster background (S1+S2+S3).

Identification of unique sequence regions and division of the group of transcripts accordingly is illustrated in FIG. 2. Each of these unique sequence regions corresponds to a segment, also termed herein a “node”.

Region 1: common to all transcripts, thus it is not considered; Region 2: specific to Transcript 1: T_{—}1 unique regions (2+6) against T_{—}2+3 unique regions (3+4); Region 3: specific to Transcripts 2+3: T_{—}2+3 unique regions (3+4) against T1 unique regions (2+6); Region 4: specific to Transcript 3: T_{—}3 unique regions (4) against T1+2 unique regions (2+5+6); Region 5: specific to Transcript 1+2: T_{—}1+2 unique regions (2+5+6) against T3 unique regions (4); Region 6: specific to Transcript 1: same as region 2.

### EXAMPLE 5

### Identification of Cancer Specific Splice Variants of Genes Over Expressed in Cancer

A search for EST supported (no mRNA) regions for genes of:

(i) known cancer markers

(ii) Genes shown to be over-expressed in cancer in published micro-array experiments.

Reliable EST supported-regions were defined as supported by minimum of one of the following:

(i) 3 spliced ESTs; or

(ii) 2 spliced ESTs from 2 libraries;

(iii) 10 unspliced ESTs from 2 libraries, or

(iv) 3 libraries.

### EXAMPLE 6

### Diseases and Conditions that May be Diagnosed with One or More Variant(s) According to the Present Invention

### Ovarian Cancer

Certain splice variants described herein are potential markers for ovarian cancer. Ovarian cancer markers according to the present invention which may also optionally have this utility include but are not limited to: T29749, F13779 or variants as described herein or markers related thereto. Other conditions that may be diagnosed by these markers or variants of them include but are not limited to the presence, risk and/or extent of the following:

1. The identification of a metastasis of unknown origin which originated from a primary ovarian cancer, for example gastric carcinoma (such as Krukenberg tumor), breast cancer, colorectal carcinoma and pancreatic carcinoma.

2. As a marker to distinguish between different types of ovarian cancer, therefore potentially affect treatment choice (e.g. discrimination between epithelial tumors and germ cell tumors).

3. As a tool in the assessment of abdominal mass and in particular in the differential diagnosis between a benign and malignant ovarian cysts.

4. As a tool for the assessment of infertility.

5. Other conditions that may elevate serum levels of ovary related markers. These include but are not limited to: cancers of the endometrium, cervix, fallopian tubes, pancreas, breast, lung and colon; nonmalignant conditions such as pregnancy, endometriosis, pelvic inflammatory disease and uterine fibroids.

6. Conditions which have similar symptoms, signs and complications as ovarian cancer and where the differential diagnosis between them and ovarian cancer is of clinical importance including but not limited to:
a. Non-malignant causes of pelvic mass. Including, but not limited to: benign (functional) ovarian cyst, uterine fibroids, endometriosis, benign ovarian neoplasms and inflammatory bowel lesions
b. Any condition suggestive of a malignant tumor including but not limited to anorexia, cachexia, weight loss, fever, hypercalcemia, skeletal or abdominal pain, paraneoplastic syndrome.
c. Ascites.

### Lung Cancer

Certain splice variants described herein are potential markers for lung cancer. Lung cancer markers according to the present invention which may also optionally have this utility include but are not limited to: T29749 or F13779 or variants as described herein or markers related thereto. Other conditions that may be diagnosed by these markers or variants of them include but are not limited to the presence, risk and/or extent of the following:

1. The identification of a metastasis of unknown origin which originated from a primary lung cancer.

2. The assessment of a malignant tissue residing in the lung and is from a non-lung origin, including, but not limited to: osteogenic and soft tissue sarcomas; colorectal, uterine, cervix and corpus tumors; head and neck, breast, testis and salivary gland cancers; melanoma; and bladder and kidney tumors.

3. As a marker to distinguish between different types of lung cancer, therefore potentially affect treatment choice (e.g. small cell vs. non small cell tumors).

4. As a tool in the assessment of unexplained dyspnea and/or chronic cough and/or hemoptysis.

5. As a tool in the differential diagnosis of the origin of a pleural effusion.

6. Conditions which have similar symptoms, signs and complications as lung cancer and where the differential diagnosis between them and lung cancer is of clinical importance including but not limited to:
a. Non-malignant causes of lung symptoms and signs. Symptoms and signs include, but are not limited to: lung lesions and infiltrates, wheeze, stridor.
b. Other symptoms, signs and complications suggestive of lung cancer, such as tracheal obstruction, esophageal compression, dysphagia, recurrent laryngeal nerve paralysis, hoarseness, phrenic nerve paralysis with elevation of the hemidiaphragm and Horner syndrome.
c. Any condition suggestive of a malignant tumor including but not limited to anorexia, cachexia, weight loss, fever, hypercalcemia, hypophosphatemia, hyponatremia, syndrome of inappropriate secretion of antidiuretic hormone, elevated ANP, elevated ACTH, hypokalemia, clubbing, neurologic-myopathic syndromes and thrombophlebitis.

### Breast Cancer

Certain splice variants described herein are potential markers for breast cancer. Breast cancer markers according to the present invention which may also optionally have this utility include but are not limited to: T29749 or F13779 or variants as described herein or markers related thereto. Other conditions that may be diagnosed by these markers or variants of them include but are not limited to the presence, risk and/or extent of the following:

1. The identification of a metastasis of unknown origin which originated from a primary breast cancer tumor.

2. In the assessment of lymphadenopathy, and in particular axillary lymphadenopathy.

3. As a marker to distinguish between different types of breast cancer, therefore potentially affect treatment choice (e.g. as HER-2)

4. As a tool in the assessment of palpable breast mass and in particular in the differential diagnosis between a benign and malignant breast mass.

5. As a tool in the assessment of conditions affecting breast skin (e.g. Paget's disease) and their differentiation from breast cancer.

6. As a tool in the assessment of breast pain or discomfort resulting from either breast cancer or other possible conditions (e.g. Mastitis, Mondors syndrome).

7. Other conditions not mentioned above which have similar symptoms, signs and complications as breast cancer and where the differential diagnosis between them and breast cancer is of clinical importance including but not limited to:
a. Abnormal mammogram and/or nipple retraction and/or nipple discharge due to causes other than breast cancer. Such causes include but are not limited to benign breast masses, melanoma, trauma and technical and/or anatomical variations.
b. Any condition suggestive of a malignant tumor including but not limited to anorexia, cachexia, weight loss, fever, hypercalcemia, paraneoplastic syndrome.

Lymphadenopathy, weight loss and other signs and symptoms associated with breast cancer but originate from diseases different from breast cancer including but not limited to other malignancies, infections and autoimmune diseases.

### Candidate Marker Examples Section

This section relates to examples of sequences according to the present invention, including illustrative methods of selection thereof.

The markers of the present invention were tested with regard to their expression in various cancerous and non-cancerous tissue samples.

### Actual Marker Examples

The following examples relate to specific actual marker examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The markers of the present invention were tested with regard to their expression in various cancerous and non-cancerous tissue samples. A description of the samples used in the ovarian cancer testing panel is provided in Table 1_{—}1 below. A description of the samples used in the lung cancer testing panel is provided in Table 1_{—}2 below. A description of the samples used in the breast cancer testing panel is provided in Table 1_{—}3 below. A description of the samples used in the normal tissue panel is provided in Table 1_{—}4 below. The keys for the tables 1_{—}1, 1_{—}2, and 1_{—}3 are listed in tables 1_{—}1_{—}1, 1_{—}2_{—}1, and 1_{—}3_{—}1, respectively. Tests were then performed as described in the “Materials and Experimental Procedures” section below.

TABLE I_1
Tissue samples in ovarian cancer testing panel

| Sample | | | | | | | | | |
|---|---|---|---|---|---|---|---|---|---|
| id (GCI)/ | | | | | | | | | |
| case id | RNA ID | | | | | | | | |
| (Asterand)/ | (GCI)/ | | | | | | | | |
| lot no. | Sample | | | | | | | | |
| (old | ID | | | | | Ethnic | | Menopausal | Mens |
| samples) | (Asterand) | Diag | C Stage | Tumor % | age | BG | CA125PRE | Status | Age |
| 23074 | 71900A2 | SA | 1 | 80 | 49 | CAU | | Pre-M | |
| 22653 | 70270A1 | SA | 1 | 90 | 69 | WCAU | | Post-M | |
| 18700 | 40771B1 | SA | IB | 100 | 62 | WCAU | | Post-M | |
| 93-09- | | SPC | 1B | | 67 | | | | |
| 4901 | | | | | | | | | |
| 17646 | 32667B1 | SA | IB | 100 | 68 | W | | Post-M | |
| 15644 | 22996A1 | SA | IC | 100 | 48 | CAU | | M | |
| 18701 | 40773C1 | SA | IIA | 100 | 59 | CAU | | Post-M | |
| 2O37O | | SA | IIB | 75 | 43 | WCAU | — | Pre-M | 12 |
| 7B3DP | | SA | IIB | 70 | 70 | WCAU | — | Post-M | 14 |
| 2001-08- | | PSC | 3A | | 72 | | | | |
| G011 | | | | | | | | | |
| 95-08- | | PSA | 3B | | 50 | | | | |
| G069 | | | | | | | | | |
| 99-12- | | A | 3C | | 46 | | >500 | | |
| G432 | | | | | | | | | |
| 13268 | 19832A1 | SA | IIIC | 90 | 48 | C | | Post-M | |
| 2001-12- | | SA | 3C | | 50 | | 260 | | |
| G035 | | | | | | | | | |
| 3NTIS | | SA | IIIC | 70 | 53 | WCAU | 70 | Post-M | 12 |
| CEJUS | | SA | IIIC | 70 | 53 | WCAU | 4814 | Pre-M | — |
| 5NCLK | | SA | IIIC | 70 | 54 | WCAU | 209 | Post-M | 13 |
| N0021 | | PSA | 3C | | 55 | CAU | | | |
| 1HI5H | | SA | IIIC | 90 | 61 | WCAU | 34 | Post-M | 12 |
| 7RMHZ | | SA | IIIC | 80 | 63 | WCAU | — | Post-M | 12 |
| 4WAAB | | SA | IIIA | 90 | 63 | WCAU | — | Post-M | 11 |
| 79Z67 | | SA | IIIC | 85 | 67 | WCAU | — | Post-M | 12 |
| 94-05- | | APP | 3C | | 67 | | | | |
| 7603 | | | | | | | | | |
| DDSNL | | SA | IIIC | 70 | 68 | WCAU | — | Post-M | 11 |
| DH8PH | | SA | IV | 95 | 70 | WCAU | — | Post-M | 13 |
| A503175 | | SPC | | | 41 | Asian | | | |
| A501111 | | A | | | 41 | Asian | | | |
| A406023 | | A | | | 45 | Asian | | | |
| A407068 | | A | | | 49 | Asian | | | |
| ILS-7286 | | PC | UN | | 50 | Asian | | | |
| A0106 | | A | UN | | 51 | Asian | | | |
| ILS-1431 | | PA | UN | | 52 | Asian | | | |
| A503176 | | SPC | | | 52 | Asian | | | |
| ILS-1408 | | PA | UN | | 53 | Asian | | | |
| IND- | | A | | | 59 | Asian | | | |
| 00375 | | | | | | | | | |
| A501113 | | A | | | 60 | Asian | | | |
| A407069 | | A | | | 60 | Asian | | | |
| ILS-1406 | | PA | UN | | 73 | Asian | | | |
| E2WKF | | EA | IA | 70 | 30 | WCAU | — | Pre-M | 12 |
| 5895C | | EA | IA | 95 | 39 | WCAU | — | Pre-M | 14 |
| 533DX | | EA | IA | 95 | 50 | WCAU | 190 | Pre-M | 11 |
| HZ2EY | | EA | IA | 90 | 55 | WCAU | 1078 | Pre-M | 13 |
| RWOIV | | EA | IA | 65 | 47 | WCAU | 1695 | Pre-M | 14 |
| 1U52X | | EA | IIA | 95 | 61 | WCAU | 275 | | — |
| AI7WS | | EA | IIB | 70 | 67 | WCAU | 78 | Post-M | 14 |
| 1VT3I | | EA | IIIC | 90 | 50 | WCAU | — | Pre-M | 12 |
| PZQXH | | EA | IIIC | 80 | 52 | WCAU | — | Pre-M | 11 |
| I8VHZ | | EA | IV | 90 | 68 | WCAU | — | Post-M | — |
| 98-03- | | Mixed . . . | 2 | | 38 | | >35 | | |
| G803 | | | | | | | | | |
| 95-11- | | PS & | 3C | | 49 | | | | |
| G006 | | EC | | | | | | | |
| 2002-05- | | MS & | 3C | | 56 | | | | |
| G513 | | EA | | | | | | | |
| 2002-05- | | MS & | 3C | | 64 | | | | |
| G509 | | EAM | | | | | | | |
| 95-04- | | PEA | 3C | | 68 | | | | |
| 2002 | | | | | | | | | |
| 95-10- | | MC | IA | | 44 | | | >100 | |
| G020 | | | | | | | | | |
| IMDA1 | | MA | IC | 70 | 41 | WCAU | 50 | Pre-M | 12 |
| 12742 | 18920A1 | MA | IC | 70 | 61 | C | | Post-M | |
| A0139 | | MC | IC | | 72 | Asian | | | |
| NJM4U | | MA | IIA | 80 | 51 | WCAU | | | |
| USA- | | PMC | IIIA | | 45 | C | | | |
| 00273 | | | | | | | | | |
| RAFCW | | MA | IIIA | 75 | 55 | WCAU | 95 | Post-M | 13 |
| 23177 | 72888A1 | MA | IIIC | 60 | 52 | C | | Pre-M | |
| 16103 | 29374B1 | MA | IIIC | 100 | 62 | W | | Post-M | |
| A504085 | | MA | | | 34 | Asian | | | |
| A504083 | | MA | | | 45 | Asian | | | |
| A504084 | | MA | | | 51 | Asian | | | |
| A407065 | | C | | | 27 | Asian | | | |
| 1090387 | | CNOS | | | 58 | Asian | | | |
| 2001-07- | | CCA | 1A | | 73 | | | | |
| G084 | | | | | | | | | |
| 2001-10- | | CCA | 3A | | 74 | | slightly | | |
| G002 | | | | | | | elevated | | |
| SC656 | | MBT | IA | 75 | 40 | WCAU | 138 | Pre-M | 13 |
| 3D5FO | | MBT | IA | 85 | 51 | WCAU | 19 | ? | 15 |
| 7JP3F | | MBT | IA | 75 | 56 | WCAU | 125 | Post-M | 14 |
| VNM- | | MC | | | 45 | Asian | | | |
| 00187 | | Low M | | | | | | | |
| 98-08- | | EA | 1A | | 46 | | | | |
| G001 | | of | | | | | | | |
| | | BM | | | | | | | |
| 99-10- | | BMC | | | 32 | | 6 | | |
| G442 | | | | | | | | | |
| QL1KY | | BMC | | 100 | 42 | WCAU | | | |
| 16870 | 30534A1 | BMC | | 100 | 45 | W | | Pre-M | |
| 99-01- | | BMC | | | 46 | | | | |
| G407 | | | | | | | | | |
| 943EC | | BMC | | 75 | 54 | WCAU | | | |
| JO8W7 | | BMC | | 50 | 56 | WCAU | | | |
| 17016 | 30645B1 | BSC | IA | 100 | 38 | C | | Pre-M | |
| 99-06- | | BSC | | | 57 | | | | |
| G039 | | | | | | | | | |
| DQQ2F | | BSCF | | 95 | 68 | WCAU | | | |
| 8786 | 8275A1 | BSC | | 100 | 80 | CAU | | Post-M | |
| 15690 | 23054A1 | NO- | | | 52 | CAU | | Pre-M | |
| | | BM | | | | | | | |
| 16843 | 30488A1 | NO- | | | 57 | W | | Post-M | |
| | | BM | | | | | | | |
| 16850 | 30496B1 | NO- | | | 65 | W | | Post-M | |
| | | BM | | | | | | | |
| 16848 | 30499C1 | NO- | | | 66 | CAU | | Post-M | |
| | | BM | | | | | | | |
| WPU1U | | NO- | | 0 | 32 | WC | | | |
| | | PS | | | | | | | |
| Y9VH1 | | NO- | | 0 | 35 | WCAU | | | |
| | | PS | | | | | | | |
| 76VM9 | | NO- | | 0 | 41 | WCAU | | | |
| | | PS | | | | | | | |
| DWHTZ | | NO- | | 0 | 42 | WCAU | | | |
| | | PS | | | | | | | |
| SJ2R2 | | NO- | | 0 | 43 | WCAU | | | |
| | | PS | | | | | | | |
| 9RQMN | | NO- | | 0 | 45 | WCAU | | | |
| | | PS | | | | | | | |
| TOAE5 | | NO- | | 0 | 45 | WCAU | | | |
| | | PS | | | | | | | |
| TW9PM | | NO- | | 0 | 46 | WCAU | | | |
| | | PS | | | | | | | |
| 2VND2 | | NO- | | 0 | 46 | WCAU | | | |
| | | PS | | | | | | | |
| L629F | | NO- | | 0 | 47 | WCAU | | | |
| | | PS | | | | | | | |
| XLB23 | | NO- | | 0 | 47 | WCAU | | | |
| | | PS | | | | | | | |
| IDUVY | | NO- | | 0 | 47 | WCAU | | | |
| | | PS | | | | | | | |
| ZCXAD | | NO- | | 0 | 48 | WCAU | | | |
| | | PS | | | | | | | |
| PEQ6C | | NO- | | 0 | 49 | WCAU | | | |
| | | PS | | | | | | | |
| DD73B | | NO- | | 0 | 49 | WCAU | | | |
| | | PS | | | | | | | |
| E2UF7 | | NO- | | 0 | 53 | WCAU | | | |
| | | PS | | | | | | | |
| GWXUZ | | NO- | | 0 | 53 | WCAU | | | |
| | | PS | | | | | | | |
| 4YG5P | | NO- | | 0 | 55 | WCAU | | | |
| | | PS | | | | | | | |
| FDPL9 | | NO- | | 0 | 56 | WCAU | | | |
| | | PS | | | | | | | |
| A503274 | | NO- | | | 41 | Asian | | | |
| | | PM | | | | | | | |
| A504086 | | NO- | | | 41 | Asian | | | |
| | | PM | | | | | | | |
| CG-188-7 | | NO- | | | 49 | | | | |
| | | PM | | | | | | | |
| A504087 | | NO- | | | 51 | Asian | | | |
| | | PM | | | | | | | |

| Sample | | | | | | | | | | | |
|---|---|---|---|---|---|---|---|---|---|---|---|
| id (GCI)/ | | | | | | | | | | | |
| case id | | | | | | | | | | | |
| (Asterand)/ | | | Age | | | | | | | | |
| lot no. | | Preg | at | | Oral | Oral | | | | | |
| (old | Preg | To | first | | Con | Con | Tubal | WT | HT | | Recovery |
| samples) | Times | term | child | OC | Length | Unit | ligation | (KG) | (CM) | BMI | Type |
| 23074 | 2 | 1 | | | | | | 82 | 169 | 28.7 | Surg |
| 22653 | 1 | 1 | | | | | | 80 | 167 | 28.7 | Surg |
| 18700 | 3 | 3 | | | | | | 72 | 160 | 28.1 | Surg |
| 93-09- | | | | | | | | | | | |
| 4901 | | | | | | | | | | | |
| 17646 | 9 | 2 | | | | | | 76 | 164 | 28.3 | Surg |
| 15644 | 4 | 2 | | | | | | 62 | 167 | 22 | Surg |
| 18701 | 1 | 1 | | | | | | 72 | 167 | 25.8 | Surg |
| 2O37O | 0 | 0 | 0 | NO | — | | NO | 90.7 | 168 | 32.3 | Surg |
| 7B3DP | 5 | 3 | 20 | YES | 6 | months | NO | 77.1 | 168 | 27.4 | Surg |
| 2001-08- | | | | | | | | | | | |
| G011 | | | | | | | | | | | |
| 95-08- | | | | | | | | | | | |
| G069 | | | | | | | | | | | |
| 99-12- | | | | | | | | | | | |
| G432 | | | | | | | | | | | |
| 13268 | | | | | | | | 72 | 168 | 25.5 | Surg |
| 2001-12- | | | | | | | | | | | |
| G035 | | | | | | | | | | | |
| 3NTIS | 1 | 1 | 26 | YES | 3 | months | NO | 93.4 | 175 | 30.4 | Surg |
| CEJUS | 2 | 2 | 30 | NO | — | | NO | 88 | 165 | 32.3 | Surg |
| 5NCLK | 2 | 2 | 21 | YES | 1 | years | NO | 71.7 | 170 | 24.7 | Surg |
| N0021 | | | | | | | | | | | |
| 1HI5H | 6 | 3 | 22 | NO | — | | NO | 99.8 | 160 | 39 | Surg |
| 7RMHZ | 2 | 2 | 20 | YES | 10 | years | NO | 68 | 165 | 25 | Surg |
| 4WAAB | 2 | 1 | 29 | YES | 4 | years | NO | 51 | 166 | 18.5 | Surg |
| 79Z67 | 6 | 5 | 24 | YES | 2 | years | YES | 76.2 | 163 | 28.8 | Surg |
| 94-05- | | | | | | | | | | | |
| 7603 | | | | | | | | | | | |
| DDSNL | 4 | 4 | 19 | NO | — | | NO | 59 | 155 | 24.6 | Surg |
| DH8PH | 4 | 3 | 20 | NO | — | | NO | 69.4 | 152 | 29.9 | Surg |
| A503175 | | | | | | | | | | | |
| A501111 | | | | | | | | | | | |
| A406023 | | | | | | | | | | | |
| A407068 | | | | | | | | | | | |
| ILS-7286 | | | | | | | | | | | |
| A0106 | | | | | | | | | | | |
| ILS-1431 | | | | | | | | | | | |
| A503176 | | | | | | | | | | | |
| ILS-1408 | | | | | | | | | | | |
| IND- | | | | | | | | | | | |
| 00375 | | | | | | | | | | | |
| A501113 | | | | | | | | | | | |
| A407069 | | | | | | | | | | | |
| ILS-1406 | | | | | | | | | | | |
| E2WKF | 6 | 5 | 17 | YES | 6 | years | NO | 92.5 | 168 | 32.9 | Surg |
| 5895C | 2 | 2 | 20 | NO | — | | NO | 86.2 | 170 | 29.8 | Surg |
| 533DX | 0 | — | — | YES | 2 | years | NO | 108 | 170 | 37.3 | Surg |
| HZ2EY | 0 | — | — | NO | — | | NO | 62 | 165 | 22.7 | Surg |
| RWOIV | 0 | — | — | NO | — | | NO | 105 | 168 | 37.4 | Surg |
| 1U52X | — | — | — | | — | | | 64.9 | 152 | 27.9 | Surg |
| AI7WS | 0 | — | — | NO | — | | NO | 70.3 | 175 | 22.9 | Surg |
| 1VT3I | 2 | 2 | 24 | YES | 1 | years | NO | 59.9 | 155 | 24.9 | Surg |
| PZQXH | 0 | — | — | YES | 5 | years | NO | 57.6 | 163 | 21.8 | Surg |
| I8VHZ | 2 | 2 | 27 | NO | — | | NO | 73 | 160 | 28.5 | Surg |
| 98-03- | | | | | | | | | | | |
| G803 | | | | | | | | | | | |
| 95-11- | | | | | | | | | | | |
| G006 | | | | | | | | | | | |
| 2002-05- | | | | | | | | | | | |
| G513 | | | | | | | | | | | |
| 2002-05- | | | | | | | | | | | |
| G509 | | | | | | | | | | | |
| 95-04- | | | | | | | | | | | |
| 2002 | | | | | | | | | | | |
| 95-10- | | | | | | | | | | | |
| G020 | | | | | | | | | | | |
| IMDA1 | 2 | 1 | 24 | NO | — | | | 85.3 | 168 | 30.3 | Surg |
| 12742 | 3 | 3 | | | | | | 70 | 155 | 29.1 | Surg |
| A0139 | | | | | | | | | | | |
| NJM4U | | | | | | | | 73.5 | 168 | 26.1 | Surg |
| USA- | | | | | | | | | | | |
| 00273 | | | | | | | | | | | |
| RAFCW | 4 | 3 | 22 | NO | — | | NO | 98 | 170 | 33.8 | Surg |
| 23177 | | | | | | | | 100 | 162 | 38.1 | Surg |
| 16103 | 1 | 1 | | | | | | 40 | 160 | 15.6 | Surg |
| A504085 | | | | | | | | | | | |
| A504083 | | | | | | | | | | | |
| A504084 | | | | | | | | | | | |
| A407065 | | | | | | | | | | | |
| 1090387 | | | | | | | | | | | |
| 2001-07- | | | | | | | | | | | |
| G084 | | | | | | | | | | | |
| 2001-10- | | | | | | | | | | | |
| G002 | | | | | | | | | | | |
| SC656 | 2 | 2 | 23 | NO | — | | YES | 83.9 | 160 | 32.8 | Surg |
| 3D5FO | 0 | — | — | NO | — | | NO | 79 | 163 | 29.9 | Surg |
| 7JP3F | 3 | 3 | 19 | YES | 5 | years | NO | 58 | 152 | 25.1 | Surg |
| VNM- | | | | | | | | | | | |
| 00187 | | | | | | | | | | | |
| 98-08- | | | | | | | | | | | |
| G001 | | | | | | | | | | | |
| 99-10- | | | | | | | | | | | |
| G442 | | | | | | | | | | | |
| QL1KY | | | | | | | | 72.6 | 175 | 23.6 | Surg |
| 16870 | 2 | 2 | | | | | | 100 | 169 | 35 | Surg |
| 99-01- | | | | | | | | | | | |
| G407 | | | | | | | | | | | |
| 943EC | | | | | | | | 170 | 168 | 60.5 | Surg |
| JO8W7 | | | | | | | | 71.2 | 165 | 26.1 | Surg |
| 17016 | 2 | 2 | | | | | | 64 | 172 | 21.6 | Surg |
| 99-06- | | | | | | | | | | | |
| G039 | | | | | | | | | | | |
| DQQ2F | | | | | | | | 107 | 155 | 44.5 | Surg |
| 8786 | 10 | 9 | | | | | | 0 | 0 | 0 | Surg |
| 15690 | 10 | 3 | | | | | | 97 | 167 | 34.8 | Surg |
| 16843 | 4 | 2 | | | | | | 76 | 154 | 32 | Surg |
| 16850 | 2 | 2 | | | | | | 89 | 162 | 33.9 | Surg |
| 16848 | 9 | 2 | | | | | | 76 | 158 | 30.4 | Surg |
| WPU1U | | | | | | | | 74.4 | 163 | 28.2 | Surg |
| Y9VH1 | | | | | | | | 81 | 170 | 28 | Surg |
| 76VM9 | | | | | | | | 59 | 165 | 21.6 | Surg |
| DWHTZ | | | | | | | | 92.5 | 168 | 32.9 | Surg |
| SJ2R2 | | | | | | | | 65.8 | 155 | 27.4 | Surg |
| 9RQMN | | | | | | | | 59 | 163 | 22.3 | Surg |
| TOAE5 | | | | | | | | 87 | 165 | 31.9 | Surg |
| TW9PM | | | | | | | | 70 | 173 | 23.5 | Surg |
| 2VND2 | | | | | | | | 88.5 | 168 | 31.5 | Surg |
| L629F | | | | | | | | 68 | 175 | 22.2 | Surg |
| XLB23 | | | | | | | | 65 | 175 | 21.2 | Surg |
| IDUVY | | | | | | | | 63 | 165 | 23.1 | Surg |
| ZCXAD | | | | | | | | 102 | 163 | 38.6 | Surg |
| PEQ6C | | | | | | | | 81.2 | 165 | 29.8 | Surg |
| DD73B | | | | | | | | 88 | 163 | 33.3 | Surg |
| E2UF7 | | | | | | | | 70.3 | 163 | 26.6 | Surg |
| GWXUZ | | | | | | | | 72.6 | 165 | 26.6 | Surg |
| 4YG5P | | | | | | | | 71.7 | 165 | 26.3 | Surg |
| FDPL9 | | | | | | | | 82.1 | 167 | 29.4 | Surg |
| A503274 | | | | | | | | | | | |
| A504086 | | | | | | | | | | | |
| CG-188-7 | | | | | | | | | | | |
| A504087 | | | | | | | | | | | |

TABLE 1_1_1

| Key | Full Name |
|---|---|
| A | Adenocarcinoma |
| APP | Adenocarcinoma from primary peritoneal |
| BMC | BENIGN MUCINOUS CYSTADENOMA |
| BSC | BENIGN SEROUS CYSTADENOMA |
| BSCF | BENIGN SEROUS CYSTADENOFIBROMA |
| C | Carcinoma |
| C Stage | Cancer stage |
| CAU | Caucasian |
| CCA | Clear cell adenocarcinoma |
| CNOS | Carcinoma NOS |
| EA | ENDOMETROID ADENOCARCINOMA |
| EA of BM | Endometroid adenocarcinoma of borderline malignancy |
| HT | Height |
| M | Menopausal |
| MA | MUCINOUS ADENOCARCINOMA |
| MBT | MUCINOUS BORDERLINE TUMOR |
| MC | Mucinous cystadenocarcinoma |
| MC Low M | Mucinous cystadenocarcinoma with low malignant |
| Mens. Age | Menstrual Age |
| Mixed . . . | Mixed epithelial cystadenocarcinoma with mucinous, |
| | endometrioid, squamous and papillary serous |
| MS & EA | Mixed serous and endometrioid adenocarcinoma |
| MS & EAM | Mixed serous and endometrioid adenocarcinoma |
| | of mullerian |
| NO-BM | NORMAL OVARY-BM |
| NO-PM | NORMAL OVARY-PM |
| NO-PS | NORMAL OVARY-PS |
| OC | Oral Contraceptive |
| PA | Papillary adenocarcinoma |
| PC | Papillary cystadenocarcinoma |
| PEA | Papillary endometrioid adenocarcinoma |
| PMC | Papillary mucinous cystadenocarcinoma |
| Post-M | Post-menopausal |
| Pre-M | Pre-menopausal |
| PS & EC | Papillary serous and endometrioid cystadenocarcinoma |
| PSA | Papillary serous adenocarcinoma |
| PSC | Papillary serous carcinoma |
| SA | SEROUS ADENOCARCINOMA |
| SPC | Serous papillary cystadenocarcinoma |
| W | White |
| WCAU | WHITE/CAUCASIAN |
| WT | Weight |

TABLE 1_2
Tissue samples in lung cancer testing panel

| | | | sample | | | | | | | | | |
|---|---|---|---|---|---|---|---|---|---|---|---|---|
| | | | id | | | | | | | | | |
| | | | (GCI)/ | | | | | | | | | |
| | | | case id | TISSUE | | | | | | | | |
| | | | (Asterand)/ | ID | RNA | | | | | | | |
| | | | lot | (GCI)/ | ID (GCI)/ | | | | | | | |
| | | | no. | specimen | Sample | | | | | | | |
| | Source/ | sample | (old | ID | ID | | Diag | Specimen | | | | |
| Tissue | Delivery | name | samples) | (Asterand) | (Asterand) | Diag | remarks | location | Gr | TNM | CS | Tum % |
| LC | GCI | 1-GC- | 7Z9V4 | 7Z9V4AYM | | Aden | BC | | | | IA | 80 |
| | | BAC-SIA | | | | | | | | | | |
| LC | GCI | 2-GC- | ZW2AQ | ZW2AQARP | | Aden | BC | | | | IB | 70 |
| | | BAC-SIB | | | | | | | | | | |
| LC | Bioch | 72-(44)- | A501123 | | | AC | | | 2 | UN | | |
| | | Bc-BAC | | | | | | | | | | |
| LC | Bioch | 73-(45)- | A501221 | | | AC | | | UN | UN | | |
| | | Bc-BAC | | | | | | | | | | |
| LC | GCI | 4-GC- | 3MOPL | 3MOPLA79 | | Aden | | | | | IA | 60 |
| | | Adeno- | | | | | | | | | | |
| | | SIA | | | | | | | | | | |
| LC | GCI | 5-GC- | KOJXD | KOJXDAV4 | | Aden | | | | | IA | 90 |
| | | Adeno- | | | | | | | | | | |
| | | SIA | | | | | | | | | | |
| LC | GCI | 6-GC- | X2Q44 | X2Q44A79 | | Aden | | | | | IA | 85 |
| | | Adeno- | | | | | | | | | | |
| | | SIA | | | | | | | | | | |
| LC | GCI | 7-GC- | 6BACZ | 6BACZAP5 | | Aden | | | | | IA | 60 |
| | | Adeno- | | | | | | | | | | |
| | | SIA | | | | | | | | | | |
| LC | GCI | 8-GC- | BS9AF | BS9AFA3E | | Aden | | | | | IA | 55 |
| | | Adeno- | | | | | | | | | | |
| | | SIA | | | | | | | | | | |
| LC | GCI | 9-GC- | UCLOA | UCLOAA9L | | Aden | | | | | IA | 80 |
| | | Adeno- | | | | | | | | | | |
| | | SIA | | | | | | | | | | |
| LC | GCI | 10-GC- | BVYK3 | BVYK3A7Z | | Aden | | | | | IA | 60 |
| | | Adeno- | | | | | | | | | | |
| | | SIA | | | | | | | | | | |
| LC | GCI | 11-GC- | U4DM4 | U4DM4AFZ | | Aden | | | | | IB | 65 |
| | | Adeno- | | | | | | | | | | |
| | | SIB | | | | | | | | | | |
| LC | GCI | 12-GC- | OWX5Y | OWX5YA3S | | Aden | | | | | IB | 90 |
| | | Adeno- | | | | | | | | | | |
| | | SIB | | | | | | | | | | |
| LC | GCI | 13-GC- | XYY96 | XYY96A6B | | Aden | | | | | IIA | 70 |
| | | Adeno- | | | | | | | | | | |
| | | SIIA | | | | | | | | | | |
| LC | GCI | 14-GC- | SO7B1 | SO7B1AIJ | | Aden | | | | | IIA | 70 |
| | | Adeno- | | | | | | | | | | |
| | | SIIA | | | | | | | | | | |
| LC | GCI | 15-GC- | QANSY | QANSYACD | | Aden | | | | | IIIA | 65 |
| | | Adeno- | | | | | | | | | | |
| | | SIIIA | | | | | | | | | | |
| LC | Bioch | 16-(95)- | A610063 | | | Aden | | | 1 | UN | | |
| | | BC- | | | | | | | | | | |
| | | Adeno | | | | | | | | | | |
| LC | Bioch | 17-(89)- | A609077 | | | Aden | | | 2-3 | UN | | |
| | | Bc- | | | | | | | | | | |
| | | Adeno | | | | | | | | | | |
| LC | Bioch | 18-(76)- | A609218 | | | Aden | | | 3 | UN | | |
| | | Bc- | | | | | | | | | | |
| | | Adeno | | | | | | | | | | |
| LC | Bioch | 74-(2)-Bc | A504118 | | | Aden | | | 1 | UN | | |
| | | Adeno | | | | | | | | | | |
| LC | Bioch | 76-(75)- | A609217 | | | Aden | | | 2 | UN | | |
| | | Bc- | | | | | | | | | | |
| | | Adeno | | | | | | | | | | |
| LC | Bioch | 77-(12)- | A504119 | | | Aden | | | 2 | UN | | |
| | | Bc- | | | | | | | | | | |
| | | Adeno | | | | | | | | | | |
| LC | Bioch | 78-(13)- | A504116 | | | Aden | | | 2-3 | UN | | |
| | | Bc- | | | | | | | | | | |
| | | Adeno | | | | | | | | | | |
| LC | Bioch | 79-(94)- | A610118 | | | Aden | | | 3 | UN | | |
| | | Bc- | | | | | | | | | | |
| | | Adeno | | | | | | | | | | |
| LC | Ichilov | 80-(3)-Ic- | CG-200 | | | Aden | | | UN | UN | | |
| | | Adeno | | | | | | | | | | |
| LC | Ichilov | 81-(14)- | CG-111 | | | Aden | | | UN | UN | | |
| | | Ic-Adeno | | | | | | | | | | |
| LC | Aster | 19-As-Sq- | 9220 | 9418 | 9418A1 | SCC | | | 1 | TXN0M0 | Occult | 80 |
| | | S0 | | | | | | | | | | |
| LC | GCI | 20-GC- | U2QHS | U2QHSA2N | | SCC | | | | | IA | 55 |
| | | Sq-SIA | | | | | | | | | | |
| LC | GCI | 21-GC- | TRQR7 | TRQR7ACD | | SCC | | | | | IB | 75 |
| | | Sq-SIB | | | | | | | | | | |
| LC | Aster | 22-As-Sq- | 17581 | 32603 | 32603B1 | SCC | | | 3 | T2N0M0 | IB | 90 |
| | | SIB | | | | | | | | | | |
| LC | Aster | 23-As-Sq- | 18309 | 41454 | 41454B1 | SCC | | | 2 | T2N0MX | IB | 100 |
| | | SIB | | | | | | | | | | |
| LC | Aster | 24-As-Sq- | 9217 | 9415 | 9415B1 | SCC | | | 2 | T2N0M0 | IB | 90 |
| | | SIB | | | | | | | | | | |
| LC | GCI | 25-GC- | RXQ1P | RXQ1PAEA | | SCC | | | | | IIB | 55 |
| | | Sq-SIIB | | | | | | | | | | |
| LC | GCI | 26-GC- | KB5KH | KB5KHA6X | | SCC | | | | | IIB | 65 |
| | | Sq-SIIB | | | | | | | | | | |
| LC | GCI | 27-GC- | LAYMB | LAYMBALF | | SCC | | | | | IIIA | 65 |
| | | Sq-SIIIA | | | | | | | | | | |
| LC | Ichilov | 28-(23)- | CG-109 (1) | | | SCC | | | UN | UN | | |
| | | Ic-Sq | | | | | | | | | | |
| LC | Ichilov | 29-(25)- | CG-204 | | | SCC | | | UN | UN | | |
| | | Ic-Sq | | | | | | | | | | |
| LC | Bioch | 30-(19)- | A408175 | | | SCC | | | 1 | UN | | |
| | | Bc-Sq | | | | | | | | | | |
| LC | Bioch | 31-(78)- | A607125 | | | SCC | | | 2 | UN | | |
| | | Bc-Sq | | | | | | | | | | |
| LC | Bioch | 32-(16)- | A409091 | | | SCC | | | 2 | UN | | |
| | | Bc-Sq | | | | | | | | | | |
| LC | Bioch | 33-(80)- | A609163 | | | SCC | | | 2 | UN | | |
| | | Bc-Sq | | | | | | | | | | |
| LC | Bioch | 34-(18)- | A503387 | | | SCC | | | 2-3 | UN | | |
| | | Bc-Sq | | | | | | | | | | |
| LC | Bioch | 35-(81)- | A609076 | | | SCC | | | 3 | UN | | |
| | | Bc-Sq | | | | | | | | | | |
| LC | Bioch | 82-(21)- | A503187 | | | SCC | | | 2 | UN | | |
| | | Bc-Sq | | | | | | | | | | |
| LC | Bioch | 83-(17)- | A503183 | | | SCC | | | 2 | UN | | |
| | | Bc-Sq | | | | | | | | | | |
| LC | Bioch | 84-(79)- | A609018 | | | SCC | | | 3 | UN | | |
| | | Bc-Sq | | | | | | | | | | |
| LC | Bioch | 85-(22)- | A503386 | | | SCC | | | UN | UN | | |
| | | Bc-Sq | | | | | | | | | | |
| LC | Bioch | 86-(20)- | A501121 | | | SCC | | | UN | UN | | |
| | | Bc-Sq | | | | | | | | | | |
| LC | Bioch | 87-(88)- | A609219 | | | SCC | | | UN | UN | | |
| | | Bc-Sq | | | | | | | | | | |
| LC | Bioch | 88-(100)- | A409017 | | | SCC | | | UN | UN | | |
| | | Bc-Sq | | | | | | | | | | |
| LC | Ichilov | 89-(24)- | CG-123 | | | SCC | | | UN | UN | | |
| | | Ic-Sq | | | | | | | | | | |
| LC | GCI | 36-GC- | AF8AL | AF8ALAAL | | LCC | | | | | IA | 85 |
| | | LCC-SIA | | | | | | | | | | |
| LC | GCI | 37-GC- | O62XU | O62XUA1X | | LCC | | | | | IB | 75 |
| | | LCC-SIB | | | | | | | | | | |
| LC | GCI | 38-GC- | OLOIM | OLOIMAS1 | | LCC | | | | | IB | 70 |
| | | LCC-SIB | | | | | | | | | | |
| LC | GCI | 39-GC- | 1ZWSV | 1ZWSVAB9 | | LCC | | | | | IIB | 50 |
| | | LCC-SIIB | | | | | | | | | | |
| LC | GCI | 40-GC- | 2YHOD | 2YHODA1H | | LCC | NSCC | | | | IIB | 95 |
| | | LCC-SIIB | | | | | . . — | | | | | |
| LC | GCI | 41-GC- | 38B4D | 38B4DAQK | | LCC | | | | | IIB | 90 |
| | | LCC-SIIB | | | | | | | | | | |
| LC | Bioch | 90-(39)- | A504114 | | | LCC | | | UN | UN | | |
| | | Bc-LCC | | | | | | | | | | |
| LC | Bioch | 91-(87)- | A609165 | | | LCC | | | 3 | UN | | |
| | | Bc-LCC | | | | | | | | | | |
| LC | Bioch | 92-(38)- | A504113 | | | LCC | | | UN | UN | | |
| | | Bc-LCC | | | | | | | | | | |
| LC | Bioch | 93-(82)- | A609170 | | | LCNC | | | UN | UN | | |
| | | Bc-LCC | | | | | | | | | | |
| LC | GCI | 42-GC- | QPJQL | QPJQLAF6 | | SMCC | NC | | 3 | | IB | 65 |
| | | SCC-SIB | | | | | | | | | | |
| LC | Bioch | 43-(32)- | A501391 | | | SMCC | | | | UN | | |
| | | Bc-SCC | | | | | | | | | | |
| LC | Bioch | 44-(30)- | A501389 | | | SMCC | | | 3 | UN | | |
| | | Bc-SCC | | | | | | | | | | |
| LC | Bioch | 45-(83)- | A609162 | | | SMCC | | | UN | UN | | |
| | | Bc-SCC | | | | | | | | | | |
| LC | Bioch | 46-(86)- | A608032 | | | SMCC | | | 3 | UN | | |
| | | Bc-SCC | | | | | | | | | | |
| LC | Bioch | 47-(31)- | A501390 | | | SMCC | | | | UN | | |
| | | Bc-SCC | | | | | | | | | | |
| LC | Bioch | 48-(84)- | A609167 | | | SMCC | | | UN | UN | | |
| | | Bc-SCC | | | | | | | | | | |
| LC | Bioch | 49-(85)- | A609169 | | | SMCC | | | UN | UN | | |
| | | Bc-SCC | | | | | | | | | | |
| LC | Bioch | 50-(33)- | A504115 | | | SMCC | | | | UN | | |
| | | Bc-SCC | | | | | | | | | | |
| LN | Aster | 51-As-N- | 9078 | 9275 | 9275B1 | Norm-L | PS | | | | | |
| | | PS | | | | | | | | | | |
| LN | Aster | 52-As-N- | 8757 | 8100 | 8100B1 | Norm-L | PM | (Right), | | | | |
| | | PM | | | | | | Lobe | | | | |
| | | | | | | | | Inferior | | | | |
| LN | Aster | 53-As-N- | 6692 | 6161 | 6161A1 | Norm-L | PM | | | | | |
| | | PM | | | | | | | | | | |
| LN | Aster | 54-As-N- | 7900 | 7180 | 7180F1 | Norm-L | PM | | | | | |
| | | PM | | | | | | | | | | |
| LN | Aster | 55-As-N- | 8771 | 8163 | 8163A1 | Norm-L | PM | (Left), | | | | |
| | | PM | | | | | | Lobe | | | | |
| | | | | | | | | Superior | | | | |
| LN | Aster | 56-As-N- | 13094 | 19763 | 19763A1 | Norm-L | PM | | | | | |
| | | PM | | | | | | | | | | |
| LN | Aster | 57-As-N- | 19174 | 40654 | 40654A2 | Norm-L | PM | | | | | |
| | | PM | | | | | | | | | | |
| LN | Aster | 58-As-N- | 13128 | 19642 | 19642A1 | Norm-L | PM | | | | | |
| | | PM | | | | | | | | | | |
| LN | Aster | 59-As-N- | 14374 | 20548 | 20548C1 | Norm-L | PM | (Right), | | | | |
| | | PM | | | | | | Lobe | | | | |
| | | | | | | | | Superior | | | | |
| LN | Amb | 60-(99)- | 36856 | | | N-PM | PM | | | | | |
| | | Am-N | | | | | | | | | | |
| | | PM | | | | | | | | | | |
| LN | Amb | 61-(96)- | 36853 | | | N-PM | PM | | | | | |
| | | Am-N | | | | | | | | | | |
| | | PM | | | | | | | | | | |
| LN | Amb | 62-(97)- | 36854 | | | N-PM | PM | | | | | |
| | | Am-N | | | | | | | | | | |
| | | PM | | | | | | | | | | |
| LN | Amb | 63-(93)- | 111P0103A | | | N-PM | PM- | | | | | |
| | | Am-N | | | | | ICH | | | | | |
| | | PM | | | | | | | | | | |
| LN | Amb | 64-(98)- | 36855 | | | N-PM | PM | | | | | |
| | | Am-N | | | | | | | | | | |
| | | PM | | | | | | | | | | |
| LN | Bioch | 67-(50)- | A503385 | | | N-PM | PM | | | | | |
| | | Bc-N PM | | | | | | | | | | |
| LN | Bioch | 68-(92)- | A503204 | | | N-PM | PM | | | | | |
| | | Bc-N PM | | | | | | | | | | |
| LN | Bioch | 69-(91)- | A607257 | | | N-P2- | PM | | | | | |
| | | Bc-N PM | | | | PM | | | | | | |
| LN | Bioch | 70-(90)- | A608152 | | | N-P2 | PM | | | | | |
| | | Bc-N PM | | | | PM | | | | | | |
| LN | Bioch | 71-(48)- | A503206 | | | N-PM | PM | | | | | |
| | | Bc-N PM | | | | | | | | | | |

| | | | | | | # of | | | | | | | | | | |
|---|---|---|---|---|---|---|---|---|---|---|---|---|---|---|---|---|
| | | | | | | Y. | | | | | | | | | | |
| | | | | | # Cig. | Use | # Y. | | | | | | | | Cause | |
| | | | | Smoking | Per | of | off | Sm | Sm | Dr | # | HT | | Recovery | of | Exc. |
| Tissue | Gen | age | Ethnic B | Status | day | Tobacco | Tobacco | PY? | ppl | Al | Dr | (CM) | BMI | Type | Death | Y. |
| LC | F | 63 | WCAU | Prev | 20 | 15 | 27 | N | — | Y | 0 | 165 | 25.3 | Surg | | 2001 |
| | | | | U. | | | | | | | | | | | | |
| LC | F | 56 | WCAU | Prev | 15 | 28 | 10 | Y | 1 | Y | 6 | 165 | 23 | Surg | | 2002 |
| | | | | U. | | | | | | | | | | | | |
| LC | F | 61 | | | | | | | | | | | | | | |
| LC | F | 50 | | | | | | | | | | | | | | |
| LC | M | 68 | WCAU | Nev | — | — | — | N | — | N | — | 175 | 27.3 | Surg | | 2001 |
| | | | | U. | | | | | | | | | | | | |
| LC | F | 64 | WCAU | Prev | 15 | 40 | 7 | Y | 1 | N | 0 | 157 | 19.6 | Surg | | 2003 |
| | | | | U. | | | | | | | | | | | | |
| LC | M | 58 | WCAU | Prev | 10 | 47 | 0 | Y | 2 | N | — | 170 | 24.6 | Surg | | 2004 |
| | | | | U. | | | | | | | | | | | | |
| LC | F | 65 | WCAU | Curr | 6 | 30 | — | Y | 1 | N | — | 168 | 21 | Surg | | 2004 |
| | | | | U. | | | | | | | | | | | | |
| LC | F | 59 | WCAU | Curr | 20 | 40 | — | N | — | N | — | 160 | 23.9 | Surg | | 2004 |
| | | | | U. | | | | | | | | | | | | |
| LC | F | 69 | WCAU | Curr | 30 | 52 | — | Y | 4 | N | — | 157 | 34.8 | Surg | | 2005 |
| | | | | U. | | | | | | | | | | | | |
| LC | F | 60 | WCAU | Curr | 40 | 40 | — | N | — | N | — | 163 | 31.8 | Surg | | 2002 |
| | | | | U. | | | | | | | | | | | | |
| LC | F | 68 | WCAU | Prev | 5 | 4 | 43 | N | — | N | — | 165 | 22.3 | Surg | | 2003 |
| | | | | U. | | | | | | | | | | | | |
| LC | M | 69 | WCAU | Curr | 10 | — | — | | — | N | — | 183 | 30.5 | Surg | | 2002 |
| | | | | U. | | | | | | | | | | | | |
| LC | F | 62 | WCAU | Prev | 6 | 40 | 6 | N | — | Y | 0 | 160 | 27 | Surg | | 2004 |
| | | | | U. | | | | | | | | | | | | |
| LC | M | 56 | WCAU | Curr | 30 | 25 | — | Y | 1 | N | — | 180 | 36.4 | Surg | | 2001 |
| | | | | U. | | | | | | | | | | | | |
| LC | F | 61 | WCAU | Curr | 30 | 36 | — | Y | 1 | N | — | 163 | 25.1 | Surg | | 2004 |
| | | | | U. | | | | | | | | | | | | |
| LC | F | 54 | | | | | | | | | | | | | | |
| LC | M | 62 | | | | | | | | | | | | | | |
| LC | M | 57 | | | | | | | | | | | | | | |
| LC | M | 64 | | | | | | | | | | | | | | |
| LC | M | 65 | | | | | | | | | | | | | | |
| LC | F | 74 | | | | | | | | | | | | | | |
| LC | M | 64 | | | | | | | | | | | | | | |
| LC | M | 68 | | | | | | | | | | | | | | |
| LC | F | 56 | | | | | | | | | | | | | | |
| LC | M | 68 | | | | | | | | | | | | | | |
| LC | M | 67 | CAU | Curr | 11-20 | 31-40 | | | | O | | 163 | 28.6 | Surg | | 2003 |
| | | | | U. | | | | | | | | | | | | |
| LC | F | 68 | WCAU | Prev | 10 | 20 | 0 | N | — | N | — | 157 | 22.9 | Surg | | 2004 |
| | | | | U. | | | | | | | | | | | | |
| LC | M | 62 | WCAU | Prev | 20 | 50 | 0 | Y | 5 | N | — | 175 | 25.5 | Surg | | 2005 |
| | | | | U. | | | | | | | | | | | | |
| LC | M | 73 | CAU | Prev | | | | | | O | | 170 | 22.1 | Surg | | 2004 |
| | | | | U. | | | | | | | | | | | | |
| LC | M | 66 | CAU | Prev | 11-20 | 45 | | | | P | | 178 | 33.8 | Surg | | 2005 |
| | | | | U. | | | | | | | | | | | | |
| LC | M | 65 | CAU | Curr | 6-10 | 41-50 | | | | O | | 176 | 22 | Surg | | 2002 |
| | | | | U. | | | | | | | | | | | | |
| LC | F | 44 | WCAU | Prev | 20 | 20 | 0 | Y | 2 | N | — | 155 | 22.7 | Surg | | 2004 |
| | | | | U. | | | | | | | | | | | | |
| LC | M | 68 | WCAU | Prev | 40 | 40 | 0 | Y | 2 | N | — | 170 | 23.2 | Surg | | 2004 |
| | | | | U. | | | | | | | | | | | | |
| LC | F | 58 | WCAU | Prev | 50 | 40 | 1 | Y | 2 | N | — | 173 | 27.4 | Surg | | 2004 |
| | | | | U. | | | | | | | | | | | | |
| LC | M | 65 | | | | | | | | | | | | | | |
| LC | M | 72 | | | | | | | | | | | | | | |
| LC | M | 78 | | | | | | | | | | | | | | |
| LC | M | 62 | | | | | | | | | | | | | | |
| LC | F | 68 | | | | | | | | | | | | | | |
| LC | M | 74 | | | | | | | | | | | | | | |
| LC | M | 63 | | | | | | | | | | | | | | |
| LC | M | 53 | | | | | | | | | | | | | | |
| LC | M | 52 | | | | | | | | | | | | | | |
| LC | M | 57 | | | | | | | | | | | | | | |
| LC | M | 67 | | | | | | | | | | | | | | |
| LC | M | 48 | | | | | | | | | | | | | | |
| LC | M | 64 | | | | | | | | | | | | | | |
| LC | M | 64 | | | | | | | | | | | | | | |
| LC | M | 64 | | | | | | | | | | | | | | |
| LC | M | 76 | | | | | | | | | | | | | | |
| LC | M | 45 | WCAU | Prev | 45 | 33 | 0 | Y | 2 | Y | 28 | 178 | 31.9 | Surg | | 2004 |
| | | | | U. | | | | | | | | | | | | |
| LC | F | 60 | WCAU | Prev | 30 | 45 | 0 | Y | 3 | N | — | 160 | 16.8 | Surg | | 2004 |
| | | | | U. | | | | | | | | | | | | |
| LC | M | 68 | WCAU | Prev | — | 55 | — | Y | — | N | — | 173 | 22.8 | Surg | | 2001 |
| | | | | U. | | | | | | | | | | | | |
| LC | M | 51 | WCAU | Prev | 20 | 12 | 22 | Y | 1 | N | — | 183 | 26.6 | Surg | | 2004 |
| | | | | U. | | | | | | | | | | | | |
| LC | M | 62 | WCAU | Prev | 40 | 40 | 0 | Y | 2 | Y | 12 | 185 | 23.1 | Surg | | 2004 |
| | | | | U. | | | | | | | | | | | | |
| LC | F | 70 | WCAU | Prev | 30 | 50 | — | Y | 2 | Y | 13 | 168 | 20.7 | Surg | | 2002 |
| | | | | U. | | | | | | | | | | | | |
| LC | F | 35 | | | | | | | | | | | | | | |
| LC | F | 47 | | | | | | | | | | | | | | |
| LC | M | 58 | | | | | | | | | | | | | | |
| LC | M | 68 | | | | | | | | | | | | | | |
| LC | F | 62 | WCAU | Prev | 20 | 35 | 0.15 | Y | 2 | N | — | 165 | 19.8 | Surg | | 2003 |
| | | | | U. | | | | | | | | | | | | |
| LC | M | 30 | | | | | | | | | | | | | | |
| LC | M | 34 | | | | | | | | | | | | | | |
| LC | F | 47 | | | | | | | | | | | | | | |
| LC | F | 52 | | | | | | | | | | | | | | |
| LC | F | 59 | | | | | | | | | | | | | | |
| LC | F | 59 | | | | | | | | | | | | | | |
| LC | M | 66 | | | | | | | | | | | | | | |
| LC | M | | | | | | | | | | | | | | | |
| LN | M | 22 | CAU | Nev | | | | | | NU | | 0 | 0 | Surg | | 2003 |
| | | | | U. | | | | | | | | | | | | |
| LN | F | 26 | CAU | Nev | | | | | | O | | 170 | 22.1 | Aut | CA | 2003 |
| | | | | U. | | | | | | | | | | | | |
| LN | M | 37 | CAU | Nev | | | | | | C | | 183 | 20.9 | Aut | MCE | 2002 |
| | | | | U. | | | | | | | | | | | | |
| LN | F | 76 | CAU | Prev | | | | | | | | 165 | 26.8 | Aut | CPulA | 2002 |
| | | | | U. | | | | | | | | | | | | |
| LN | M | 81 | CAU | Prev | 41 or | 31-40 | | | | O | | 183 | 30.5 | Aut | CA | 2003 |
| | | | | U. | more | | | | | | | | | | | |
| LN | M | 0 | CAU | Prev | 21-40 | 41-50 | | | | P | | 175 | 25.1 | Aut | IC | |
| | | | | U. | | | | | | | | | | | | |
| LN | F | 69 | CAU | Curr | 21-40 | 31-40 | | | | P | | 165 | 22.4 | Aut | CPul A | 2005 |
| | | | | U. | | | | | | | | | | | | |
| LN | F | 75 | CAU | | | | | | | | | 160 | 21.5 | Aut | CPul A | 2004 |
| LN | F | 75 | CAU | | | | | | | | | 175 | 32.7 | Aut | Cer A | 2004 |
| LN | M | 31 | | | | | | | | | | | | | | |
| LN | F | 43 | | | | | | | | | | | | | | |
| LN | M | 46 | | | | | | | | | | | | | | |
| LN | F | 61 | | | | | | | | | | | | | | |
| LN | F | 72 | | | | | | | | | | | | | | |
| LN | M | 28 | | | | | | | | | | | | | | |
| LN | M | 28 | | | | | | | | | | | | | | |
| LN | P2 | 24, 29 | | | | | | | | | | | | | | |
| LN | P2 | 27, 28 | | | | | | | | | | | | | | |
| LN | M | 44 | | | | | | | | | | | | | | |

TABLE 1_2_1

| Key | Full Name |
|---|---|
| # Cig. Per day | Number of Cigarettes per day |
| # Dr | Number of Drinks |
| # of Y. Use of Tobacco | Number of Years Using Tobacco |
| # Y. off Tobacco | Number of Years Off Tobacco |
| AC | Alveolus carcinoma |
| Aden | ADENOCARCINOMA |
| Amb | Ambion |
| Aster | Asterand |
| Aut | Autopsy |
| BC | BRONCHIOLOALVEOLAR CARCINOMA |
| Bioch | Biochain |
| C | Current Use |
| CA | Cardiac arrest |
| CAU | Caucasian |
| Cer A | Cerebrovascular accident |
| CPul A | Cardiopulmonary arrest |
| CS | Cancer Stage |
| Curr U. | Current Use |
| Diag | Diagnosis |
| Dr Al | Drink Alcohol? |
| Exc Y. | Excision Year |
| Gen | Gender |
| Gr | Grade |
| Height | HT |
| IC | Ischemic cardiomyopathy |
| LC | Lung Cancer |
| LCC | LARGE CELL CARCINOMA |
| LCNC | Large Cell Neuroendocrine Carcinoma |
| LN | Lung Normal |
| MCE | Massive cerebral edema |
| N | No |
| NC | NEUROENDOCRINE CARCINOMA |
| Nev. U. | Never Used |
| Norm-L | Normal Lung |
| N-P2-PM | Normal (Pool 2)-PM |
| N-PM | Normal-PM |
| NSCC . . . | NON-SMALL CELL CARCINOMA WITH |
| | SARCOMUTOUS TRANSFORMTAIO |
| NU | Never used |
| O | Occasional Use |
| P | Previous Use |
| P2 | Pool 2 |
| Prev U. | Previous Use |
| SCC | Squamous Cell Carcinoma |
| Sm P Y? | Have people at home smoked in past 15 yr |
| Sm ppl | If yes, how many? |
| SMCC | SMALL CELL CARCINOMA |
| SMOKE_GROWING_UP | Did people smoke at |
| | home while growing up |
| Surg | Surgical |
| Tum % | Tumor Percentage |
| WCAU | White Caucasian |
| Y | Yes |

TABLE 1_3
Tissue samples in Breast cancer testing panel

| | | | sample_id | | | | | | | |
|---|---|---|---|---|---|---|---|---|---|---|
| | | | (GCI)/ | | | | | | | |
| | | | case id | | | | | | | |
| | | | (Asterand)/ | | | | | | | |
| | | | lot no. | TISSUE_ID | Sample | | | | | |
| | Source/ | sample | (old | (GCI)/specimen | ID | | | | C | |
| Tissue | Delivery | name | samples) | ID(Asterand) | (Asterand) | SampDIAG | Grade | TNM | Stage | Tum % |
| BC_in- | Aster | 1-As-DCIS | 19723 | 42509 | 42509A1 | DCIS | High | T1aN0M0 | 0 | 100 |
| situ | | S0 | | | | | Grade | | | |
| BC | GCI | 2-GC-IDC | 5IRTK | 5IRTKAXT | | IDC | | | I | 75 |
| | | SI | | | | | | | | |
| BC | ABS | 3-(42)-AB- | 6005020031T | | | IDC | 3 | T1cN0Mx | I | |
| | | IDC SI | | | | | | | | |
| BC | ABS | 4-(7)-AB- | 7263T | | | IDC | 2 | T1N0M0 | I | |
| | | IDC SI | | | | | | | | |
| BC | GCI | 5-GC-IDC | DSI52 | DSI52AH3 | | IDC | | | I | 50 |
| | | SI | | | | | | | | |
| BC | GCI | 6-GC-IDC | S2GBY | S2GBYAGC | | IDC | | | I | 55 |
| | | SI | | | | | | | | |
| BC | GCI | 7-GC-IDC | POPHP | POPHPAZ4 | | IDC | | | I | 65 |
| | | SI | | | | | | | | |
| BC | GCI | 8-GC-IDC | I2YLE | I2YLEACP | | IDC | | | I | 65 |
| | | SI | | | | | | | | |
| BC | Aster | 9-As-IDC SI | 17959 | 31225 | 31225A1 | IDC | 2 | T1NXM0 | I | 90 |
| BC | ABS | 10-(12)-AB- | 1432T | | | IDC | 2 | T2N0M0 | IIA | — |
| | | IDC SIIA | | | | | | | | |
| BC | Aster | 11-As-IDC | 17138 | 30697 | 30697A1 | IDC | 3 | T2NXM0 | IIA | 90 |
| | | SIIA | | | | | | | | |
| BC | GCI | 12-GC-IDC | YSZ67 | YSZ67A48 | | IDC | | | IIA | 70 |
| | | SIIA | | | | | | | | |
| BC | ABS | 13-(6)-AB- | 7238T | | | IDC | 1 | T2N0M0 | IIA | |
| | | IDC SIIA | | | | | | | | |
| BC | ABS | 14-(26)-AB- | 7249T | | | IDC | 3 | T2N0M0 | IIA | |
| | | IDC SIIA | | | | | | | | |
| BC | GCI | 15-GC-IDC | UT3SE | UT3SEAQY | | IDC | | | IIA | 80 |
| | | SIIA | | | | | | | | |
| BC | GCI | 16-GC-IDC | PVSYX | PVSYXA72 | | IDC | | | IIA | 65 |
| | | SIIA | | | | | | | | |
| BC | GCI | 17-GC-IDC | GETCV | GETCVAY2 | | IDC | | | IIA | 55 |
| | | SIIA | | | | | | | | |
| BC | ABS | 18-(27)-AB- | 4907020072T | | | IDC | 3 | T2N0Mx | IIA | |
| | | IDC SIIA | | | | | | | | |
| BC | GCI | 19-GC-IDC | SE5BK | SE5BKAEQ | | IDC | | | IIB | 55 |
| | | SIIB | | | | | | | | |
| BC | GCI | 20-GC-IDC | OLKL4 | OLKL4AO6 | | IDC | | | IIB | 60 |
| | | SIIB | | | | | | | | |
| BC | GCI | 21-GC-IDC | VK1EJ | VK1EJAQE | | IDC | | | IIB | 60 |
| | | SIIB | | | | | | | | |
| BC | GCI | 22-GC-IDC | 3Z5Z4 | 3Z5Z4ANH | | IDC | | | IIB | 85 |
| | | SIIB | | | | | | | | |
| BC | ABS | 23-(13)-AB- | A0133T | | | IDC | 2 | T2N1aMx | IIB | |
| | | IDC SIIB | | | | | | | | |
| BC | GCI | 24-GC-IDC | J5MPN | J5MPNA9Q | | IDC | | | IIB | 55 |
| | | SIIB | | | | | | | | |
| BC | GCI | 25-GC-IDC | 54NTA | 54NTAAKT | | IDC | | | IIB | 70 |
| | | SIIB | | | | | | | | |
| BC | GCI | 27-GC-IDC | RD3F9 | RD3F9AFQ | | IDC | | | IIIA | 90 |
| | | SIIIA | | | | | | | | |
| BC | ABS | 28-(17)-AB- | 4904020036T | | | IDC | 2-3 | T3N1Mx | IIIA | |
| | | IDC SIIIA | | | | | | | | |
| BC | ABS | 29-(16)-AB- | 4904020032T | | | IDC | 2 | T3N1Mx | IIIA | |
| | | IDC IIIA | | | | | | | | |
| BC | ABS | 30-(15)-AB- | 7259T | | | IDC | 2 | T3N1M0 | IIIA | |
| | | IDC SIIIA | | | | | | | | |
| BC | GCI | 31-GC-IDC | YOLOF | YOLOFARG | | IDC | | | IIIA | 85 |
| | | SIIIA | | | | | | | | |
| BC | GCI | 32-GC-IDC | 4W2NY | 4W2NYAC1 | | IDC | | | IIIB | 50 |
| | | SIIIB | | | | | | | | |
| BC | GCI | 33-GC-IDC | YQ1WW | YQ1WWAUV | | IDC | | | IIIB | 60 |
| | | SIIIB | | | | | | | | |
| BC | GCI | 34-GC-IDC | KIOE7 | KIOE7AI9 | | IDC | | | IIIB | 55 |
| | | SIIIB | | | | | | | | |
| BC | Bioch | 70-(43)-Bc- | A609183 | | | IDC | 2 | | | |
| | | IDC | | | | | | | | |
| BC | Bioch | 71-(54)-Bc- | A605353 | | | IDC | 2 | | | |
| | | IDC | | | | | | | | |
| BC | ABS | 72-(55)-Bc- | A609179 | | | IDC | 2 | | | |
| | | IDC | | | | | | | | |
| BC | Bioch | 73-(47)-Bc- | A609221 | | | IDC | 2 | | | |
| | | IDC | | | | | | | | |
| BC | Bioch | 74-(48)-Bc- | A609222 | | | IDC | 2 | | | |
| | | IDC | | | | | | | | |
| BC | Bioch | 75-(53)-Bc- | A605151 | | | IDC | 2 | | | |
| | | IDC | | | | | | | | |
| BC | Bioch | 76-(61)-Bc- | A610029 | | | IDC | 2 | | | |
| | | IDC | | | | | | | | |
| BC | Bioch | 77-(46)-Bc- | A609177 | | | Carc | 2 | | | |
| | | Carci | | | | | | | | |
| BC | Ichilov | 78-(62)-Bc- | A609194 | | | IDC | 2 | | | |
| | | IDC | | | | | | | | |
| BC | Amb | 79-(32)-AB- | 7116T | | | MC | | T2N0M0 | IIA | |
| | | Muc Carci | | | | | | | | |
| | | SIIA | | | | | | | | |
| BC | GCI | 80-(49)-Bc- | A609223 | | | IDC | 2 | | | |
| | | IDC | | | | | | | | |
| BC | GCI | 81-(45)-Bc- | A609181 | | | IDC | 2 | | | |
| | | IDC | | | | | | | | |
| BC | GCI | 82-(50)-Bc- | A609224 | | | IDC | 2 | | | |
| | | IDC | | | | | | | | |
| BC | Bioch | 83-(44)-Bc- | A609198 | | | IDC | 2 | | | |
| | | IDC | | | | | | | | |
| BC | Bioch | 84-(51)-Bc- | A605361 | | | IDC | 1 | | | |
| | | IDC | | | | | | | | |
| BC | Amb | 85-(31)-Ic- | CG-154 | | | IDC | | | | |
| | | IDC | | | | | | | | |
| BC | Aster | 35-As-ILC | 17090 | 30738 | 30738A1 | ILC | | T1cNXM0 | I | 100 |
| | | SI | | | | | | | | |
| BC | GCI | 36-GC-ILC | I35US | I35USA9G | | ILC | | | IIA | 60 |
| | | SIIA | | | | | | | | |
| BC | GCI | 37-GC-ILC | IS84Y | IS84YAAY | | ILC | | | IIB | 65 |
| | | SIIB | | | | | | | | |
| BC | Bioch | 38-(52)-Bc- | A605360 | | | ILC | 1 | | | |
| | | ILC | | | | | | | | |
| BB | Aster | 39-As-Ben | 11975 | 15478 | 15478B1 | FIBR | | | | 100 |
| BB | GCI | 40-GC-Ben | ZT15M | ZT15MAMR | | FIBR | | | | 100 |
| BB | GCI | 41-GC-Ben | NNP3Q | NNP3QA4V | | FIBR | | | | 95 |
| BB | GCI | 42-GC-Ben | QK8IY | QK8IYALU | | FIBR | | | | 100 |
| BN-PS | GCI | 43-GC-N | 83LO7 | 83LO7NEH | | NB-PS | | | | |
| | | PS | | | | | | | | |
| BN-PS | GCI | 45-GC-N | O6JBJ | O6JBJNT1 | | NB-PS | | | | |
| | | PS | | | | | | | | |
| BN-PS | GCI | 46-GC-N | E6UDD | E6UDDNCF | | NB-PS | | | | |
| | | PS | | | | | | | | |
| BN-PS | GCI | 47-GC-N | DHLR1 | DHLR1NIQ | | NB-PS | | | | |
| | | PS | | | | | | | | |
| BN-PS | GCI | 48-GC-N | JHQEH | JHQEHN4D | | NB-PS | | | | |
| | | PS | | | | | | | | |
| BN-PS | Amb | 49-(63)-Am- | 26486 | | | NB-PS | | | | |
| | | N PS | | | | | | | | |
| BN-PS | GCI | 50-GC-N | ONBFK | ONBFKNO2 | | NB-PS | | | | |
| | | PS | | | | | | | | |
| BN-PS | GCI | 51-GC-N | TG6J6 | TG6J6NNA | | NB-PS | | | | |
| | | PS | | | | | | | | |
| BN-PS | Aster | 52-GC-N | 14398 | 20021 | 20021D1 | NB-PS | | | | |
| | | PS | | | | | | | | |
| BN-PS | GCI | 54-CC-N | AJGXV | AJGXVNFC | | NB-PS | | | | |
| | | PS | | | | | | | | |
| BN-PS | GCI | 56-GC-N | HLCZX | HLCZXNLS | | NB-PS | | | | |
| | | PS | | | | | | | | |
| BN-PS | GCI | 58-GC-N | FGV8P | FGV8PNQ6 | | NB-PS | | | | |
| | | PS | | | | | | | | |
| BN-? | Aster | 59-As-N PS | 9264 | 9486 | 9486A1 | NB-PS | | | | |
| BN-PM | Bioch | 60-(57)-Bc- | A609233 | A609233 | | NB-PM | | | | |
| | | N PM | | | | | | | | |
| BN-PM | Bioch | 61-(59)-Bc- | A607155 | A607155 | | NB-PM | | | | |
| | | N PM | | | | | | | | |
| BN-PM | Bioch | 62-(60)-Bc- | A609234 | A609234 | | NB-PM | | | | |
| | | N PM | | | | | | | | |
| BN-PM | Amb | 63-(66)-Am- | 36678 | 36678 | | NB-PM | | | | |
| | | N PM | | | | | | | | |
| BN-PM | Amb | 64-(64)-Am- | 23036 | 23036 | | NB-PM | | | | |
| | | N PM | | | | | | | | |
| BN-PM | Amb | 65-(65)-Am | 31410 | 31410 | | NB-PM | | | | |
| | | N PM | | | | | | | | |
| BN-PM | Amb | 66-(67)-Am- | 073P010602086A | 073P010602086A | | NB-PM | | | | |
| | | N PM | | | | | | | | |
| BN-PM | Bioch | 67-(58)-Bc- | A609232 | A609232 | | NB-PM | | | | |
| | | N PM | | | | | | | | |
| BN-PM | Aster | 68-As-N | 8662 | 8766 | 8766B1 | NB-PM | | | | |
| | | PM | | | | | | | | |
| BN-PM | Aster | 69-As-N | 8457 | 7928 | 7926M1 | NB-PM | | | | |
| | | PM | | | | | | | | |

| | | | | | | | | | Age | | | |
|---|---|---|---|---|---|---|---|---|---|---|---|---|
| | | | | | | | | # | of | | | Year |
| | | | WT | HT | | | # of | Live | first | Br | Recovery | of |
| Tissue | age | MS | (KG) | (CM) | BMI | Ethnic B | Preg. | Bir | child | child | Type | birth |
| BC_in- | 39 | Pre-M | 102 | 157 | 41.4 | CAU | 2 | 1 | | | Surg | |
| situ | | | | | | | | | | | | |
| BC | 39 | Pre-M | 48.1 | 147 | 22.2 | WCAU | 1 | 0 | — | — | Surg | 1962 |
| BC | 42 | | | | | | | | | | | |
| BC | 43 | | | | | | | | | | | |
| BC | 50 | Post-M | 113.4 | 175 | 36.9 | WCAU | 2 | 2 | 17 | 0 | Surg | 1951 |
| BC | 56 | Post-M | 57.6 | 168 | 20.5 | WCAU | 0 | — | — | — | Surg | 1945 |
| BC | 57 | Post-M | 76.2 | 165 | 28.0 | WCAU | 2 | 2 | 17 | 0 | Surg | 1944 |
| BC | 60 | Post-M | 68 | 140 | 34.8 | WCAU | 1 | 1 | 23 | 0 | Surg | 1942 |
| BC | 65 | Post-M | 70 | 168 | 24.8 | CAU | 3 | 2 | | | Aut | |
| BC | 46 | | | | | | | | | | | |
| BC | 46 | Pre-M | 69 | 174 | 22.8 | CAU | 1 | 1 | | | Surg | |
| BC | 46 | Pre-M | 76.2 | 165 | 28.0 | WCAU | 1 | 0 | 0 | 0 | Surg | 1956 |
| BC | 60 | | | | | | | | | | | |
| BC | 60 | | | | | | | | | | | |
| BC | 67 | Post-M | 113.4 | 168 | 40.4 | WCAU | 3 | 1 | 34 | 0 | Surg | 1938 |
| BC | 70 | Pre-M | 79.4 | 163 | 30.0 | WCAU | 2 | 2 | 20 | 0 | Surg | 1932 |
| BC | 70 | Post-M | 72.6 | 163 | 27.5 | WCAU | 2 | 2 | 21 | 0 | Surg | 1931 |
| BC | 91 | | | | | | | | | | | |
| BC | 41 | Pre-M | 61.2 | 165 | 22.5 | WCAU | 0 | — | — | — | Surg | 1960 |
| BC | 46 | Pre-M | 111.1 | 168 | 39.5 | WCAU | 2 | 2 | 24 | 2 | Surg | 1955 |
| BC | 54 | Post-M | 72.6 | 168 | 25.8 | WCAU | 0 | — | — | — | Surg | 1947 |
| BC | 60 | Post-M | 80 | 163 | 30.3 | WCAU | 1 | 1 | 22 | 0 | Surg | 1942 |
| BC | 63 | | | | | | | | | | | |
| BC | 64 | Post-M | 68.5 | 157 | 27.6 | WCAU | 4 | 3 | 25 | 3 | Surg | 1938 |
| BC | 67 | Post-M | 56.7 | 160 | 22.1 | WCAU | 5 | 5 | 20 | 0 | Surg | 1934 |
| BC | 41 | ? | 63.5 | 157 | 25.6 | WCAU | 1 | 1 | 25 | 0 | Surg | 1962 |
| BC | 42 | | | | | | | | | | | |
| BC | 49 | | | | | | | | | | | |
| BC | 59 | | | | | | | | | | | |
| BC | 62 | Post-M | 82.6 | 160 | 32.2 | WCAU | 3 | 3 | 20 | 0 | Surg | 1943 |
| BC | 39 | Pre-M | 54.4 | 163 | 20.6 | WCAU | 2 | 2 | 26 | 0 | Surg | 1962 |
| BC | 62 | Pre-M | 78 | 163 | 29.5 | WCAU | 0 | — | — | — | Surg | 1940 |
| BC | 65 | Post-M | 73.5 | 157 | 29.6 | WCAU | 2 | 2 | 18 | 2 | Surg | 1939 |
| BC | 40 | | | | | | | | | | | |
| BC | 41 | | | | | | | | | | | |
| BC | 42 | | | | | | | | | | | |
| BC | 42 | | | | | | | | | | | |
| BC | 44 | | | | | | | | | | | |
| BC | 44 | | | | | | | | | | | |
| BC | 46 | | | | | | | | | | | |
| BC | 48 | | | | | | | | | | | |
| BC | 51 | | | | | | | | | | | |
| BC | 54 | | | | | | | | | | | |
| BC | 54 | | | | | | | | | | | |
| BC | 58 | | | | | | | | | | | |
| BC | 69 | | | | | | | | | | | |
| BC | 77 | | | | | | | | | | | |
| BC | 79 | | | | | | | | | | | |
| BC | 83 | | | | | | | | | | | |
| BC | 50 | | 94 | 170 | 32.5 | W | 2 | 2 | | | Surg | |
| BC | 70 | | 77.1 | 178 | 24.4 | WCAU | — | — | — | — | Surg | 1932 |
| BC | 67 | Post-M | 62.6 | 163 | 23.7 | WCAU | 2 | 2 | 16 | 0 | Surg | 1934 |
| BC | 60 | | | | | | | | | | | |
| BB | 24 | Pre-M | 80 | 164 | 29.7 | CAU | 2 | 0 | | | Surg | |
| BB | 34 | | 57.6 | 165 | 21.1 | WCAU | | | | | Surg | 1967 |
| BB | 54 | | 56.7 | 157 | 22.9 | WCAU | | | | | Surg | 1948 |
| BB | 41 | | 59 | 173 | 19.7 | WCAU | | | | | Surg | 1960 |
| BN-PS | 32 | | 78.5 | 155 | 32.7 | WCAU | | | | | Surg | 1969 |
| BN-PS | 38 | | 67.1 | 173 | 22.5 | WCAU | | | | | Surg | 1963 |
| BN-PS | 40 | | 99.8 | 170 | 34.5 | WCAU | | | | | Surg | 1961 |
| BN-PS | 40 | | 90.7 | 168 | 32.3 | WCAU | | | | | Surg | 1961 |
| BN-PS | 41 | | 65.3 | 157 | 26.3 | WCAU | | | | | Surg | 1960 |
| BN-PS | 43 | | | | | | | | | | | |
| BN-PS | 45 | | 81.6 | 165 | 30.0 | WCAU | | | | | Surg | 1956 |
| BN-PS | 46 | | 90.7 | 173 | 30.4 | WCAU | | | | | Surg | 1955 |
| BN-PS | 49 | Pre-M | 68 | 165 | 25.0 | CAU | 3 | 3 | | | Surg | |
| BN-PS | 52 | | 70 | 168 | 24.8 | WCAU | | | | | Surg | 1949 |
| BN-PS | 54 | | 67 | 163 | 25.2 | WCAU | | | | | Surg | 1947 |
| BN-PS | 61 | | 106.6 | 168 | 37.9 | WCAU | | | | | Surg | 1940 |
| BN-? | 0 | | 0 | 0 | 0.0 | | | | | | | |
| BN-PM | 34 | | | | | | | | | | Aut | |
| BN-PM | 35 | | | | | | | | | | Aut | |
| BN-PM | 36 | | | | | | | | | | Aut | |
| BN-PM | 45 | | | | | | | | | | Aut | |
| BN-PM | 57 | | | | | | | | | | Aut | |
| BN-PM | 63 | | | | | | | | | | Aut | |
| BN-PM | 64 | | | | | | | | | | Aut | |
| BN-PM | 65 | | | | | | | | | | Aut | |
| BN-PM | 74 | | 64 | 157 | 26.0 | CAU | | | | | Aut | |
| BN-PM | 87 | | 59 | 165 | 21.7 | CAU | | | | | Aut | |

TABLE 1_3_1

| Key | Full Name |
|---|---|
| # Live Bir | # Live Births |
| # of Preg | Number of Pregnancies |
| Amb | Ambion |
| Aster | Asterand |
| Aut | Autopsy |
| BB | Breast Benign |
| BC | Breast Cancer |
| Bioch | Biochain |
| BN | Breast Normal |
| BN-PM | Breast_Normal-PM |
| BN-PS | Breast_Normal-PS |
| Breastfeed child | Br Child |
| C Stage | Cancer Stage |
| Carc | Carcinoma |
| CAU | Caucasian |
| DCIS | Ductal Carcinoma In Situ |
| Ethnic B | Ethnic Background |
| FIBR | FIBROADENOMA |
| IDC | INFILTRATING DUCTAL CARCINOMA |
| ILC | INFILTRATING LOBULAR CARCINOMA |
| M | Menopausal |
| MC | Mucinous carcinoma |
| MS | Menopausal Status |
| NB-PM | NORMAL BREAST-PM |
| NB-PS | NORMAL BREAST-PS |
| Post-M | Post-Menopausal |
| Pre-M | Pre-Menopausal |
| Samp DIAG | Sample Diagnosis |
| Surg | Surgical |
| Tum % | Percentage of Tumor |
| W | White |
| WCAU | White Caucasian |

TABLE 1_4
Tissue samples in normal panel:

| | Lot no. | Source | Tissue | Pathology | Sex/Age |
|---|---|---|---|---|---|
| 1-Am-Colon (C71) | 071P10B | Ambion | Colon | PM IC bleed | F/43 |
| 2-B-Colon (C69) | A411078 | Biochain | Colon | PM-Pool of 10 | M (26-78)&F(53-77) |
| 3-Cl-Colon (C70) | 1110101 | Clontech | Colon | PM-Pool of 3 | M&F (20-50) |
| | | | | sudden death | |
| 4-Am-Small | 091P0201A | Ambion | Small Intestine | PM ICH | M/85 |
| Intestine | | | | | |
| 5-B-Small | A501158 | Biochain | Small Intestine | PM | M/63 |
| Intestine | | | | | |
| 6-B-Rectum | A605138 | Biochain | Rectum | PM | M/25 |
| 7-B-Rectum | A610297 | Biochain | Rectum | PM | M/24 |
| 8-B-Rectum | A610298 | Biochain | Rectum | PM | M/27 |
| 9-Am-Stomach | 110P04A | Ambion | Stomach | PM GSW | M/16 |
| 10-B-Stomach | A501159 | Biochain | Stomach | PM | M/24 |
| 11-B-Esophagus | A603814 | Biochain | Esophagus | PM | M/26 |
| 12-B-Esophagus | A603813 | Biochain | Esophagus | PM | M/41 |
| 13-Am-Pancreas | 071P25C | Ambion | Pancreas | PM MVA | F/25 |
| 14-CG-Pancreas | CG-255-2 | Ichilov | Pancreas | PM | M/75 |
| 15-B-Lung | A409363 | Biochain | Lung | PM-Pool of 5 | M(24-28)&F62 |
| 16-Am-Lung | 111P0103A | Ambion | Lung | PM ICH | F/61 |
| (L93) | | | | | |
| 17-B-Lung (L92) | A503204 | Biochain | Lung | PM | M/28 |
| 19-B-Ovary (O48) | A504087 | Biochain | Ovary | PM | F/51 |
| 20-B-Ovary (O46) | A504086 | Biochain | Ovary | PM | F/41 |
| 75-G-Ovary | L629FRV1 | GCI | Ovary | PS DIGESTIVE | F/47 |
| | | | | HEMORRHAGE | |
| | | | | (ALCOHOLISM) | |
| 76-G-Ovary | DWHTZRQX | GCI | Ovary | PS | F/42 |
| | | | | LEIOMYOMAS | |
| 77-G-Ovary | FDPL9NJ6 | GCI | Ovary | PS VAGINAL | F/56 |
| | | | | BLEEDING | |
| 78-G-Ovary | GWXUZN5M | GCI | Ovary | PS ABNORMAL | F/53 |
| | | | | PAP SMEARS | |
| 21-Am-Cervix | 101P0101A | Ambion | Cervix | PM Surgery | F/40 |
| 23-B-Cervix | A504089 | Biochain | Cervix | PM-Pool of 5 | F (36-55) |
| 24-B-Uterus | A411074 | Biochain | Uterus | PM-Pool of 10 | F (32-53) |
| 25-B-Uterus | A409248 | Biochain | Uterus | PM | F/35 |
| 26-B-Uterus | A504090 | Biochain | Uterus | PM-Pool of 5 | F(40-53) |
| 28-Am-Bladder | 071P02C | Ambion | Bladder | PM GSW | M/28 |
| 29-B-Bladder | A504088 | Biochain | Bladder | PM-Pool of 5 | M(26-44)&F30 |
| 30-Am-Placenta | 021P33A | Ambion | Placenta | PB | F/33 |
| 31-B-Placenta | A410165 | Biochain | Placenta | PB | F/26 |
| 32-B-Placenta | A411073 | Biochain | Placenta | PB-Pool of 5 | F(24-30) |
| 33-B-Breast (B59) | A607155 | Biochain | Breast | PM | F/36 |
| 34-Am-Breast | 26486 | Ambion | Breast | PS bilateral | F/43 |
| (B63) | | | | breast reduction | |
| 35-Am-Breast | 23036 | Ambion | Breast | PM lung cancer | F/57 |
| (B64) | | | | | |
| 36-Cl-Prostate | 1070317 | Clontech | Prostate | PM-Pool of 47 | M (14-57) |
| (P53) | | | | sudden death | |
| 37-Am-Prostate | 061P04A | Ambion | Prostate | PM IC bleed | M/47 |
| (P42) | | | | | |
| 38-Am-Prostate | 25955 | Ambion | Prostate | PM head trauma | M/62 |
| (P59) | | | | | |
| 39-Am-Testis | 111P0104A | Ambion | Testis | PM GSW | M/25 |
| 40-B-Testis | A411147 | Biochain | Testis | PM | M/74 |
| 41-Cl-Testis | 1110320 | Clontech | Testis | PM-Pool of 45 | M (14-64) |
| | | | | sudden death | |
| 42-CG-Adrenal | CG-184-10 | Ichilov | Adrenal | PM | F/81 |
| 43-B-Adrenal | A610374 | Biochain | Adrenal | PM | F/83 |
| 44-B-Heart | A411077 | Biochain | Heart | PM-Pool of 5 | M(23-70) |
| 45-CG-Heart | CG-255-9 | Ichilov | Heart focal | PM | M/75 |
| | | | fibrosis | | |
| 46-CG-Heart | CG-227-1 | Ichilov | Heart | PM | F/36 |
| 47-Am-Liver | 081P0101A | Ambion | Liver | PM ICH | M/64 |
| 48-CG-Liver | CG-93-3 | Ichilov | Liver | PM | F/19 |
| 49-CG-Liver | CG-124-4 | Ichilov | Liver of fetus | PM | fetus |
| 50-Cl-BM | 1110932 | Clontech | Bone Marrow | PM-Pool of 8 | M&F (22-65) |
| | | | | sudden death | |
| 51-CGEN-Blood | WBC#5 | CGEN | Blood | — | M |
| 52-CGEN-Blood | WBC#4 | CGEN | Blood | — | M |
| 53-CGEN-Blood | WBC#3 | CGEN | Blood | — | M |
| 54-CG-Spleen | CG-267 | Ichilov | Spleen | PM | F/25 |
| 55-CG-Spleen | 111P0106B | Ambion | Spleen | PM GSW | M/25 |
| 56-CG-Spleen | A409246 | Biochain | Spleen | PM | F/12 |
| 57-CG-Thymus | CG-98-7 | Ichilov | Thymus | PM | F/28 |
| 58-Am-Thymus | 101P0101A | Ambion | Thymus | PM head injury | M/14 |
| 59-B-Thymus | A409278 | Biochain | Thymus | PM | M/28 |
| 60-B-Thyroid | A610287 | Biochain | Thyroid | PM | M/27 |
| 61-B-Thyroid | A610286 | Biochain | Thyroid | PM | M/24 |
| 62-CG-Thyroid | CG-119-2 | Ichilov | Thyroid | PM | F/66 |
| 63-Cl-Salivary | 1070319 | Clontech | Salivary Gland | PM-Pool of 24 | M&F 15-60 |
| Gland | | | | sudden death | |
| 64-Am-Kidney | 111P0101B | Ambion | Kidney | PM ICH | M 60 |
| 65-Cl-Kidney | 1110970 | Clontech | Kidney | PM-Pool of 14 | M&F 18-59 |
| | | | | sudden death | |
| 66-B-Kidney | A411080 | Biochain | Kidney | PM-Pool of 5 | M24-46 |
| 67-CG-Cerebellum | CG-183-5 | Ichilov | Cerebellum | PM | M/74 |
| 68-CG-Cerebellum | CG-212-5 | Ichilov | Cerebellum | PM | M/54 |
| 69-B-Brain | A411322 | Biochain | Brain | PM | M/28 |
| 70-Cl-Brain | 1120022 | Clontech | Brain | PM | — |
| 71-B-Brain | A411079 | Biochain | Brain | PM-Pool of 2 | M27-28 |
| 72-CG-Brain | CG-151-1 | Ichilov | Brain | PM | F/86 |
| 73-Am-Skeletal | 101P013A | Ambion | Skeletal | PM head injury | F/28 |
| Muscle | | | Muscle | | |
| 74-Cl-Skeletal | 1061038 | Clontech | Skeletal | PM-Pool of 2 | M&F 43-46 |
| Muscle | | | Muscle | sudden death | |

### Materials and Experimental Procedures

### RNA Preparation—

RNA was obtained from ABS (Wilmington, Del. 19801, USA, http://www.absbioreagents.com), BioChain Inst. Inc. (Hayward, Calif. 94545 USA www.biochain.com), GOG for ovary samples—Pediatic Cooperative Human Tissue Network, Gynecologic Oncology Group Tissue Bank, Children Hospital of Columbus (Columbus Ohio 43205 USA), Clontech (Franklin Lakes, N.J. USA 07417, www.eclontech.com), Ambion (Austin, Tex. 78744 USA, http://www.ambion.com), Asternad (Detroit, Mich. 48202-3420, USA, www.asterand.com), and from Genomics Collaborative Inc.a Division of Seracare (Cambridge, Mass. 02139, USA, www.genomicsinc.com). Alternatively, RNA was generated from tissue samples using TRI-Reagent (Molecular Research Center), according to Manufacturer's instructions. Tissue and RNA samples were obtained from patients or from postmortem. Total RNA samples were treated with DNaseI (Ambion).

RT PCR—Purified RNA (1 μg) was mixed with 150 ng Random Hexamer primers (Invitrogen) and 500 μM DNTP in a total volume of 15.6 μl. The mixture was incubated for 5 min at 65° C. and then quickly chilled on ice. Thereafter, 5 μl of 5× SuperscriptII first strand buffer (Invitrogen), 2.4 μl 0.1M DTT and 40 units RNasin (Promega) were added, and the mixture was incubated for 10 min at 25° C., followed by further incubation at 42° C. for 2 min. Then, 1 μl (200 units) of SuperscriptII (Invitrogen) was added and the reaction (final volume of 2541) was incubated for 50 min at 42° C. and then inactivated at 70° C. for 15 min. The resulting cDNA was diluted 1:20 in TE buffer (10 mM Tris pH=8, 1 mM EDTA pH=8).

Real-Time RT-PCR analysis—cDNA (5 μl), prepared as described above, was used as a template in Real-Time PCR reactions using the SYBR Green I assay (PE Applied Biosystem) with specific primers and UNG Enzyme (Eurogentech or ABI or Roche). The amplification was effected as follows: 50° C. for 2 min, 95° C. for 10 min, and then 40 cycles of 95° C. for 15 sec, followed by 60° C. for 1 min. Detection was performed by using the PE Applied Biosystem SDS 7000. The cycle in which the reactions achieved a threshold level (Ct) of fluorescence was registered and was used to calculate the relative transcript quantity in the RT reactions. The relative quantity was calculated using the equation Q=efficiencŷ-Ct. The efficiency of the PCR reaction was calculated from a standard curve, created by using serial dilutions of several reverse transcription (RT) reactions. To minimize inherent differences in the RT reaction, the resulting relative quantities were normalized to normalization factor calculated in the following methods:

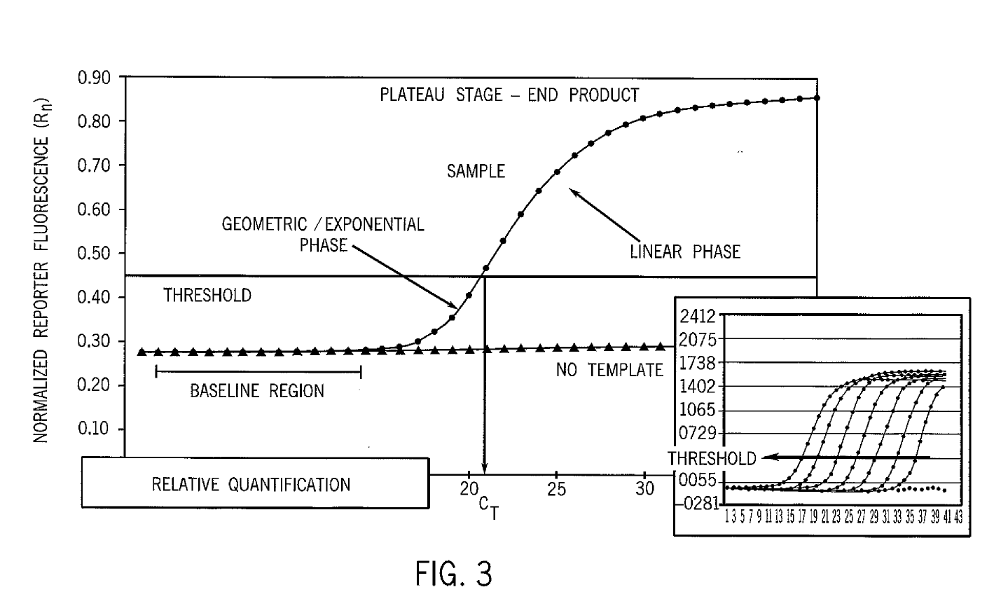

The expression of several housekeeping (HSKP) genes was checked on every panel. The relative quantity (O) of each housekeeping gene in each sample, calculated as described above, was divided by the median quantity of this gene in all panel samples to obtain the “relative Q rel to MED”. Then, for each sample the median of the “relative Q rel to MED” of the selected housekeeping genes was calculated and served as normalization factor of this sample for further calculations. Schematic summary of quantitative real-time PCR analysis is presented in FIG. 3. As shown, the x-axis shows the cycle number. The CT=Threshold Cycle point, which is the cycle that the amplification curve crosses the fluorescence threshold that was set in the experiment. This point is a calculated cycle number in which PCR products signal is above the background level (passive dye ROX) and still in the Geometric/Exponential phase (as shown, once the level of fluorescence crosses the measurement threshold, it has a geometrically increasing phase, during which measurements are most accurate, followed by a linear phase and a plateau phase; for quantitative measurements, the latter two phases do not provide accurate measurements). The y-axis shows the normalized reporter fluorescence. It should be noted that this type of analysis provides relative quantification.

The sequences of the housekeeping genes measured in all the examples on ovarian cancer panel were as follows:

SDHA (GenBank Accession No. NM_{—}004168 (SEQ ID NO:1);

SDHA Forward primer (SEQ ID NO: 2):
TGGGAACAAGAGGGCATCTG
SDHA Reverse primer (SEQ ID NO: 3):
CCACCACTGCATCAAATTCATG
SDHA-amplicon (SEQ ID NO: 4):
TGGGAACAAGAGGGCATCTGCTAAAGTTTCAGATTCCATTTCTGCTCAGT
ATCCAGTAGTGGATCATGAATTTGATGCAGTGGTGG

PBGD (GenBank Accession No. BC019323 (SEQ ID NO:5))),

PBGD Forward primer (SEQ ID NO: 6):
TGAGAGTGATTCGCGTGGG
PBGD Reverse primer (SEQ ID NO: 7):
CCAGGGTACGAGGCTTTCAAT
PBGD-amplicon (SEQ ID NO: 8):
TGAGAGTGATTCGCGTGGGTACCCGCAAGAGCCAGCTTGCTCGCATACAG
ACGGACAGTGTGGTGGCAACATTGAAAGCCTCGTACCCTGG

HPRT1 (GenBank Accession No. NM_{—}000194 (SEQ ID NO:9))),

HPRT1 Forward primer (SEQ ID NO: 10):
TGACACTGGCAAAACAATGCA
HPRT1 Reverse primer (SEQ ID NO: 11):
GGTCCTTTTCACCAGCAAGCT
HPRT1-amplicon (SEQ ID NO: 12):
TGACACTGGCAAAACAATGCAGACTTTGCTTTCCTTGGTCAGGCAGTATA
ATCCAAAGATGGTCAAGGTCGCAAGCTTGCTGGTGAAAAGGACC

GAPDH (GenBank Accession No. BC026907 (SEQ ID NO:13),)

GAPDH Forward primer (SEQ ID NO: 14):
TGCACCACCAACTGCTTAGC
GAPDH Reverse primer (SEQ ID NO: 15):
CCATCACGCCACAGTTTCC
GAPDH-amplicon (SEQ ID NO: 16):
TGCACCACCAACTGCTTAGCACCCCTGGCCAAGGTCATCCATGACAACTT
TGGTATCGTGGAAGGACTCATGACCACAGTCCATGCCATCACTGCCACCC
AGAAGACTGTGGATGG

The sequences of the housekeeping genes measured in all the examples on colon cancer tissue testing panel were as follows:

PBGD (GenBank Accession No. BC019323 (SEQ ID NO:5))),

PBGD Forward primer (SEQ ID NO: 6):
TGAGAGTGATTCGCGTGGG
PBGD Reverse primer (SEQ ID NO: 7):
CCAGGGTACGAGGCTTTCAAT
PBGD-amplicon (SEQ ID NO: 8):
TGAGAGTGATTCGCGTGGGTACCCGCAAGAGCCAGCTTGCTCGCATACAG
ACGGACAGTGTGGTGGCAACATTGAAAGCCTCGTACCCTGG

HPRT1 (GenBank Accession No. NM_{—}000194 (SEQ ID NO:9))),

HPRT1 Forward primer (SEQ ID NO: 10):
TGACACTGGCAAAACAATGCA
HPRT1 Reverse primer (SEQ ID NO: 11):
GGTCCTTTTCACCAGCAAGCT
HPRT1-amplicon (SEQ ID NO: 12):
TGACACTGGCAAAACAATGCAGACTTTGCTTTCCTTGGTCAGGCAGTATA
ATCCAAAGATGGTCAAGGTCGCAAGCTTGCTGGTGAAAAGGACC

G6PD (GenBank Accession No. NM_{—}000402 (SEQ ID NO: 17))

G6PD Forward primer (SEQ ID NO: 18):
gaggccgtcaccaagaacat
G6PD Reverse primer (SEQ ID NO: 19):
ggacagccggtcagagctc
G6PD-amplicon (SEQ ID NO:20):
gaggccgtcaccaagaacattcacgagtcctgcatgagccagataggctg
gaaccgcatcatcgtggagaagcccttcgggagggacctgcagagctctg
accggctgtcc

RPS27A (GenBank Accession No. NM_{—}002954 (SEQ ID NO:21),)

RPS27A Forward primer (SEQ ID NO: 22):
CTGGCAAGCAGCTGGAAGAT
RPS27A Reverse primer (SEQ ID NO: 23):
TTTCTTAGCACCACCACGAAGTC
RPS27A-amplicon (SEQ ID NO: 24):
CTGGCAAGCAGCTGGAAGATGGACGTACTTTGTCTGACTACAATATTCAA
AAGGAGTCTACTCTTCATCTTGTGTTGAGACTTCGTGGTGGTGCTAAGAA
A

The sequences of the housekeeping genes measured in all the examples in the lung panel were as follows:

Ubiquitin (GenBank Accession No. BC000449 (SEQ ID NO:25))

Ubiquitin Forward primer (SEQ ID NO: 26):
ATTTGGGTCGCGGTTCTTG
Ubiquitin Reverse primer (SEQ ID NO: 27):
TGCCTTGACATTCTCGATGGT
Ubiquitin-amplicon (SEQ ID NO: 28)
ATTTGGGTCGCGGTTCTTGTTTGTGGATCGCTGTGATCGTCACTTGACAA
TGCAGATCTTCGTGAAGACTCTGACTGGTAAGACCATCACCCTCGAGGTT
GAGCCCAGTGACACCATCGAGAATGTCAAGGCA

SDHA (GenBank Accession No. NM_{—}004168 (SEQ ID NO:1))

SDHA Forward primer (SEQ ID NO: 2):
TGGGAACAAGAGGGCATCTG
SDHA Reverse primer (SEQ ID NO: 3):
CCACCACTGCATCAAATTCATG
SDHA-amplicon (SEQ ID NO: 4):
TGGGAACAAGAGGGCATCTGCTAAAGTTTCAGATTCCATTTCTGCTCAGT
ATCCAGTAGTGGATCATGAATTTGATGCAGTGGTGG

PBGD (GenBank Accession No. BC019323 (SEQ ID NO:5))),

PBGD Forward primer (SEQ ID NO: 6):
TGAGAGTGATTCGCGTGGG
PBGD Reverse primer (SEQ ID NO: 7):
CCAGGGTACGAGGCTTTCAAT
PBGD-amplicon (SEQ ID NO: 8):
TGAGAGTGATTCGCGTGGGTACCCGCAAGAGCCAGCTTGCTCGCATACAG
ACGGACAGTGTGGTGGCAACATTGAAAGCCTCGTACCCTGG

HPRT1 (GenBank Accession No. NM_{—}000194 (SEQ ID NO:9))),

HPRT1 Forward primer (SEQ ID NO: 10):
TGACACTGGCAAAACAATGCA
HPRT1 Reverse primer (SEQ ID NO: 11):
GGTCCTTTTCACCAGCAAGCT
HPRT1-amplicon (SEQ ID NO: 12):
TGACACTGGCAAAACAATGCAGACTTTGCTTTCCTTGGTCAGGCAGTATA
ATCCAAAGATGGTCAAGGTCGCAAGCTTGCTGGTGAAAGGACC

The sequences of the housekeeping genes measured in all the examples on breast cancer panel were as follows:

G6PD (GenBank Accession No. NM_{—}000402 (SEQ ID NO: 17))

G6PD Forward primer (SEQ ID NO: 18):
gaggccgtcaccaagaacat
G6PD Reverse primer (SEQ ID NO: 19):
ggacagccggtcagagctc
G6PD-amplicon (SEQ ID NO:20):
gaggccgtcaccaagaacattcacgagtcctgcatgagccagataggctg
gaaccgcatcatcgtggagaagcccttcgggagggacctgcagagctctg
accggctgtcc

SDHA (GenBank Accession No. NM_{—}004168 (SEQ ID NO: 1))

SDHA Forward primer (SEQ ID NO: 2):
TGGGAACAAGAGGGCATCTG
SDHA Reverse primer (SEQ ID NO: 3):
CCACCACTGCATCAAATTCATG
SDHA-amplicon (SEQ ID NO: 4):
TGGGAACAAGAGGGCATCTGCTAAAGTTTCAGATTCCATTTCTGCTCAGT
ATCCAGTAGTGGATCATGAATTTGATGCAGTGGTGG

PBGD (GenBank Accession No. BC019323 (SEQ ID NO:5))),

PBGD Forward primer (SEQ ID NO: 6):
TGAGAGTGATTCGCGTGGG
PBGD Reverse primer (SEQ ID NO: 7):
CCAGGGTACGAGGCTTTCAAT
PBGD-amplicon (SEQ ID NO: 8):
TGAGAGTGATTCGCGTGGGTACCCGCAAGAGCCAGCTTGCTCGCATACAG
ACGGACAGTGTGGTGGCAACATTGAAAGCCTCGTACCCTGG

HPRT1 (GenBank Accession No. NM_{—}000194 (SEQ ID NO:9))),

HPRT1 Forward primer (SEQ ID NO: 10):
TGACACTGGCAAAACAATGCA
HPRT1 Reverse primer (SEQ ID NO: 11):
GGTCCTTTTCACCAGCAAGCT
HPRT1-amplicon (SEQ ID NO: 12):
TGACACTGGCAAAACAATGCAGACTTTGCTTTCCTTGGTCAGGCAGTATA
ATCCAAAGATGGTCAAGGTCGCAAGCTTGCTGGTGAAAAGGACC

The sequences of the housekeeping genes measured in all the examples on normal tissue samples panel were as follows:

RPL19 (GenBank Accession No. NM_{—}000981 (SEQ ID NO:29))

RPL19Forward primer (SEQ ID NO: 30):
TGGCAAGAAGAAGGTCTGGTTAG
RPL19Reverse primer (SEQ ID NO: 31):
TGATCAGCCCATCTTTGATGAG
RPL19-amplicon (SEQ ID NO: 32):
TGGCAAGAAGAAGGTCTGGTTAGACCCCAATGAGACCAATGAAATCGCCA
ATGCCAACTCCCGTCAGCAGATCCGGAAGCTCATCAAAGATGGGCTGATC
A

TATA box (GenBank Accession No. NM_{—}003194 (SEQ ID NO:33))),

TATA box Forward primer (SEQ ID NO: 34):
CGGTTTGCTGCGGTAATCAT
TATA box Reverse primer (SEQ ID NO: 35):
TTTCTTGCTGCCAGTCTGGAC
TATA box-amplicon (SEQ ID NO: 36):
CGGTTTGCTGCGGTAATCATGAGGATAAGAGAGCCACGAACCACGGCACT
GATTTTCAGTTCTGGGAAAATGGTGTGCACAGGAGCCAAGAGTGAAGAAC
AGTCCAGACTGGCAGCAAGAAA

Ubiquitin (GenBank Accession No. BC000449 (SEQ ID NO:25))

Ubiquitin Forward primer (SEQ ID NO: 26):
ATTTGGGTCGCGGTTCTTG
Ubiquitin Reverse primer (SEQ ID NO: 27):
TGCCTTGACATTCTCGATGGT
Ubiquitin-amplicon (SEQ ID NO: 28)
ATTTGGGTCGCGGTTCTTGTTTGTGGATCGCTGTGATCGTCACTTGACAA
TGCAGATCTTCGTGAAGACTCTGACTGGTAAGACCATCACCCTCGAGGTT
GAGCCCAGTGACACCATCGAGAATGTCAAGGCA

SDHA (GenBank Accession No. NM_{—}004168 (SEQ ID NO:1))

SDHA Forward primer (SEQ ID NO: 2):
TGGGAACAAGAGGGCATCTG
SDHA Reverse primer (SEQ ID NO: 3):
CCACCACTGCATCAAATTCATG
SDHA-amplicon (SEQ ID NO: 4):
TGGGAACAAGAGGGCATCTGCTAAAGTTTCAGATTCCATTTCTGCTCAGT
ATCCAGTAGTGGATCATGAATTTGATGCAGTGGTGG

### Oligonucleotide-Based Micro-Array Experiment Protocol

### Microarray Fabrication

Microarrays (chips) were printed by pin deposition using the MicroGrid II MGII 600 robot from BioRobotics Limited (Cambridge, UK). 50-mer oligonucleotides target sequences were designed by Compugen Ltd (Tel-Aviv, IL) as described by A. Shoshan et al, “Optical technologies and informatics”, Proceedings of SPIE. Vol 4266, pp. 86-95 (2001). The designed oligonucleotides were synthesized and purified by desalting with the Sigma-Genosys system (The Woodlands, TX, US) and all of the oligonucleotides were joined to a C6 amino-modified linker at the 5′ end, or being attached directly to CodeLink slides (Cat #25-6700-01. Amersham Bioscience, Piscataway, N.J., US). The 50-mer oligonucleotides, forming the target sequences, were first suspended in Ultra-pure DDW (Cat # 01-866-1A Kibbutz Beit-Haemek, Israel) to a concentration of 50 μM. Before printing the slides, the oligonucleotides were resuspended in 300 mM sodium phosphate (pH 8.5) to final concentration of 150 mM and printed at 35-40% relative humidity at 21° C.

Each slide contained a total of 9792 features in 32 subarrays. Of these features, 4224 features were sequences of interest according to the present invention and negative controls that were printed in duplicate. An additional 288 features (96 target sequences printed in triplicate) contained housekeeping genes from Human Evaluation Library2, Compugen Ltd, Israel. Another 384 features are E. coli spikes 1-6, which are oligos to E-Coli genes which are commercially available in the Array Control product (Array control—sense oligo spots, Ambion Inc. Austin, Tex. Cat #1781, Lot #112K06).

### Post-Coupling Processing of Printed Slides

After the spotting of the oligonucleotides to the glass (CodeLink) slides, the slides were incubated for 24 hours in a sealed saturated NaCl humidification chamber (relative humidity 70-75%).

Slides were treated for blocking of the residual reactive groups by incubating them in blocking solution at 50° C. for 15 minutes (10 ml/slide of buffer containing 0.1M Tris, 50 mM ethanolamine, 0.1% SDS). The slides were then rinsed twice with Ultra-pure DDW (double distilled water). The slides were then washed with wash solution (10 ml/slide. 4×SSC, 0.1% SDS)) at 50° C. for 30 minutes on the shaker. The slides were then rinsed twice with Ultra-pure DDW, followed by drying by centrifugation for 3 minutes at 800 rpm.

Next, in order to assist in automatic operation of the hybridization protocol, the slides were treated with Ventana Discovery hybridization station barcode adhesives. The printed slides were loaded on a Bio-Optica (Milan, Italy) hematology staining device and were incubated for 10 minutes in 50 ml of 3-Aminopropyl Triethoxysilane (Sigma A3648 lot #122K589). Excess fluid was dried and slides were then incubated for three hours in 20 mm/Hg in a dark vacuum desiccator (Pelco 2251, Ted Pella, Inc. Redding CA).

The following protocol was then followed with the Genisphere 900—RP (random primer), with mini elute columns on the Ventana Discovery HybStation™, to perform the microarray experiments. Briefly, the protocol was performed as described with regard to the instructions and information provided with the device itself. The protocol included cDNA synthesis and labeling. cDNA concentration was measured with the TBS-380 (Turner Biosystems. Sunnyvale, Calif.) PicoFlour, which is used wi108-USth the OliGreen ssDNA Quantitation reagent and kit.

Hybridization was performed with the Ventana Hybridization device, according to the provided protocols (Discovery Hybridization Station Tuscon AZ).

The slides were then scanned with GenePix 4000B dual laser scanner from Axon Instruments Inc, and analyzed by GenePix Pro 5.0 software.

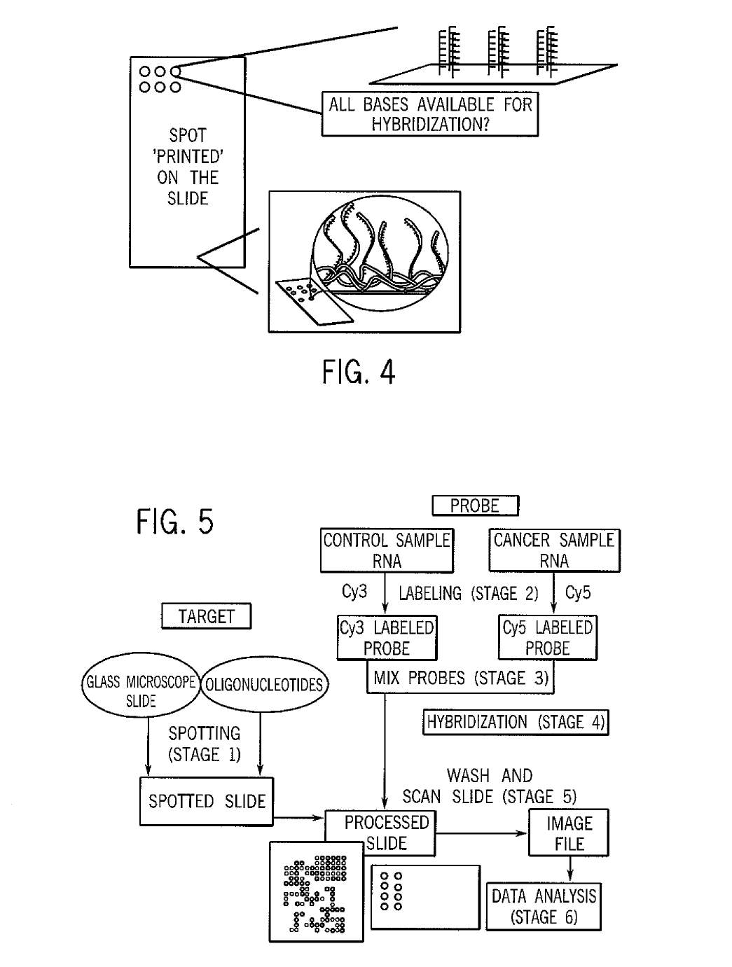

Schematic summary of the oligonucleotide based microarray fabrication and the experimental flow is presented in FIGS. 4 and 5.

Briefly, as shown in FIG. 4, DNA oligonucleotides at 25 uM were deposited (printed) onto Amersham ‘CodeLink’ glass slides generating a well defined ‘spot’. These slides are covered with a long-chain, hydrophilic polymer chemistry that creates an active 3-D surface that covalently binds the DNA oligonucleotides 5′-end via the C6-amine modification. This binding ensures that the full length of the DNA oligonucleotides is available for hybridization to the cDNA and also allows lower background, high sensitivity and reproducibility.

FIG. 5 shows a schematic method for performing the microarray experiments. It should be noted that stages on the left-hand or right-hand side may optionally be performed in any order, including in parallel, until stage 4 (hybridization). Briefly, on the left-hand side, the target oligonucleotides are being spotted on a glass microscope slide (although optionally other materials could be used) to form a spotted slide (stage 1). On the right hand side, control sample RNA and cancer sample RNA are Cy3 and Cy5 labeled, respectively (stage 2), to form labeled probes. It should be noted that the control and cancer samples come from corresponding tissues (for example, normal prostate tissue and cancerous prostate tissue). Furthermore, the tissue from which the RNA was taken is indicated below in the specific examples of data for particular clusters, with regard to overexpression of an oligonucleotide from a “chip” (microarray), as for example “prostate” for chips in which prostate cancerous tissue and normal tissue were tested as described above. In stage 3, the probes are mixed. In stage 4, hybridization is performed to form a processed slide. In stage 5, the slide is washed and scanned to form an image file, followed by data analysis in stage 6.

### Actual Marker Examples

### Description for Cluster T29749

Cluster T29749 features 4 transcript(s) and 29 segment(s) of interest, the names for which are given in Tables 2 and 3, respectively, the sequences themselves are given at the end of the application. The selected protein variants are given in table 4.

TABLE 2
Transcripts of interest
Transcript Name

T29749_T1 (SEQ ID NO: 38)
T29749_T7 (SEQ ID NO: 39)
T29749_T14 (SEQ ID NO: 40)
T29749_T20 (SEQ ID NO: 41)

TABLE 3
Segments of interest
Segment Name

T29749_N0 (SEQ ID NO: 42)
T29749_N29 (SEQ ID NO: 43)
T29749_N33 (SEQ ID NO: 44)
T29749_N34 (SEQ ID NO: 45)
T29749_N40 (SEQ ID NO: 46)
T29749_N41 (SEQ ID NO: 47)
T29749_N42 (SEQ ID NO: 48)
T29749_N46 (SEQ ID NO: 49)
T29749_N2 (SEQ ID NO: 50)
T29749_N3 (SEQ ID NO: 51)
T29749_N4 (SEQ ID NO: 52)
T29749_N7 (SEQ ID NO: 53)
T29749_N8 (SEQ ID NO: 54)
T29749_N9 (SEQ ID NO: 55)
T29749_N10 (SEQ ID NO: 56)
T29749_N11 (SEQ ID NO: 57)
T29749_N12 (SEQ ID NO: 58)
T29749_N15 (SEQ ID NO: 59)
T29749_N17 (SEQ ID NO: 60)
T29749_N18 (SEQ ID NO: 61)
T29749_N22 (SEQ ID NO: 62)
T29749_N23 (SEQ ID NO: 63)
T29749_N24 (SEQ ID NO: 64)
T29749_N30 (SEQ ID NO: 65)
T29749_N31 (SEQ ID NO: 66)
T29749_N36 (SEQ ID NO: 67)
T29749_N38 (SEQ ID NO: 68)
T29749_N44 (SEQ ID NO: 69)
T29749_N50 (SEQ ID NO: 70)

TABLE 4
Proteins of interest

| Protein Name | Corresponding Transcript(s) |
|---|---|
| T29749_P0 (SEQ ID NO: 74) | T29749_T1 (SEQ ID NO: 38) |
| T29749_P13 (SEQ ID NO: 75) | T29749_T20 (SEQ ID NO: 41) |
| T29749_P34 (SEQ ID NO: 76) | T29749_T7 (SEQ ID NO: 39) |
| T29749_P40 (SEQ ID NO: 77) | T29749_T14 (SEQ ID NO: 40) |

These sequences are variants of the known protein Ribonucleoside-diphosphate reductase M2 chain (SwissProt accession identifier RIR21UHUMAN; known also according to the synonyms EC 1.17.4.1; Ribonucleotide reductase small chain), referred to herein as the previously known protein.

Protein Ribonucleoside-diphosphate reductase M2 chain is known or believed to have the following function(s): Provides the precursors necessary for DNA synthesis. Protein Ribonucleoside-diphosphate reductase M2 chain localization is believed to be Cytoplasmic.

The following GO Annotation(s) apply to the previously known protein. The following annotation(s) were found: DNA replication, which are annotation(s) related to Biological Process; ribonucleoside-diphosphate reductase activity, which are annotation(s) related to Molecular Function; and cytoplasm, which are annotation(s) related to Cellular Component.

The GO assignment relies on information from one or more of the SwissProt/TremB1 Protein knowledgebase, available from <http://www.expasy.ch/sprot/>; or Locuslink, available from <http://www.ncbi.nlm.nih.gov/projects/LocusLink/>.

Cluster T29749 can be used as a diagnostic marker according to overexpression of transcripts of this cluster in cancer. Preferably, Cluster T29749 known wild type and Cluster T29749 variants can be used as a diagnostic marker for Ovarian cancer; Lung cancer; breast cancer; or colon cancer. Cluster T29749 known wild type protein can be used for Immunoassays or for any enzymatic assay.

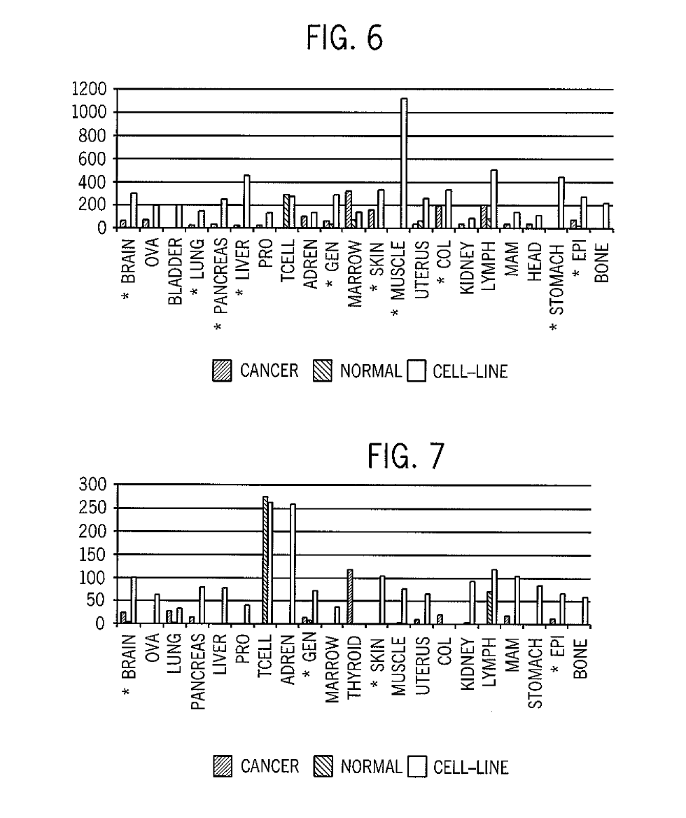

Expression of T29749 transcripts in normal tissues is also given according to the previously described methods. The term “number” in table 5 and the numbers on the y-axis of the FIG. 6 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 6 and Table 5. This cluster is overexpressed (at least at a minimum level) in the following pathological conditions: brain malignant tumors, lung malignant tumors, pancreas carcinoma, hepatocellular carcinoma, a mixture of malignant tumors from different tissues, skin malignancies, myosarcoma, colorectal cancer, gastric carcinoma and epithelial malignant tumors.

TABLE 5
Normal tissue distribution

| Name of Tissue | Number |
|---|---|
| brain | 3 |
| ovary | 7 |
| bladder | 0 |
| lung | 2 |
| pancreas | 4 |
| liver | 4 |
| prostate | 3 |
| T cells | 278 |
| adrenal | 0 |
| general | 24 |
| bone marrow | 62 |
| skin | 0 |
| muscle | 5 |
| uterus | 49 |
| colon | 0 |
| kidney | 2 |
| lymph nodes | 65 |
| breast | 4 |
| head and neck | 0 |
| stomach | 0 |
| epithelial | 6 |
| bone | 0 |

TABLE 6
P values and ratios for expression in cancerous tissue

| Name of Tissue | P1 | P2 | SP1 | R3 | SP2 | R4 |
|---|---|---|---|---|---|---|
| brain | 3.5e−01 | 1.1e−03 | 4.8e−04 | 5.4 | 5.2e−17 | 22.2 |
| ovary | 6.7e−01 | 5.6e−01 | 2.2e−01 | 2.4 | 7.1e−02 | 2.4 |
| bladder | N/A | 3.4e−01 | N/A | N/A | 2.1e−01 | 2.4 |
| lung | 1.6e−01 | 8.5e−03 | 4.1e−01 | 2.6 | 1.1e−03 | 8.0 |
| pancreas | 5.2e−01 | 3.7e−01 | 1.8e−01 | 2.7 | 8.5e−04 | 3.3 |
| liver | 3.3e−01 | 1.3e−01 | N/A | N/A | 1.5e−03 | 4.6 |
| prostate | 7.9e−01 | 5.4e−01 | 4.5e−01 | 1.7 | 9.8e−02 | 2.3 |
| T cells | 5.0e−01 | 6.7e−01 | 1.0e+00 | 0.5 | 8.1e−01 | 0.9 |
| adrenal | 3.8e−01 | 1.6e−01 | 2.1e−01 | 3.4 | 1.5e−01 | 3.6 |
| general | 4.0e−03 | 4.3e−16 | 8.6e−05 | 2.0 | 1.9e−56 | 6.3 |
| bone marrow | 5.6e−01 | 5.2e−01 | 4.1e−01 | 3.6 | 2.9e−01 | 2.0 |
| skin | 1.2e−01 | 2.8e−03 | 1.5e−01 | 12.1 | 1.3e−08 | 16.6 |
| muscle | 9.2e−01 | 4.8e−01 | N/A | N/A | 5.0e−12 | 3.1 |
| uterus | 3.0e−01 | 7.4e−02 | 8.8e−01 | 0.7 | 8.3e−02 | 1.3 |
| colon | 4.9e−03 | 1.9e−03 | 8.8e−03 | 5.6 | 1.8e−03 | 6.2 |
| kidney | 5.9e−01 | 3.9e−01 | 3.3e−01 | 2.2 | 1.6e−01 | 2.9 |
| lymph nodes | 2.6e−01 | 7.1e−02 | 1.1e−01 | 2.0 | 5.2e−05 | 2.9 |
| breast | 6.1e−01 | 8.6e−02 | 6.9e−01 | 1.3 | 2.5e−01 | 2.1 |
| head and neck | 2.1e−01 | 1.7e−01 | N/A | N/A | 5.7e−01 | 1.7 |
| stomach | 9.2e−01 | 4.8e−02 | N/A | N/A | 4.3e−04 | 7.1 |
| epithelial | 9.8e−05 | 9.8e−13 | 3.2e−07 | 6.2 | 6.1e−30 | 18.3 |
| bone | N/A | 1.7e−01 | N/A | N/A | 2.7e−02 | 3.7 |

As noted above, cluster T29749 features 4 transcript(s), which were listed in Table 2 above. These transcript(s) encode for protein(s) which are variant(s) of protein Ribonucleoside-diphosphate reductase M2 chain. A description of each variant protein according to the present invention is now provided.

Variant protein T29749_P0 (SEQ ID NO:74) according to the present invention has an amino acid sequence as given at the end of the application; it is encoded by transcript(s) (SEQ ID NO:38). An alignment is given to the known protein (Ribonucleoside-diphosphate reductase M2 chain) at the end of the application. One or more alignments to one or more previously published protein sequences are given at the end of the application. A brief description of the relationship of the variant protein according to the present invention to each such aligned protein is as follows:

1. Comparison report between T29749_P0 (SEQ ID NO:74) and RIR2HUMAN (SEQ ID NO. 134):
A. An isolated chimeric polypeptide encoding for T29749_P0 (SEQ ID NO:74), comprising a first amino acid sequence being at least 70%, optionally at least 80%, preferably at least 85%, more preferably at least 90% and most preferably at least 95%, homologous to a polypeptide having the sequence MGRVGGMAQPMGRAGAPKPMGRAGSARRGRFKGCWSEGSPVHPVPAVLSWLLALLR CAAT (SEQ ID NO: 128) corresponding to amino acids 1-60 of T29749-P0 (SEQ ID NO:74), and a second amino acid sequence being at least 90% or 95% homologous to MLSLRVPLAPITDPQQLQLSPLKGLSLVDKENTPPALSGTRVLASKTARRIFQEPTEPKTK AAAPGVEDEPLLRENPRRFVIFPIEYHDIWQMYKKAEASFWTAEEVDLSKDIQHWESLK PEERYFISHVLAFFAASDGIVNENLVERFSQEVQITEARCFYGFQLAMENIHSEMYSLLIIDT YIKDPKEREFLFNAIETMPCVKKKADWALRWIGDKEATYGERVVAFAAVEGIFFSGSFA SIFWLKKRGLMPGLTFSNELISRDEGLHCDFACLMFKHLVHIKPSEERVREIIINAVRIEQE FLTEALPVKLIGMNCTLMKQYIEFVADRLMLELGFSKVFRVENPFDFMENISLEGKTNFF EKRVGEYQRMGVMSSPTENSFTLDADF corresponding to amino acids 1-389 of RIR2_HUMAN (SEQ ID NO:134), which also corresponds to amino acids 61-449 of T29749_P0 (SEQ ID NO:74), wherein said first amino acid sequence and second amino acid sequence are contiguous and in a sequential order.
B. An isolated polypeptide encoding for a head of T29749_P0 (SEQ ID NO:74), comprising a polypeptide being at least 70%, optionally at least about 80%, preferably at least about 85%, more preferably at least about 90% and most preferably at least about 95% homologous to the sequence MGRVGGMAQPMGRAGAPKPMGRAGSARRGRFKGCWSEGSPVHPVPAVLSWLLALLR CAAT (SEQ ID NO: 128) of T29749_P0 (SEQ ID NO:74).

2. Comparison report between T29749_P0 (SEQ ID NO:74) and NP_{—}001025 (SEQ ID NO:71):
A. An isolated chimeric polypeptide encoding for T29749_P0 (SEQ ID NO:74), comprising a first amino acid sequence being at least 70%, optionally at least 80%, preferably at least 85%, more preferably at least 90% and most preferably at least 95%, homologous to a polypeptide having the sequence MGRVGGMAQPMGRAGAPKPMGRAGSARRGRFKGCWSEGSPVHPVPAVLSWLLALLR CAAT (SEQ ID NO: 128) corresponding to amino acids 1-60 of T29749_P0 (SEQ ID NO:74), and a second amino acid sequence being at least 90% or 95% homologous to MLSLRVPLAPITDPQQLQLSPLKGLSLVDKENTPPALSGTRVLASKTARRIFQEPTEPKTK AAAPGVEDEPLLRENPRRFVIFPIEYHDIWQMYKKAEASFWTAEEVDLSKDIQHWESLK PEERYFISHVLAFFAASDGIVNENLVERFSQEVQITEARCFYGFQIAMENIHSEMYSLLIDT YIKDPKEREFLFNAIETMPCVKKKADWALRWIGDKEATYGERVVAFAAVEGIFFSGSFA SIFWLKKRGLMPGLTFSNELISRDEGLHCDFACLMFKHLVHKPSEERVREIIINAVRIEQE FLTEALPVKLIGMNCTLMKQYIEFVADRLMLELGFSKVFRVENPFDFMENISLEGKTNFF EKRVGEYQRMGVMSSPTENSFTLDADF corresponding to amino acids 1-389 of NP_{—}001025 (SEQ ID NO:71), which also corresponds to amino acids 61-449 of T29749_P0 (SEQ ID NO:74), wherein said first amino acid sequence and second amino acid sequence are contiguous and in a sequential order.
B. An isolated polypeptide encoding for a head of T29749_P0 (SEQ ID NO:74), comprising a polypeptide being at least 70%, optionally at least about 80%, preferably at least about 85%, more preferably at least about 90% and most preferably at least about 95% homologous to the sequence MGRVGGMAQPMGRAGAPKPMGRAGSARRGRFKGCWSEGSPVHPVPAVLSWLLALLR CAAT (SEQ ID NO: 128) of T29749_P0 (SEQ ID NO:74).

Variant protein T29749_P0 (SEQ ID NO:74) also has the following non-silent SNPs (Single Nucleotide Polymorphisms) as listed in Table 7, (given according to their position(s) on the amino acid sequence, with the alternative amino acid(s) listed; the last column indicates whether the SNP is known or not; the presence of known SNPs in variant protein T29749_P0 (SEQ ID NO:74) sequence provides support for the deduced sequence of this variant protein according to the present invention).

TABLE 7
Amino acid mutations

| SNP position(s) on | | |
|---|---|---|
| amino acid sequence | Alternative amino acid(s) | Previously known SNP? |
| 59 | A -> S | Yes |
| 102 | V -> | No |
| 136 | N -> | No |
| 140 | F -> | No |
| 167 | V -> G | Yes |
| 213 | V -> G | No |
| 248 | K -> Q | No |
| 299 | S -> | No |
| 314 | G -> | No |
| 440 | E -> * | Yes |

Variant protein T29749_P0 (SEQ ID NO:74) is encoded by the following transcript(s): T29749_T (SEQ ID NO:38), for which the sequence(s) is/are given at the end of the application. The coding portion of transcript T29749_T1 (SEQ ID NO:38) is shown in bold; this coding portion starts at position 292 and ends at position 1638. The transcript also has the following SNPs as listed in Table 8 (given according to their position on the nucleotide sequence, with the alternative nucleic acid listed; the last column indicates whether the SNP is known or not; the presence of known SNPs in variant protein T29749_P0 (SEQ ID NO:74) sequence provides support for the deduced sequence of this variant protein according to the present invention).

TABLE 8
Nucleic acid SNPs

| SNP position(s) on nucleotide | Alternative | |
|---|---|---|
| sequence | nucleic acid(s) | Previously known SNP? |
| 466 | G -> T | Yes |
| 597 | C -> | No |
| 699 | C -> | No |
| 709 | T -> | No |
| 791 | T -> G | Yes |
| 792 | G -> T | No |
| 801 | C -> G | Yes |
| 929 | T -> G | No |
| 1033 | A -> C | No |
| 1188 | T -> C | No |
| 1188 | T -> | No |
| 1233 | C -> | No |
| 1609 | G -> T | Yes |
| 1672 | -> T | No |
| 1673 | T -> | No |
| 1913 | C -> T | Yes |
| 2042 | -> T | No |
| 2054 | A -> G | No |
| 2286 | G -> T | Yes |
| 2534 | T -> C | Yes |
| 2886 | T -> | No |
| 2902 | T -> G | No |
| 2953 | T -> G | No |
| 2977 | T -> G | No |
| 3197 | T -> G | Yes |
| 3324 | A -> G | Yes |
| 3592 | A -> G | Yes |

Variant protein T29749_P13 (SEQ ID NO:75) according to the present invention has an amino acid sequence as given at the end of the application; it is encoded by transcript(s) T29749_T20 (SEQ ID NO:41). An alignment is given to the known protein (Ribonucleoside-diphosphate reductase M2 chain) at the end of the application. One or more alignments to one or more previously published protein sequences are given at the end of the application. A brief description of the relationship of the variant protein according to the present invention to each such aligned protein is as follows:

1. Comparison repot between T29749_P13 (SEQ ID NO:75) and RIR2_HUMAN (SEQ ID NO:134):
A. An isolated chimeric polypeptide encoding for T29749_P13 (SEQ ID NO:75), comprising a first amino acid sequence being at least 70%, optionally at least 80%, preferably at least 85%, more preferably at least 90% and most preferably at least 95%, homologous to a polypeptide having the sequence MLQLLIPYVPT (SEQ ID NO: 129) corresponding to amino acids 1-11 of T29749_P13 (SEQ ID NO:75), and a second amino acid sequence being at least 90% or 95% homologous to REFLFNAIETMPCVKKKADWALRWIGDKEATYGERVVAFAAVEGIFFSGSFASIFWLKK RGLMPGLTFSNELISRDEGLHCDFACLMFKHLVBKPSEERVREIIINAVRIEQEFLTEALP VKLIGMNCTLMKQYIEFVADRLMLELGFSKVFRVENPFDFMENISLEGKTNFFEKRVGE YQRMGVMSSPTENSFTLDADF corresponding to amino acids 190-389 of RIR2_HUMAN (SEQ ID NO:134), which also corresponds to amino acids 12-211 of T29749_P13 (SEQ ID NO:75), wherein said first amino acid sequence and second amino acid sequence are contiguous and in a sequential order.
B. An isolated polypeptide encoding for a head of T29749_P13 (SEQ ID NO:75), comprising a polypeptide being at least 70%, optionally at least about 80%, preferably at least about 85%, more preferably at least about 90% and most preferably at least about 95% homologous to the sequence MLQLLIPYVPT (SEQ ID NO: 129) of T29749_P13 (SEQ ID NO:75).

2. Comparison report between T29749_P13 (SEQ ID NO:75) and Q8N6S3_HUMAN (SEQ ID NO:73):

A. An isolated chimeric polypeptide encoding for T29749_P13 (SEQ ID NO:75), comprising a first amino acid sequence being at least 70%, optionally at least 80%, preferably at least 85%, more preferably at least 90% and most preferably at least 95%, homologous to a polypeptide having the sequence MLQLLIPYVPTREFLFNAIETMPCVKKKADWALRWIGDK (SEQ ID NO: 130) corresponding to amino acids 1-39 of T29749_P13 (SEQ ID NO:75), and a second amino acid sequence being at least 90% or 95% homologous to EATYGERVVAFAAVEGIFFSGSFASIFWLKKRGLMPGLTFSNELISRDEGLHCDFACLMF KHLVHKPSEERVREIIINAVRIEQEFLTEALPVKLIGMNCTLMKQYIEFVADRLMLELGFS KVFRVENPFDFMENISLEGKTNFFEKRVGEYQRMGVMSSPTENSFTLDADF corresponding to amino acids 1-172 of Q8N6S3_HUMAN (SEQ ID NO:73), which also corresponds to amino acids 40-211 of T29749_P13 (SEQ ID NO:75), wherein said first amino acid sequence and second amino acid sequence are contiguous and in a sequential order.
B. An isolated polypeptide encoding for a head of T29749_P13 (SEQ ID NO:75), comprising a polypeptide being at least 70%, optionally at least about 80%, preferably at least about 85%, more preferably at least about 90% and most preferably at least about 95% homologous to the sequence MLQLLIPYVPTREFLFNAIETMPCVKKKADWALRWIGDK (SEQ ID NO: 130) of T29749_P13 (SEQ ID NO:75).

3. Comparison report between T29749_P13 (SEQ ID NO:75) and NP_{—}001025 (SEQ ID NO:71):
A. An isolated chimeric polypeptide encoding for T29749_P13 (SEQ ID NO:75), comprising a first amino acid sequence being at least 70%, optionally at least 80%, preferably at least 85%, more preferably at least 90% and most preferably at least 95%, homologous to a polypeptide having the sequence MLQLLIPYVPT (SEQ ID NO: 129) corresponding to amino acids 1-11 of T29749_P13 (SEQ ID NO:75), and a second amino acid sequence being at least 90% or 95% homologous to REFLFNAIETMPCVKKKADWALRWIGDKEATYGERVVAFAAVEGIFFSGSFASIFWLKK RGLMPGLTFSNELISRDEEGLHCDFACLMFKHLVHKPSEERVRENAVRIEQEFLTEALP VKLIGMNCTLMKQYIEFVADRLMLELGFSKVFRVENPFDFMENISLEGKTNFFEKRVGE YQRMGVMSSPTENSFTLDADF corresponding to amino acids 190-389 of NP_{—}001025 (SEQ ID NO:71), which also corresponds to amino acids 12-211 of T29749_P13 (SEQ ID NO:75), wherein said first amino acid sequence and second amino acid sequence are contiguous and in a sequential order.
B. An isolated polypeptide encoding for a head of T29749_P13 (SEQ ID NO:75), comprising a polypeptide being at least 70%, optionally at least about 80%, preferably at least about 85%, more preferably at least about 90% and most preferably at least about 95% homologous to the sequence MLQLLIPYVPT (SEQ ID NO: 129) of T29749_P13 (SEQ ID NO:75).

The localization of the variant protein was determined according to results from a number of different software programs and analyses, including analyses from SignalP and other specialized programs. The variant protein is believed to be located as follows with regard to the cell: intracellularly.

Variant protein T29749_P13 (SEQ ID NO:75) also has the following non-silent SNPs (Single Nucleotide Polymorphisms) as listed in Table 9, (given according to their position(s) on the amino acid sequence, with the alternative amino acid(s) listed; the last column indicates whether the SNP is known or not; the presence of known SNPs in variant protein T29749_P13 (SEQ ID NO:75) sequence provides support for the deduced sequence of this variant protein according to the present invention).

TABLE 9
Amino acid mutations

| SNP position(s) on | | |
|---|---|---|
| amino acid sequence | Alternative amino acid(s) | Previously known SNP? |
| 61 | S -> | No |
| 76 | G -> | No |
| 202 | E -> * | Yes |

Variant protein T29749_P13 (SEQ ID NO:75) is encoded by the following transcript(s): T29749_T20 (SEQ ID NO:41), for which the sequence(s) is/are given at the end of the application. The coding portion of transcript T29749_T20 (SEQ ID NO:41) is shown in bold; this coding portion starts at position 329 and ends at position 961. The transcript also has the following SNPs as listed in Table 10 (given according to their position on the nucleotide sequence, with the alternative nucleic acid listed; the last column indicates whether the SNP is known or not; the presence of known SNPs in variant protein T29749_P13 (SEQ ID NO:75) sequence provides support for the deduced sequence of this variant protein according to the present invention).

TABLE 10
Nucleic acid SNPs

| SNP position(s) on nucleotide | Alternative | |
|---|---|---|
| sequence | nucleic acid(s) | Previously known SNP? |
| 511 | T -> C | No |
| 511 | T -> | No |
| 556 | C -> | No |
| 932 | G -> T | Yes |
| 995 | -> T | No |
| 996 | T -> | No |
| 1236 | C -> T | Yes |
| 1365 | -> T | No |
| 1377 | A -> G | No |

Variant protein T29749_P34 (SEQ ID NO:76) according to the present invention has an amino acid sequence as given at the end of the application; it is encoded by transcript(s) T29749_T7 (SEQ ID NO:39). An alignment is given to the known protein (Ribonucleoside-diphosphate reductase M2 chain) at the end of the application. One or more alignments to one or more previously published protein sequences are given at the end of the application. A brief description of the relationship of the variant protein according to the present invention to each such aligned protein is as follows:

1. Comparison report between T29749_P34 (SEQ ID NO:76) and RIR2_HUMAN (SEQ ID NO:134):
A. An isolated chimeric polypeptide encoding for T29749_P34 (SEQ ID NO:76), comprising a first amino acid sequence being at least 90% or 95% homologous to MLSLRVPLAPITDPQQLQLSPLKGLSLVDKENTPPALSGTRVLASKTARRIFQEPTEPKTK AAAPGVEDEPLLRENPRRFVIFPIEYIDIWQMYKKAEASFWTAEEVDLSKDIQHWESLK PEERYFISHIVLAFFAASDGIVNENLVERFSQEVQITEARCFYGFQIAMENIHSEMYSLLIDT YIKDPKEREFLFNAIETMPCVKKKADWALRWIGDKEATYGERVVAFAAVEGIFFSGSFA SIFWLKKRGLMPGLTFSNELISRDEGLHCDFACLMFKHLVHKPSEERVREIIINAVRIEQE FLTEALPVKLIGMNCTLMKQYIEFVADRLMLELGFSKV corresponding to amino acids 1-340 of RIR2_HUMAN (SEQ ID NO:134), which also corresponds to amino acids 1-340 of T29749_P34 (SEQ ID NO:76), and a second amino acid sequence being at least 70%, optionally at least 80%, preferably at least 85%, more preferably at least 90% and most preferably at least 95% homologous to a polypeptide having the sequence LGDREVQSRWSPGPRGDSTPVREMETNHPPSVRG (SEQ ID NO: 131) corresponding to amino acids 341-374 of T29749_P34 (SEQ ID NO:76), wherein said first amino acid sequence and second amino acid sequence are contiguous and in a sequential order.
B. An isolated polypeptide encoding for an edge portion of T29749_P34 (SEQ ID NO:76), comprising an amino acid sequence being at least 70%, optionally at least about 80%, preferably at least about 85%, more preferably at least about 90% and most preferably at least about 95% homologous to the sequence LGDREVQSRWSPGPRGDSTPVREMETNHPPSVRG (SEQ ID NO: 131) of T29749_P34 (SEQ ID NO:76).

2. Comparison report between T29749_P34 (SEQ ID NO:76) and NP_{—}001025 (SEQ ID NO:71):
A. An isolated chimeric polypeptide encoding for T29749_P34 (SEQ ID NO:76), comprising a first amino acid sequence being at least 90% or 95% homologous to MLSLRVPLAPITDPQQLQLSPLKGLSLVDKENTPPALSGTRVLASKTARRIFQEPTEPKTK AAAPGVEDEPLLRENPRRFVIFPIEYHDIWQMYKKAEASFWTAEEVDLSKDIQHWESLK PEERYFISHVLAFFAASDGIVNENLVERFSQEVQITEARCFYGFQIAMENIHSEMYSLLIDT YIKDPKEREFLFNAEETMPCVKKKADWALRWIGDKEATYGERVVAFAAVEGIFFSGSFA SIFWLKKRGLMPGLTFSNELISRDEGLHCDFACLMFKHLVHKPSEERVREIIINAVRIEQE FLTEALPVKLIGMNCTLMKQYIEFVADRLMLELGFSKV corresponding to amino acids 1-340 of NP_{—}001025 (SEQ ID NO:71), which also corresponds to amino acids 1-340 of T29749_P34 (SEQ ID NO:76), and a second amino acid sequence being at least 70%, optionally at least 80%, preferably at least 85%, more preferably at least 90% and most preferably at least 95% homologous to a polypeptide having the sequence LGDREVQSRWSPGPRGDSTPVREMETNHPPSVRG (SEQ ID NO: 131) corresponding to amino acids 341-374 of T29749_P34 (SEQ ID NO:76), wherein said first amino acid sequence and second amino acid sequence are contiguous and in a sequential order.
B. An isolated polypeptide encoding for an edge portion of T29749_P34 (SEQ ID NO:76), comprising an amino acid sequence being at least 70%, optionally at least about 80%, preferably at least about 85%, more preferably at least about 90% and most preferably at least about 95% homologous to the sequence LGDREVQSRWSPGPRGDSTPVREMETNHPPSVRG (SEQ ID NO: 131) of T29749_P34 (SEQ ID NO:76).

The localization of the variant protein was determined according to results from a number of different software programs and analyses, including analyses from SignalP and other specialized programs. The variant protein is believed to be located as follows with regard to the cell: intracellularly.

Variant protein T29749_P34 (SEQ ID NO:76) also has the following non-silent SNPs (Single Nucleotide Polymorphisms) as listed in Table 11, (given according to their position(s) on the amino acid sequence, with the alternative amino acid(s) listed; the last column indicates whether the SNP is known or not; the presence of known SNPs in variant protein T29749_P34 (SEQ ID NO:76) sequence provides support for the deduced sequence of this variant protein according to the present invention).

TABLE 11
Amino acid mutations

| SNP position(s) on | | |
|---|---|---|
| amino acid sequence | Alternative amino acid(s) | Previously known SNP? |
| 42 | V -> | No |
| 76 | N -> | No |
| 80 | F -> | No |
| 107 | V -> G | Yes |
| 153 | V -> G | No |
| 188 | K -> Q | No |
| 239 | S -> | No |
| 254 | G -> | No |

Variant protein T29749_P34 (SEQ ID NO:76) is encoded by the following transcript(s): (SEQ ID NO:39), for which the sequence(s) is/are given at the end of the application. The coding portion of transcript T29749_T7 (SEQ ID NO:39) is shown in bold; this coding portion starts at position 472 and ends at position 1593. The transcript also has the following SNPs as listed in Table 12 (given according to their position on the nucleotide sequence, with the alternative nucleic acid listed; the last column indicates whether the SNP is known or not; the presence of known SNPs in variant protein T29749_P34 (SEQ ID NO:76) sequence provides support for the deduced sequence of this variant protein according to the present invention).

TABLE 12
Nucleic acid SNPs

| SNP position(s) on nucleotide | Alternative | |
|---|---|---|
| sequence | nucleic acid(s) | Previously known SNP? |
| 466 | G -> T | Yes |
| 597 | C -> | No |
| 699 | C -> | No |
| 709 | T -> | No |
| 791 | T -> G | Yes |
| 792 | G -> T | No |
| 801 | C -> G | Yes |
| 929 | T -> G | No |
| 1033 | A -> C | No |
| 1188 | T -> C | No |
| 1188 | T -> | No |
| 1233 | C -> | No |

Variant protein T29749_P40 (SEQ ID NO:77) according to the present invention has an amino acid sequence as given at the end of the application; it is encoded by transcript(s) T29749_T14 (SEQ ID NO:40). An alignment is given to the known protein (Ribonucleoside-diphosphate reductase M2 chain) at the end of the application. One or more alignments to one or more previously published protein sequences are given at the end of the application. A brief description of the relationship of the variant protein according to the present invention to each such aligned protein is as follows:

1. Comparison report between T29749_P40 (SEQ ID NO:77) and RIR2_HUMAN (SEQ ID NO:134):
A. An isolated chimeric polypeptide encoding for T29749_P40 (SEQ ID NO:77), comprising a first amino acid sequence being at least 90% or 95% homologous to MLSLRVPLAPITDPQQLQLSPLKGLSLVDKENTPPALSGTRVLASKTARRIFQEPTEPKTK AAAPGVEDEPLLRENPRRFVIFPIEYHDIWQMYKKAEASFWTAEEVDLSKDIQHWESLK PEERYFISHVLAFFAASDGWNENLVERFSQEVQITEARCFYGFQIAMENIHSEMYSLLIDT YIKDPKEREFLFNAIETMPCVKKKADWALRWIGDKEATYGERVVAFAAVEGIFFSGSFA SIFWLKKRGLMPGLTFSNELISRDE corresponding to amino acids 1-266 of RIR2_HUMAN (SEQ ID NO:134), which also corresponds to amino acids 1-266 of T29749_P40 (SEQ ID NO:77), and a second amino acid sequence being at least 70%, optionally at least 80%, preferably at least 85%, more preferably at least 90% and most preferably at least 95% homologous to a polypeptide having the sequence VSLSQIIG (SEQ ID NO: 132) corresponding to amino acids 267-274 of T29749_P40 (SEQ ID NO:77), wherein said first amino acid sequence and second amino acid sequence are contiguous and in a sequential order.
B. An isolated polypeptide encoding for an edge portion of T29749_P40 (SEQ ID NO:77), comprising an amino acid sequence being at least 70%, optionally at least about 80%, preferably at least about 85%, more preferably at least about 90% and most preferably at least about 95% homologous to the sequence VSLSQIIG (SEQ ID NO: 132) of T29749_P40 (SEQ ID NO:77).

2. Comparison report between T29749_P40 (SEQ ID NO:77) and NP_{—}001025 (SEQ ID NO:71):

A. An isolated chimeric polypeptide encoding for T29749_P40 (SEQ ID NO:77), comprising a first amino acid sequence being at least 90% or 95% homologous to MLSLRVPLAPITDPQQLQLSPLKGLSLVDKENTPPALSGTRVLASKTARRIFQEPTEPKTK AAAPGVEDEPLLRENPRRFVIFPIEYHDIWQMYKKAEASFWTAEEVDLSKDIQHWESLK PEERYFISHVLAFFAASDGIVNENLVERFSQEVQITEARCFYGFQLAMENIHSEMYSLLIDT YTKDPKEREFLFNAIETMPCVKKKADWALRWIGDKEATYGERVVAFAAVEGIFFSGSFA SIFWLKKRGLMPGLTFSNELISRDE corresponding to amino acids 1-266 of NP_{—}001025 (SEQ ID NO:71), which also corresponds to amino acids 1-266 of T29749_P40 (SEQ ID NO:77), and a second amino acid sequence being at least 70%, optionally at least 80%, preferably at least 85%, more preferably at least 90% and most preferably at least 95% homologous to a polypeptide having the sequence VSLSQIIG (SEQ ID NO: 132) corresponding to amino acids 267-274 of T29749_P40 (SEQ ID NO:77), wherein said first amino acid sequence and second amino acid sequence are contiguous and in a sequential order.
B. An isolated polypeptide encoding for an edge portion of T29749_P40 (SEQ ID NO:77), comprising an amino acid sequence being at least 70%, optionally at least about 80%, preferably at least about 85%, more preferably at least about 90% and most preferably at least about 95% homologous to the sequence VSLSQIIG (SEQ ID NO: 132) of T29749_P40 (SEQ ID NO:77).

The localization of the variant protein was determined according to results from a number of different software programs and analyses, including analyses from SignalP and other specialized programs. The variant protein is believed to be located as follows with regard to the cell: intracellularly.

Variant protein T29749_P40 (SEQ ID NO:77) also has the following non-silent SNPs (Single Nucleotide Polymorphisms) as listed in Table 13, (given according to their position(s) on the amino acid sequence, with the alternative amino acid(s) listed; the last column indicates whether the SNP is known or not; the presence of known SNPs in variant protein T29749_P40 (SEQ ID NO:77) sequence provides support for the deduced sequence of this variant protein according to the present invention).

TABLE 13
Amino acid mutations

| SNP position(s) on | | |
|---|---|---|
| amino acid sequence | Alternative amino acid(s) | Previously known SNP? |
| 42 | V -> | No |
| 76 | N -> | No |
| 80 | F -> | No |
| 107 | V -> G | Yes |
| 153 | V -> G | No |
| 188 | K -> Q | No |
| 239 | S -> | No |
| 254 | G -> | No |

Variant protein T29749_P40 (SEQ ID NO:77) is encoded by the following transcript(s): (SEQ ID NO:40), for which the sequence(s) is/are given at the end of the application. The coding portion of transcript T29749_T14 (SEQ ID NO:40) is shown in bold; this coding portion starts at position 472 and ends at position 1293. The transcript also has the following SNPs as listed in Table 14 (given according to their position on the nucleotide sequence, with the alternative nucleic acid listed; the last column indicates whether the SNP is known or not; the presence of known SNPs in variant protein T29749_P40 (SEQ ID NO:77) sequence provides support for the deduced sequence of this variant protein according to the present invention).

TABLE 14
Nucleic acid SNPs

| SNP position(s) on nucleotide | Alternative | |
|---|---|---|
| sequence | nucleic acid(s) | Previously known SNP? |
| 466 | G -> T | Yes |
| 597 | C -> | No |
| 699 | C -> | No |
| 709 | T -> | No |
| 791 | T -> G | Yes |
| 792 | G -> T | No |
| 801 | C -> G | Yes |
| 929 | T -> G | No |
| 1033 | A -> C | No |
| 1188 | T -> C | No |
| 1188 | T -> | No |
| 1233 | C -> | No |

As noted above, cluster T29749 features 29 segment(s), which were listed in Table 3 above and for which the sequence(s) are given at the end of the application. These segment(s) are portions of nucleic acid sequence(s) which are described herein separately because they are of particular interest. A description of each segment according to the present invention is now provided.

Segment cluster T29749_NO (SEQ ID NO:42) according to the present invention is supported by 133 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 15 below describes the starting and ending position of this segment on each transcript.

TABLE 15
Segment location on transcripts

| | Segment | Segment |
|---|---|---|
| Transcript name | starting position | ending position |
| T29749_T1 (SEQ ID NO: 38) | 1 | 570 |
| T29749_T14 (SEQ ID NO: 40) | 1 | 570 |
| T29749_T7 (SEQ ID NO: 39) | 1 | 570 |

Segment cluster T29749_N29 (SEQ ID NO:43) according to the present invention is supported by 2 libraries. The number of libraries was determined as previously described. This segment can be found in the following transcript(s): T29749_T20 (SEQ ID NO:41). Table 16 below describes the starting and ending position of this segment on each transcript.

TABLE 16
Segment location on transcripts

| | Segment | Segment |
|---|---|---|
| Transcript name | starting position | ending position |
| T29749_T20 (SEQ ID NO: 41) | 1 | 360 |

Segment cluster T29749_N33 (SEQ ID NO:44) according to the present invention is supported by 124 libraries. The number of libraries was determined as previously described. This segment can be found in the following transcript(s); T29749_T1 (SEQ ID NO:38), T29749_T14 (SEQ ID NO:40), T29749_T20 (SEQ ID NO:41) and T29749_T7 (SEQ ID NO:39). Table 17 below describes the starting and ending position of this segment on each transcript.

TABLE 17
Segment location on transcripts

| | Segment | Segment |
|---|---|---|
| Transcript name | starting position | ending position |
| T29749_T1 (SEQ ID NO: 38) | 1136 | 1269 |
| T29749_T14 (SEQ ID NO: 40) | 1136 | 1269 |
| T29749_T20 (SEQ ID NO: 41) | 459 | 592 |
| T29749_T7 (SEQ ID NO: 39) | 1136 | 1269 |

Segment cluster T29749_N34 (SEQ ID NO:45) according to the present invention is supported by 2 libraries. The number of libraries was determined as previously described. This segment can be found in the following transcript(s): T29749_T14 (SEQ ID NO:40). Table 18 below describes the starting and ending position of this segment on each transcript.

TABLE 18
Segment location on transcripts

| | Segment | Segment |
|---|---|---|
| Transcript name | starting position | ending position |
| T29749_T14 (SEQ ID NO: 40) | 1270 | 1410 |

Segment cluster T29749_N40 (SEQ ID NO:46) according to the present invention is supported by 148 libraries. The number of libraries was determined as previously described. This segment can be found in the following transcript(s): T29749_T1 (SEQ ID NO:38) and T29749_T20 (SEQ ID NO:41). Table 19 below describes the starting and ending position of this segment on each transcript.

TABLE 19
Segment location on transcripts

| | Segment | Segment |
|---|---|---|
| Transcript name | starting position | ending position |
| T29749_T1 (SEQ ID NO: 38) | 1489 | 1748 |
| T29749_T20 (SEQ ID NO: 41) | 812 | 1071 |

Segment cluster T29749_N41 (SEQ ID NO:47) according to the present invention is supported by 141 libraries. The number of libraries was determined as previously described. This segment can be found in the following transcript(s): T29749_T1 (SEQ ID NO:38) and T29749_T20 (SEQ ID NO:41). Table 20 below describes the starting and ending position of this segment on each transcript.

TABLE 20
Segment location on transcripts

| | Segment | Segment |
|---|---|---|
| Transcript name | starting position | ending position |
| T29749_T1 (SEQ ID NO: 38) | 1749 | 2110 |
| T29749_T20 (SEQ ID NO: 41) | 1072 | 1380 |

Segment cluster T29749_N42 (SEQ ID NO:48) according to the present invention is supported by 101 libraries. The number of libraries was determined as previously described. This segment can be found in the following transcript(s): T29749_T1 (SEQ ID NO:38). Table 21 below describes the starting and ending position of this segment on each transcript,

TABLE 21
Segment location on transcripts

| | Segment | Segment |
|---|---|---|
| Transcript name | starting position | ending position |
| T29749_T1 (SEQ ID NO: 38) | 2111 | 3682 |

Segment cluster T29749_N46 (SEQ ID NO:49) according to the present invention is supported by 2 libraries. The number of libraries was determined as previously described. This segment can be found in the following transcript(s): T29749_T7 (SEQ ID NO:39). Table 22 below describes the starting and ending position of this segment on each transcript.

TABLE 22
Segment location on transcripts

| | Segment | Segment |
|---|---|---|
| Transcript name | starting position | ending position |
| T29749_T7 (SEQ ID NO: 39) | 1590 | 1710 |

According to an optional embodiment of the present invention, short segments related to the above cluster are also provided. These segments are up to about 120 bp in length, and so are included in a separate description.

Segment cluster T29749_N2 (SEQ ID NO:50) according to the present invention is supported by 131 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 23 below describes the starting and ending position of this segment on each transcript.

TABLE 23
Segment location on transcripts

| | Segment | Segment |
|---|---|---|
| Transcript name | starting position | ending position |
| T29749_T1 (SEQ ID NO: 38) | 571 | 598 |
| T29749_T14 (SEQ ID NO: 40) | 571 | 598 |
| T29749_T7 (SEQ ID NO: 39) | 571 | 598 |

Segment cluster T29749_N3 (SEQ ID NO:51) according to the present invention is supported by 135 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 24 below describes the starting and ending position of this segment on each transcript.

TABLE 24
Segment location on transcripts

| | Segment | Segment |
|---|---|---|
| Transcript name | starting position | ending position |
| T29749_T1 (SEQ ID NO: 38) | 599 | 634 |
| T29749_T14 (SEQ ID NO: 40) | 599 | 634 |
| T29749_T7 (SEQ ID NO: 39) | 599 | 634 |

Segment cluster T29749_N4 (SEQ ID NO:52) according to the present invention is supported by 131 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 25 below describes the starting and ending position of this segment on each transcript.

TABLE 25
Segment location on transcripts

| | Segment | Segment |
|---|---|---|
| Transcript name | starting position | ending position |
| T29749_T1 (SEQ ID NO: 38) | 635 | 645 |
| T29749_T14 (SEQ ID NO: 40) | 635 | 645 |
| T29749_T7 (SEQ ID NO: 39) | 635 | 645 |

Segment cluster T29749_N7 (SEQ ID NO:53) according to the present invention is supported by 134 libraries. The number of libraries was determined as previously described. This segment can be found in the following transcript(s); T29749_T1 (SEQ ID NO:38), T29749_T14 (SEQ ID NO:40) and T29749_T7 (SEQ ID NO:39). Table 26 below describes the starting and ending position of this segment on each transcript.

TABLE 26
Segment location on transcripts

| | Segment | Segment |
|---|---|---|
| Transcript name | starting position | ending position |
| T29749_T1 (SEQ ID NO: 38) | 646 | 664 |
| T29749_T14 (SEQ ID NO: 40) | 646 | 664 |
| T29749_T7 (SEQ ID NO: 39) | 646 | 664 |

Segment cluster T29749_N8 (SEQ ID NO:54) according to the present invention is supported by 133 libraries. The number of libraries was determined as previously described. This segment can be found in the following transcript(s); T29749_T1 (SEQ ID NO:38), T29749_T14 (SEQ ID NO:40) and T29749_T7 (SEQ ID NO:39). Table 27 below describes the starting and ending position of this segment on each transcript.

TABLE 27
Segment location on transcripts

| | Segment | Segment |
|---|---|---|
| Transcript name | starting position | ending position |
| T29749_T1 (SEQ ID NO: 38) | 665 | 684 |
| T29749_T14 (SEQ ID NO: 40) | 665 | 684 |
| T29749_T7 (SEQ ID NO: 39) | 665 | 684 |

Segment cluster T29749 N9 (SEQ ID NO:55) according to the present invention is supported by 131 libraries. The number of libraries was determined as previously described. This segment can be found in the following transcript(s); T29749_T1 (SEQ ID NO:38)₃, T29749_T14 (SEQ ID NO:40) and T29749_T7 (SEQ ID NO:39). Table 28 below describes the starting and ending position of this segment on each transcript.

TABLE 28
Segment location on transcripts

| | Segment | Segment |
|---|---|---|
| Transcript name | starting position | ending position |
| T29749_T1 (SEQ ID NO: 38) | 685 | 694 |
| T29749_T14 (SEQ ID NO: 40) | 685 | 694 |
| T29749_T7 (SEQ ID NO: 39) | 685 | 694 |

Segment cluster T29749_N10 (SEQ ID NO:56) according to the present invention is supported by 133 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 29 below describes the starting and ending position of this segment on each transcript.

TABLE 29
Segment location on transcripts

| | Segment | Segment |
|---|---|---|
| Transcript name | starting position | ending position |
| T29749_T1 (SEQ ID NO: 38) | 695 | 699 |
| T29749_T14 (SEQ ID NO: 40) | 695 | 699 |
| T29749_T7 (SEQ ID NO: 39) | 695 | 699 |

Segment cluster T29749_N11 (SEQ ID NO:57) according to the present invention is supported by 133 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 30 below describes the starting and ending position of this segment on each transcript.

TABLE 30
Segment location on transcripts

| | Segment | Segment |
|---|---|---|
| Transcript name | starting position | ending position |
| T29749_T1 (SEQ ID NO: 38) | 700 | 721 |
| T29749_T14 (SEQ ID NO: 40) | 700 | 721 |
| T29749_T7 (SEQ ID NO: 39) | 700 | 721 |

Segment cluster T29749_N12 (SEQ ID NO:58) according to the present invention is supported by 143 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 31 below describes the starting and ending position of this segment on each transcript.

TABLE 31
Segment location on transcripts

| | Segment | Segment |
|---|---|---|
| Transcript name | starting position | ending position |
| T29749_T1 (SEQ ID NO: 38) | 722 | 789 |
| T29749_T14 (SEQ ID NO: 40) | 722 | 789 |
| T29749_T7 (SEQ ID NO: 39) | 722 | 789 |

Segment cluster T29749_N15 (SEQ ID NO:59) according to the present invention is supported by 142 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 32 below describes the starting and ending position of this segment on each transcript.

TABLE 32
Segment location on transcripts

| | Segment | Segment |
|---|---|---|
| Transcript name | starting position | ending position |
| T29749_T1 (SEQ ID NO: 38) | 790 | 811 |
| T29749_T14 (SEQ ID NO: 40) | 790 | 811 |
| T29749_T7 (SEQ ID NO: 39) | 790 | 811 |

Segment cluster T29749_N17 (SEQ ID NO:60) according to the present invention is supported by 146 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 33 below describes the starting and ending position of this segment on each transcript.

TABLE 33
Segment location on transcripts

| | Segment | Segment |
|---|---|---|
| Transcript name | starting position | ending position |
| T29749_T1 (SEQ ID NO: 38) | 812 | 832 |
| T29749_T14 (SEQ ID NO: 40) | 812 | 832 |
| T29749_T7 (SEQ ID NO: 39) | 812 | 832 |

Segment cluster T29749_N18 (SEQ ID NO:61) according to the present invention is supported by 149 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 34 below describes the starting and ending position of this segment on each transcript.

TABLE 34
Segment location on transcripts

| | Segment | Segment |
|---|---|---|
| Transcript name | starting position | ending position |
| T29749_T1 (SEQ ID NO: 38) | 833 | 906 |
| T29749_T14 (SEQ ID NO: 40) | 833 | 906 |
| T29749_T7 (SEQ ID NO: 39) | 833 | 906 |

Segment cluster T29749_N22 (SEQ ID NO:62) according to the present invention is supported by 147 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 35 below describes the starting and ending position of this segment on each transcript.

TABLE 35
Segment location on transcripts

| | Segment | Segment |
|---|---|---|
| Transcript name | starting position | ending position |
| T29749_T1 (SEQ ID NO: 38) | 907 | 959 |
| T29749_T14 (SEQ ID NO: 40) | 907 | 959 |
| T29749_T7 (SEQ ID NO: 39) | 907 | 959 |

Segment cluster T29749_N23 (SEQ ID NO:63) according to the present invention is supported by 142 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 36 below describes the starting and ending position of this segment on each transcript.

TABLE 36
Segment location on transcripts

| | Segment | Segment |
|---|---|---|
| Transcript name | starting position | ending position |
| T29749_T1 (SEQ ID NO: 38) | 960 | 985 |
| T29749_T14 (SEQ ID NO: 40) | 960 | 985 |
| T29749_T7 (SEQ ID NO: 39) | 960 | 985 |

Segment cluster T29749_N24 (SEQ ID NO:64) according to the present invention is supported by 134 libraries. The number of libraries was determined as previously described. This segment can be found in the following transcript(s): T29749_T1 (SEQ ID NO:38), T29749_T14 (SEQ ID NO:40) and T29749_T7 (SEQ ID NO:39). Table 37 below describes the starting and ending position of this segment on each transcript.

TABLE 37
Segment location on transcripts

| | Segment | Segment |
|---|---|---|
| Transcript name | starting position | ending position |
| T29749_T1 (SEQ ID NO: 38) | 986 | 1040 |
| T29749_T14 (SEQ ID NO: 40) | 986 | 1040 |
| T29749_T7 (SEQ ID NO: 39) | 986 | 1040 |

Segment cluster T29749_N30 (SEQ ID NO:65) according to the present invention is supported by 3 libraries. The number of libraries was determined as previously described. This segment can be found in the following transcript(s): T29749_T20 (SEQ ID NO:41). Table 38 below describes the starting and ending position of this segment on each transcript.

TABLE 38
Segment location on transcripts

| | Segment | Segment |
|---|---|---|
| Transcript name | starting position | ending position |
| T29749_T20 (SEQ ID NO: 41) | 361 | 363 |

Segment cluster T29749_N31 (SEQ ID NO:66) according to the present invention is supported by 130 libraries. The number of libraries was determined as previously described. This segment can be found in the following transcript(s): T29749_T1 (SEQ ID NO:38), T29749_T14 (SEQ ID NO:40), T29749_T20 (SEQ ID NO:41) and T29749_T7 (SEQ ID NO:39). Table 39 below describes the starting and ending position of this segment on each transcript.

TABLE 39
Segment location on transcripts

| | Segment | Segment |
|---|---|---|
| Transcript name | starting position | ending position |
| T29749_T1 (SEQ ID NO: 38) | 1041 | 1135 |
| T29749_T14 (SEQ ID NO: 40) | 1041 | 1135 |
| T29749_T20 (SEQ ID NO: 41) | 364 | 458 |
| T29749_T7 (SEQ ID NO: 39) | 1041 | 1135 |

Segment cluster T29749_N36 (SEQ ID NO:67) according to the present invention is supported by 113 libraries. The number of libraries was determined as previously described. This segment can be found in the following transcript(s): T29749_T1 (SEQ ID NO:38), T29749_T20 (SEQ ID NO:41) and T29749_T7 (SEQ ID NO:39). Table 40 below describes the starting and ending position of this segment on each transcript.

TABLE 40
Segment location on transcripts

| | Segment | Segment |
|---|---|---|
| Transcript name | starting position | ending position |
| T29749_T1 (SEQ ID NO: 38) | 1270 | 1374 |
| T29749_T20 (SEQ ID NO: 41) | 593 | 697 |
| T29749_T7 (SEQ ID NO: 39) | 1270 | 1374 |

Segment cluster T29749_N38 (SEQ ID NO:68) according to the present invention is supported by 121 libraries. The number of libraries was determined as previously described. This segment can be found in the following transcript(s): T29749_T1 (SEQ ID NO:38), T29749_T20 (SEQ ID NO:41) and T29749_T7 (SEQ ID NO:39). Table 41 below describes the starting and ending position of this segment on each transcript.

TABLE 41
Segment location on transcripts

| | Segment | Segment |
|---|---|---|
| Transcript name | starting position | ending position |
| T29749_T1 (SEQ ID NO: 38) | 1375 | 1488 |
| T29749_T20 (SEQ ID NO: 41) | 698 | 811 |
| T29749_T7 (SEQ ID NO: 39) | 1375 | 1488 |

Segment cluster T29749_N44 (SEQ ID NO:69) according to the present invention is supported by 3 libraries. The number of libraries was determined as previously described. This segment can be found in the following transcript(s): T29749_T7 (SEQ ID NO:39). Table 42 below describes the starting and ending position of this segment on each transcript.

TABLE 42
Segment location on transcripts

| | Segment | Segment |
|---|---|---|
| Transcript name | starting position | ending position |
| T29749_T7 (SEQ ID NO: 39) | 1489 | 1589 |

Segment cluster T29749_N50 (SEQ ID NO:70) according to the present invention is supported by 1 libraries. The number of libraries was determined as previously described. This segment can be found in the following transcript(s): T29749_T7 (SEQ ID NO:39). Table 43 below describes the starting and ending position of this segment on each transcript.

TABLE 43
Segment location on transcripts

| | Segment | Segment |
|---|---|---|
| Transcript name | starting position | ending position |
| T29749_T7 (SEQ ID NO: 39) | 1711 | 1732 |

Microarray (chip) data is also available for this segment as follows. As described above with regard to the cluster itself various oligonucleotides were tested for being differentially expressed in various disease conditions, particularly cancer. The following oligonucleotides were found to hit segment T29749-seg 31 (SEQ ID NO: 66), shown in Table 44.

TABLE 44
Oligonucleotides related to this segment

| Oligonucleotide | Overexpressed | Chip |
|---|---|---|
| name | in cancers | reference |
| T29749_0_43_0 | Breast cancer | BRS |
| (SEQ ID NO: 37) | | |
| T29749_0_43_0 | Colon cancer | COL |
| (SEQ ID NO: 37) | | |
| T29749_0_43_0 | Lung cancer | LUN |
| (SEQ ID NO: 37) | | |

>T29749_0_43_0 (SEQ ID NO: 37)
TGCCTTGTGTCAAGAAGAAGGCAGACTGGGCCTTGCGCTGGATTGGGGAC

Variant protein alignment to the previously known protein:

### Alignment of: T29749_P0 (SEQ ID NO:74)×RIR2_HUMAN (SEQ ID NO:134):

Total length: 449
Matching length: 389

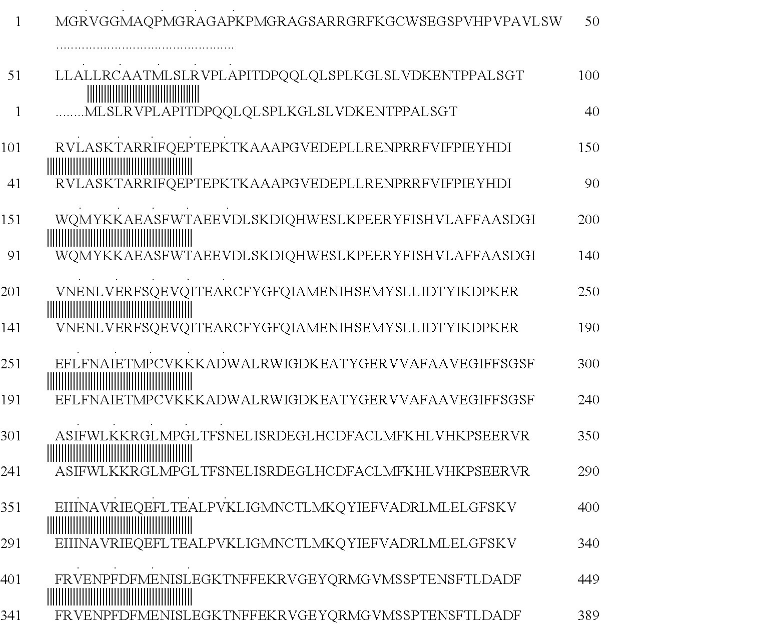

Alignment of. T29749_P0 (SEQ ID NO:74)×NP_{—}001025 (SEQ ID NO:71):
Total length: 449
Matching length: 389

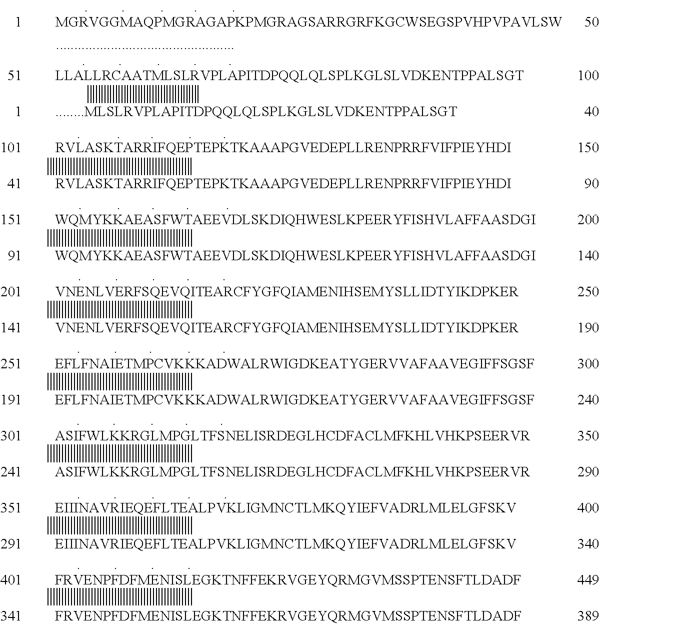

### Alignment of: T29749_P13 (SEQ ID NO:75)×RIR2_HUMAN (SEQ ID NO:134):

Total length: 400
Matching length: 200

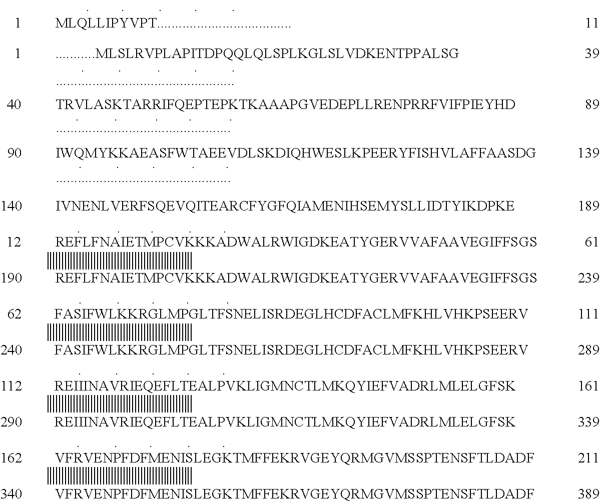

### Alignment of: T29749_P13 (SEQ ID NO:75)×Q8N6S3_HUMAN (SEQ ID NO:73):

Total length: 211
Matching length: 172

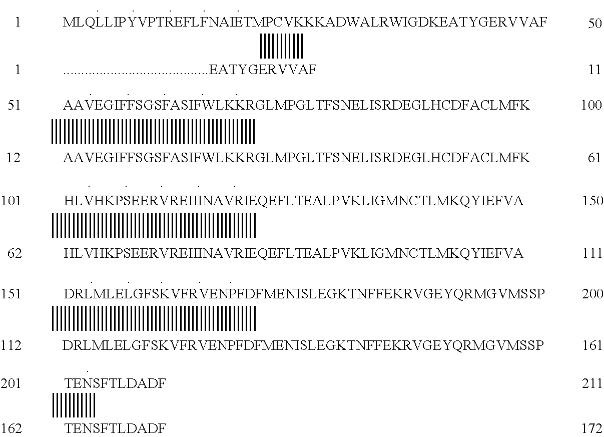

### Alignment of: T29749_P13 (SEQ ID NO:75)—NP_{—}001025 (SEQ ID NO:71):

Total length: 400
Matching length: 200

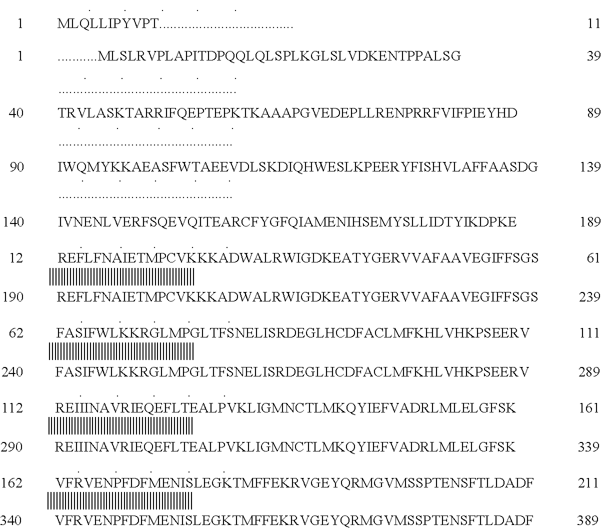

### Alignment of: T29749_P34 (SEQ ID NO:76)×RIR2_HUMAN (SEQ ID NO:134):

Total length: 423
Matching length: 340

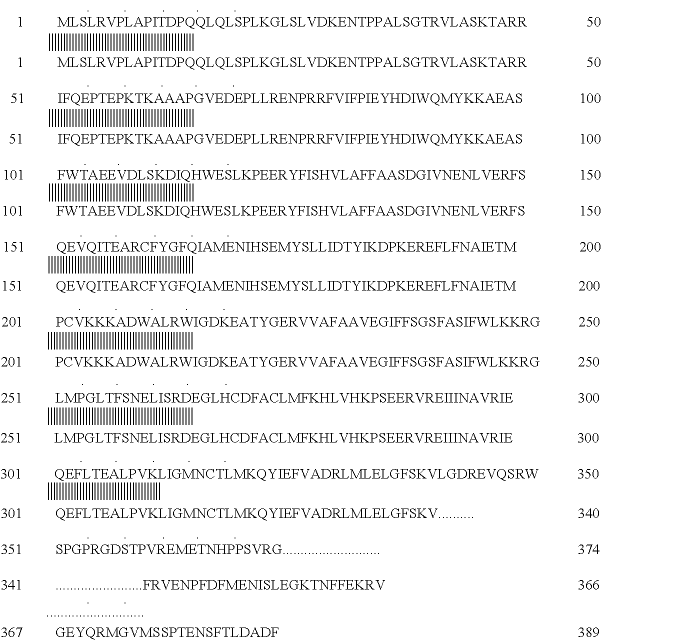

### Alignment of: T29749_P34 (SEQ ID NO:76)×NP_{—}001025 (SEQ ID NO:71):

Total length: 423
Matching length: 340

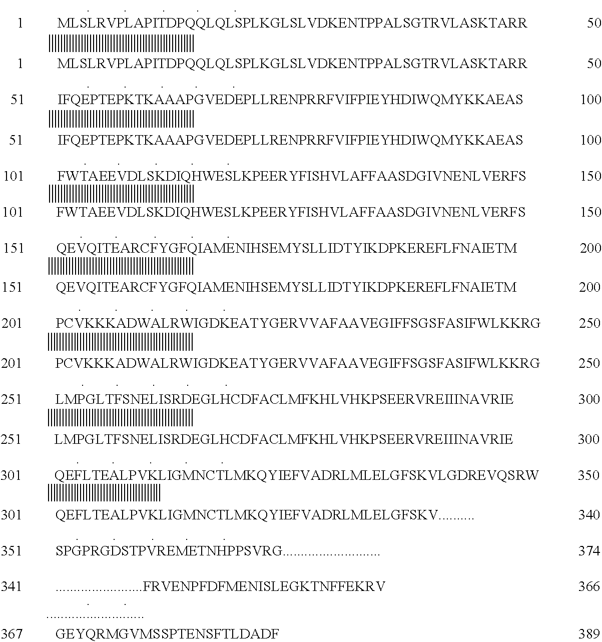

### Alignment of: T29749_P40 (SEQ ID NO:77)×RIR2_HUMAN (SEQ ID NO:134):

Total length: 397
Matching length: 266

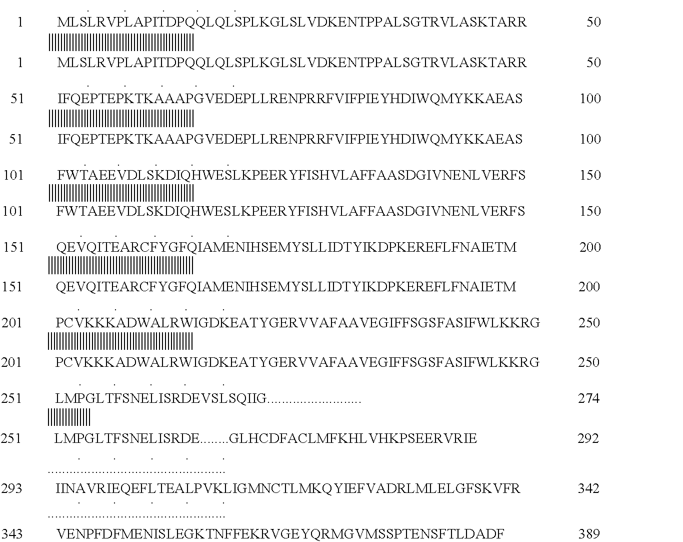

### Alignment of: T29749_P40 (SEQ ID NO:77)×NP_{—}001025 (SEQ ID NO:71):

Total length: 397
Matching length: 266

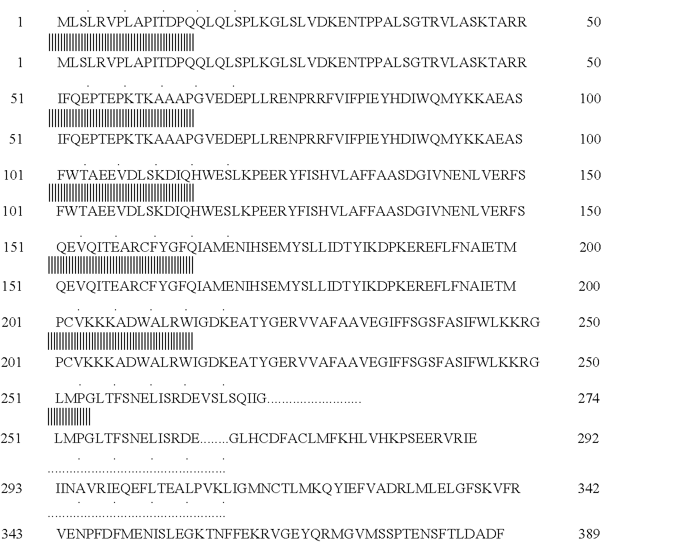

Expression of ribonucleotide reductase M2 polypeptide (RRM2) T29749 transcripts which are detectable by amplicon as depicted in sequence name T29749_seg22-24WT (SEQ ID NO: 80) in normal and cancerous Lung tissues

Expression of ribonucleotide reductase M2 polypeptide (RRM2) transcripts detectable by or according to seg22-24WT—T29749_seg22-24WT (SEQ ID NO: 80) amplicon and primers T29749_seg22-24WTF (SEQ ID NO: 78) and T29749_seg22-24WTR (SEQ ID NO: 79) was measured by real time PCR. In parallel the expression of several housekeeping genes —HPRT1 (GenBank Accession No. NM_{—}000194 (SEQ ID NO:9); amplicon—HPRT1-amplicon (SEQ ID NO: 12)), PBGD (GenBank Accession No. BC019323 (SEQ ID NO:5); amplicon—PBGD-amplicon (SEQ ID NO:8)), SDHA (GenBank Accession No. NM_{—}004168 (SEQ ID NO:1); amplicon—SDHA-amplicon (SEQ ID NO: 4)) and Ubiquitin (GenBank Accession No. BC000449 (SEQ ID NO:25); amplicon—Ubiquitin-amplicon (SEQ ID NO: 28)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}2 above), to obtain a value of fold up-regulation for each sample relative to median of the normal samples.

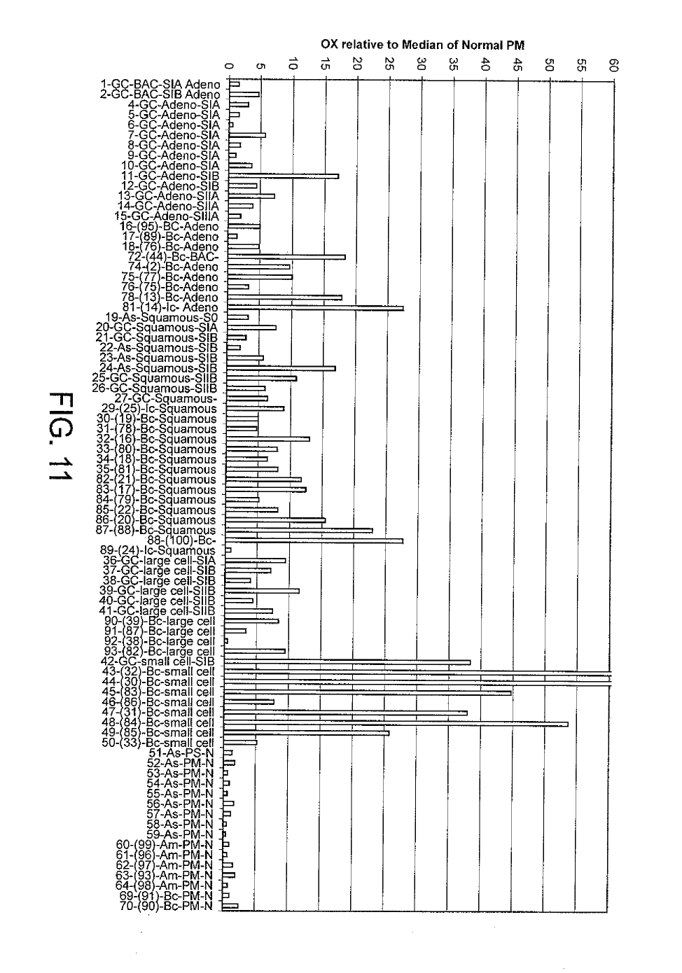

FIG. 8 is a histogram showing over expression of the above-indicated ribonucleotide reductase M2 polypeptide (RRM2) transcripts in cancerous Lung samples relative to the normal samples.

As is evident from FIG. 8, the expression of ribonucleotide reductase M2 polypeptide (RRM2) transcripts detectable by the above amplicon in adenocarcinoma, squamous cell carcinoma, large cell carcinoma, small cell carcinoma and non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}2 above). Notably an over-expression of at least 5 fold was found in 49 out of 57 non-small cell carcinoma samples, specifically in 18 out of 23 adenocarcinoma samples, in 22 out of 24 squamous cell carcinoma samples, in 9 out of 10 large cell carcinoma samples. Also over-expression was observed in 9 out of 9 small cell carcinoma samples.

Statistical analysis was applied to verify the significance of these results, as described below.

The P value for the difference in the expression levels of ribonucleotide reductase M2 polypeptide (RRM2) transcripts detectable by the above amplicon in Lung adenocarcinoma samples versus the normal tissue samples was determined by T test as 2.08e-002. The P value for the difference in the expression levels of ribonucleotide reductase M2 polypeptide (RRM2) transcripts detectable by the above amplicon in Lung squamous cell carcinoma samples versus the normal tissue samples was determined by T test as 1.33e-005. The P value for the difference in the expression levels of ribonucleotide reductase M2 polypeptide (RRM2) transcripts detectable by the above amplicon in Lung large cell carcinoma samples versus the normal tissue samples was determined by T test as 2.57e-004. The P value for the difference in the expression levels of ribonucleotide reductase M2 polypeptide (RRM2) transcripts detectable by the above amplicon in Lung small cell carcinoma samples versus the normal tissue samples was determined by T test as 1.02e-002. The P value for the difference in the expression levels of ribonucleotide reductase M2 polypeptide (RRM2) transcripts detectable by the above amplicon in Lung non-small cell carcinoma samples versus the normal tissue samples was determined by T test as 3.77e-005.

Threshold of 5 fold over expression was found to differentiate between adenocarcinoma and normal samples with P value of 6.02e-005 as checked by exact Fisher test. Threshold of 5 fold over expression was found to differentiate between squamous cell carcinoma and normal samples with P value of 5.3e-007 as checked by exact Fisher test. Threshold of 5 fold over expression was found to differentiate between large cell carcinoma and normal samples with P value of 1.55e-004 as checked by exact Fisher test. Threshold of 5 fold over expression was found to differentiate between small cell carcinoma and normal samples with P value of 2.69e-005 as checked by exact Fisher test. Threshold of 5 fold over expression was found to differentiate between non-small cell carcinoma and normal samples with P value of 7.88e-008 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T29749_seg22-24WTF (SEQ ID NO: 78) forward primer; and T29749_seg22-24WTR (SEQ ID NO: 79) reverse primer.

The present invention also preferably encompasses any amplicon obtained through the use of any suitable primer pair; for example, for the above experiment, the following amplicon was obtained as a non-limiting illustrative example only of a suitable amplicon: T29749_seg22-24WT (SEQ ID NO: 80).

Forward Primer (T29749_seg22-24WTF
(SEQ ID NO: 78)):
>T29749_seg22-24WTF (SEQ ID NO: 78)
TCAGATTACAGAAGCCCGCTG
Reverse Primer (T29749_seg22-24WTR
(SEQ ID NO: 79)):
>T29749_seg22-24WTR (SEQ ID NO: 79)
TGTAAGTGTCAATAAGAAGACTATACATTTCAG
Amplicon (T29749_seg22-24WT (SEQ ID NO: 80)):
>T29749_seg22-24WT (SEQ ID NO: 80)
TCAGATTACAGAAGCCCGCTGTTTCTATGGCTTCCAAATTGCCATGGAAA
ACATACATTCTGAAATGTATAGTCTTCTTATTGACACTTACA

Expression of ribonucleotide reductase M2 polypeptide (RRM2) T29749 transcripts which are detectable by amplicon as depicted in sequence name T29749_seg22-24WT (SEQ ID NO: 80) in different normal tissues.

Expression of ribonucleotide reductase M2 polypeptide (RRM2) transcripts detectable by or according to seg22-24WT—T29749_seg22-24WT (SEQ ID NO: 80) amplicon and primers T29749_seg22-24WTF (SEQ ID NO: 78) and T29749_seg22-24WTR (SEQ ID NO: 79) was measured by real time PCR. In parallel the expression of several housekeeping genes —SDHA (GenBank Accession No. NM_{—}004168 (SEQ ID NO:1); amplicon—SDHA-amplicon (SEQ ID NO: 4)), Ubiquitin (GenBank Accession No. BC000449 (SEQ ID NO:25); amplicon—Ubiquitin-amplicon (SEQ ID NO: 28)) and TATA box (GenBank Accession No. NM_{—}003194 (SEQ ID NO:33); TATA amplicon (SEQ ID NO: 36)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the lung samples (sample numbers 26, 28, 29 and 30, Table 1_{—}4 above), to obtain a value of relative expression of each sample relative to median of the lung samples.

Forward Primer (T29749_seg22-24WTF
(SEQ ID NO: 78)):
>T29749_seg22-24WTF (SEQ ID NO: 78)
TCAGATTACAGAAGCCCGCTG
Reverse Primer (T29749_seg22-24WTR
(SEQ ID NO: 79)):
>T29749_seg22-24WTR (SEQ ID NO: 79)
TGTAAGTGTCAATAAGAAGACTATACATTTCAG
Amplicon (T29749_seg22-24WT (SEQ ID NO: 80)):
>T29749_seg22-24WT (SEQ ID NO: 80)
TCAGATTACAGAAGCCCGCTGTTTCTATGGCTTCCAAATTGCCATGGAAA
ACATACATTCTGAAATGTATAGTCTTCTTATTGACACTTACA

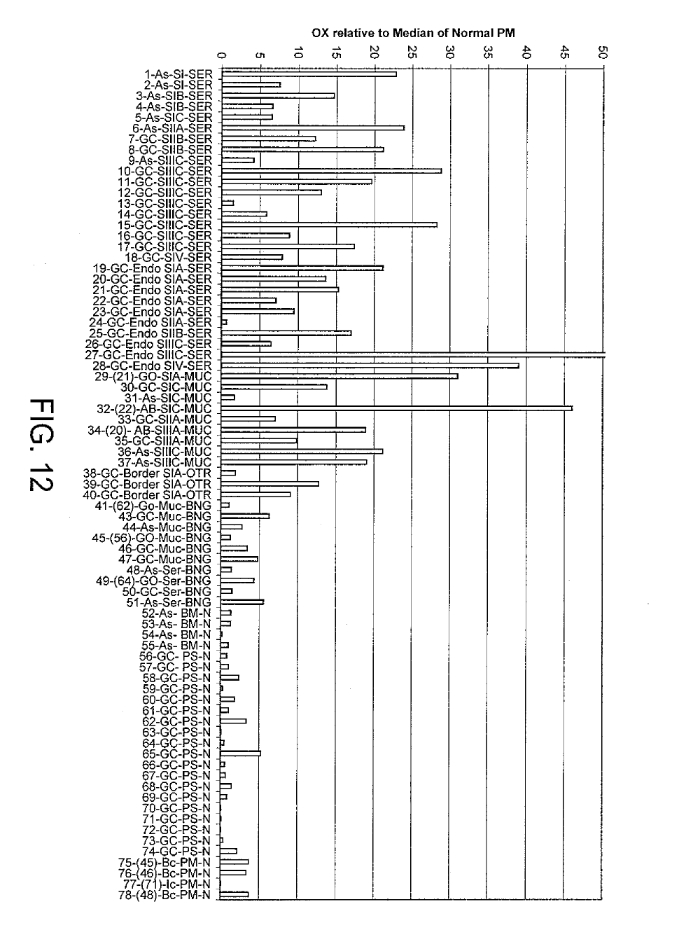

FIG. 9 demonstrates Expression of Homo sapiens ribonucleotide reductase M2 polypeptide (RRM2) T29749 transcripts which are detectable by amplicon as depicted in sequence name T29749_seg22-24WT (SEQ ID NO: 80) in different normal tissues.

Expression of Homo sapiens ribonucleotide reductase M2 polypeptide (RRM2) T29749 transcripts which are detectable by amplicon as depicted in sequence name T29749_seg29 (SEQ ID NO: 83) in normal and cancerous Breast tissues

Expression of Homo sapiens ribonucleotide reductase M2 polypeptide (RRM2) transcripts detectable by or according to seg29—T29749_seg29 (SEQ ID NO: 83) amplicon and primers T29749_seg29F (SEQ ID NO: 81) and T29749_seg29R (SEQ ID NO: 82) was measured by real time PCR In parallel the expression of four housekeeping genes—G6PD (GenBank Accession No. NM_{—}000402 (SEQ ID NO. 17); G6PD amplicon (SEQ ID NO: 20)), RPL19 (GenBank Accession No. NM_{—}000981 (SEQ ID NO:29); RPL19 amplicon (SEQ ID NO: 32)), PBGD (GenBank Accession No. BC019323 (SEQ ID NO:5); amplicon—PBGD-amplicon (SEQ ID NO: 8)) and SDHA (GenBank Accession No. NM_{—}004168 (SEQ ID NO:1); amplicon—SDHA-amplicon (SEQ ID NO: 4)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the normal samples (sample numbers 43, 45, 46, 47, 48, 49, 50, 51, 52, 54, 56, 58, 59, 60, 61, 62, 63, 64, 65, 66, 67, 68 and 69, Table 1_{—}3 above), to obtain a value of fold up-regulation for each sample relative to median of the normal samples.

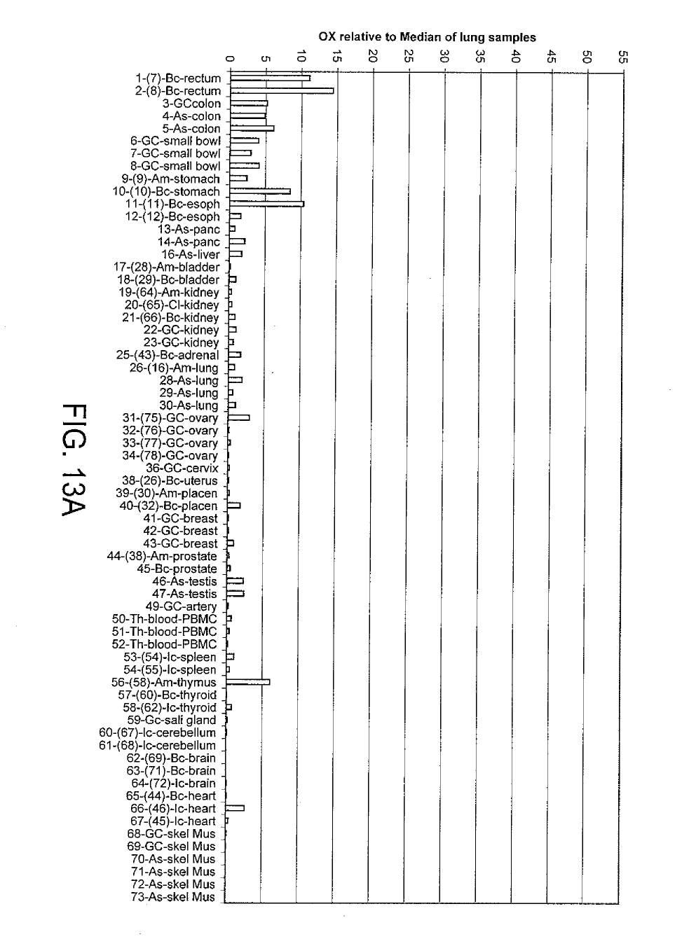

FIG. 10 is a histogram showing over expression of the above-indicated Homo sapiens ribonucleotide reductase M2 polypeptide (RRM2) transcripts in cancerous Breast samples relative to the normal samples.

As is evident from FIG. 10, the expression of Homo sapiens ribonucleotide reductase M2 polypeptide (RRM2) transcripts detectable by the above amplicon in cancer samples was significantly higher than in the non-cancerous samples (sample numbers 43, 45, 46, 47, 48, 49, 50, 51, 52, 54, 56, 58, 59, 60, 61, 62, 63, 64, 65, 66, 67, 68 and 69, Table 1_{—}3 above). Notably an over-expression of at least 10 fold was found in 26 out of 37 adenocarcinoma samples.

Statistical analysis was applied to verify the significance of these results, as described below.

The P value for the difference in the expression levels of Homo sapiens ribonucleotide reductase M2 polypeptide (RRM2) transcripts detectable by the above amplicon in Breast cancer samples versus the normal tissue samples was determined by T test as 4.27e-07.

Threshold of 10 fold over expression was found to differentiate between cancer and normal samples with P value of 2.77e-08 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T29749_seg29F (SEQ ID NO: 81) forward primer; and T29749_seg29R (SEQ ID NO: 82) reverse primer.

The present invention also preferably encompasses any amplicon obtained through the use of any suitable primer pair; for example, for the above experiment, the following amplicon was obtained as a non-limiting illustrative example only of a suitable amplicon: T29749_seg29 (SEQ ID NO: 83).

Forward Primer (T29749_seg29F (SEQ ID NO: 81)):
AACATTTCGGTGTGAGTTCTTCC
Reverse Primer (T29749_seg29R (SEQ ID NO: 82)):
GACCTCCGATTACCAACTGCTAGA
Amplicon (T29749_seg29 (SEQ ID NO: 83)):
AACATTTCGGTGTGAGTTCTTCCTTTAGGAAGAGGATTGGCAAATACTTG
AATTTGGCCTTTGTCCCAGAGCTCTTATCTAGCAGTTGGTAATCGGAG
GTC

Expression of ribonucleotide reductase M2 polypeptide (RRM2) T29749 transcripts which are detectable by amplicon as depicted in sequence name T29749_seg29 (SEQ ID NO: 83) in normal and cancerous Lung tissues.

Expression of ribonucleotide reductase M2 polypeptide (RRM2) transcripts detectable by or according to seg29—T29749_seg29 (SEQ ID NO: 83) amplicon and primers T29749_seg29F (SEQ ID NO: 81) and T29749_seg29R (SEQ ID NO: 82) was measured by real time PCR. In parallel the expression of several housekeeping genes —HPRT1 (GenBank Accession No. NM_{—}000194 (SEQ ID NO:9); amplicon—HPRTl-amplicon (SEQ ID NO: 12)), PBGD (GenBank Accession No. BC019323 (SEQ ID NO:5); amplicon—PBGD-amplicon (SEQ ID NO:8)), SDHA (GenBank Accession No. NM_{—}004168 (SEQ ID NO:1); amplicon—SDHA-amplicon (SEQ ID NO: 4)) and Ubiquitin (GenBank Accession No. BC000449 (SEQ ID NO:25); amplicon—Ubiquitin-amplicon (SEQ ID NO: 28)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}2 above), to obtain a value of fold up-regulation for each sample relative to median of the normal samples.

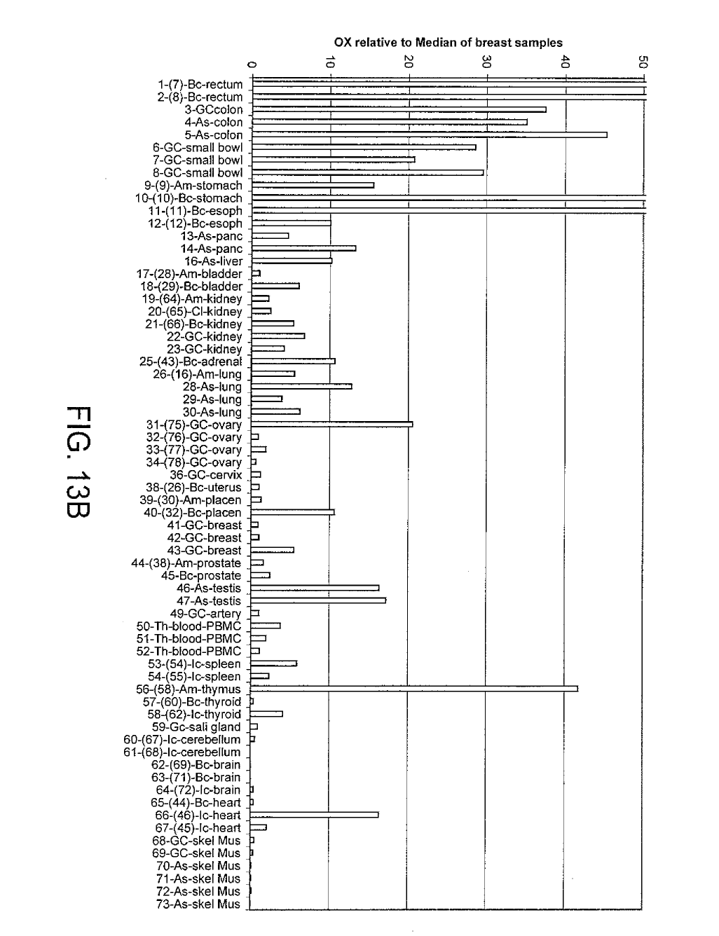

FIG. 11 is a histogram showing over expression of the above-indicated ribonucleotide reductase M2 polypeptide (RRM2) transcripts in cancerous Lung samples relative to the normal samples.

As is evident from FIG. 11, the expression of ribonucleotide reductase M2 polypeptide (RRM2) transcripts detectable by the above amplicon in adenocarcinoma, squamous cell carcinoma, large cell carcinoma, small cell carcinoma and non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}2 above). Notably an over-expression of at least 5 fold was found in 32 out of 57 non-small cell carcinoma samples, specifically in 8 out of 23 adenocarcinoma samples, in 18 out of 24 squamous cell carcinoma samples, in 6 out of 10 large cell carcinoma samples. Also over-expression was observed in 9 out of 9 small cell carcinoma samples.

Statistical analysis was applied to verify the significance of these results, as described below.

The P value for the difference in the expression levels of ribonucleotide reductase M2 polypeptide (RRM2) transcripts detectable by the above amplicon in Lung adenocarcinoma samples versus the normal tissue samples was determined by T test as 8.48e-004. The P value for the difference in the expression levels of ribonucleotide reductase M2 polypeptide (RRM2) transcripts detectable by the above amplicon in Lung squamous cell carcinoma samples versus the normal tissue samples was determined by T test as 3.08e-006. The P value for the difference in the expression levels of ribonucleotide reductase M2 polypeptide (RRM2) transcripts detectable by the above amplicon in Lung large cell carcinoma samples versus the normal tissue samples was determined by T test as 6.91e-004. The P value for the difference in the expression levels of ribonucleotide reductase M2 polypeptide (RRM2) transcripts detectable by the above amplicon in Lung small cell carcinoma samples versus the normal tissue samples was determined by T test as 2.81e-003. The P value for the difference in the expression levels of ribonucleotide reductase M2 polypeptide (RRM2) transcripts detectable by the above amplicon in Lung non-small cell carcinoma samples versus the normal tissue samples was determined by T test as 1.12e-010.

Threshold of 5 fold over expression was found to differentiate between adenocarcinoma and normal samples with P value of 7.97e-003 as checked by exact Fisher test. Threshold of 5 fold over expression was found to differentiate between squamous cell carcinoma and normal samples with P value of 1.19e-006 as checked by exact Fisher test. Threshold of 5 fold over expression was found to differentiate between large cell carcinoma and normal samples with P value of 9.12e-004 as checked by exact Fisher test. Threshold of 5 fold over expression was found to differentiate between small cell carcinoma and normal samples with P value of 4.89e-007 as checked by exact Fisher test. Threshold of 5 fold over expression was found to differentiate between non-small cell carcinoma and normal samples with P value of 1.96e-005 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T29749_seg29F (SEQ ID NO: 81) forward primer; and T29749_seg29R (SEQ ID NO: 82) reverse primer.

The present invention also preferably encompasses any amplicon obtained through the use of any suitable primer pair; for example, for the above experiment, the following amplicon was obtained as a non-limiting illustrative example only of a suitable amplicon: T29749_seg29 (SEQ ID NO: 83).

Forward Primer (T29749_seg29F (SEQ ID NO:81)):
>T29749_seg29F (SEQ ID NO:81)
AACATTTCGGTGTGAGTTCTTCC
Reverse Primer (T29749_seg29R (SEQ ID NO:82)):
>T29749_seg29R (SEQ ID NO:82)
GACCTCCGATTACCAACTGCTAGA
Amplicon (T29749_seg29 (SEQ ID NO:83)):
>T29749_seg29 (SEQ ID NO:83)
AACATTTCGGTGTGAGTTCTTCCTTTAGGAAGAGGATTGGCAAATACTTG
AATTTGGCCTTTGTCCCAGAGCTCTTATCTAGCAGTTGGTAATCGGAGGT
C

Expression of Homo sapiens ribonucleotide reductase M2 polypeptide (RRM2) T29749 transcripts which are detectable by amplicon as depicted in sequence name T29749_seg29 (SEQ ID NO: 83) in normal, benign and cancerous ovary tissues

Expression of Homo sapiens ribonucleotide reductase M2 polypeptide (RRM2) transcripts detectable by or according to seg29—T29749_seg29 (SEQ ID NO: 83) amplicon and primers T29749_seg29F (SEQ ID NO: 81) and T29749_seg29R (SEQ ID NO: 82) was measured by real time PCR. In parallel the expression of four housekeeping genes —SDHA (GenBank Accession No. NM_{—}004168 (SEQ ID NO: 1); amplicon—SDHA-amplicon (SEQ ID NO: 4)), HPRT1 (GenBank Accession No. NM_{—}000194 (SEQ ID NO:9); amplicon—HPRT1-amplicon (SEQ ID NO: 12)), and PBGD (GenBank Accession No. BC019323 (SEQ ID NO:5); amplicon—PBGD-amplicon (SEQ ID NO:8)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the normal samples (sample numbers 52-78, Table 1_{—}1 above), to obtain a value of fold up-regulation for each sample relative to median of the normal samples

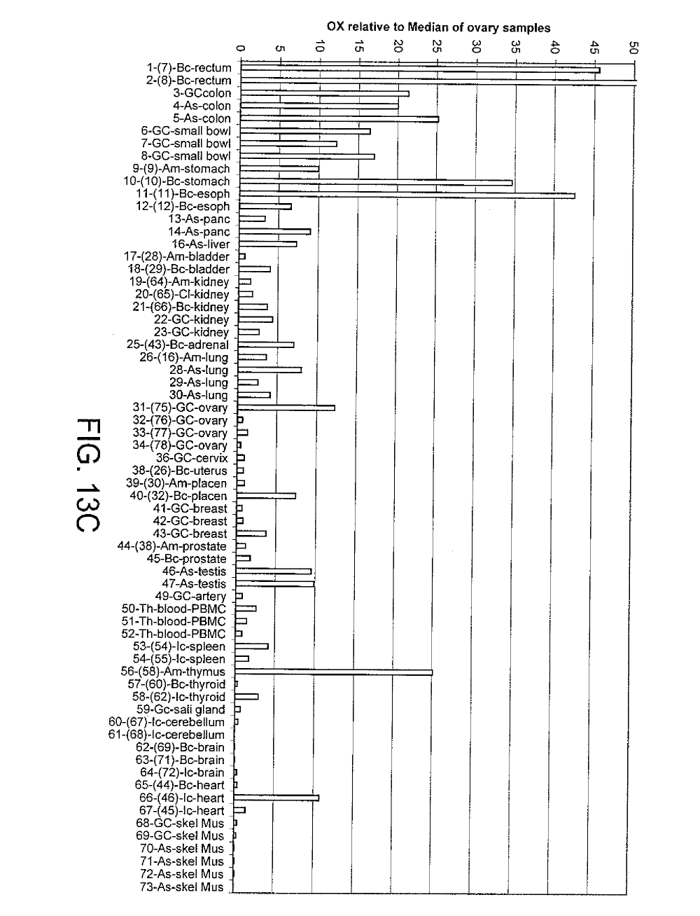

FIG. 12 is a histogram showing over expression of the above-indicated Homo sapiens ribonucleotide reductase M2 polypeptide (RRM2) transcripts in cancerous ovary samples relative to the normal samples.

As is evident from FIG. 12, the expression of Homo sapiens ribonucleotide reductase M2 polypeptide (RRM2) transcripts detectable by the above amplicon in adenocarcinoma samples was significantly higher than in the non-cancerous samples (sample numbers 41, 43-78, Table 1_{—}1 above). Notably an over-expression of at least 5 fold was found in in 35 out of 40 adenocarcinoma samples, specifically in 16 out of 18 serous carcinoma samples, in 9 out of 10 endometroid-type adenocarcinoma samples, and in 8 out of 9 mucinous carcinoma samples.

Statistical analysis was applied to verify the significance of these results, as described below.

Statistical analysis was applied to verify the significance of these results, as described below.

The P value for the difference in the expression levels of Homo sapiens ribonucleotide reductase M2 polypeptide (RRM2) transcripts detectable by the above amplicon in Ovary adenocarcinoma samples versus the non cancerous tissue samples was determined by T test as 8.17e-04. Threshold of 5 fold over expression was found to differentiate between adeonocarcinoma and non-cancerous samples with P value of 3.80e-13 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: T29749_seg29F (SEQ ID NO: 81) forward primer; and T29749_seg29R (SEQ ID NO: 82) reverse primer.

The present invention also preferably encompasses any amplicon obtained through the use of any suitable primer pair; for example, for the above experiment, the following amplicon was obtained as a non-limiting illustrative example only of a suitable amplicon: T29749_seg29 (SEQ ID NO: 83).

Forward Primer (T29749_seg29F (SEQ ID NO:81)):
>T29749_seg29F (SEQ ID NO:81)
AACATTTCGGTGTGAGTTCTTCC
Reverse Primer (T29749_seg29R (SEQ ID NO:82)):
>T29749_seg29R (SEQ ID NO:82)
GACCTCCGATTACCAACTGCTAGA
Amplicon (T29749_seg29 (SEQ ID NO:83)):
>T29749_seg29 (SEQ ID NO:83)
AACATTTCGGTGTGAGTTCTTCCTTTAGGAAGAGGATTGGCAAATACTTG
AATTTGGCCTTTGTCCCAGAGCTCTTATCTAGCAGTTGGTAATCGGAGGT
C

Expression of ribonucleotide reductase M2 polypeptide (RRM2) T29749 transcripts which are detectable by amplicon as depicted in sequence name T29749_seg29 (SEQ ID NO: 83) in different normal tissues.

Expression of ribonucleotide reductase M2 polypeptide (RRM2) transcripts detectable by or according to seg29—T29749_seg29 (SEQ ID NO: 83) amplicon and primers T29749_seg29F (SEQ ID NO: 81) and T29749_seg29R (SEQ ID NO: 82) was measured by real time PCR. In parallel the expression of several housekeeping genes —SDHA (GenBank Accession No. NM_{—}004168 (SEQ ID NO: 1); amplicon—SDHA-amplicon (SEQ ID NO: 4)), Ubiquitin (GenBank Accession No. BC000449 (SEQ ID) NO:25); amplicon—Ubiquitin-amplicon (SEQ ID NO: 28)) and TATA box (GenBank Accession No. NM_{—}003194 (SEQ ID NO:33); TATA amplicon (SEQ ID NO: 36)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the lung samples (sample numbers 26, 28, 29 and 30, Table 1_{—}4 above), to obtain a value of relative expression of each sample relative to median of the lung samples as presented in figure A, by the median of the quantities of the breast samples (sample numbers 41, 42, and 43, Table 1_{—}4 above), to obtain a value of relative expression of each sample relative to median of the breast samples as presented in figure B, by the median of the quantities of the ovary samples (sample numbers 31, 32, 33 and 34, Table 1_{—}4 above), to obtain a value of relative expression of each sample relative to median of the ovary samples as presented in figure C.

Forward Primer (T29749_seg29F (SEQ ID NO:81)):
>T29749_seg29F (SEQ ID NO:81)
AACATTTCGGTGTGAGTTCTTCC
Reverse Primer (T29749_seg29R (SEQ ID NO:82)):
>T29749_seg29R (SEQ ID NO:82)
GACCTCCGATTACCAACTGCTAGA
Amplicon (T29749_seg29 (SEQ ID NO:83)):
>T29749_seg29 (SEQ ID NO:83)
AACATTTCGGTGTGAGTTCTTCCTTTAGGAAGAGGATTGGCAAATACTTG
AATTTGGCCTTTGTCCCAGAGCTCTTATCTAGCAGTTGGTAATCGGAGGT
C

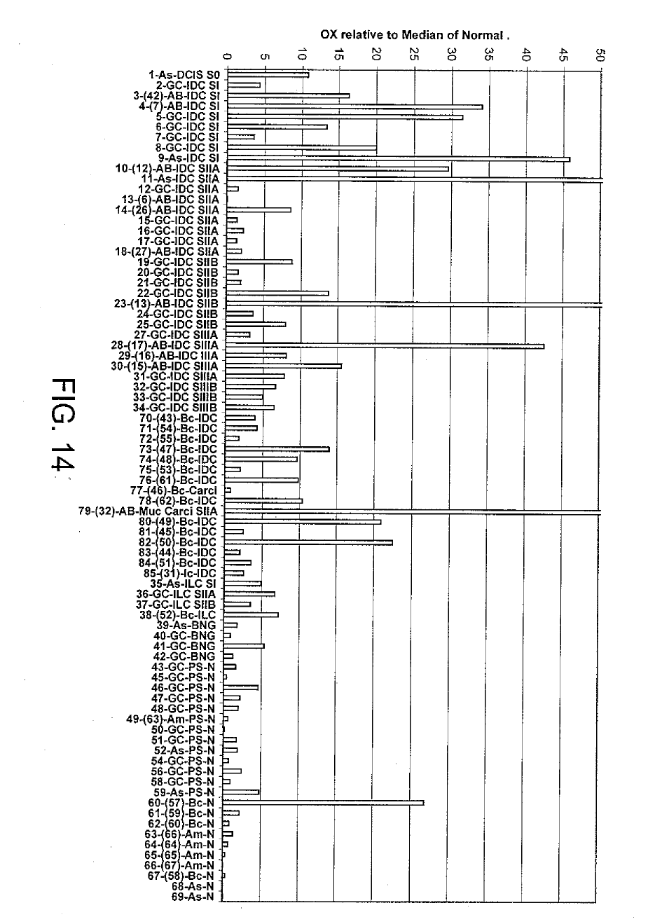

FIG. 13 demonstrates Expression of Homo sapiens ribonucleotide reductase M2 polypeptide (RRM2) T29749 transcripts which are detectable by amplicon as depicted in sequence name T29749_seg29 (SEQ ID NO: 83) in different normal tissues. FIG. 13A represents relative expression of each sample relative to median of the lung samples; FIG. 13B represents relative expression of each sample relative to median of the breast samples; and FIG. 13C represents relative expression of each sample relative to median of the ovary samples.

### Description for Cluster F13779

Cluster F13779 features 2 transcript(s) and 34 segment(s) of interest, the names for which are given in Tables 45 and 46, respectively, the sequences themselves are given at the end of the application. The selected protein variants are given in table 47.

TABLE 45
Transcripts of interest
Transcript Name

F13779_T0 (SEQ ID NO: 85)
F13779_T16 (SEQ ID NO: 86)

TABLE 46
Segments of interest
Segment Name

F13779_N0 (SEQ ID NO: 87)
F13779_N9 (SEQ ID NO: 88)
F13779_N11 (SEQ ID NO: 89)
F13779_N13 (SEQ ID NO: 90)
F13779_N32 (SEQ ID NO: 91)
F13779_N33 (SEQ ID NO: 92)
F13779_N34 (SEQ ID NO: 93)
F13779_N35 (SEQ ID NO: 94)
F13779_N39 (SEQ ID NO: 95)
F13779_N41 (SEQ ID NO: 96)
F13779_N43 (SEQ ID NO: 97)
F13779_N44 (SEQ ID NO: 98)
F13779_N45 (SEQ ID NO: 99)
F13779_N47 (SEQ ID NO: 100)
F13779_N6 (SEQ ID NO: 101)
F13779_N7 (SEQ ID NO: 102)
F13779_N15 (SEQ ID NO: 103)
F13779_N17 (SEQ ID NO: 104)
F13779_N18 (SEQ ID NO: 105)
F13779_N20 (SEQ ID NO: 106)
F13779_N22 (SEQ ID NO: 107)
F13779_N25 (SEQ ID NO: 108)
F13779_N26 (SEQ ID NO: 109)
F13779_N27 (SEQ ID NO: 110)
F13779_N28 (SEQ ID NO: 111)
F13779_N29 (SEQ ID NO: 112)
F13779_N30 (SEQ ID NO: 113)
F13779_N31 (SEQ ID NO: 114)
F13779_N36 (SEQ ID NO: 115)
F13779_N37 (SEQ ID NO: 116)
F13779_N38 (SEQ ID NO: 117)
F13779_N40 (SEQ ID NO: 118)
F13779_N42 (SEQ ID NO: 119)
F13779_N46 (SEQ ID NO: 120)

TABLE 47
Proteins of interest

| Protein Name | Corresponding Transcript(s) |
|---|---|
| F13779_P0 (SEQ ID NO: 121) | F13779_T0 (SEQ ID NO: 85) |
| F13779_P5 (SEQ ID NO: 124) | F13779_T16 (SEQ ID NO: 86) |

These sequences are variants of the known protein FLJ11029 protein (SwissProt accession identifier Q96HE9_HUMAN (SEQ ID NO:135)), referred to herein as the previously known protein.

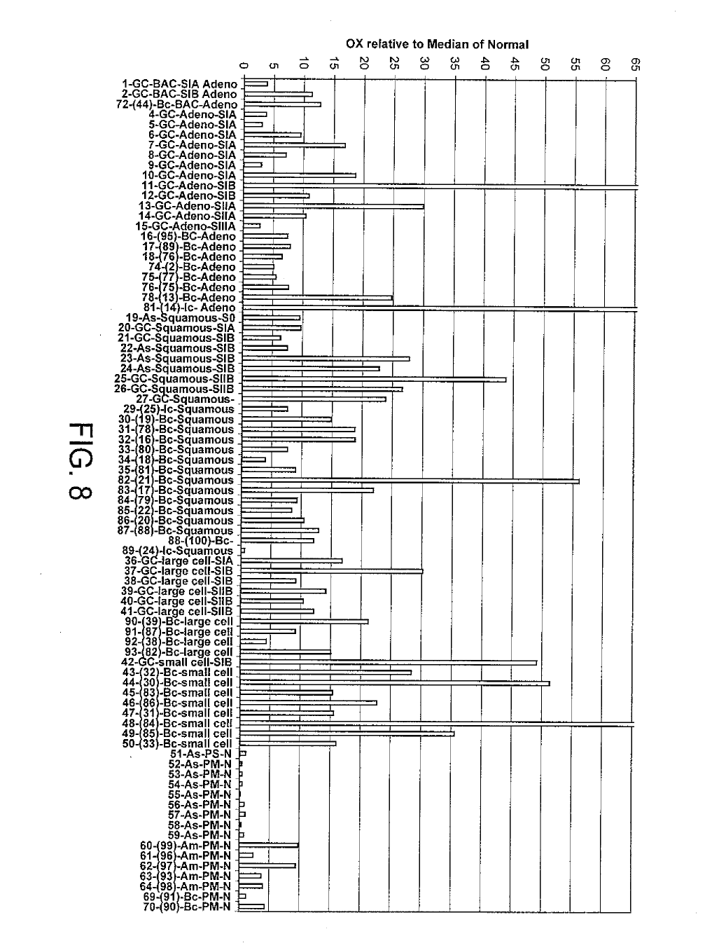

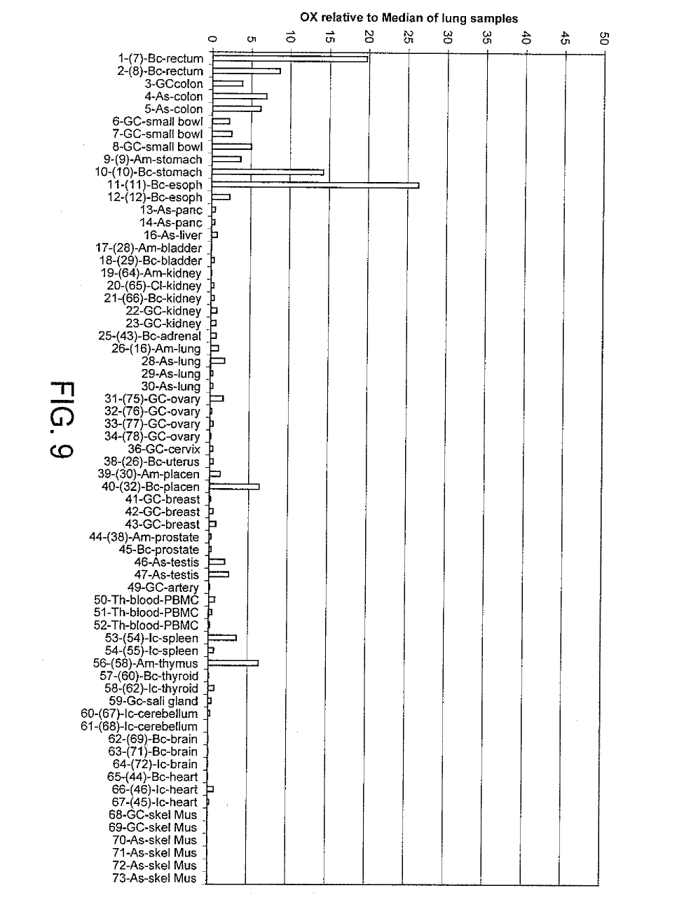

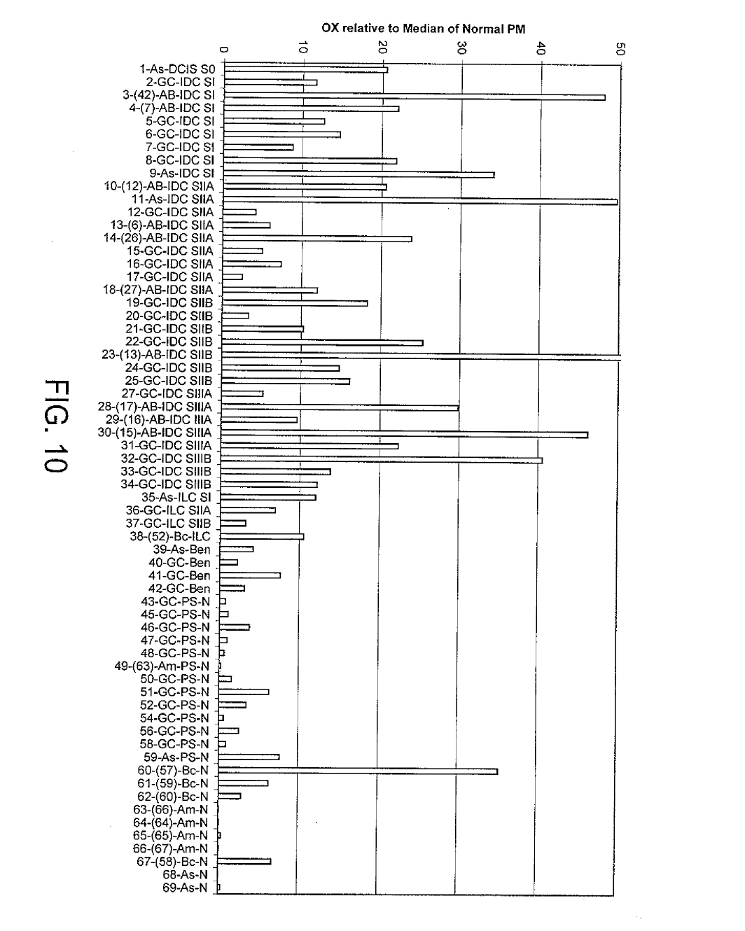

Cluster F13779 can be used as a diagnostic marker according to overexpression of transcripts of this cluster in cancer. Preferably, Cluster F13779 known wild type and Cluster F13779 variants can be used as a diagnostic marker for Lung cancer. Expression of F13779 transcripts in normal tissues is also given according to the previously described methods. The term “number” in table 48 and the numbers on the y-axis of the FIG. 7 refer to weighted expression of ESTs in each category, as “parts per million” (ratio of the expression of ESTs for a particular cluster to the expression of all ESTs in that category, according to parts per million).

Overall, the following results were obtained as shown with regard to the histograms in FIG. 7 and Table 48. This cluster is overexpressed (at least at a minimum level) in the following pathological conditions: brain malignant tumors, a mixture of malignant tumors from different tissues, skin malignancies and epithelial malignant tumors.

TABLE 48
Normal tissue distribution

| Name of Tissue | Number |
|---|---|
| brain | 2 |
| ovary | 0 |
| lung | 2 |
| pancreas | 0 |
| liver | 0 |
| prostate | 0 |
| T cells | 278 |
| adrenal | 0 |
| general | 7 |
| bone marrow | 0 |
| Thyroid | 0 |
| skin | 0 |
| muscle | 3 |
| uterus | 0 |
| colon | 0 |
| kidney | 2 |
| lymph nodes | 73 |
| breast | 0 |
| stomach | 0 |
| epithelial | 0 |
| bone | 0 |

TABLE 49
P values and ratios for expression in cancerous tissue

| Name of Tissue | P1 | P2 | SP1 | R3 | SP2 | R4 |
|---|---|---|---|---|---|---|
| brain | 2.3e−02 | 3.1e−03 | 1.1e−01 | 5.0 | 8.8e−06 | 6.0 |
| ovary | N/A | 6.5e−01 | N/A | N/A | 5.9e−01 | 1.6 |
| lung | 4.6e−01 | 3.4e−01 | 1.7e−01 | 3.2 | 8.9e−02 | 3.4 |
| pancreas | 3.1e−01 | 1.6e−01 | 4.2e−01 | 2.4 | 7.6e−02 | 3.7 |
| liver | N/A | 2.0e−01 | N/A | N/A | 3.4e−01 | 2.4 |
| prostate | 7.1e−01 | 5.7e−01 | N/A | N/A | 5.6e−01 | 1.7 |
| T cells | 5.0e−01 | 6.7e−01 | 1.0e+00 | 0.5 | 8.1e−01 | 0.9 |
| adrenal | N/A | 4.3e−01 | N/A | N/A | 2.8e−01 | 2.8 |
| general | 1.7e−02 | 7.7e−09 | 5.3e−02 | 1.9 | 2.1e−15 | 5.4 |
| bone marrow | N/A | 4.9e−01 | N/A | N/A | 5.4e−01 | 2.0 |
| Thyroid | 5.7e−01 | 5.7e−01 | 4.6e−01 | 1.9 | 4.6e−01 | 1.9 |
| skin | N/A | 1.1e−01 | N/A | N/A | 3.2e−03 | 3.2 |
| muscle | 9.2e−01 | 4.8e−01 | N/A | N/A | 1.6e−01 | 3.4 |
| uterus | 4.4e−01 | 1.2e−01 | 6.6e−01 | 1.5 | 2.1e−01 | 2.3 |
| colon | 2.0e−01 | 2.6e−01 | 6.9e−01 | 1.7 | 7.7e−01 | 1.5 |
| kidney | 9.6e−01 | 7.0e−01 | N/A | N/A | 2.3e−01 | 2.1 |
| lymph nodes | 8.6e−01 | 6.5e−01 | 1.0e+00 | 0.2 | 4.3e−01 | 0.7 |
| breast | 8.0e−01 | 5.8e−01 | 6.9e−01 | 1.5 | 2.5e−01 | 2.1 |
| stomach | 3.3e−01 | 1.0e−01 | N/A | N/A | 2.0e−01 | 2.5 |
| epithelial | 9.7e−03 | 5.5e−06 | 3.2e−03 | 7.5 | 2.4e−10 | 18.4 |
| bone | N/A | 2.8e−01 | N/A | N/A | 3.4e−01 | 2.4 |

As noted above, cluster F13779 features 2 transcript(s), which were listed in Table 45 above. These transcript(s) encode for protein(s) which are variant(s) of protein FLJ11029 protein. A description of each variant protein according to the present invention is now provided.

Variant protein F13779_P0 (SEQ ID NO:121) according to the present invention has an amino acid sequence as given at the end of the application; it is encoded by transcript(s) F13779_T0 (SEQ ID NO:85). An alignment is given to the known protein (FLJ11029 protein) at the end of the application. One or more alignments to one or more previously published protein sequences are given at the end of the application. A brief description of the relationship of the variant protein according to the present invention to each such aligned protein is as follows:

1. Comparison report between F13779_P0 (SEQ ID NO:121) and Q9NUZ7_HUMAN (SEQ ID NO:123):
A. An isolated chimeric polypeptide encoding for F13779_P0 (SEQ ID NO:121), comprising a first amino acid sequence being at least 90% or 95% homologous to MPKFKQRRRKLKAKAERLFKKKEASHFQSKLITPPPPPPSPERVGISSIDISQSRSWLTSS WNFNFPNIRDAIKLWTNRVWSIYSWCQNCITQSLEVLKDTIFPSRICHRELYSVKQQFCIL ESKLCKLQEALKTISESSSCPSCGQTCHMSGKLTNVPACVLITPGDSKAVLPPTLPQPASH FPPPPPPPPLPPPPPPLAPVLLRKPSLAKALQAGPLKKDGPMQITVKDLLTVKLKKTQSLD EKRKLIPSPKARNPLVTVSDLQHVTLKPNSKVLSTRVTNVLITPGKSQMDLRKLLRKVD VER corresponding to amino acids 1-306 of Q9NUZ7_HUMAN (SEQ ID NO: 123), which also corresponds to amino acids 1-306 of F13779_P0 (SEQ ID NO:121), a bridging amino acid S corresponding to amino acid 307 of F13779_P0 (SEQ ID NO:121), and a second amino acid sequence being at least 90% or 95% homologous to PGGTPLTNKENMETGTGLTPVMTQALRRKFQLAHPRSPTPTLPLSTSSFDEQN corresponding to amino acids 308-360 of Q9NUZ7_HUMAN (SEQ ID NO:123), which also corresponds to amino acids 308-360 of F13779_P0 (SEQ ID NO:121), wherein said first amino acid sequence, bridging amino acid and second amino acid sequence are contiguous and in a sequential order.

2. Comparison report between F13779_P0 (SEQ ID NO:121) and Q96HE9_HUMAN (SEQ ID NO:135):
A. An isolated chimeric polypeptide encoding for F13779_P0 (SEQ ID NO:121), comprising a first amino acid sequence being at least 90% or 95% homologous to MPKFKQRRRKLKAKAERLFKKKEASHFQSKLITPPPPPPSPERVGISSIDISQSRSWLTSS WNFNFPNIRDAIKLWTNRVWSIYSWCQNCITQSLEVLKDTIFPSRICHRELYSVKQQFCIL ESKLCKLQEALKTISESSSCPSCGQTCHMSGKLTNVPACVLITPGDSKAVLPPTLPQPASH FPPPPPPPPLPPPPPPLAPVLLRKPSLAKALQAGPLKKDGPMQITVKDLLTVKLKKTQSLD EKRKLIPSPKARNPLVTVSDLQHVTLKPNSKVLSTRVTNVLITPGKSQMDLRKLLRKVD VER corresponding to amino acids 1-306 of NP_{—}060774 (SEQ ID NO:123), which also corresponds to amino acids 1-306 of F13779_P0 (SEQ ID NO:121), a bridging amino acid S corresponding to amino acid 307 of F13779_P0 (SEQ ID NO:121), and a second amino acid sequence being at least 90% or 95% homologous to PGGTPLTNKENMETGTGLTPVMTQALRRKFQLAHPRSPTPTLPLSTSSFDEQN corresponding to amino acids 308-360 of NP_{—}060774 (SEQ ID NO:123), which also corresponds to amino acids 308-360 of F13779_P0 (SEQ ID NO:121), wherein said first amino acid sequence, bridging amino acid and second amino acid sequence are contiguous and in a sequential order.

3. Comparison report between F13779_P0 (SEQ ID NO:121) and NP_{—}060774 (SEQ ID NO:123):
A. An isolated chimeric polypeptide encoding for F13779_P0 (SEQ ID NO:121), comprising a first amino acid sequence being at least 90% or 95% homologous to MPKFKQRRRKLKAKAERLFKKKEASHFQSKLITPPPPPPSPERVGISSIDISQSRSWLTSS WNFNFPNIRDAIKLWTNRVWSTYSWCQNCITQSLEVLKDTIFPSRICHRELYSVKQQFCIL ESKLCKLQEALKTISESSSCPSCGQTCHMSGKLTNVPACVLITPGDSKAVLPPTLPQPASH FPPPPPPPPLPPPPPPLAPVLLRKPSLAKALQAGPLKKDGPMQITVKDLLTVKLKKTQSLD EKRKLIPSPKARNPLVTVSDLQHVTLKPNSKVLSTRVTNVLITPGKSQMDLRKLLRKVD VER corresponding to amino acids 1-306 of NP_{—}060774 (SEQ ID NO:123), which also corresponds to amino acids 1-306 of F13779_P0 (SEQ ID NO:121), a bridging amino acid S corresponding to amino acid 307 of F13779_P0 (SEQ ID NO:121), and a second amino acid sequence being at least 90% or 95% homologous to PGGTPLTNKENMETGTGLTPVMTQALRRKFQLAHPRSPTPTLPLSTSSFDEQN corresponding to amino acids 308-360 of NP_{—}060774 (SEQ ID NO:123), which also corresponds to amino acids 308-360 of F13779_P0 (SEQ ID NO:121), wherein said first amino acid sequence, bridging amino acid and second amino acid sequence are contiguous and in a sequential order.

4. Comparison report between F13779_P0 (SEQ ID NO:121) and Q9NXE9_HUMAN (SEQ ID NO: 122):
A. An isolated chimeric polypeptide encoding for F13779_P0 (SEQ ID NO:121), comprising a first amino acid sequence being at least 70%, optionally at least 80%, preferably at least 85%, more preferably at least 90% and most preferably at least 95%, homologous to a polypeptide having the sequence MPKFKQRRRKLKAKAERLFKKKEASHFQSKLITPPPPPPSPERVGISSIDISQSRSWLTSS WNFNFPNIRDAIKLWTNRVWSIYSWCQNCITQSLEVLKDTIFPSRICHRELYSVKQQFCIL ESKLCKLQEALKTISESSSCPSCGQTCH (SEQ ID NO: 133) corresponding to amino acids 1-150 of F13779_P0 (SEQ ID NO:121), and a second amino acid sequence being at least 90% or 95% homologous to MSGKLTNVPACVLITPGDSKAVLPPTLPQPASHFPPPPPPPPLPPPPPPLAPVLLRKPSLAK ALQAGPLKKDGPMQITVKDLLTVKLKKTQSLDEKRKLIPSPKARNPLVTVSDLQHVTLK PNSKVLSTRVTNVLITPGKSQMDLRKLLRKVDVERSPGGTPLTNKENMETGTGLTPVMT QALRRKFQLAHPRSPTPTLPLSTSSFDEQN corresponding to amino acids 1-210 of Q9NXE9_HUMAN (SEQ ID NO:122), which also corresponds to amino acids 151-360 of F13779_P0 (SEQ ID NO:121), wherein said first amino acid sequence and second amino acid sequence are contiguous and in a sequential order.
B. An isolated polypeptide encoding for a head of F13779_P0 (SEQ ID NO:121), comprising a polypeptide being at least 70%, optionally at least about 80%, preferably at least about 85%, more preferably at least about 90% and most preferably at least about 95% homologous to the sequence MPKFKQRRRKLKAKAERLFKKKEASBFQSKLITPPPPPPSPERVGISSIDISQSRSWLTSS WNFNFPNIRDAIKLWTNRVWSIYSWCQNCITQSLEVLKDTIFPSRICHRELYSVKQQFCIL ESKLCKLQEALKTISESSSCPSCGQTCH (SEQ ID NO: 133) of F13779_P0 (SEQ ID NO:121).

The localization of the variant protein was determined according to results from a number of different software programs and analyses, including analyses from SignalP and other specialized programs. The variant protein is believed to be located as follows with regard to the cell: intracellularly.

Variant protein F13779_P0 (SEQ ID NO:121) also has the following non-silent SNPs (Single Nucleotide Polymorphisms) as listed in Table 50, (given according to their position(s) on the amino acid sequence, with the alternative amino acid(s) listed; the last column indicates whether the SNP is known or not; the presence of known SNPs in variant protein F13779_P0 (SEQ ID NO:121) sequence provides support for the deduced sequence of this variant protein according to the present invention).

TABLE 50
Amino acid mutations

| SNP position(s) on amino acid | Alternative | |
|---|---|---|
| sequence | amino acid(s) | Previously known SNP? |
| 122 | L -> S | No |
| 122 | L -> W | No |
| 194 | P -> A | No |
| 195 | P -> | No |
| 280 | R -> Q | Yes |

Variant protein F13779_P0 (SEQ ID NO:121) is encoded by the following transcript(s): F13779_T0 (SEQ ID NO:85), for which the sequence(s) is/are given at the end of the application. The coding portion of transcript F13779_T0 (SEQ ID NO:85) is shown in bold; this coding portion starts at position 286 and ends at position 1365. The transcript also has the following SNPs as listed in Table 51 (given according to their position on the nucleotide sequence, with the alternative nucleic acid listed; the last column indicates whether the SNP is known or not; the presence of known SNPs in variant protein F13779_P0 (SEQ ID NO:121) sequence provides support for the deduced sequence of this variant protein according to the present invention).

TABLE 51
Nucleic acid SNPs

| SNP position(s) on amino acid | Alternative | |
|---|---|---|
| sequence | amino acid(s) | Previously known SNP? |
| 650 | T -> G | No |
| 650 | T -> C | No |
| 865 | C -> G | No |
| 868 | C -> | No |
| 1124 | G -> A | Yes |
| 1446 | A -> G | No |
| 1609 | G -> A | No |
| 1658 | G -> A | No |
| 1696 | G -> A | Yes |
| 2476 | G -> A | Yes |
| 2597 | C -> T | Yes |
| 3017 | G -> A | No |
| 3025 | A -> G | No |
| 3079 | G -> A | No |
| 3160 | C -> T | Yes |
| 3196 | A -> G | No |
| 3198 | G -> A | No |
| 3205 | A -> G | No |
| 3328 | A -> G | No |
| 3397 | A -> G | No |
| 3440 | A -> C | Yes |
| 3448 | A -> C | Yes |
| 3452 | A -> G | Yes |
| 3501 | A -> G | No |
| 3514 | A -> G | Yes |
| 3571 | -> A | No |
| 3686 | G -> T | Yes |
| 4005 | T -> C | Yes |
| 4297 | T -> C | Yes |
| 4469 | G -> A | Yes |
| 4543 | A -> T | Yes |
| 4887 | C -> A | Yes |
| 4947 | G -> A | No |
| 5001 | G -> A | No |
| 5369 | G -> A | Yes |
| 5400 | C -> T | Yes |
| 5883 | C -> T | Yes |
| 5921 | C -> T | Yes |
| 6242 | C -> A | Yes |

Variant protein F13779_P5 (SEQ ID NO:124) according to the present invention has an amino acid sequence as given at the end of the application; it is encoded by transcript(s) F13779_T16 (SEQ ID NO:86). An alignment is given to the known protein (FLJ11029 protein) at the end of the application. One or more alignments to one or more previously published protein sequences are given at the end of the application. A brief description of the relationship of the variant protein according to the present invention to each such aligned protein is as follows:

1. Comparison report between F13779_P5 (SEQ ID NO:124) and Q9NUZ7_HUMAN (SEQ ID NO:123):
A. An isolated chimeric polypeptide encoding for F13779_P5 (SEQ ID NO:124), comprising a first amino acid sequence being at least 90% or 95% homologous to MPKFKQRRRKLKAKAERLFKKKEASHFQSKLITPPPPPPSPERVGISSIDISQSRSWLTSS WNFNPNIRDAIKLWTNRVWSWSWCQNCITQSLEVLKDTIFPSRICHRELYSVKQQFCIL ESKLCKLQEALKTISESSSCPSCGQTCHMSGKLTNVPACVLITPGDSKAVLPPTLPQPASH FPPPPPPPPLPPPPPPLAPVLLRKPSLAKALQAGPLKKDGPMQITVKDLLTVKLKKTQSLD EKRKLIPSPKARNPLVTVSDLQHVTLKPNSKVLSTRVTNVL corresponding to amino acids 1-285 of Q9NUZ7_HUMAN (SEQ ID NO: 123), which also corresponds to amino acids 1-285 of F13779_P5 (SEQ ID NO:124), and a second amino acid sequence being at least 70%, optionally at least 80%, preferably at least 85%, more preferably at least 90% and most preferably at least 95% homologous to a polypeptide having the sequence M corresponding to amino acids 286-286 of F13779_P5 (SEQ ID NO. 124), wherein said first amino acid sequence and second amino acid sequence are contiguous and in a sequential order.
B. An isolated polypeptide encoding for an edge portion of F13779_P5 (SEQ ID NO:124), comprising an amino acid sequence being at least 70%, optionally at least about 80%, preferably at least about 85%, more preferably at least about 90% and most preferably at least about 95% homologous to the sequence M of F13779_P5 (SEQ ID NO:124).

2. Comparison report between F13779_P5 (SEQ ID NO:124) and Q96HE9_HUMAN (SEQ ID NO:135):
A. An isolated chimeric polypeptide encoding for F13779_P5 (SEQ ID NO:124), comprising a first amino acid sequence being at least 90% or 95% homologous to MPKFKQRRRKLKAKAERLFKKKEASHFQSKLITPPPPPPSPERVGISSIDISQSRSWLTSS WNFNFPNIRDAIKLWTNRVWSIYSWCQNCITQSLEVLKDTIFPSRICHRELYSVKQQFCIL ESKLCKLQEALKTISESSSCPSCGQTCHMSGKLTNVPACVLITPGDSKAVLPPTLPQPASH FPPPPPPPPLPPPPPPLAPVLLRKPSLAKALQAGPLKKDGPMQITVKDLLTVKLKKTQSLD EKRKLIPSPKARNPLVTVSDLQHVTLKPNSKVLSTRVTNVL corresponding to amino acids 1-285 of Q96HE9_HUMAN (SEQ ID NO:135), which also corresponds to amino acids 1-285 of F13779_P5 (SEQ ID NO:124), and a second amino acid sequence being at least 70%, optionally at least 80%, preferably at least 85%, more preferably at least 90% and most preferably at least 95% homologous to a polypeptide having the sequence M corresponding to amino acids 286-286 of F13779_P5 (SEQ ID NO:124), wherein said first amino acid sequence and second amino acid sequence are contiguous and in a sequential order.
B. An isolated polypeptide encoding for an edge portion of F13779_P5 (SEQ if) NO:124), comprising an amino acid sequence being at least 70%, optionally at least about 80%, preferably at least about 85%, more preferably at least about 90% and most preferably at least about 95% homologous to the sequence M of F13779_P5 (SEQ ID NO: 124).

3. Comparison report between F13779_P5 (SEQ ID NO:124) and NP_{—}060774 (SEQ ID NO:123):
A. An isolated chimeric polypeptide encoding for F13779_P5 (SEQ ID NO:124), comprising a first amino acid sequence being at least 90% or 95% homologous to MPKFKQRRRKLKAKAERLFKKKEASHFQSKLITPPPPPPSPERVGISSIDISQSRSWLTSS WNFNFPNIRDAIKLWTNRVWSIYSWCQNCITQSLEVLKDTIFPSRICHRELYSVKQQFCIL ESKLCKLQEALKTISESSSCPSCGQTCHMSGKLTNVPACVLITPGDSKAVLPPTLPQPASH FPPPPPPPPLPPPPPPLAPVLLRKPSLAKALQAGPLKKDGPMQITVKDLLTVKLKKTQSLD EKRKLIPSPKARNPLVTVSDLQHVTLKPNSKVLSTRVTNVL corresponding to amino acids 1-285 of NP_{—}060774 (SEQ ID NO:123), which also corresponds to amino acids 1-285 of F13779_P5 (SEQ ID NO:124), and a second amino acid sequence being at least 70%, optionally at least 80%, preferably at least 85%, more preferably at least 90% and most preferably at least 95% homologous to a polypeptide having the sequence M corresponding to amino acids 286-286 of F13779_P5 (SEQ ID NO:124), wherein said first amino acid sequence and second amino acid sequence are contiguous and in a sequential order.
B. An isolated polypeptide encoding for an edge portion of F13779_P5 (SEQ ID NO:124), comprising an amino acid sequence being at least 70%, optionally at least about 80%, preferably at least about 85%, more preferably at least about 90% and most preferably at least about 95% homologous to the sequence M of F13779_P5 (SEQ ID NO: 124).

The localization of the variant protein was determined according to results from a number of different software programs and analyses, including analyses from SignalP and other specialized programs. The variant protein is believed to be located as follows with regard to the cell: intracellularly.

Variant protein F13779_P5 (SEQ ID NO:124) also has the following non-silent SNPs (Single Nucicotide Polymorphisms) as listed in Table 52, (given according to their position(s) on the amino acid sequence, with the alternative amino acid(s) listed; the last column indicates whether the SNP is known or not; the presence of known SNPs in variant protein F13779_P5 (SEQ ID NO: 124) sequence provides support for the deduced sequence of this variant protein according to the present invention).

TABLE 52
Amino acid mutations

| SNP position(s) on amino acid | Alternative | |
|---|---|---|
| sequence | amino acid(s) | Previously known SNP? |
| 122 | L -> S | No |
| 122 | L -> W | No |
| 194 | P -> A | No |
| 195 | P -> | No |
| 280 | R -> Q | Yes |

Variant protein F13779_P5 (SEQ ID NO: 124) is encoded by the following transcript(s): F13779_T16 (SEQ ID NO:86), for which the sequence(s) is/are given at the end of the application. The coding portion of transcript F13779_T16 (SEQ ID NO:86) is shown in bold; this coding portion starts at position 286 and ends at position 1143. The transcript also has the following SNPs as listed in Table 53 (given according to their position on the nucleotide sequence, with the alternative nucleic acid listed; the last column indicates whether the SNP is known or not; the presence of known SNPs in variant protein F13779_P5 (SEQ ID NO:124) sequence provides support for the deduced sequence of this variant protein according to the present invention).

TABLE 53
Nucleic acid SNPs

| SNP position(s) on amino acid | Alternative | |
|---|---|---|
| sequence | amino acid(s) | Previously known SNP? |
| 650 | T -> G | No |
| 650 | T -> C | No |
| 865 | C -> G | No |
| 868 | C -> | No |
| 1124 | G -> A | Yes |

As noted above, cluster F13779 features 34 segment(s), which were listed in Table 46 above and for which the sequence(s) are given at the end of the application. These segment(s) are portions of nucleic acid sequence(s) which are described herein separately because they are of particular interest. A description of each segment according to the present invention is now provided.

Segment cluster F13779_N0 (SEQ ID NO:87) according to the present invention is supported by 30 libraries. The number of libraries was determined as previously described. This segment can be found in the following transcript(s): F13779_T0 (SEQ ID NO:85) and F13779_T16 (SEQ ID NO:86). Table 54 below describes the starting and ending position of this segment on each transcript.

TABLE 54
Segment location on transcripts

| | Segment | Segment |
|---|---|---|
| Transcript name | starting position | ending position |
| F13779_T0 (SEQ ID NO: 85) | 1 | 280 |
| F13779_T16 (SEQ ID NO: 86) | 1 | 280 |

Segment cluster F13779_N9 (SEQ ID NO:88) according to the present invention is supported by 35 libraries. The number of libraries was determined as previously described. This segment can be found in the following transcript(s): F13779_T0 (SEQ ID NO:85) and F13779_T16 (SEQ ID NO. 86). Table 55 below describes the starting and ending position of this segment on each transcript.

TABLE 55
Segment location on transcripts

| | Segment | Segment |
|---|---|---|
| Transcript name | starting position | ending position |
| F13779_T0 (SEQ ID NO: 85) | 414 | 564 |
| F13779_T16 (SEQ ID NO: 86) | 414 | 564 |

Segment cluster F13779_N11 (SEQ ID NO:89) according to the present invention is supported by 36 libraries. The number of libraries was determined as previously described. This segment can be found in the following transcript(s): F13779_T0 (SEQ ID NO:85) and F13779_T16 (SEQ ID NO:86). Table 56 below describes the starting and ending position of this segment on each transcript.

TABLE 56
Segment location on transcripts

| | Segment | Segment |
|---|---|---|
| Transcript name | starting position | ending position |
| F13779_T0 (SEQ ID NO: 85) | 565 | 687 |
| F13779_T16 (SEQ ID NO: 86) | 565 | 687 |

Segment cluster F13779_N13 (SEQ ID NO:90) according to the present invention is supported by 40 libraries. The number of libraries was determined as previously described. This segment can be found in the following transcript(s): F13779_T0 (SEQ ID NO:85) and F13779_T16 (SEQ ID NO:86). Table 57 below describes the starting and ending position of this segment on each transcript.

TABLE 57
Segment location on transcripts

| | Segment | Segment |
|---|---|---|
| Transcript name | starting position | ending position |
| F13779_T0 (SEQ ID NO: 85) | 688 | 930 |
| F13779_T16 (SEQ ID NO: 86) | 688 | 930 |

Segment cluster F13779_N32 (SEQ ID NO:91) according to the present invention is supported by 24 libraries. The number of libraries was determined as previously described. This segment can be found in the following transcript(s): F13779_T0 (SEQ ID NO:85). Table 58 below describes the starting and ending position of this segment on each transcript.

TABLE 58
Segment location on transcripts

| | Segment | Segment |
|---|---|---|
| | starting | ending |
| Transcript name | position | position |
| F13779_T0 (SEQ ID NO: 85) | 1515 | 2272 |

Segment cluster F13779_N33 (SEQ ID NO:92) according to the present invention is supported by 20 libraries. The number of libraries was determined as previously described. This segment can be found in the following transcript(s): F13779_T0 (SEQ ID NO:85). Table 59 below describes the starting and ending position of this segment on each transcript.

TABLE 59
Segment location on transcripts

| | Segment | Segment |
|---|---|---|
| | starting | ending |
| Transcript name | position | position |
| F13779_T0 (SEQ ID NO: 85) | 2273 | 2642 |

Segment cluster F13779_N34 (SEQ ID NO:93) according to the present invention is supported by 18 libraries. The number of libraries was determined as previously described. This segment can be found in the following transcript(s): F13779_T0 (SEQ ID NO:85). Table 16 below describes the starting and ending position of this segment on each transcript.

TABLE 16
Segment location on transcripts

| | Segment | Segment |
|---|---|---|
| | starting | ending |
| Transcript name | position | position |
| F13779_T0 (SEQ ID NO: 85) | 2643 | 2878 |

Segment cluster F13779_N35 (SEQ ID NO:94) according to the present invention is supported by 12 libraries. The number of libraries was determined as previously described. This segment can be found in the following transcript(s): F13779_T0 (SEQ ID NO:85). Table 60 below describes the starting and ending position of this segment on each transcript.

TABLE 60
Segment location on transcripts

| | Segment | Segment |
|---|---|---|
| | starting | ending |
| Transcript name | position | position |
| F13779_T0 (SEQ ID NO: 85) | 2879 | 3063 |

Segment cluster F13779_N39 (SEQ ID NO:95) according to the present invention is supported by 25 libraries. The number of libraries was determined as previously described. This segment can be found in the following transcript(s): F13779_T0 (SEQ ID NO:85). Table 61 below describes the starting and ending position of this segment on each transcript.

TABLE 61
Segment location on transcripts

| | Segment | Segment |
|---|---|---|
| | starting | ending |
| Transcript name | position | position |
| F13779_T0 (SEQ ID NO: 85) | 3266 | 3402 |

Segment cluster F13779_N41 (SEQ ID NO:96) according to the present invention is supported by 27 libraries. The number of libraries was determined as previously described. This segment can be found in the following transcript(s): F13779_T0 (SEQ ID NO:85). Table 62 below describes the starting and ending position of this segment on each transcript.

TABLE 62
Segment location on transcripts

| | Segment | Segment |
|---|---|---|
| | starting | ending |
| Transcript name | position | position |
| F13779_T0 (SEQ ID NO: 85) | 3415 | 3536 |

Segment cluster F13779_N43 (SEQ ID NO:97) according to the present invention is supported by 36 libraries. The number of libraries was determined as previously described. This segment can be found in the following transcript(s): F13779_T0 (SEQ ID NO:85). Table 63 below describes the starting and ending position of this segment on each transcript.

TABLE 63
Segment location on transcripts

| | Segment | Segment |
|---|---|---|
| | starting | ending |
| Transcript name | position | position |
| F13779_T0 (SEQ ID NO: 85) | 3542 | 3893 |

Segment cluster F13779_N44 (SEQ ID NO:98) according to the present invention is supported by 20 libraries. The number of libraries was determined as previously described. This segment can be found in the following transcript(s): F13779_T0 (SEQ ID NO:85). Table 64 below describes the starting and ending position of this segment on each transcript.

TABLE 64
Segment location on transcripts

| | Segment | Segment |
|---|---|---|
| | starting | ending |
| Transcript name | position | position |
| F13779_T0 (SEQ ID NO: 85) | 3894 | 4176 |

Segment cluster F13779_N45 (SEQ ID NO:99) according to the present invention is supported by 43 libraries. The number of libraries was determined as previously described. This segment can be found in the following transcript(s): F13779_T0 (SEQ ID NO:85). Table 65 below describes the starting and ending position of this segment on each transcript.

TABLE 65
Segment location on transcripts

| | Segment | Segment |
|---|---|---|
| | starting | ending |
| Transcript name | position | position |
| F13779_T0 (SEQ ID NO: 85) | 4177 | 6251 |

Segment cluster F13779_N47 (SEQ ID NO:100) according to the present invention is supported by 10 libraries. The number of libraries was determined as previously described. This segment can be found in the following transcript(s): F13779_T0 (SEQ ID NO:85). Table 66 below describes the starting and ending position of this segment on each transcript.

TABLE 66
Segment location on transcripts

| | Segment | Segment |
|---|---|---|
| | starting | ending |
| Transcript name | position | position |
| F13779_T0 (SEQ ID NO: 85) | 6264 | 6444 |

According to an optional embodiment of the present invention, short segments related to the above cluster are also provided. These segments are up to about 120 bp in length, and so are included in a separate description.

Segment cluster F13779_N6 (SEQ ID NO:101) according to the present invention is supported by 35 libraries. The number of libraries was determined as previously described. This segment can be found in the following transcript(s): F13779_T0 (SEQ ID NO:85) and F13779_T16 (SEQ ID NO:86). Table 67 below describes the starting and ending position of this segment on each transcript.

TABLE 67
Segment location on transcripts

| | Segment | Segment |
|---|---|---|
| | starting | ending |
| Transcript name | position | position |
| F13779_T0 (SEQ ID NO: 85) | 281 | 375 |
| F13779_T16 (SEQ ID NO: 86) | 281 | 375 |

Segment cluster F13779_N7 (SEQ ID NO:102) according to the present invention is supported by 33 libraries. The number of libraries was determined as previously described. This segment can be found in the following transcript(s): F13779_T0 (SEQ ID NO:85) and F13779_T16 (SEQ ID NO:86). Table 68 below describes the starting and ending position of this segment on each transcript.

TABLE 68
Segment location on transcripts

| | Segment | Segment |
|---|---|---|
| | starting | ending |
| Transcript name | position | position |
| F13779_T0 (SEQ ID NO: 85) | 376 | 413 |
| F13779_T16 (SEQ ID NO: 86) | 376 | 413 |

Segment cluster F13779_N15 (SEQ ID NO:103) according to the present invention is supported by 33 libraries. The number of libraries was determined as previously described. This segment can be found in the following transcript(s): F13779_T0 (SEQ ID NO:85) and F13779_T16 (SEQ ID NO:86). Table 69 below describes the starting and ending position of this segment on each transcript.

TABLE 69
Segment location on transcripts

| | Segment | Segment |
|---|---|---|
| | starting | ending |
| Transcript name | position | position |
| F13779_T0 (SEQ ID NO: 85) | 931 | 1029 |
| F13779_T16 (SEQ ID NO: 86) | 931 | 1029 |

Segment cluster F13779_N17 (SEQ ID NO:104) according to the present invention is supported by 35 libraries. The number of libraries was determined as previously described. This segment can be found in the following transcript(s): F13779_T0 (SEQ ID NO:85) and F13779_T16 (SEQ ID NO:86). Table 70 below describes the starting and ending position of this segment on each transcript.

TABLE 70
Segment location on transcripts

| | Segment | Segment |
|---|---|---|
| | starting | ending |
| Transcript name | position | position |
| F13779_T0 (SEQ ID NO: 85) | 1030 | 1142 |
| F13779_T16 (SEQ ID NO: 86) | 1030 | 1142 |

Segment cluster F13779_N18 (SEQ ID NO:105) according to the present invention is supported by 1 libraries. The number of libraries was determined as previously described. This segment can be found in the following transcript(s): F13779_T16 (SEQ ID NO:86). Table 71 below describes the starting and ending position of this segment on each transcript.

TABLE 71
Segment location on transcripts

| | Segment | Segment |
|---|---|---|
| | starting | ending |
| Transcript name | position | position |
| F13779_T16 (SEQ ID NO: 86) | 1143 | 1220 |

Segment cluster F13779_N20 (SEQ ID NO:106) according to the present invention is supported by 27 libraries. The number of libraries was determined as previously described. This segment can be found in the following transcript(s): F13779_T0 (SEQ ID NO:85). Table 72 below describes the starting and ending position of this segment on each transcript.

TABLE 72
Segment location on transcripts

| | Segment | Segment |
|---|---|---|
| Transcript name | starting position | ending position |
| F13779_T0 | 1143 | 1202 |
| (SEQ ID NO: 85) | | |

Segment cluster F13779_N22 (SEQ ID NO:107) according to the present invention is supported by 27 libraries. The number of libraries was determined as previously described. This segment can be found in the following transcript(s): F13779_T0 (SEQ ID NO:85). Table 73 below describes the starting and ending position of this segment on each transcript.

TABLE 73
Segment location on transcripts

| | Segment | Segment |
|---|---|---|
| Transcript name | starting position | ending position |
| F13779_T0 | 1203 | 1299 |
| (SEQ ID NO: 85) | | |

Segment cluster F13779_N25 (SEQ ID NO:108) according to the present invention is supported by 25 libraries. The number of libraries was determined as previously described. This segment can be found in the following transcript(s); F13779_T0 (SEQ ID NO:85). Table 74 below describes the starting and ending position of this segment on each transcript.

TABLE 74
Segment location on transcripts

| | Segment | Segment |
|---|---|---|
| Transcript name | starting position | ending position |
| F13779_T0 | 1300 | 1318 |
| (SEQ ID NO: 85) | | |

Segment cluster F13779_N26 (SEQ ID NO:109) according to the present invention is supported by 24 libraries. The number of libraries was determined as previously described. This segment can be found in the following transcript(s): F13779_T0 (SEQ ID NO:85). Table 75 below describes the starting and ending position of this segment on each transcript.

TABLE 75
Segment location on transcripts

| | Segment | Segment |
|---|---|---|
| Transcript name | starting position | ending position |
| F13779_T0 | 1319 | 1335 |
| (SEQ ID NO: 85) | | |

Segment cluster F13779_N27 (SEQ ID NO:110) according to the present invention is supported by 24 libraries. The number of libraries was determined as previously described. This segment can be found in the following transcript(s): F13779_T0 (SEQ ID NO:85). Table 76 below describes the starting and ending position of this segment on each transcript.

TABLE 76
Segment location on transcripts

| | Segment | Segment |
|---|---|---|
| Transcript name | starting position | ending position |
| F13779_T0 | 1336 | 1393 |
| (SEQ ID NO: 85) | | |

Segment cluster F13779_N28 (SEQ ID NO:111) according to the present invention is supported by 19 libraries. The number of libraries was determined as previously described. This segment can be found in the following transcript(s): F13779_T0 (SEQ ID NO:85). Table 77 below describes the starting and ending position of this segment on each transcript.

TABLE 77
Segment location on transcripts

| | Segment | Segment |
|---|---|---|
| Transcript name | starting position | ending position |
| F13779_T0 | 1394 | 1447 |
| (SEQ ID NO: 85) | | |

Segment cluster F13779_N29 (SEQ ID NO:112) according to the present invention is supported by 12 libraries. The number of libraries was determined as previously described. This segment can be found in the following transcript(s): F13779_T0 (SEQ ID NO:85). Table 78 below describes the starting and ending position of this segment on each transcript.

TABLE 78
Segment location on transcripts

| | Segment | Segment |
|---|---|---|
| Transcript name | starting position | ending position |
| F13779_T0 | 1448 | 1471 |
| (SEQ ID NO: 85) | | |

Segment cluster F13779_N30 (SEQ ID NO:113) according to the present invention is supported by 11 libraries. The number of libraries was determined as previously described. This segment can be found in the following transcript(s): F13779_T0 (SEQ ID NO:85). Table 79 below describes the starting and ending position of this segment on each transcript.

TABLE 79
Segment location on transcripts

| | Segment | Segment |
|---|---|---|
| Transcript name | starting position | ending position |
| F13779_T0 | 1472 | 1480 |
| (SEQ ID NO: 85) | | |

Segment cluster F13779_N31 (SEQ ID NO: 114) according to the present invention is supported by 10 libraries. The number of libraries was determined as previously described. This segment can be found in the following transcript(s): F13779 TO (SEQ ID NO:85). Table 80 below describes the starting and ending position of this segment on each transcript.

TABLE 80
Segment location on transcripts

| | Segment | Segment |
|---|---|---|
| Transcript name | starting position | ending position |
| F13779_T0 | 1481 | 1514 |
| (SEQ ID NO: 85) | | |

Segment cluster F13779_N36 (SEQ ID NO:115) according to the present invention is supported by 10 libraries. The number of libraries was determined as previously described. This segment can be found in the following transcript(s): F13779_T0 (SEQ ID NO:85). Table 81 below describes the starting and ending position of this segment on each transcript.

TABLE 81
Segment location on transcripts

| | Segment | Segment |
|---|---|---|
| Transcript name | starting position | ending position |
| F13779_T0 | 3064 | 3172 |
| (SEQ ID NO: 85) | | |

Segment cluster F13779_N37 (SEQ ID NO:116) according to the present invention is supported by 13 libraries. The number of libraries was determined as previously described. This segment can be found in the following transcript(s): F13779_T0 (SEQ ID NO:85). Table 82 below describes the starting and ending position of this segment on each transcript.

TABLE 82
Segment location on transcripts

| | Segment | Segment |
|---|---|---|
| Transcript name | starting position | ending position |
| F13779_T0 | 3173 | 3197 |
| (SEQ ID NO: 85) | | |

Segment cluster F13779_N38 (SEQ ID NO:117) according to the present invention is supported by 25 libraries. The number of libraries was determined as previously described. This segment can be found in the following transcript(s): F13779_T0 (SEQ ID NO:85). Table 83 below describes the starting and ending position of this segment on each transcript.

TABLE 83
Segment location on transcripts

| | Segment | Segment |
|---|---|---|
| Transcript name | starting position | ending position |
| F13779_T0 | 3198 | 3265 |
| (SEQ ID NO: 85) | | |

Segment cluster F13779_N40 (SEQ ID NO:118) according to the present invention is supported by 23 libraries. The number of libraries was determined as previously described. This segment can be found in the following transcript(s): F13779_T0 (SEQ ID NO:85). Table 84 below describes the starting and ending position of this segment on each transcript.

TABLE 84
Segment location on transcripts

| | Segment | Segment |
|---|---|---|
| Transcript name | starting position | ending position |
| F13779_T0 | 3403 | 3414 |
| (SEQ ID NO: 85) | | |

Segment cluster F13779_N42 (SEQ ID NO:119) according to the present invention is supported by 22 libraries. The number of libraries was determined as previously described. This segment can be found in the following transcript(s): F13779_T0 (SEQ ID NO:85). Table 85 below describes the starting and ending position of this segment on each transcript.

TABLE 85
Segment location on transcripts

| | Segment | Segment |
|---|---|---|
| Transcript name | starting position | ending position |
| F13779_T0 | 3537 | 3541 |
| (SEQ ID NO: 85) | | |

Segment cluster F13779_N46 (SEQ ID NO:120) according to the present invention is supported by 7 libraries. The number of libraries was determined as previously described. This segment can be found in the following transcript(s): F13779_T0 (SEQ ID NO:85). Table 86 below describes the starting and ending position of this segment on each transcript.

TABLE 86
Segment location on transcripts

| | Segment | Segment |
|---|---|---|
| Transcript name | starting position | ending position |
| F13779_T0 | 6252 | 6263 |
| (SEQ ID NO: 85) | | |

Microarray (chip) data is also available for this segment as follows. As described above with regard to the cluster itself, various oligonucleotides were tested for being differentially expressed in various disease conditions, particularly cancer The following oligonucleotides were found to hit segment F13779-seg 6 (SEQ ID NO: 101), shown in Table 87.

TABLE 87
Oligonucleotides related to this segment

| | Overexpressed in | Chip |
|---|---|---|
| Oligonucleotide name | cancers | reference |
| F13779_0_45_3 | Breast cancer | BRS |
| (SEQ ID NO:84) | | |
| F13779_0_45_3 | Colon cancer | COL |
| (SEQ ID NO:84) | | |
| F13779_0_45_3 | Lung cancer | LUN |
| (SEQ ID NO:84) | | |

>F13779_0_45_3 (SEQ ID NO:84) TCATGCCCAAGTTCAAACAACGAAGACGAAAGCTAAAAGCCAAAGCCGAA

Variant protein alignment to the previously known protein:

### Alignment of: F13779_P0 (SEQ ID NO:121)×Q9NUZ7_HUMAN (SEQ ID NO:123):

Total length: 360
Matching length: 360

| | . . . . . | |
|---|---|---|
| 1 | MPKFKQRRRKLKAKAERLFKKKEASHFQSKLITPPPPPPSPERVGISSID | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MPKFKQRRRKLKAKAERLFKKKEASHFQSKLITPPPPPPSPERVGISSID | 50 |
| | . . . . . | |
| 51 | ISQSRSWLTSSWNFNFPNIRDAIKLWTNRVWSIYSWCQNCITQSLEVLKD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ISQSRSWLTSSWNFNFPNIRDAIKLWTNRVWSIYSWCQNCITQSLEVLKD | 100 |
| | . . . . . | |
| 101 | TIFPSRICHRELYSVKQQFCILESKLCKLQEALKTISESSSCPSCGQTCH | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | TIFPSRICHRELYSVKQQFCILESKLCKLQEALKTISESSSCPSCGQTCH | 150 |
| | . . . . . | |
| 151 | MSGKLTNVPACVLITPGDSKAVLPPTLPQPASHFPPPPPPPPLPPPPPPL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | MSGKLTNVPACVLITPGDSKAVLPPTLPQPASHFPPPPPPPPLPPPPPPL | 200 |
| | . . . . . | |
| 201 | APVLLRKPSLAKALQAGPLKKDGPMQITVKDLLTVKIKKTQSLDEKRKLI | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | APVLLRKPSLAKALQAGPLKKDGPMQITVKDLLTVKIKKTQSLDEKRKLI | 250 |
| | . . . . . | |
| 251 | PSPKARNPLVTVSDLQHVTLKPNSKVLSTRVTNVLITPGKSQMDLRKLLR | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | PSPKARNPLVTVSDLQHVTLKYNSKVLSTRVTNYLITPGKSQMDLRLLLR | 300 |
| | . . . . . | |
| 301 | KVDVERSPGGTPLTNKENMETGTGLTPVMTQALRRKFQLARPRSPTPTLP | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | KVDVERRPGGTPLTNKENMETGTGLTPVMTQALRRKFQLARPRSPTPTLP | 350 |
| | . | |
| 351 | LSTSSFDEQN | 360 |
| | \|\|\|\|\|\|\|\|\|\| | |
| 351 | LSTSSFDEQN | 360 |

Alignment of. F13779_P0 (SEQ ID NO:121)×Q96HE9_HUMAN (SEQ ID NO:135):
Total length: 360
Matching length: 360

| | . . . . . | |
|---|---|---|
| 1 | MPKFKQRRRKLKAKAERLFKKKEASHFQSKLITPPPPPPSPERVGISSD | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MPKFKQRRRKLKAKAERLFKKKEASHFQSKLITPPPPPPSPERVGISSD | 50 |
| | . . . . . | |
| 51 | ISQSRSWLTSSWNFNFPNIRDAIKLWTNRVWSIYSWCQNCITQSLEVLKD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ISQSRSWLTSSWNFNFPNIRDATKIWTNRVWSIYSWCQNCITQSLEVLKD | 100 |
| | . . . . . | |
| 101 | TIFPSRICHRELYSVKQQFCILESKLCKLQEALKTISESSSCPSCGQTCH | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | TTFPSRICHRELYSVKQQFCILESKLCKLQEALKTISESSSCPSCGQTCH | 150 |
| | . . . . . | |
| 151 | MSGKLTNVPACVLITPGDSKAVLPPTLPQPASHFPPPPPPPPLPPPPPPL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | MSGKLTNVPACVLITPGDSKAVLPPTLPQPASHFPPPPPPPPLPPPPPPL | 200 |
| | . . . . . | |
| 201 | APVLLRKPSLAKALQAGPLKKDGPMQITVKDLLTVKLKKTQSLDEKRKLI | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | APVLLRKPSLAKALQAGPLKKDGPMQITVKDLLTVKLKKTQSLDEKRKLI | 250 |
| | . . . . . | |
| 251 | PSPKARNPLVTVSDLQHVTLKPNSKVLSTRVTNVLITPGKSQMDLRKLLR | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | PSPKARNPLVTVSDLQHVTLKPNSKVLSTRVTNVLITPGKSQMDLRKLLR | 300 |
| | . . . . . | |
| 301 | KVDVERSPGGTPLTNKENMETGTGLTPVMTQALRRKFQLAHPRSPTPTLP | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | KVDVERRPGGTPLTNKENMETGTGLTPVMTQALRRKFQLAHPRSPTPTLP | 350 |
| | . | |
| 351 | LSTSSFDEQN | 360 |
| | \|\|\|\|\|\|\|\|\|\| | |
| 351 | LSTSSFDEQN | 360 |

### Alignment of: F13779_P0 (SEQ ID NO:121)×NP_{—}060774 (SEQ ID NO:123):

Total length: 360
Matching length: 360

| | . . . . . | |
|---|---|---|
| 1 | MPKFKQRRRKLKAKAERLFKKKEASHFQSKLITPPPPPPSPERVGISSD | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MPKFKQRRRKLKAKAERLFKKKEASHFQSKLITPPPPPPSPERVGISSD | 50 |
| | . . . . . | |
| 51 | ISQSRSWLTSSWNFNFPNIRDAIKLWTNRVWSIYSWCQNCITQSLEVLKD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ISQSRSWLTSSWNFNFPNIRDATKIWTNRVWSIYSWCQNCITQSLEVLKD | 100 |
| | . . . . . | |
| 101 | TIFPSRICHRELYSVKQQFCILESKLCKLQEALKTISESSSCPSCGQTCH | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | TTFPSRICHRELYSVKQQFCILESKLCKLQEALKTISESSSCPSCGQTCH | 150 |
| | . . . . . | |
| 151 | MSGKLTNVPACVLITPGDSKAVLPPTLPQPASHFPPPPPPPPLPPPPPPL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | MSGKLTNVPACVLITPGDSKAVLPPTLPQPASHFPPPPPPPPLPPPPPPL | 200 |
| | . . . . . | |
| 201 | APVLLRKPSLAKALQAGPLKKDGPMQITVKDLLTVKLKKTQSLDEKRKLI | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | APVLLRKPSLAKALQAGPLKKDGPMQITVKDLLTVKLKKTQSLDEKRKLI | 250 |
| | . . . . . | |
| 251 | PSPKARNPLVTVSDLQHVTLKPNSKVLSTRVTNVLITPGKSQMDLRKLLR | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 251 | PSPKARNPLVTVSDLQHVTLKPNSKVLSTRVTNVLITPGKSQMDLRKLLR | 300 |
| | . . . . . | |
| 301 | KVDVERSPGGTPLTNKENMETGTGLTPVMTQALRRKFQLAHPRSPTPTLP | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 301 | KVDVERRPGGTPLTNKENMETGTGLTPVMTQALRRKFQLAHPRSPTPTLP | 350 |
| | . | |
| 351 | LSTSSFDEQN | 360 |
| | \|\|\|\|\|\|\|\|\|\| | |
| 351 | LSTSSFDEQN | 360 |

Alignment of. F13779_P0 (SEQ ID NO:121)×Q9NXE9_HUMAN (SEQ ID NO:122):
Total length: 360
Matching length: 210

| | . . . . . | |
|---|---|---|
| 1 | MPKFKQRRRKLKAKAERLFKKKEASHFQSKLITPPPPPPSPERVGISSID | 50 |
| | .................................................. | |
| | . . . . . | |
| 51 | ISQSRSWLTSSWNFNFPNIRDAIKLWTNRVWSIYSWCQNCITQSLEVLKD | 100 |
| | .................................................. | |
| | . . . . . | |
| 101 | TIFPSRICHRELYSVKQQFCILESKLCKLQEALKTISESSSCPSCGQTCH | 150 |
| | .................................................. | |
| | . . . . . | |
| 151 | MSGKLTNVPACVLITPGDSKAVLPPTLPQPASHFPPPPPPPPLPPPPPPL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MSGKLTNVPACVLITPGDSKAVLPPTLPQPASHFPPPPPPPPLPPPPPPL | 50 |
| | . . . . . | |
| 201 | APVLLRKPSLAKALQAGPLKKDGPMQITVKDLLTVKLKKTQSLDEKRKLI | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | APVLLRKPSLAKALQAGPLKKDGPMQITVKDLLTVKLKKTQSLDEKRKLI | 100 |
| | . . . . . | |
| 251 | PSPKARNPLVTVSDLQHVTLKPNSKVLSTRVTNVLITPGKSQMDLRKLLR | 300 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | PSPKAkNPLVTVSDLQHVTLKPNSKVLSTRVTNVLITPGKSQMDLRKLLR | 150 |
| | . . . . . | |
| 301 | KVDVERSPGGTPLTNKENMETGTGLTPVMTQALRRKFQLAHPRSPTPTLP | 350 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | KVDVERSPGGTPLTNKENMETGTGLTPVMTQALRRKFQLAHPRSPTPTLP | 200 |
| | . | |
| 351 | LSTSSFDEQN | 360 |
| | \|\|\|\|\|\|\|\|\|\| | |
| 201 | LSTSSFDEQN | 210 |

### Alignment of: F13779_P5 (SEQ ID NO: 124)×Q9NUZ7_HUMAN (SEQ ID NO: 123):

Total length: 361
Matching length: 285

| | . . . . . | |
|---|---|---|
| 1 | MPKFKQRRRKLKAKAERLFKKKEASHFQSKLLTPPPPPPSPERVGISSD | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MPKFKQRRRLLKAKAERLFKKKEASHFQSKLLTPPPPPPSPERVGISSD | 50 |
| | . . . . . | |
| 51 | ISQSRSWLTSSWNENFPNIRDAIKLWTNRVWSLYSWCQNCITQSLEVLKD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ISQSRSWLTSSWNFNFPNIRDAIKLWTNRVWSIYSWCQNCITQSLEVLKD | 100 |
| | . . . . . | |
| 101 | TIFPSRICHRELYSVKQQFCILESKLCKLQEALKTLSESSSCPSCGQTCH | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | TIFPSRICHRELYSVKQQFCILESKLCKLQEALKTISESSSCPSCGQTCH | 150 |
| | . . . . . | |
| 151 | MSGKLTNVPACVLLTPGDSKAVLPPTLPQPASHFPPPPPPPPLPPPPPPL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | MSGKLTNVPACVLITPGDSKAVLPPTLPQPASHFPPPPPPPPLPPPPPPL | 200 |
| | . . . . . | |
| 201 | APVLLRKPSLAKALQAGPLKKDGPMQITVKDLLTVKLKKTQSLDEKRKLI | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | APVLLRKPSLAKALQAGPLKKDGPMQITVKDLLTVKLKKTQSLDEKRKLI | 250 |
| | . . . . . | |
| 251 | PSPKARNPLVTVSDLQHVTLKPNSKVLSTRVTNVLM.............. | 286 |
| | \|\|\|\|\|\|\|\|\|\|\|\|\|\|\|\|\|\|\|\|\|\|\|\|\|\|\|\|\|\|\|\|\|\|\| | |
| 251 | PSPKARNPLVTVSDLQHVTLKPNSKVLSTRVTNVL.ITPGKSQMDLRKLL | 299 |
| | . . . . . | |
| | .................................................. | |
| 300 | RKVDVERRPGGTPLTNKENMETGTGLTPVMTQALRRKFQLAHPRSPTPTL | 349 |
| | . | |
| | ........... | |
| 350 | PLSTSSFDEQN | 360 |

### Alignment of: F13779_P5 (SEQ ID NO:124)×Q96HE9_HUMAN (SEQ ID NO:135):

Total length. 361
Matching length: 285

| | . . . . . | |
|---|---|---|
| 1 | MPKFKQRRRKLKAKAERLFKKKEASHFQSKLITPPPPPPSPERVGISSID | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MPKFKQRRRKLKAKAERLFKKKEASHFQSKLITPPPPPPSPERVGISSID | 50 |
| | . . . . . | |
| 51 | ISQSRSWLTSSWNFNFPNIRDAIKLWTNRVWSIYSWCQNCITQSLEVLKD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ISQSRSWLTSSWNFNFPNIRDAIKLWTNRVWSIYSWCQNCITQSLEVLKD | 100 |
| | . . . . . | |
| 101 | TIFPSRICHRELYSVKQQFCILESKLCKLQEALKTISESSSCPSCGQTCH | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | TIFPSRICHRELYSVKQQFCILESKLCKLQEALKTISESSSCPSCGQTCH | 150 |
| | . . . . . | |
| 151 | MSGKLTNVPACVLITPGDSKAVLPPTLPQPASHFPPPPPPPPLPPPPPPL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | MSGKLTNVPACVLITPGDSKAVLPPTLPQPASHFPPPPPPPPLPPPPPPL | 200 |
| | . . . . . | |
| 201 | APVLLRKPSLAKALQAGPLKKDGPMQITVKDLLTVKLKKTQSLDEKRKLI | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | APVLLRKPSLAKALQAGPLKKDGPMQITVKDLLTVKLKKTQSLDEKRKLI | 250 |
| | . . . . . | |
| 251 | PSPKARNPLVTVSDLQHVTLKPNSKVLSTRVTNVLM.............. | 286 |
| | \|\|\|\|\|\|\|\|\|\|\|\|\|\|\|\|\|\|\|\|\|\|\|\|\|\|\|\|\|\|\|\|\|\|\| | |
| 251 | PSPKARNPLVTVSDLQHVTLKPNSKVLSTRVTNVL.ITPGKSQMDLRKLL | 299 |
| | . . . . . | |
| | ................................................. | |
| 300 | RKVDVERSPGGTPLTNKENMETGTGLTPVMTQALRRKFQLAHPRSPTPTL | 349 |
| | . | |
| | ........... | |
| 350 | PLSTSSFDEQN | 360 |

### Alignment of: F13779_P5 (SEQ ID NO:124)×NP_{—}060774 (SEQ II) NO:123):

Total length: 361
Matching length: 285

| | . . . . . | |
|---|---|---|
| 1 | MPKFKQRRRKLKAKAERLFKKKEASHFQSKLITPPPPPPSPERVGISSID | 50 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 1 | MPKFKQRRRKLKAKAERLFKKKEASHFQSKLITPPPPPPSPERVGISSID | 50 |
| | . . . . . | |
| 51 | ISQSRSWLTSSWNFNFPNIRDAIKLWTNRVWSIYSWCQNCITQSLEVLKD | 100 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 51 | ISQSRSWLTSSWNFNFPNIRDAIKLWTNRVWSIYSWCQNCITQSLEVLKD | 100 |
| | . . . . . | |
| 101 | TIFPSRICHRELYSVKQQFCILESKLCKLQEALKTISESSSCPSCGQTCH | 150 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 101 | TIFPSRICHRELYSVKQQFCILESKLCKLQEALKTISESSSCPSCGQTCH | 150 |
| | . . . . . | |
| 151 | MSGKLTNVPACVLITPGDSKAVLPPTLPQPASHFPPPPPPPPLPPPPPPL | 200 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 151 | MSGKLTNVPACVLITPGDSKAVLPPTLPQPASHFPPPPPPPPLPPPPPPL | 200 |
| | . . . . . | |
| 201 | APVLLRKPSLAKALQAGPLKKDGPMQITVKDLLTVKLKKTQSLDEKRKLI | 250 |
| | \|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\|\| | |
| 201 | APVLLRKPSLAKALQAGPLKKDGPMQITVKDLLTVKLKKTQSLDEKRKLI | 250 |
| | . . . . . | |
| 251 | PSPKARNPLVTVSDLQHVTLKPNSKVLSTRVTNVLM.............. | 286 |
| | \|\|\|\|\|\|\|\|\|\|\|\|\|\|\|\|\|\|\|\|\|\|\|\|\|\|\|\|\|\|\|\|\|\|\| | |
| 251 | PSPKARNPLVTVSDLQHVTLKPNSKVLSTRVTNVL.ITPGKSQMDLRKLL | 299 |
| | . . . . . | |
| | ................................................. | |
| 300 | RKVDVERSPGGTPLTNKENMETGTGLTPVMTQALRRKFQLAHPRSPTPTL | 349 |
| | . | |
| | ........... | |
| 350 | PLSTSSFDEQN | 360 |

Expression of Homo sapiens hypothetical protein FLJ11029 F13779 transcripts which are detectable by amplicon as depicted in sequence name F13779_seg17-18 (SEQ ID NO: 127) in normal and cancerous Breast tissues

Expression of Homo sapiens hypothetical protein FLJ11029 transcripts detectable by or according to seg17-18—F13779_seg17-18 (SEQ ID NO: 127) amplicon and primers F13779_seg17-18F (SEQ ID NO: 125) and F13779_seg17-18R (SEQ ID NO: 126) was measured by real time PCR. In parallel the expression of several housekeeping genes—G6PD (GenBank Accession No. NM_{—}000402 (SEQ ID NO:17); G6PD amplicon (SEQ ID NO: 20)), RPL19 (GenBank Accession No. NM_{—}000981 (SEQ ID NO:29); RPL19 amplicon (SEQ ID NO: 32)), PBGD (GenBank Accession No. BC019323 (SEQ ID NO:5); amplicon—PBGD-amplicon (SEQ ID NO:8)) and SDHA (GenBank Accession No. NM_{—}004168 (SEQ ID NO:1); amplicon —SDHA-amplicon (SEQ ID NO: 4)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the normal samples (sample numbers 43, 46, 47, 48, 49, 51, 52, 54, 56, 58, 59, 60, 61, 62, 63, 64, 65 and 67, Table 1_{—}3 above), to obtain a value of fold up-regulation for each sample relative to median of the normal samples.

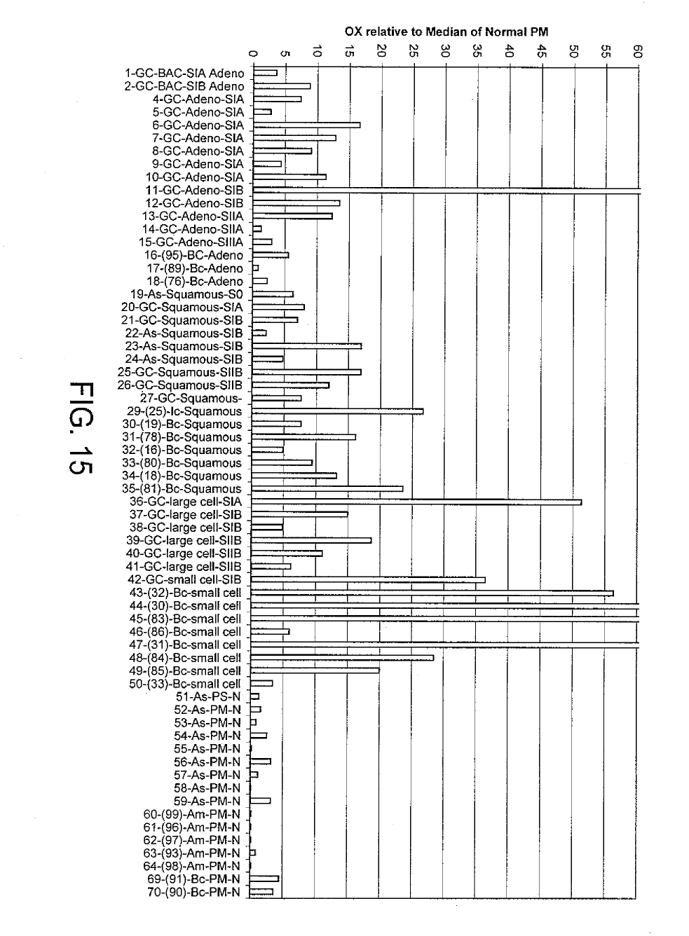

FIG. 14 is a histogram showing over expression of the above-indicated Homo sapiens hypothetical protein FLJ11029 transcripts in cancerous Breast samples relative to the normal samples.

As is evident from FIG. 14, the expression of Homo sapiens hypothetical protein FLJ11029 transcripts detectable by the above amplicon in cancer samples was significantly higher than in the non-cancerous samples (sample numbers 43, 46, 47, 48, 49, 51, 52, 54, 56, 58, 59, 60, 61, 62, 63, 64, 65 and 67, Table 1_{—}3 above). Notably an over-expression of at least 5 fold was found in 29 out of 53 adenocarcinoma samples.

Statistical analysis was applied to verify the significance of these results, as described below.

The P value for the difference in the expression levels of Homo sapiens hypothetical protein FLJ11029 transcripts detectable by the above amplicon in Breast cancer samples versus the normal tissue samples was determined by T test as 7.02e-04.

Threshold of 5 fold over expression was found to differentiate between cancer and normal samples with P value of 1.43e-005 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: F13779_seg17-18F (SEQ ID NO: 125) forward primer; and F13779_seg17-18R (SEQ ID NO: 126) reverse primer.

The present invention also preferably encompasses any amplicon obtained through the use of any suitable primer pair; for example, for the above experiment, the following amplicon was obtained as a non-limiting illustrative example only of a suitable amplicon: F13779_seg17-18 (SEQ ID NO: 127).

Forward Primer (F13779_seg17-18F (SEQ ID NO:125)):
>F13779seg_17-18F (SEQ ID NO:125)
CGTCTCTGACTTGCAGCATGTTA
Reverse Primer (F13779_seg17-18R (SEQ ID NO:126)):
>F13779_seg17-18R (SEQ ID NO:126)
CAACAATATCATTCTTTTAAGTAGTAGTAGAGCATA
Amplicon (F13779_seg17-18 (SEQ ID NO:127)):
>F13779seg_17-18 (SEQ ID NO:127)
CGTCTCTGACTTGCAGCATGTTACCCTGAAACCTAACTCCAAAGTGTTAT
CGACTCGAGTTACAAACGTCTTAATGTAAGGAACTGCACAGTACTATGCT
CTACTACTACTTAAAAGAATGATATTGTTG

Expression of Homo sapiens hypothetical protein FLJ11029 F13779 transcripts which are detectable by amplicon as depicted in sequence name F13779_seg17-18 (SEQ ID NO: 127) in normal and cancerous Lung tissues

Expression of Homo sapiens hypothetical protein FLJ11029 transcripts detectable by or according to seg17-18—F13779_seg17-18 (SEQ ID NO: 127) amplicon and primers F13779_seg17-18F (SEQ ID NO: 125) and F13779_seg17-18R (SEQ ID NO: 126) was measured by real time PCR. In parallel the expression of several housekeeping genes —HPRT1 (GenBank Accession No. NM_{—}000194 (SEQ ID NO:9); amplicon—HPRT1-amplicon (SEQ ID NO: 12)), PBGD (GenBank Accession No. BC019323 (SEQ ID NO:5); amplicon—PBGD-amplicon (SEQ ID NO:8)), SDHA (GenBank Accession No. NM_{—}004168 (SEQ ID NO:1); amplicon—SDHA-amplicon (SEQ ID NO: 4)) and Ubiquitin (GenBank Accession No. BC000449 (SEQ ID NO:25); amplicon—Ubiquitin-amplicon (SEQ ID NO: 28)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the normal samples (sample numbers 51, 52, 53, 54, 55, 56, 57, 58, 59, 60, 61, 62, 63, 64, 69 and 70, Table 1_{—}2 above), to obtain a value of fold up-regulation for each sample relative to median of the normal samples.

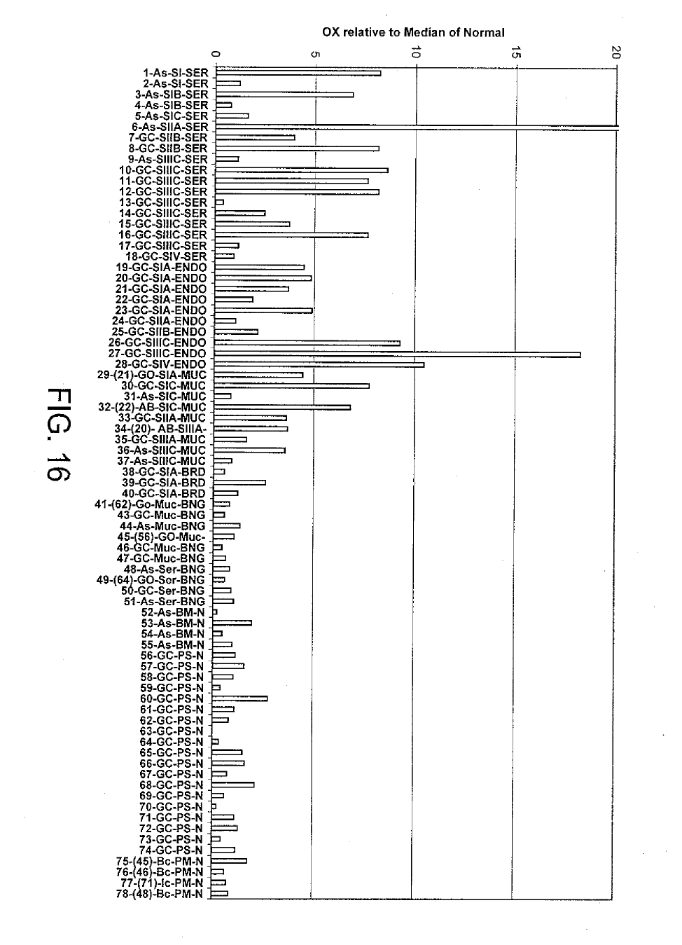

FIG. 15 is a histogram showing over expression of the above-indicated Homo sapiens hypothetical protein FLJ11029 transcripts in cancerous Lung samples relative to the normal samples.

As is evident from FIG. 15, the expression of Homo sapiens hypothetical protein FLJ11029 transcripts detectable by the above amplicon in adenocarcinoma, squamous cell carcinoma, large cell carcinoma, small cell carcinoma and non-small cell carcinoma samples was significantly higher than in the non-cancerous samples (sample numbers 51, 52, 53, 54, 55, 56, 57, 58, 59, 60, 61, 62, 63, 64, 69 and 70, Table 1_{—}2 above). Notably an over-expression of at least 5 fold was found in 28 out of 39 non-small cell carcinoma samples, specifically in 10 out of 17 adenocarcinoma samples, in 13 out of 16 squamous cell carcinoma samples, in 5 out of 6 large cell carcinoma samples. Also over-expression was observed in 8 out of 9 small cell carcinoma samples.

Statistical analysis was applied to verify the significance of these results, as described below.

The P value for the difference in the expression levels of Homo sapiens hypothetical protein FLJ11029 transcripts detectable by the above amplicon in Lung squamous cell carcinoma samples versus the normal tissue samples was determined by T test as 3.72e-005. The P value for the difference in the expression levels of Homo sapiens hypothetical protein FLJ11029 transcripts detectable by the above amplicon in Lung small cell carcinoma samples versus the normal tissue samples was determined by T test as 8.06c-003. The P value for the difference in the expression levels of Homo sapiens hypothetical protein FLJ11029 transcripts detectable by the above amplicon in Lung non-small cell carcinoma samples versus the normal tissue samples was determined by T test as 1.55e-004.

Threshold of 5 fold over expression was found to differentiate between adenocarcinoma and normal samples with P value of 2.10e-004 as checked by exact Fisher test. Threshold of 5 fold over expression was found to differentiate between squamous cell carcinoma and normal samples with P value of 1.61e-006 as checked by exact Fisher test. Threshold of 5 fold over expression was found to differentiate between large cell carcinoma and normal samples with P value of 2.28e-004 as checked by exact Fisher test. Threshold of 5 fold over expression was found to differentiate between small cell carcinoma and normal samples with P value of 8.32e-006 as checked by exact Fisher test. Threshold of 5 fold over expression was found to differentiate between non-small cell carcinoma and normal samples with P value of 4.38e-007 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: F13779_seg17-18F (SEQ ID NO: 125) forward primer; and F13779_seg17-18R (SEQ ID NO: 126) reverse primer.

The present invention also preferably encompasses any amplicon obtained through the use of any suitable primer pair; for example, for the above experiment, the following amplicon was obtained as a non-limiting illustrative example only of a suitable amplicon: F13779_seg17-18 (SEQ ID NO: 127).

Forward Primer (F13779_seg17-18F (SEQ ID NO:125)):
>F13779seg_17-18F (SEQ ID NO:125)
CGTCTCTGACTTGCAGCATGTTA
Reverse Primer (F13779_seg17-18R (SEQ ID NO:126)):
>F13779_seg17-18R (SEQ ID NO:126)
CAACAATATCATTCTTTTAAGTAGTAGTAGAGCATA
Amplicon (F13779_seg17-18 (SEQ ID NO:127)):
>F13779seg_17-18 (SEQ ID NO:127)
CGTCTCTGACTTGCAGCATGTTACCCTGAAACCTAACTCCAAAGTGTTAT
CGACTCGAGTTACAAACGTCTTAATGTAAGGAACTGCACAGTACTATGCT
CTACTACTACTTAAAAGAATGATATTGTTG

Expression of sapiens hypothetical protein FLJ11029 F13779 transcripts which are detectable by amplicon as depicted in sequence name F13779_seg17-18 (SEQ ID NO: 127) in normal and cancerous Ovary tissues

Expression of sapiens hypothetical protein FLJ11029 transcripts detectable by or according to seg17-18—F13779_seg17-18 (SEQ ID NO: 127) amplicon and primers F13779_seg17-18F (SEQ ID NO: 125) and F13779_seg17-18R (SEQ ID NO: 126) was measured by real time PCR. In parallel the expression of several housekeeping genes —SDHA (GenBank Accession No. NM_{—}004168 (SEQ ID NO:1); amplicon—SDHA-amplicon (SEQ ID NO: 4)), HPRT1 (GenBank Accession No. NM_{—}000194 (SEQ ID NO:9); amplicon—HPRT1-amplicon (SEQ ID NO: 12)) and G6PD (GenBank Accession No. NM_{—}000402 (SEQ ID NO:17); G6PD amplicon (SEQ ID NO: 20)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the normal samples (sample numbers 52, 53, 54, 55, 56, 57, 58, 59, 60, 61, 62, 64, 65 and 66, Table 1-1 above), to obtain a value of fold up-regulation for each sample relative to median of the normal samples.

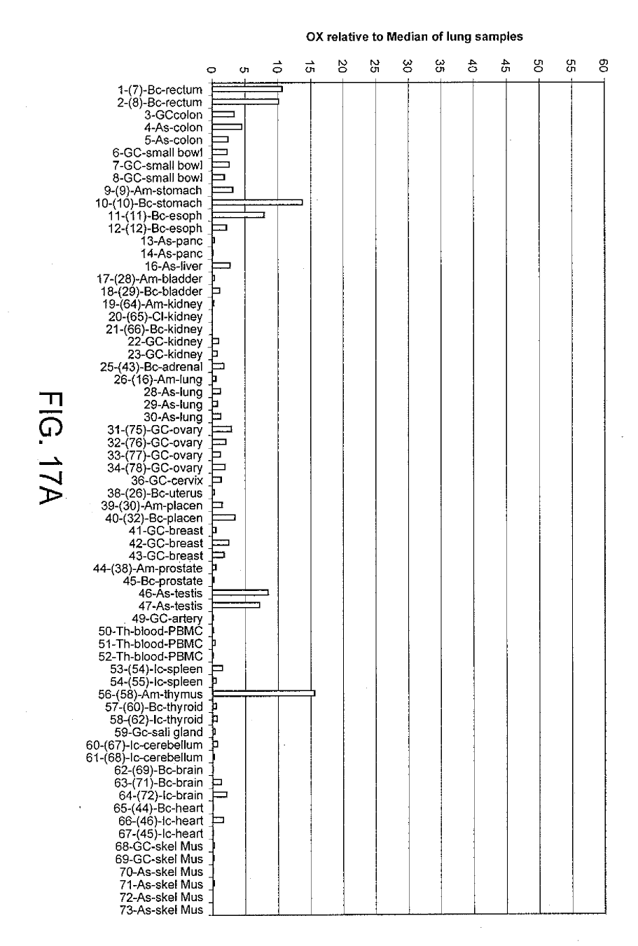

FIG. 16 is a histogram showing over expression of the above-indicated sapiens hypothetical protein FLJ11029 transcripts in cancerous Ovary samples relative to the normal samples.

As is evident from FIG. 16, the expression of sapiens hypothetical protein FLJ11029 transcripts detectable by the above amplicon in serous carcinoma and adenocarcinoma samples was significantly higher than in the non-cancerous samples (sample numbers 52, 53, 54, 55, 56, 57, 58, 59, 60, 61, 62, 64, 65 and 66, Table 1_{—}1 above) and was higher in a few mucinous carcinoma samples than in the non-cancerous samples. Notably an over-expression of at least 5 fold was found in 8 out of 18 serous carcinoma samples, in 2 out of 9 mucinous carcinoma samples and in 3 out of 10 adenocarcinoma samples.

Statistical analysis was applied to verify the significance of these results, as described below.

The P value for the difference in the expression levels of sapiens hypothetical protein FLJ11029 transcripts detectable by the above amplicon in Ovary serous carcinoma samples versus the normal tissue samples was determined by T test as 1.65e-003. The P value for the difference in the expression levels of sapiens hypothetical protein FLJ11029 transcripts detectable by the above amplicon in Ovary mucinous carcinoma samples versus the normal tissue samples was determined by T test as 5.58e-003. The P value for the difference in the expression levels of sapiens hypothetical protein FLJ11029 transcripts detectable by the above amplicon in Ovary adenocarcinoma samples versus the normal tissue samples was determined by T test as 6.95e-003. The P value for the difference in the expression levels of sapiens hypothetical protein FLJ11029 transcripts detectable by the above amplicon in Ovary endometriod samples versus the normal tissue samples was determined by T test as 3.41e-006.

Threshold of 5 fold over expression was found to differentiate between serous carcinoma and normal samples with P value of 2.47e-004 as checked by exact Fisher test. Threshold of 5 fold over expression was found to differentiate between adenocarcinoma and normal samples with P value of 1.68e-002 as checked by exact Fisher test. Threshold of 5 fold over expression was found to differentiate between endometriod and normal samples with P value of 3.40e-004 as checked by exact Fisher test.

The above values demonstrate statistical significance of the results.

Primer pairs are also optionally and preferably encompassed within the present invention; for example, for the above experiment, the following primer pair was used as a non-limiting illustrative example only of a suitable primer pair: F13779_seg17-18F (SEQ ID NO: 125) forward primer; and F13779_seg17-1SR (SEQ ID NO: 126) reverse primer.

The present invention also preferably encompasses any amplicon obtained through the use of any suitable primer pair; for example, for the above experiment, the following amplicon was obtained as a non-limiting illustrative example only of a suitable amplicon: F13779_seg17-18 (SEQ ID NO: 127).

Forward Primer (F13779_seg17-18F (SEQ ID NO:125)):
>F13779seg_17-18F (SEQ ID NO:125)
CGTCTCTGACTTGCAGCATGTTA
Reverse Primer (F13779_seg17-18R (SEQ ID NO:126)):
>F13779_seg17-18R (SEQ ID NO:126)
CAACAATATCATTCTTTTAAGTAGTAGTAGAGCATA
Amplicon (F13779_seg17-18 (SEQ ID NO:127)):
>F13779seg_17-18 (SEQ ID NO:127)
CGTCTCTGACTTGCAGCATGTTACCCTGAAACCTAACTCCAAAGTGTTAT
CGACTCGAGTTACAAACGTCTTAATGTAAGGAACTGCACAGTACTATGCT
CTACTACTACTTAAAAGAATGATATTGTTG

Expression of Homo sapiens hypothetical protein FLJ11029 F13779 transcripts which are detectable by amplicon as depicted in sequence name F13779_seg17-18 (SEQ ID NO: 127) in different normal tissues

Expression of Homo sapiens hypothetical protein FLJ11029 transcripts detectable by or according to seg17-18—F13779_seg17-18 (SEQ ID NO: 127) amplicon and primers F13779_seg17-18F (SEQ II) NO: 125) and F13779_seg17-18R (SEQ ID NO: 126) was measured by real time PCR. In parallel the expression of several housekeeping genes —SDHA (GenBank Accession No. NM_{—}004168 (SEQ ID) NO:1); amplicon—SDHA-amplicon (SEQ ID NO: 4)), Ubiquitin (GenBank Accession No. BC000449 (SEQ ID NO:25); amplicon—Ubiquitin-amplicon (SEQ ID NO: 28)), RPL19 (GenBank Accession No. NM_{—}000981 (SEQ ID NO:29); RPL19 amplicon (SEQ ID NO: 32)) and TATA box (GenBank Accession No. NM_{—}003194 (SEQ ID NO:33); TATA amplicon (SEQ ID NO: 36)) was measured similarly. For each RT sample, the expression of the above amplicon was normalized to the normalization factor calculated from the expression of these house keeping genes as described in normalization method in the “materials and methods” section. The normalized quantity of each RT sample was then divided by the median of the quantities of the lung samples (sample numbers 26, 28, 29 and 30, Table 1_{—}4 above), to obtain a value of relative expression of each sample relative to median of the lung samples as presented in figure A, by the median of the quantities of the breast samples (sample numbers 41, 42, and 43, Table 1_{—}4 above), to obtain a value of relative expression of each sample relative to median of the breast samples as presented in figure B, by the median of the quantities of the ovary samples (sample numbers 31, 32, 33 and 34, Table 1_{—}4 above), to obtain a value of relative expression of each sample relative to median of the ovary samples as presented in figure C.

Forward Primer (F13779_seg17-18F (SEQ ID NO:125)):
>F13779seg_17-18F (SEQ ID NO:125)
CGTCTCTGACTTGCAGCATGTTA
Reverse Primer (F13779_seg17-18R (SEQ ID NO:126)):
>F13779_seg17-18R (SEQ ID NO:126)
CAACAATATCATTCTTTTAAGTAGTAGTAGAGCATA
Amplicon (F13779_seg17-18 (SEQ ID NO:127)):
>F13779seg_17-18 (SEQ ID NO:127)
CGTCTCTGACTTGCAGCATGTTACCCTGAAACCTAACTCCAAAGTGTTAT
CGACTCGAGTTACAAACGTCTTAATGTAAGGAACTGCACAGTACTATGCT
CTACTACTACTTAAAAGAATGATATTGTTG

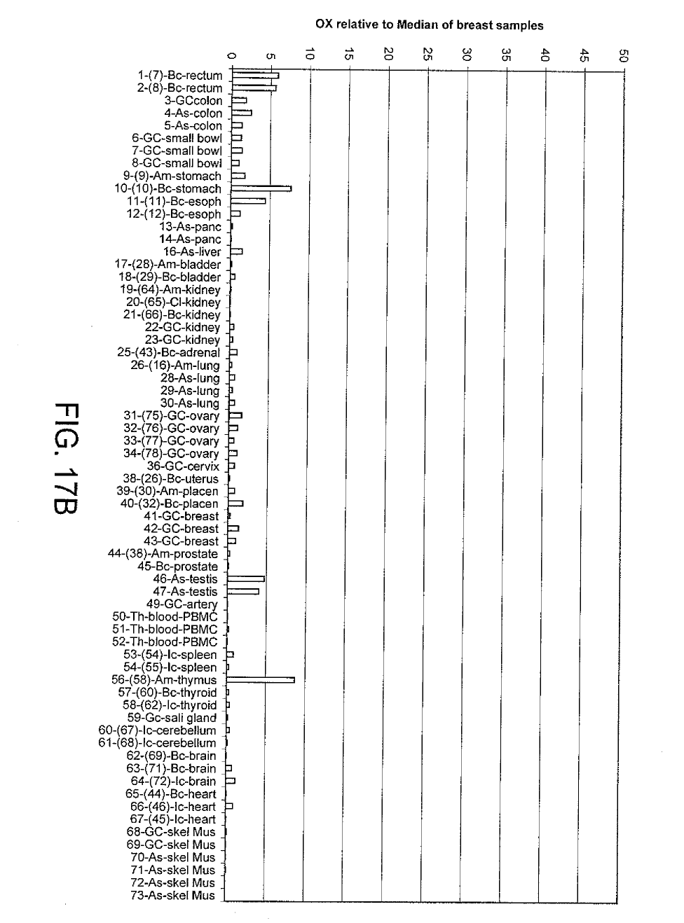

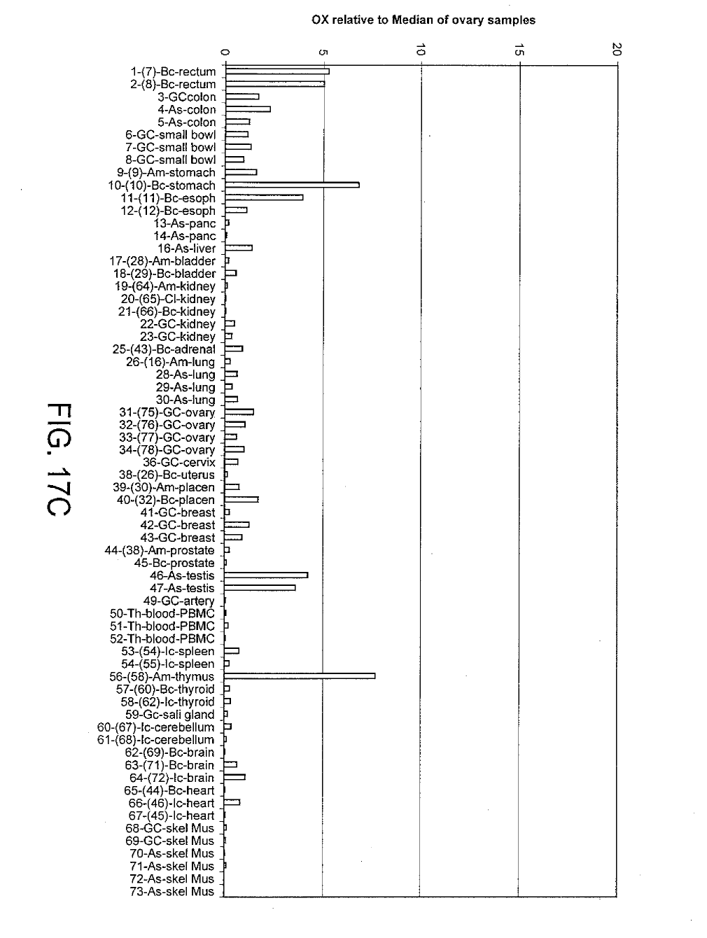

FIG. 17 presents a histogram showing over expression of the Homo sapiens hypothetical protein FLJ11029 (FLJ11029) F13779 transcripts which are detectable by amplicon as depicted in sequence name F13779_seg17-18 (SEQ ID NO: 127) in different normal tissues. FIG. 17A presents the expression of each sample relative to median of the lung samples; FIG. 17B presents the expression of each sample relative to median of the breast samples; FIG. 17C presents the expression of each sample relative to median of the ovary samples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An isolated polynucleotide comprising a nucleic acid sequence set forth in a member selected from the group consisting of SEQ ID NOs:38-41, 85, 86, or a sequence at least about 95% identical thereto.

2. An isolated polynucleotide comprising a nucleic acid sequence as set forth in any one of
SEQ ID NOs:42-70, 87-120.

3. An isolated polypeptide comprising an amino acid sequence set forth in a member selected from the group consisting of SEQ ID NOs:74-77, 121 and 124, or a sequence at least about 95% homologous thereto.

4. An isolated oligonucleotide comprising an amplicon selected from the group consisting of SEQ ID NOs:80, 83, 127.

5. A primer pair, comprising a pair of isolated oligonucleotides capable of amplifying said amplicon of claim 4.

6. The primer pair of claim 5 comprising a pair of isolated oligonucleotides having a sequence selected from the group consisting of SEQ ID NOs:78 and 79; 81 and 82; 125 and 126.

7. A kit for detecting a disease, comprising a marker as set forth in any one of SEQ ID NOs: 38-41, 42-70, 80, 83, 85-120, 127, and a detecting agent for detecting said marker.

8. The kit of claim 7, wherein said detecting agent comprises at least one nucleotide probe or primer.

9. The kit of claim 8, wherein said detecting agent comprises at least one oligonucleotide capable of selectively hybridizing to a nucleic acid sequence as set forth in any one of SEQ ID NOs: 38-41, 42-70, 80, 83, 85-120, 127.

10. The kit of claim 9, wherein said oligonucleotide is selected from the group consisting of
SEQ ID NOs: 37, 84.

11. The kit of claim 8, wherein said detecting agent comprises at least one primer pair capable of selectively amplifying a nucleic acid sequence as set forth in any one of SEQ ID NOs: 38-41, 42-70, 80, 83, 85-120, 127.

12. The kit of claim 11, wherein said primer pair is selected from the group consisting of SEQ ID NOs: 78, 79, 81, 82, 125, 126.

13. The kit of claim 11, wherein said kit further comprises at least one reagent for performing a NAT (nucleic acid amplification technology)-based assay.

14. The kit of claim 13, wherein said NAT-based assay is selected from the group consisting of a PCR, Real-Time PCR, LCR, Self-sustained synthetic reaction, Q-Beta replicase, Cycling probe reaction, Branched DNA, RFLP analysis, DGGE/TGGE, Single-strand conformation polymorphism, Dideoxy fingerprinting, microarrays, Fluorescence in situ hybridization or Comparative genomic hybridization.

15. A kit for detecting a disease, comprising a marker as set forth in any one of SEQ ID NOs: 74-77, 121 and 124 and an antibody for detecting said marker.

16. The kit of claim 15, wherein said kit further comprises at least one reagent for performing an immunoassay.

17. The kit of claim 16, wherein said immunoassay is selected from the group consisting of an ELISA, a RIA, a slot blot, immunohistochemical assay, FACS, a radio-imaging assay or a Western blot.

18. A method for screening for a cancerous disease, disorder or condition in a subject, comprising detecting in the subject or in a sample obtained from said subject a polynucleotide having a sequence as set forth in any one of SEQ. ID NOs: 38-41, 42-70, 80, 83, 85-120, 127.

19. The method of claim 18, wherein screening for a disease comprises detecting the presence or seventy of the disease, disorder or condition, or prognosis of the subject, or treatment selection for said subject, or treatment monitoring.

20. The method of claim 18, wherein the cancer is invasive or metastatic.

21. The method of claim 18, wherein said cancerous disease, disorder or condition comprises lung cancer and said polynucleotide has a sequence selected from the group consisting of SEQ ID NOs: 38-41, 42-70, 80, 83, 85-120, 127.

22. The method of claim 18, wherein said cancerous disease, disorder or condition comprises breast cancer and said polynueleotide has a sequence selected from the group consisting of SEQ ID NOs: 38-41, 42-70, 80, 83, 85-120, 127.

23. The method of claim 18, wherein said cancerous disease, disorder or condition comprises ovarian cancer and said polynucleotide has a sequence selected from the group consisting of SEQ ID NOs: 38-41, 42-70, 80, 83, 85-120, 127.

24. The method of claim 18, wherein screening for a disease comprises detecting the presence or severity of the disease, disorder or condition, or prognosis of the subject, or treatment selection for said subject, or treatment monitoring.

25. A method for screening for a disease, disorder or condition in a subject, comprising detecting a polypeptide having a sequence as set forth in SEQ ID NOs: 74-77, 121 and 124.

26. The method of claim 25, wherein screening for a disease comprises detecting the presence or severity of the disease, disorder or condition, or prognosis of the subject, or treatment selection for said subject, or treatment monitoring of said subject.

27. The method of claim 25, wherein the disease is lung cancer, breast cancer or ovarian cancer.

28. The method of claim 27, wherein said cancer is invasive or metastatic.

29. The method of claim 25, wherein said detecting is conducted by immunoassay.

30. The method of claim 29, wherein the immunoassay utilizes an antibody which specifically interacts with said polypeptide.

31. The method of claim 25, wherein said cancerous disease, disorder or condition comprises ovarian cancer and said polypeptide has a sequence selected from the group consisting of SEQ ID NOs:74-77, 121 and 124.

32. The method of claim 25, wherein said cancerous disease, disorder or condition comprises lung cancer and said polypeptide has a sequence selected from the group consisting of SEQ ID NOs:74-77, 121 and 124.

33. The method of claim 25, wherein said cancerous disease, disorder or condition comprises breast cancer and said polypeptide has a sequence selected from the group consisting of SEQ ID NOs:74-77, 121 and 124.

34. The method of claim 25, wherein screening for a disease comprises detecting the presence or severity of the disease, disorder or condition, or prognosis of the subject, or treatment selection for said subject, or treatment monitoring of said subject.

35. The method of claim 34, wherein said detecting is conducted by immunoassay.

36. The method of claim 35, wherein the immunoassay utilizes an antibody which specifically interacts with said polypeptide.
